(19) United States
(12) Patent Application Publication
(10) Pub. No.: US 2022/0175811 A1
(43) Pub. Date: Jun. 9, 2022

(54) STABLE FORMULATIONS OF CYCLIC DINUCLEOTIDE STING AGONIST COMPOUNDS AND METHODS OF USE THEREOF

(71) Applicants: Margaet BRNELL, Rahway, NJ (US); Erin N. GUIDRY, Kenilworth, NJ (US); Yash KAPOOR, Kenilworth, NJ (US); Eric A. KEMP, West point, PA (US); Ellen C. MINNIHAN, Cambridge, MA (US); Sachin MILLTAL, Kenilworth, NJ (US); Elizabeth PIERSON, Rahway, NJ (US); Makhail REIBARKH, Rahway, NJ (US); Tammy W. SHEN, Penllyn, PA (US); Ericka R. WALSH, Kenilworth, NJ (US); Paul L. WALSH, Rahway, NJ (US); Merck Sharp & Dohme Corp., Rahway, NJ (US)

(72) Inventors: Margaret Brunell, Sayerville, NJ (US); Erin N. Guidry, Cranford, NJ (US); Yash Kapoor, Chalfont, PA (US); Eric A. Kemp, Souderton, PA (US); Ellen C. Minnihan, Cambridge, MA (US); Sachin Mittal, Bridgewater, NJ (US); Elizabeth Pierson, Cranford, NJ (US); Mikhail Reibarkh, Cranford, NJ (US); Tammy W. Shen, Penllyn, PA (US); Erika R. Walsh, North Plainfield, NJ (US); Paul L. Walsh, North Plainfield, NJ (US)

(73) Assignee: Merck Sharp & Dohme Corp., Rahway, NJ (US)

(21) Appl. No.: 17/441,087

(22) PCT Filed: Mar. 24, 2020
(86) PCT No.: PCT/US2020/024372

§ 371 Date: Sep. 20, 2021

Related U.S. Application Data
(60) Provisional application No. 62/982,443, filed on Feb. 27, 2020.
(60) Provisional application No. 62/826,000, filed on Mar. 29, 2019.

(51) Int. Cl. A61K 31/7084; A61K 47/26; A61K 47/22; A61K 47/20; A61K 47/18

(52) U.S. Cl. CPC A61K 31/7084; A61K 47/26; A61K 47/183; A61K 47/20; A61K 47/22

(57) ABSTRACT

The invention relates to stable formulations of cyclic dinucleotide STING agonist compounds or pharmaceutically acceptable salts thereof. The invention further provides methods for treating various cancers with stable formulations of the invention. In some embodiments of the methods of the invention, the formulations are administered to a subject by intratumoral or subcutaneous administration.

### FIELD OF THE INVENTION

The invention relates to stable formulations comprising cyclic dinucleotide compounds that are STING (Stimulator of Interferon Genes) agonists that activate the STING pathway. Also provided are methods of treating various cancers and chronic infections with the formulations of the invention.

### BACKGROUND OF THE INVENTION

Compounds that induce type I interferon activity have great potential as anti-viral and anti-cancer agents (see T. R. Vargas et al., Rationale for STING-Targeted Cancer Immunotherapy, 75 Eur. J. Cancer 85-97 (2017); L. Corrales et al., Direct Activation of STING in the Tumor Microenvironment Leads to Potent and Systemic Tumor Regression and Immunity, 11 Cell Reports 1018-30 (2015); Glen N. Barber, STING: infection, inflammation and cancer, 15 Nat. Rev. Immunol. 760-770 (2015); E. Curran et al., STING Pathway Activation Stimulates Potent Immunity Against Myeloid Leukemia, 15 Cell Reports 2357-66 (2016). There is a growing body of data demonstrating that the cGAS-STING cGAS (cyclic GMP-AMP synthase-STING) cytosolic DNA sensory pathway has a significant capacity to induce type I interferons. Thus, the development of STING activating agents is rapidly taking an important place in today's anti-tumor therapy landscape.

Cyclic dinucleotide (CDN) compounds that are STING agonists for use in human subjects must be stored prior to use and transported to the point of administration. Reproducibly attaining a desired level of drug in a subject requires that the drug be stored in a formulation that maintains the potency of the drug. The need exists for stable formulations of cyclic dinucleotide STING agonist compounds for pharmaceutical use, e.g., for treating various cancers and infectious diseases. Preferably, such formulations will exhibit a long shelf-life, be stable when stored and transported, and will be amenable to intratumoral administration.

### SUMMARY OF THE INVENTION

The present disclosure relates to pharmaceutical formulations comprising cyclic dinucleotide STING agonist compounds, pharmaceutically acceptable aqueous carriers, pharmaceutically acceptable tonicity modifiers, pharmaceutically acceptable buffering agents, pharmaceutically acceptable antioxidants, and pharmaceutically acceptable metal chelators. Other embodiments, aspects and features of the present invention are either further described in or will be apparent from the ensuing description, examples and appended claims.

### BRIEF DESCRIPTION OF THE DRAWINGS

FIG. 1 depicts titration curves for formulations of Compound A, with 10, 25, and 50 mM histidine, according to Example 2, Table 20.

FIG. 2 depicts titration curves for formulations with 10, 25, and 50 mM histidine alone, according to Example 7, Table 20.

FIG. 3 depicts comparisons of titration curves for formulations with 10, 25, and 50 mM histidine alone and for formulations of Compound A, with 10, 25, and 50 mM histidine, according to Example 7, Table 20.

### DETAILED DESCRIPTION OF THE INVENTION

The instant disclosure provides pharmaceutical formulations comprising cyclic dinucleotide STING agonist compounds, pharmaceutically acceptable tonicity modifiers, pharmaceutically acceptable buffering agents, pharmaceutically acceptable antioxidants, and pharmaceutically acceptable metal chelators. These pharmaceutical formulations are useful for methods of treatment of cancer or an immune disorder or immune condition that comprise intravenous (IV), intratumoral (IT), or subcutaneous (SC) administration to a patient in need thereof. The formulations of the invention address the issues of stability and solubility associated with formulations comprising cyclic dinucleotide STING agonist compounds in aqueous solutions. The invention further provides formulations comprising cyclic dinucleotide STING agonist compounds with potential for room temperature storage and enablement of terminal sterilization.

The formulations of the invention are useful for intratumoral (IT) delivery to a patient in need thereof. In order to deliver maximum therapeutic benefits to patients, it is desirable that formulations for IT delivery have adequate stability during storage and administration.

### DEFINITIONS AND ABBREVIATIONS

As used throughout the specification and appended claims, the following abbreviations apply:
AIDS Acquired Immunodeficiency Syndrome
AML Acute Myeloid Leukemia
API Active Pharmaceutical Ingredient
CDN Cyclic Dinucleotide
CML Chronic Myelogenouse Leukemia
DCA Dichloroacetic acid
DCM Dichloromethane
DDTT (E)-N,N-dimethyl-N′-(3-thioxo-3H-1,2,4-dithiazol-5-yl)formimidamide, N′-(3-thioxo-3H-1,2,4-dithiazol-5-yl)-N,N-dimethylmethanimidamide
DFS Disease Free Survival
DMOCP 2-chloro-5,5-dimethyl-1,3,2-dioxaphosphineane 2-oxide
DMTr 4,4′-Dimethoxytrityl Protecting Group
DP Drug Product, i.e., API formulation
DS Drug Substance, i.e., API
DTPA Diethylenetriaminepentacetic Acid
EDTA Ethylene diamine tetraacetic acid
Et₃SiH Triethylsilane
HBV Hepatitis B Virus
HCV Hepatitis C Virus
HIV Human Immunodeficiency Virus
HNSCC Head and Neck Squamous Cell Carcinoma
HPLC High Performance Liquid Chromatography
IT Intratumoral
IV Intravenous
MDS Myelodysplastic Syndrome
MeCN Acetonitrile, CH₃CN, ACN
MeNH₂ Methylamine, CH₃NH₂
MPN Myeloproliferative Neoplasm
NT Not Tested
OS Overall Survival
PFS Progression Free Survival
PG Protecting Group
PVDR Polyvinylidene Difluoride
Py Pyridine
SC Subcutaneous
sPNET Suprantentorial Primordial Neuroectodermal Tumors
STING STimulator of INterferon Genes
TBA-Br Tertbutylammonium Bromide
t-BuNH₂ tert-Butyl amine
t-BuOOH tert-Butyl peroxide
TEA.3HF Triethylamine trihydrofluoride
TEAA Triethylammonium acetate
TFA Trifluoroacetic acid
UHPLC Ultra High Performance Liquid Chromatography
USP United States Pharmacopeia
VEGF Vascular Endothelial Growth Factor
WFI Water for Injections

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the invention to a subject having an immune condition or cancerous condition, or diagnosed with a cancer or pathogenic infection (e.g., viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, halting or delaying the growth of a tumor or blood cancer or progression of disease such as cancer, stabilization of disease, inhibiting the growth or survival of tumor cells, eliminating or reducing the size of one or more cancerous lesions or tumors, decreasing the level of one or more tumor markers, ameliorating, abrogating the clinical manifestations of disease, reducing the severity or duration of the clinical symptoms of disease such as cancer, prolonging the survival of a patient relative to the expected survival in a similar untreated patient, inducing complete or partial remission of a cancerous condition or other disease.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (see, Wolfgang A. Weber, Assessing Tumor Response to Therapy, 50:5(Suppl.) J. NUCL. MED. 1S-10S (May 2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the invention is any of progression free survival (PFS), disease free survival (DFS) or overall survival (OS). PFS, also referred to as “Time to Tumor Progression” indicates the length of time during and after treatment that the cancer does not grow and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the invention may not be effective in achieving a positive therapeutic effect in every patient, it should do so in a statistically significant number of subjects as determined by any statistical test known in the art, such as the Student's t-test, the chi2-test, the U-test according to Mann and Whitney, the Kruskal-Wallis test (H-test), Jonckheere-Terpstra-test, or the Wilcoxon-test. See generally, Introduction to Statistical Methods for Clinical Trials (Chapman & Hall/CRC Texts in Statistical Science, 1^{st} edition, Thomas D. Cook & David L. DeMets, eds., 2007.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient. Those “in need of treatment” include those patients that may benefit from treatment with the formulations of the invention, e.g. a patient suffering from cancer or an immune condition.

The term “pharmaceutically effective amount” or “effective amount” means an amount whereby sufficient therapeutic composition or formulation is introduced to a patient to treat a diseased or condition. One skilled in the art recognizes that this level may vary according the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

A “chemotherapeutic agent” is a chemical compound useful in the treatment of cancer. Anti-PD-1 antibodies can be used with any one or more suitable chemotherapeutic agent. Examples of such chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphor-amide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

The term “buffer” encompasses those agents that maintain the solution pH of the formulations of the invention in an acceptable range.

The term “pharmaceutical formulation” refers to preparations that are in such form as to permit the active ingredients to be effective. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe”, e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the United States Pharmacopeia or another generally recognized pharmacopeia for use in animals, and more particularly in humans.

A “stable” formulation is one in which the cyclic dinucleotide STING agonist compound therein essentially retains its physical stability and/or chemical stability upon storage. Stability can be measured at a selected temperature for a selected time period. For example, in one embodiment, a stable formulation is a formulation with no significant changes observed at a refrigerated temperature (2° C. to 8° C.) for at least 12 months. In another embodiment, a stable formulation is a formulation with no significant changes observed at a refrigerated temperature (2° C. to 8° C.) for at least 18 months. In another embodiment, stable formulation is a formulation with no significant changes observed at room temperature (23° C. to 27° C.) for at least 3 months. In another embodiment, stable formulation is a formulation with no significant changes observed at room temperature (23° C. to 27° C.) for at least 6 months. In another embodiment, stable formulation is a formulation with no significant changes observed at room temperature (23° C. to 27° C.) for at least 12 months. In another embodiment, stable formulation is a formulation with no significant changes observed at room temperature (23° C. to 27° C.) for at least 18 months. Typically, the concentration, pH and osmolality of the formulation have no more than +/−10% change. Potency is typically within 90-110% of the target potency value.

The term “isotonic” means that the formulation of interest has essentially the same osmotic pressure as human blood. Isotonic formulations will generally have an osmotic pressure from about 270 mOsmol/kg to about 328 mOsmol/kg. Slightly hypotonic pressure is 250 mOsmol/kg to about 269 mOsmol/kg and slightly hypertonic pressure is 328 mOsmol/kg to about 350 mOsmol/kg. Osmotic pressure can be measured, for example, using a vapor pressure or ice-freezing type osmometer. One osmole (Osmol) is one gram molecular weight (1 mole) of any non-dissociable substance (such as glucose) that contains 6.02×10²³ particles and contributes to a solution's osmotic pressure. To convert mOsmol/kg to mmol/L, multiply mOsmol by the number of dissociable particles per molecule.

A “non-reducing sugar” is a sugar not capable of acting as a reducing agent because it does not contain or cannot be converted to contain a free aldehyde group or a free ketone group. Examples of non-reducing sugars include but are not limited to dissacharrides, such as sucrose and trehalose.

Pharmaceutical Formulations of this Disclosure

The instant disclosure provides pharmaceutical formulations comprising comprising cyclic dinucleotide STING agonist compounds, pharmaceutically acceptable aqueous carriers, pharmaceutically acceptable tonicity modifiers, pharmaceutically acceptable stabilizing excipients, and pharmaceutically acceptable buffering agents, and optionally additional pharmaceutically acceptable ingredients. These pharmaceutical formulations are useful for methods of treatment of cancer or of an immune disorder or immune condition that comprise IV, IT, or SC administration to a patient in need thereof. The formulations of the invention address the issues of chemical instability and insufficient solubility in known aqueous formulations of cyclic dinucleotide STING agonist compounds. The invention further provides formulations comprising cyclic dinucleotide STING agonist compounds with potential for room temperature storage and enablement of terminal sterilization.

### Cyclic Dinucleotide STING Agonist Compounds

The disclosure provides pharmaceutical formulations comprising cyclic dinucleotide STING agonist compounds (or pharmaceutically acceptable salts thereof) as the active pharmaceutical ingredient (API), as well as methods for using the formulations of the disclosure. Any cyclic dinucleotide STING agonist compound or pharmaceutically acceptable salt thereof may be used in the formulations disclosed herein. In embodiments, the cyclic dinucleotide STING agonist compound is selected from the group consisting of compounds of formula (I′):

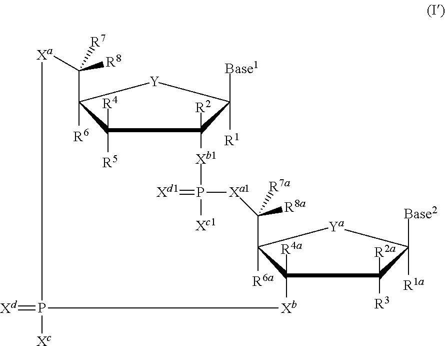

or a pharmaceutically acceptable salt, hydrate, solvate, or prodrug thereof, wherein Base¹ and Base² are each independently selected from the group consisting of

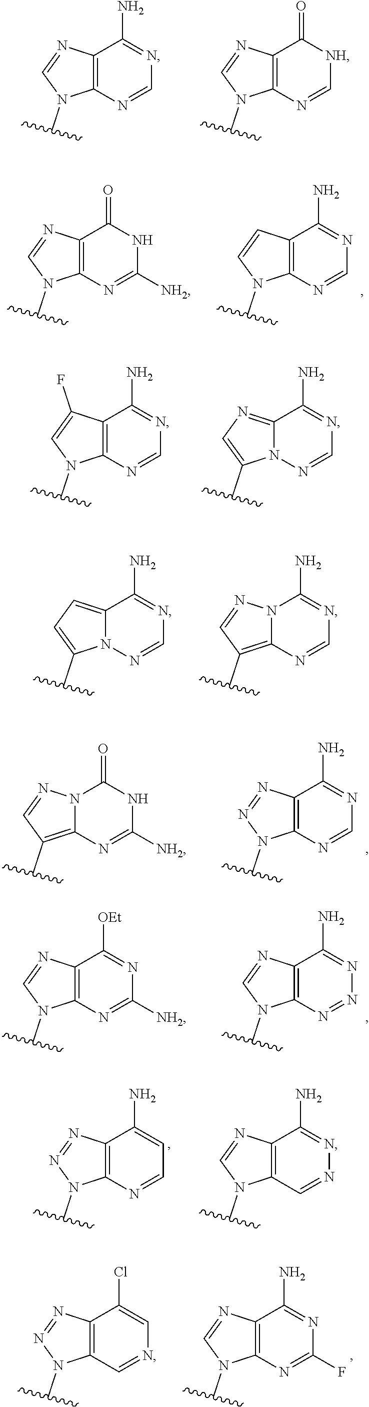

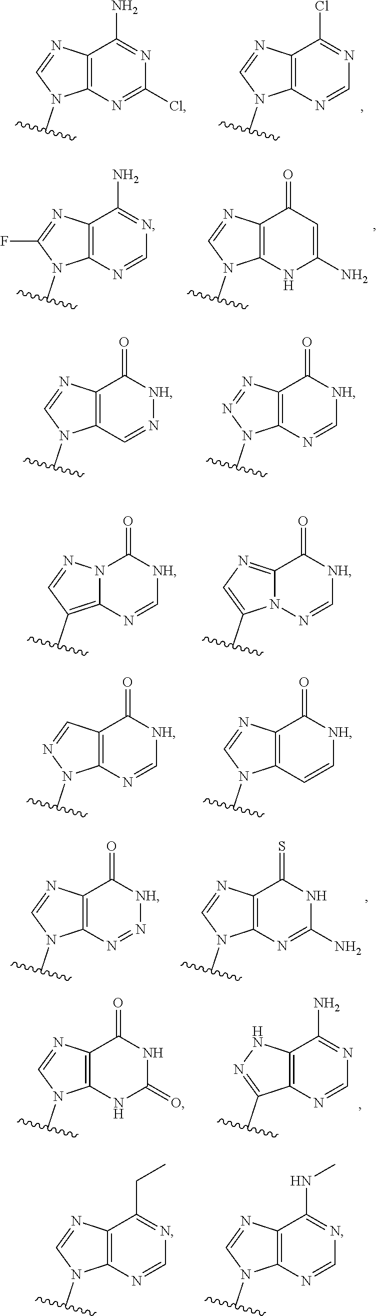

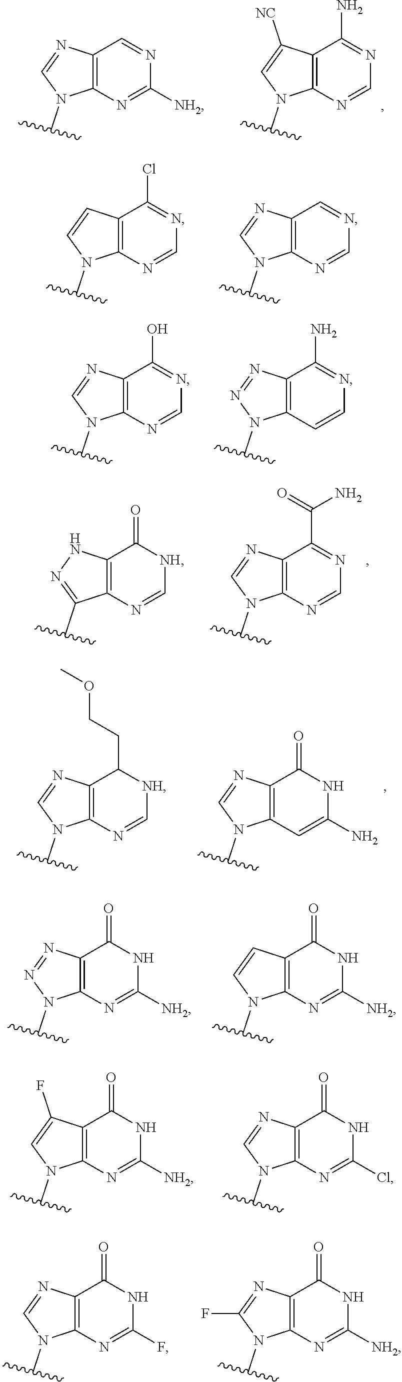

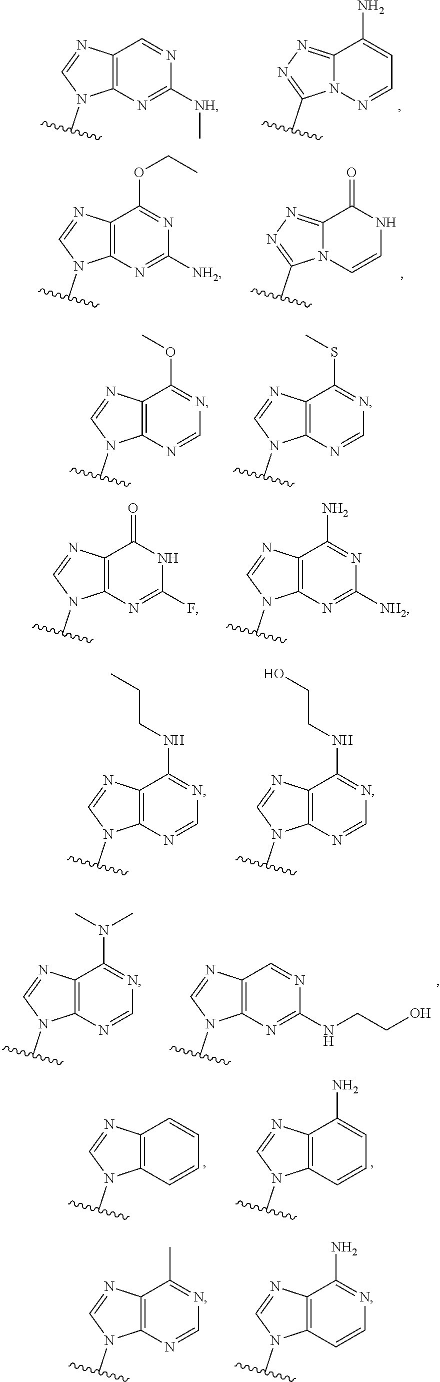

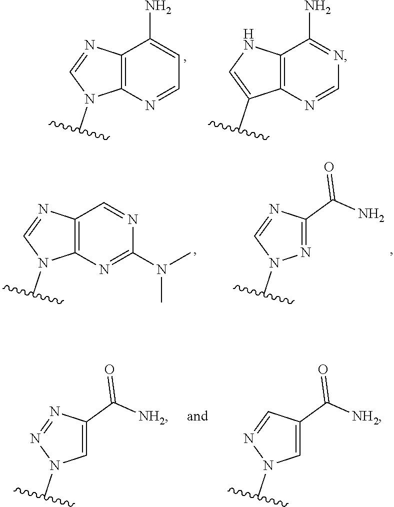

where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each independently selected from the group consisting of O, and S; X^{b} and X^{b1} are each independently selected from the group consisting of O, and S; X^{c} and X^{c1} are each independently selected from the group consisting of OR⁹, SR⁹, and NR⁹R⁹; X^{d} and X^{d1} are each independently selected from the group consisting of O and S; R¹ and R^{1a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R¹ and R^{1a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R² and R^{2a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R² and Rea C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R³ is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁴ and R^{4a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁴ and R^{4a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁵ is selected from the group consisting of H, F, Cl, Br, I, OH, CN, NH₂, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁵ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, NR⁹R⁹, and N3; R⁶ and R^{6a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁶ and R^{6a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁷ and R^{7a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁷ and R^{7a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁸ and R^{8a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁸ and R^{8a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N3; each R⁹ is independently selected from the group consisting of H, C₁-C₂₀ alkyl,

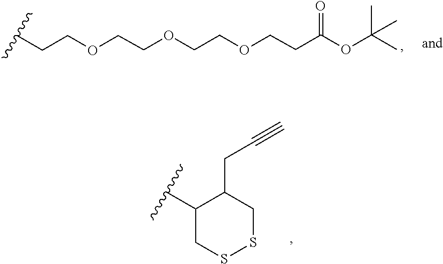

where each R⁹ C₁-C₂₀ alkyl is optionally substituted by 0 to 3 substituents independently selected from the group consisting of OH, —O—C₁-C₂₀ alkyl, —S—C(O)C₁-C₆ alkyl, and C(O)OC₁-C₆ alkyl; optionally R^{1a} and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{1a} and R³ are connected to form —O—C₁-C₆ alkylene, —O—C₂-C₆ alkenylene, or —O—C₂-C₆ alkynylene, said O is bound at the R³ position; optionally Rea and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{2a} and R³ are connected to form —O—C₁-C₆ alkylene, —O—C₂-C₆ alkenylene, or —O—C₂-C₆ alkynylene, said O is bound at the R³ position; optionally R³ and R^{6a} are connected to form —O—C₁-C₆ alkylene, —O—C₂-C₆ alkenylene, or —O—C₂-C₆ alkynylene, such that where R³ and R^{6a} are connected to form —O—C₁-C₆ alkylene, —O—C₂-C₆ alkenylene, or —O—C₂-C₆ alkynylene, said O is bound at the R³ position; optionally R⁴ and R⁵ are connected to form are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R⁴ and R⁵ are connected to form —O—C₁-C₆ alkylene, —O—C₂-C₆ alkenylene, or —O—C₂-C₆ alkynylene, said O is bound at the R⁵ position; optionally R⁵ and R⁶ are connected to form —O—C₁-C₆ alkylene, —O—C₂-C₆ alkenylene, or —O—C₂-C₆ alkynylene, such that where R⁵ and R⁶ are connected to form —O—C₁-C₆ alkylene, —O—C₂-C₆ alkenylene, or —O—C₂-C₆ alkynylene, said O is bound at the R⁵ position; optionally R⁷ and R⁸ are connected to form C₁-C₆ alkylene, C₂-C₆ alkenylene, or C₂-C₆ alkynylene; and optionally R^{7a} and R^{8a} are connected to form C₁-C₆ alkylene, C₂-C₆ alkenylene, or C₂-C₆ alkynylene; and providing that when Y and Y^{a} are each O, X^{a} and X^{a1} are each O, X^{b} and X^{b1} are each O, and X^{c} and X^{c1} are each OH or SH, X^{d} and X^{d1} are each O, R¹ and R^{1a} are each H, R² is H, R⁶ and R^{6a} are each H, R⁷ and R^{7a} are each H, R⁸ and R^{8a} are each H, and Base¹ and Base² are each selected from the group consisting of

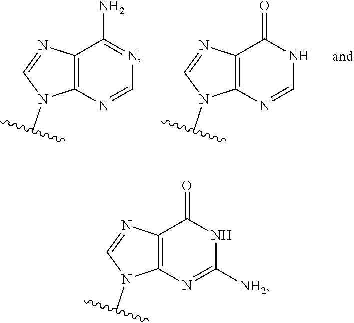 [SMILES: *n1cnc2c(N)ncnc21 ; *n1cnc2c(=O)[nH]cnc21 ; *n1cnc2c(=O)[nH]c(N)nc21]

R⁵ and R³ are not both selected from the group consisting of H, F, and OH. In particular embodiments, the cyclic dinucleotide STING agonist compound is selected from the group consisting of

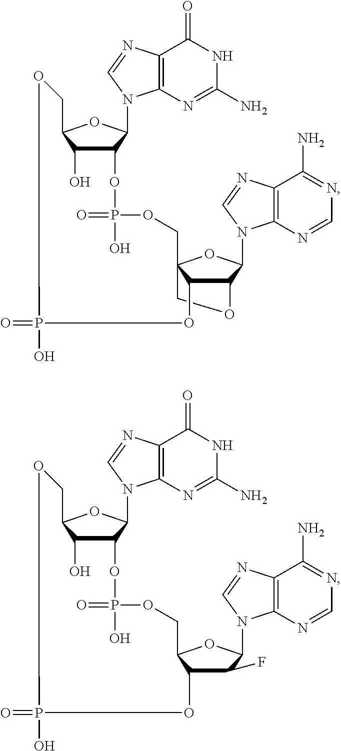

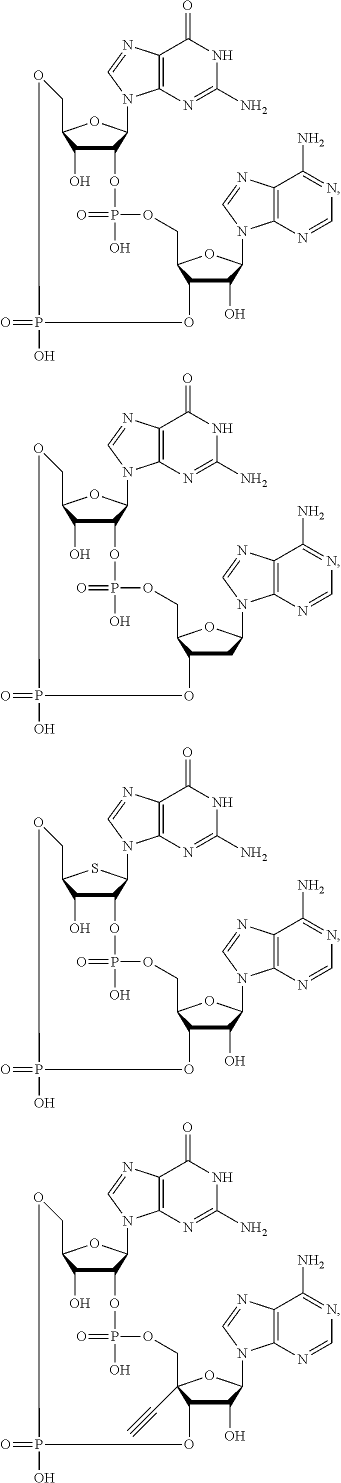

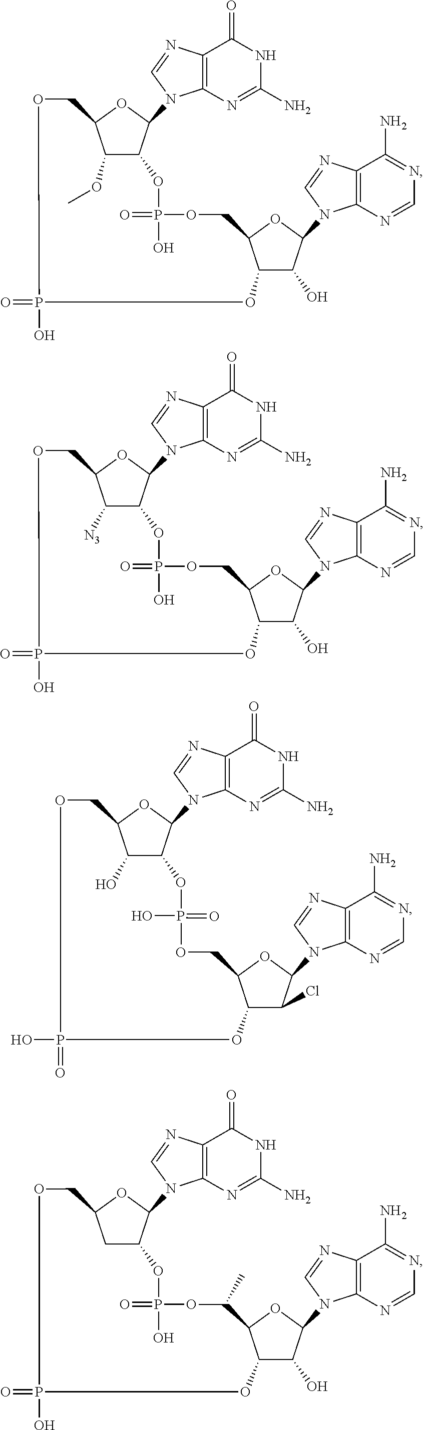 [SMILES: CO[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; [N-]=[N+]=N[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1nc2c(ncn2[C@@H]2O[C@@H]3COP(=O)(O)O[C@H]4[C@H](Cl)[C@H](n5cnc6c(N)ncnc65)O[C@@H]4COP(=O)(O)O[C@@H]2[C@@H]3O)c(=O)[nH]1 ; C[C@H]1OP(=O)(O)O[C@@H]2C[C@@H](COP(=O)(O)O[C@H]3[C@@H](O)[C@H](n4cnc5c(N)ncnc54)O[C@@H]31)O[C@H]2n1cnc2c(=O)[nH]c(N)nc21]

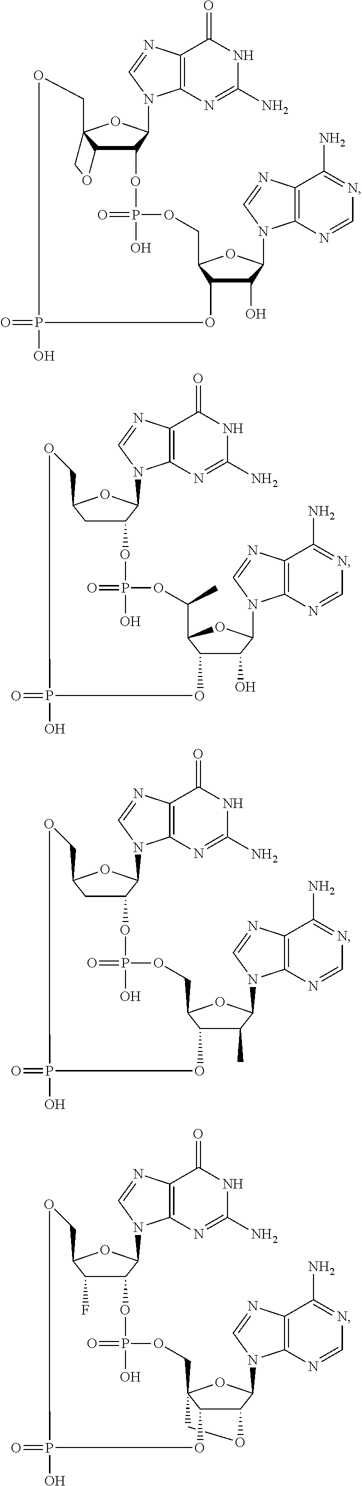

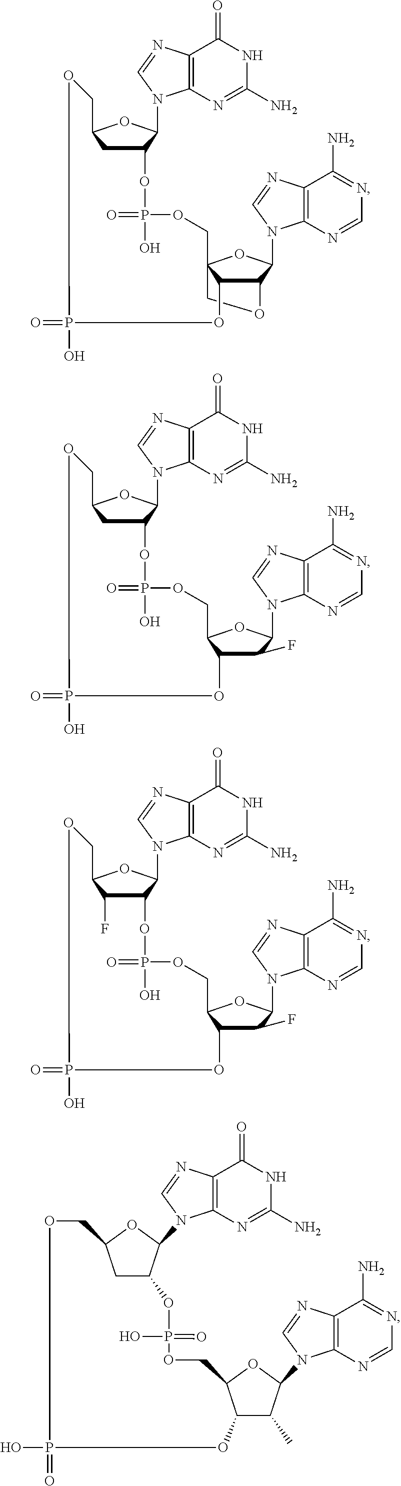

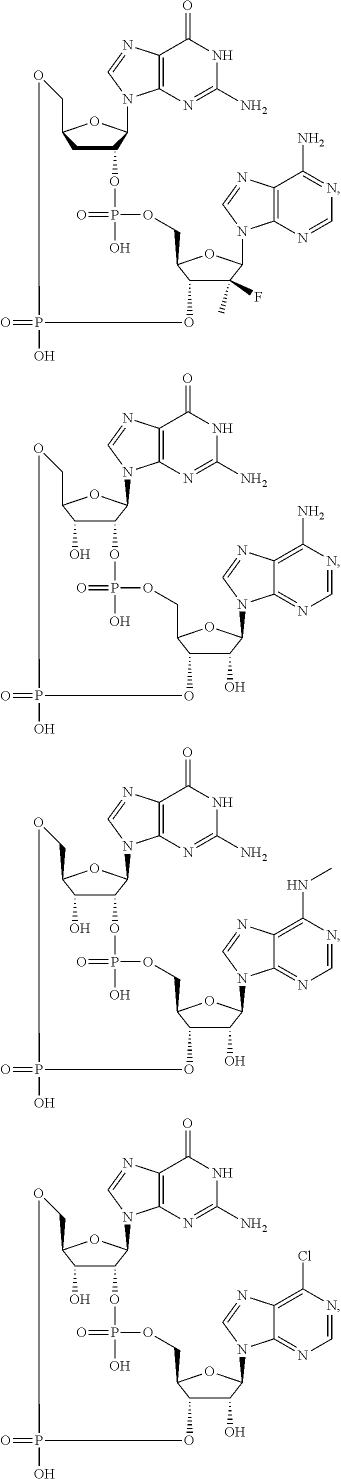

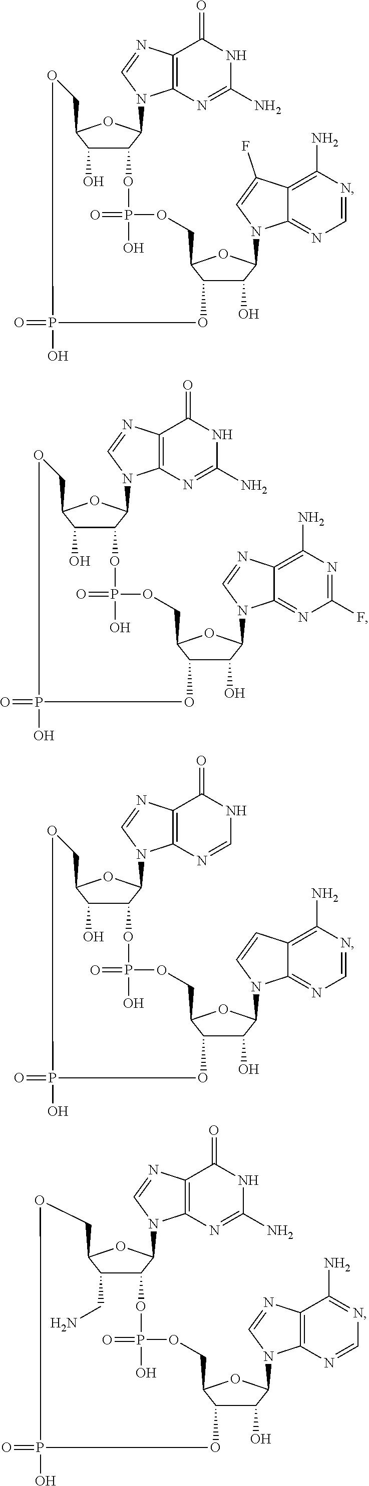 [SMILES: Nc1nc2c(ncn2[C@@H]2O[C@@H]3COP(=O)(O)O[C@H]4[C@@H](O)[C@H](n5cc(F)c6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)nc(F)nc65)O[C@@H]4COP(=O)(O)O[C@@H]2[C@@H]3O)c(=O)[nH]1 ; Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21 ; NC[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21]

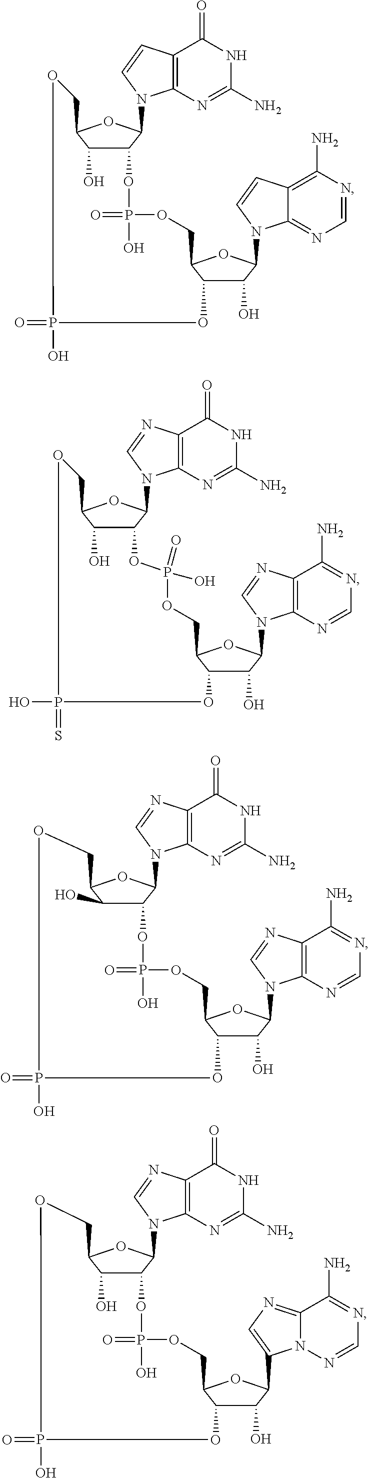 [SMILES: Nc1nc2c(ccn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(O)(=S)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3O)c(=O)[nH]1 ; Nc1nc2c(ncn2[C@@H]2O[C@@H]3COP(=O)(O)O[C@H]4[C@@H](O)[C@H](c5cnc6c(N)ncnn56)O[C@@H]4COP(=O)(O)O[C@@H]2[C@@H]3O)c(=O)[nH]1]

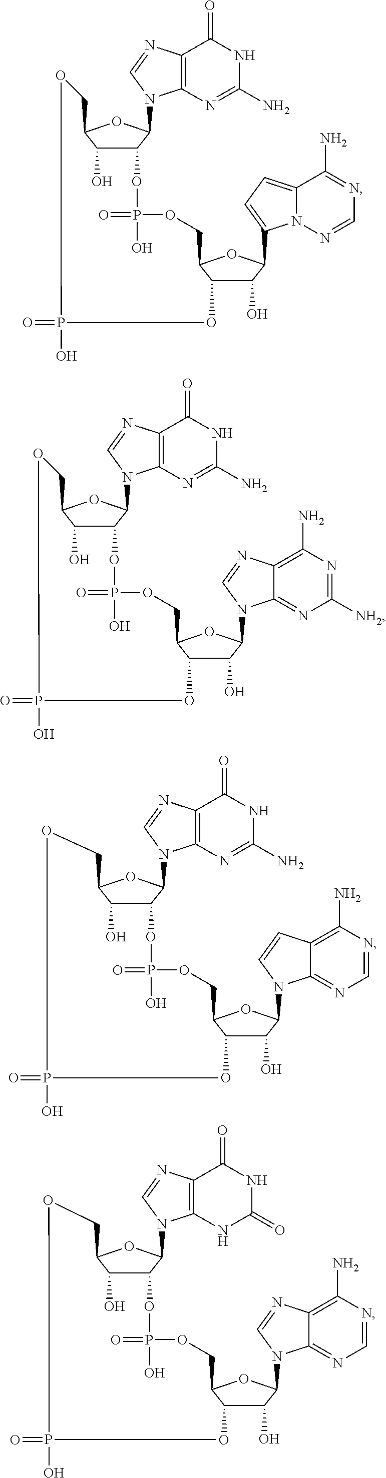 [SMILES: Nc1nc2c(ncn2[C@@H]2O[C@@H]3COP(=O)(O)O[C@H]4[C@@H](O)[C@H](c5ccc6c(N)ncnn56)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(=O)[nH]c21]

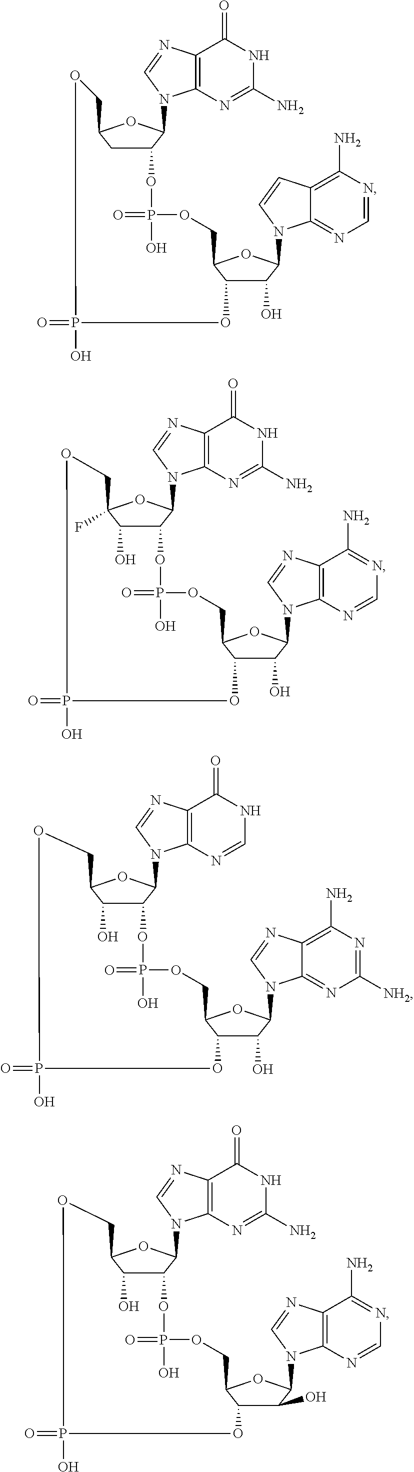 [SMILES: Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2C3)c(=O)[nH]1 ; Nc1nc2c(ncn2[C@@H]2O[C@]3(F)COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]cnc43)c2n1 ; Nc1nc2c(ncn2[C@@H]2O[C@@H]3COP(=O)(O)O[C@H]4[C@H](O)[C@H](n5cnc6c(N)ncnc65)O[C@@H]4COP(=O)(O)O[C@@H]2[C@@H]3O)c(=O)[nH]1]

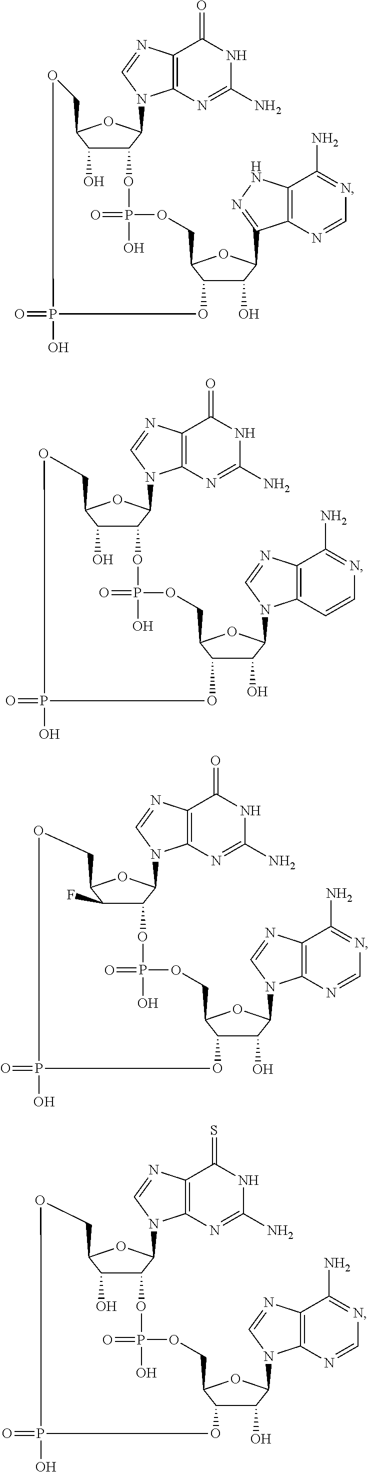 [SMILES: Nc1nc2c(ncn2[C@@H]2O[C@@H]3COP(=O)(O)O[C@H]4[C@@H](O)[C@H](c5n[nH]c6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)nccc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3F)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=S)[nH]1]

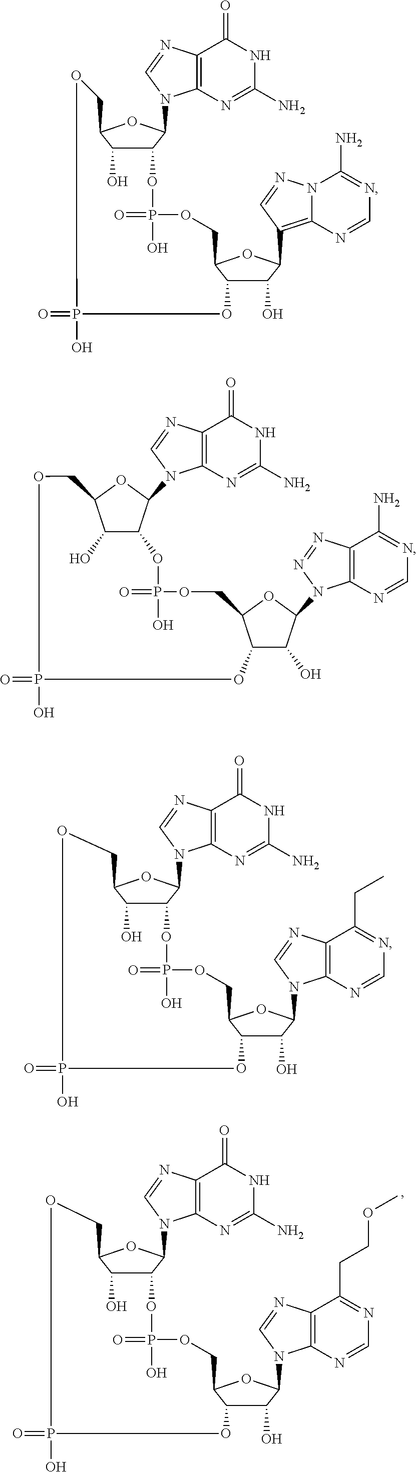 [SMILES: Nc1nc2c(ncn2[C@@H]2O[C@@H]3COP(=O)(O)O[C@H]4[C@@H](O)[C@H](c5cnn6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)ncnc65)O[C@@H]4COP(=O)(O)O[C@@H]2[C@@H]3O)c(=O)[nH]1 ; CCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; COCCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21]

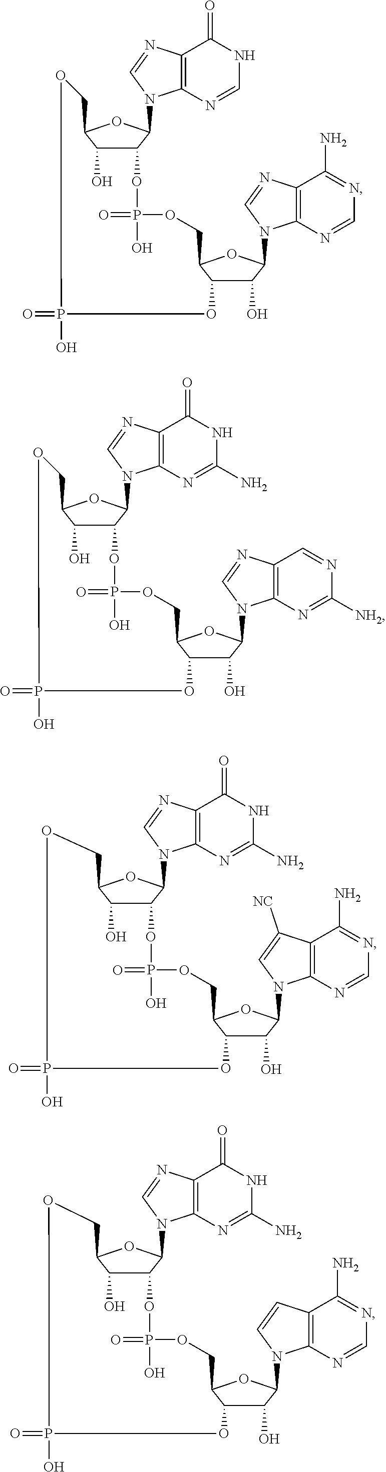 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21 ; Nc1ncc2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1 ; N#Cc1cn([C@@H]2O[C@@H]3COP(=O)(O)O[C@@H]4[C@H](O)[C@@H](COP(=O)(O)O[C@H]3[C@H]2O)O[C@H]4n2cnc3c(=O)[nH]c(N)nc32)c2ncnc(N)c12 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1]

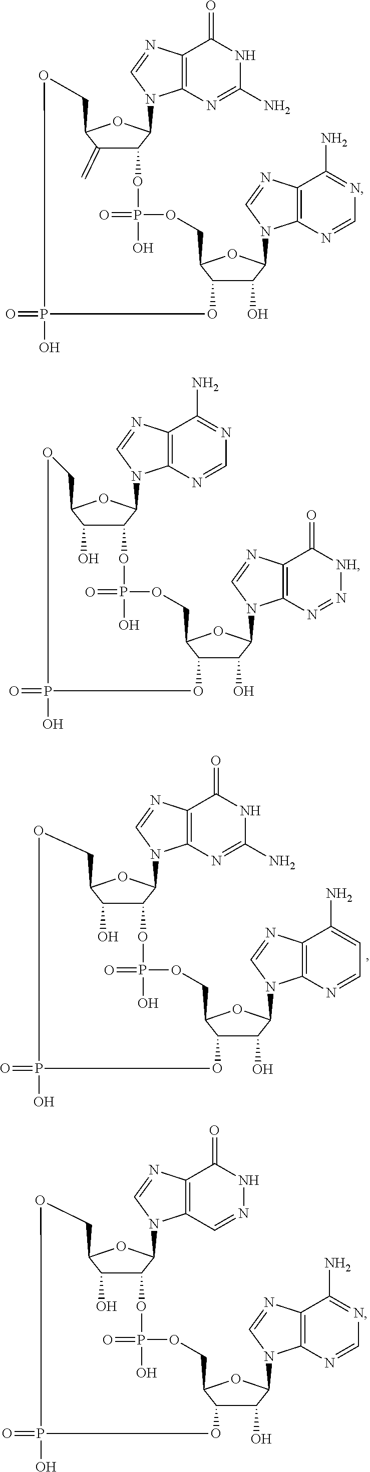 [SMILES: C=C1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1ncnc2c1ncn2[C@@H]1O[C@@H]2COP(=O)(O)O[C@H]3[C@@H](O)[C@H](n4cnc5c(=O)[nH]nnc54)O[C@@H]3COP(=O)(O)O[C@@H]1[C@@H]2O ; Nc1nc2c(ncn2[C@@H]2O[C@@H]3COP(=O)(O)O[C@H]4[C@@H](O)[C@H](n5cnc6c(N)ccnc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ncc21]

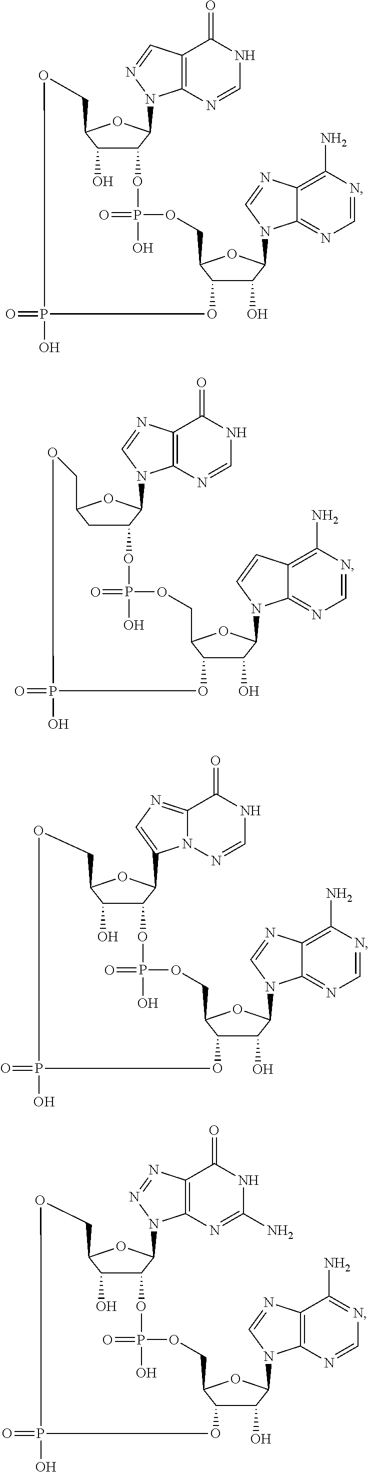 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1ncc2c(=O)[nH]cnc21 ; Nc1ncnc2c1ccn2[C@@H]1OC2COP(=O)(O)O[C@@H]3CC(COP(=O)(O)O[C@H]2[C@H]1O)O[C@H]3n1cnc2c(=O)[nH]cnc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1cnc2c(=O)[nH]cnn12 ; Nc1nc2c(nnn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1]

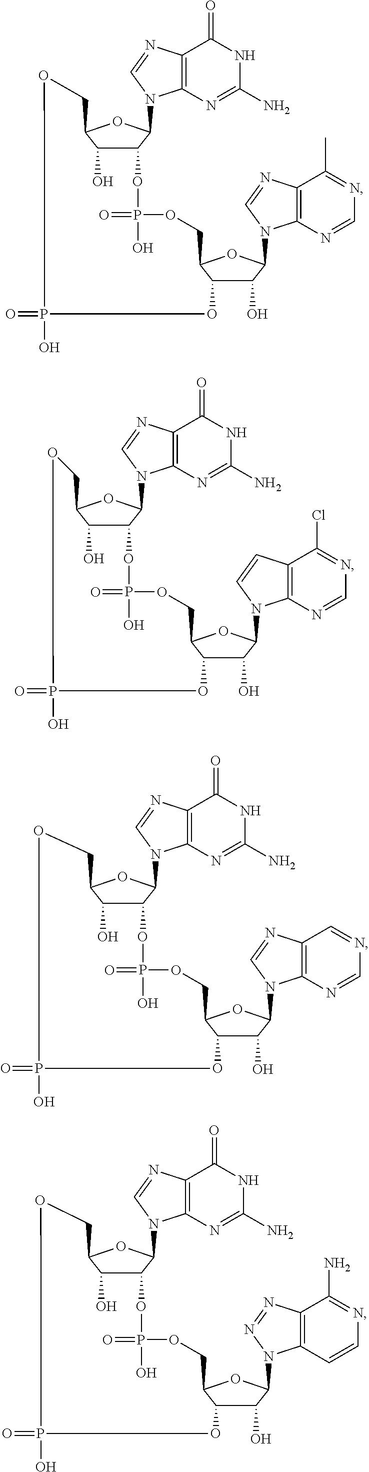 [SMILES: Cc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2O[C@@H]3COP(=O)(O)O[C@H]4[C@@H](O)[C@H](n5ccc6c(Cl)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6cncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)nccc65)O[C@@H]4COP(=O)(O)O[C@@H]2[C@@H]3O)c(=O)[nH]1]

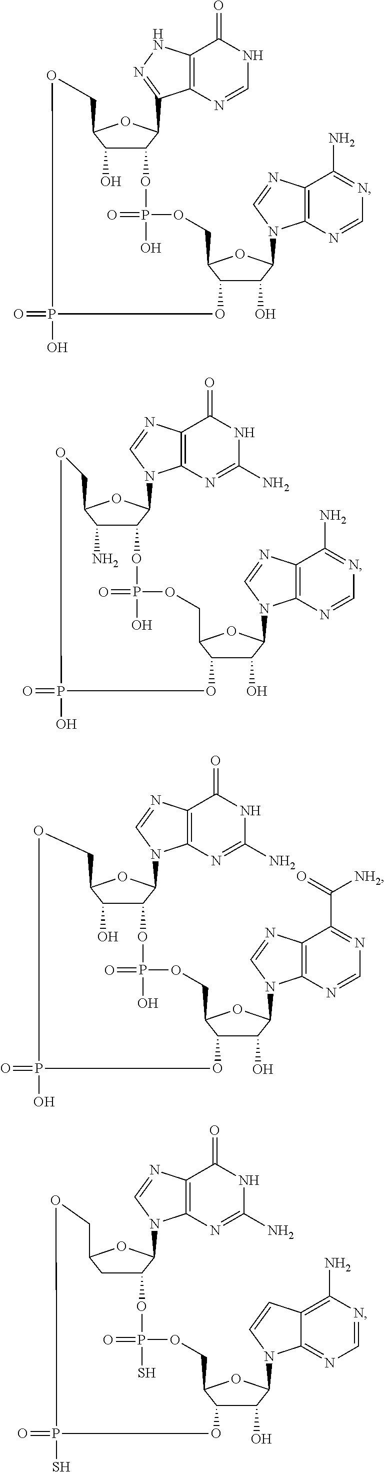 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1n[nH]c2c(=O)[nH]cnc12 ; Nc1nc2c(ncn2[C@@H]2O[C@@H]3COP(=O)(O)O[C@@H]4C(COP(=O)(O)O[C@@H]2[C@@H]3N)O[C@@H](n2cnc3c(N)ncnc32)[C@@H]4O)c(=O)[nH]1 ; NC(=O)c1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1]

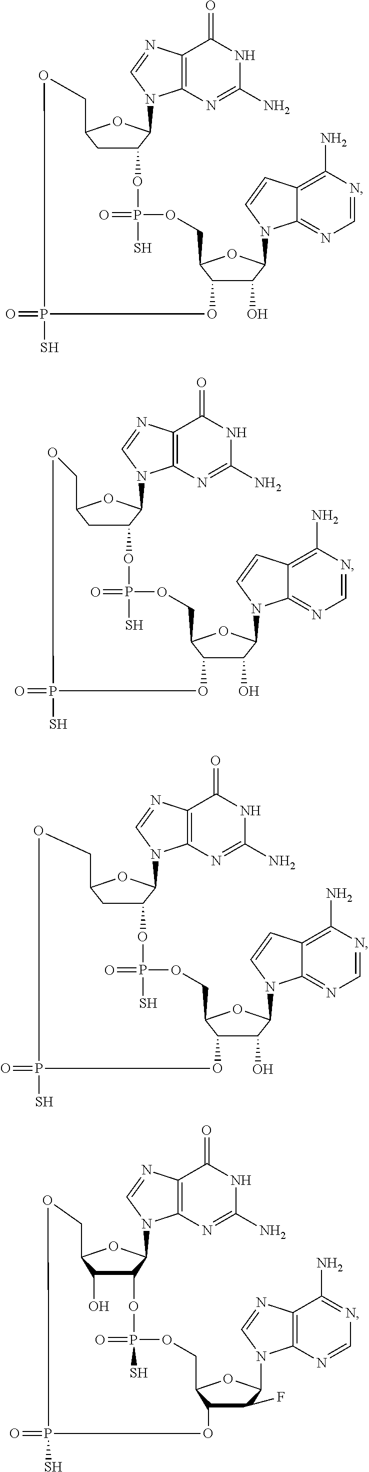

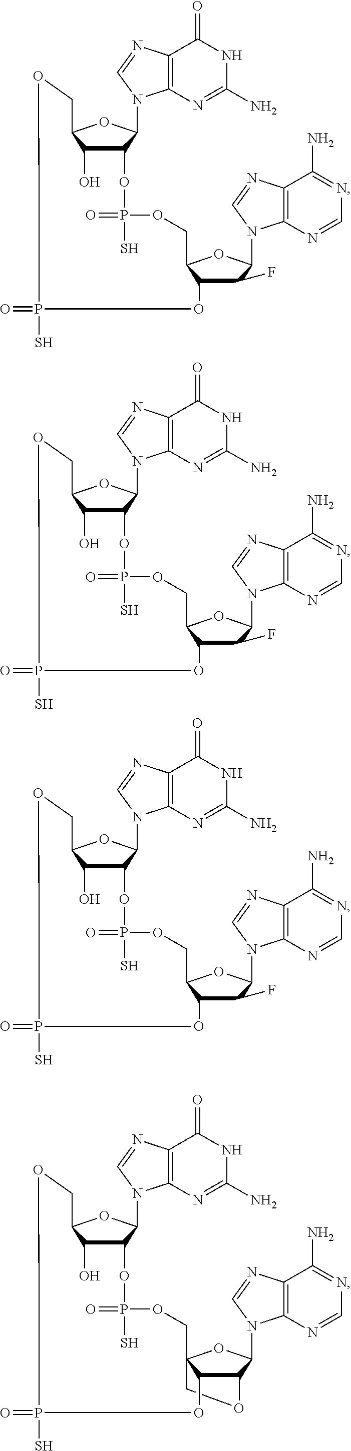

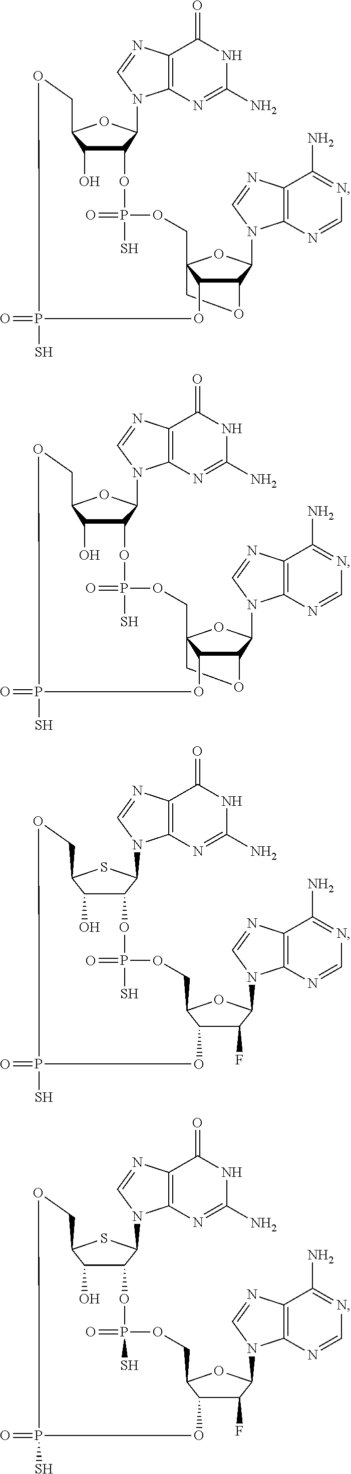 [SMILES: Nc1nc2c(ncn2[C@@H]2O[C@@H]3COP(=O)(S)OC4[C@@H]5OC[C@]4(COP(=O)(S)O[C@H]2C3O)O[C@H]5n2cnc3c(N)ncnc32)c(=O)[nH]1 ; Nc1nc2c(ncn2[C@@H]2O[C@@H]3COP(=O)(S)OC4[C@@H]5OC[C@]4(COP(=O)(S)O[C@H]2C3O)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1]

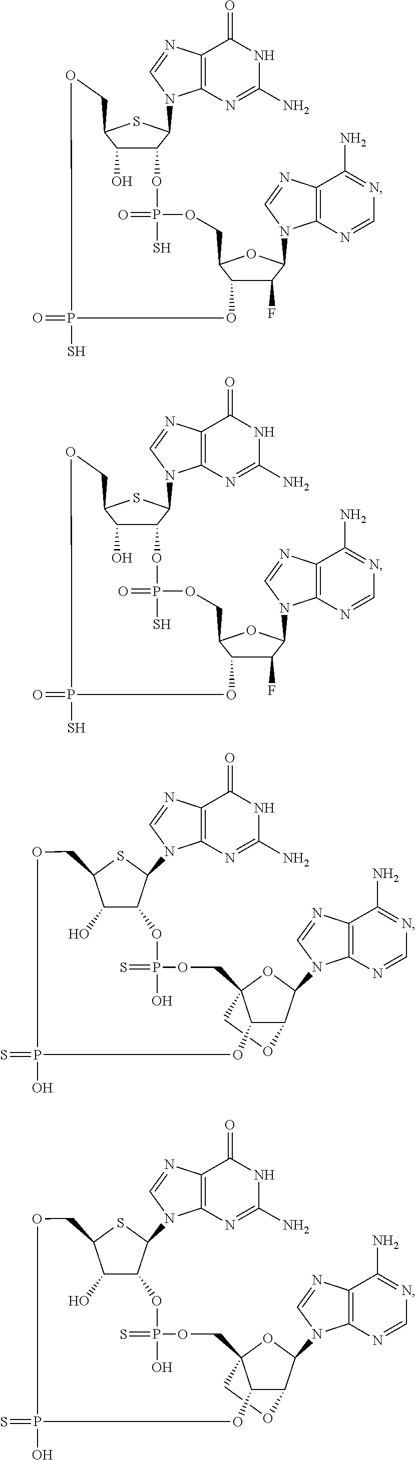 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1]

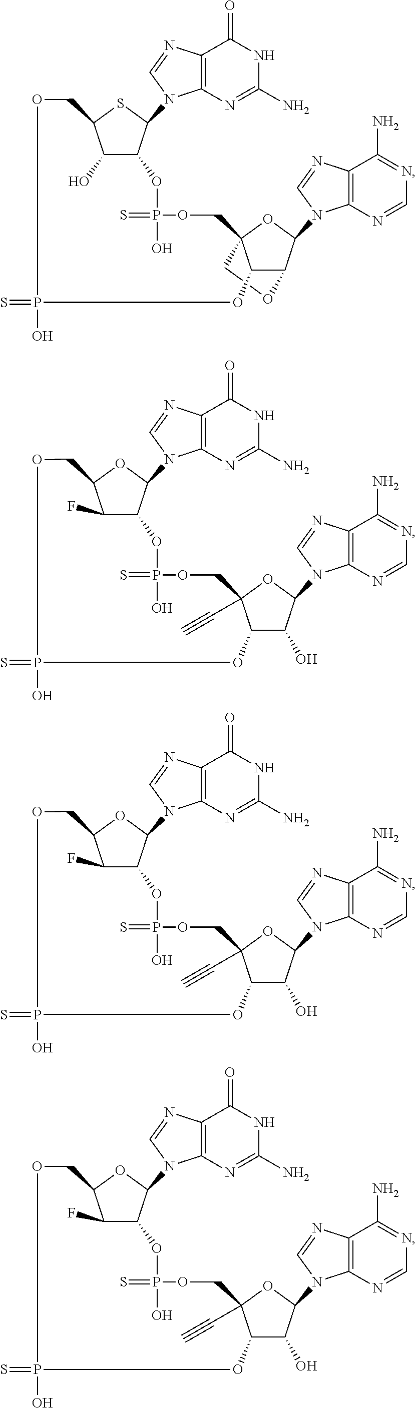

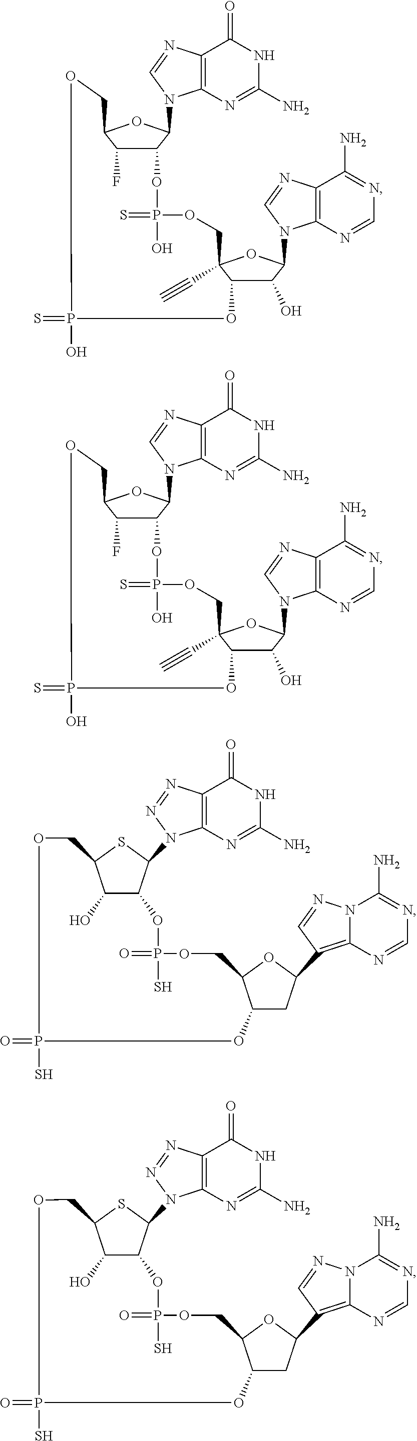 [SMILES: C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1]

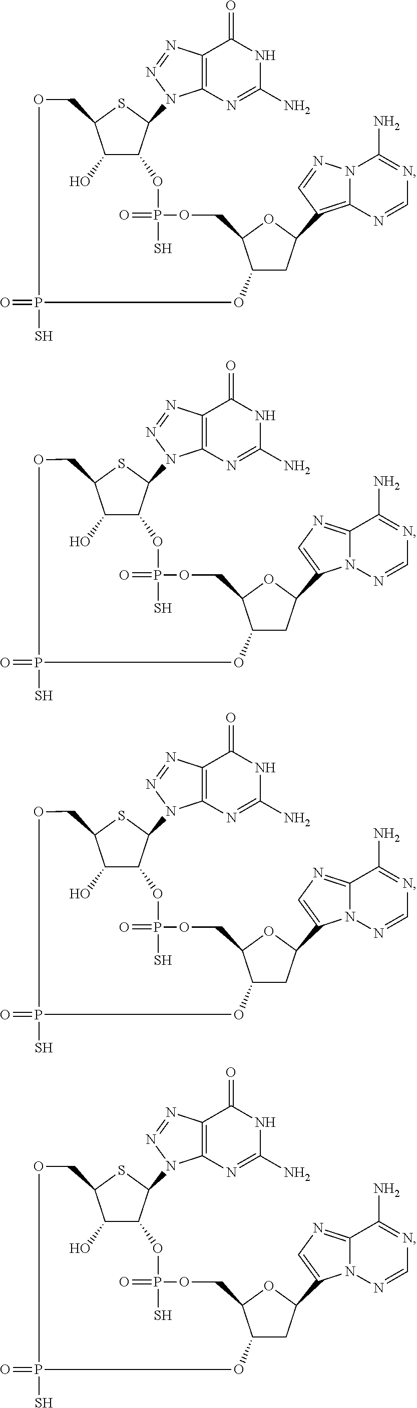 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1]

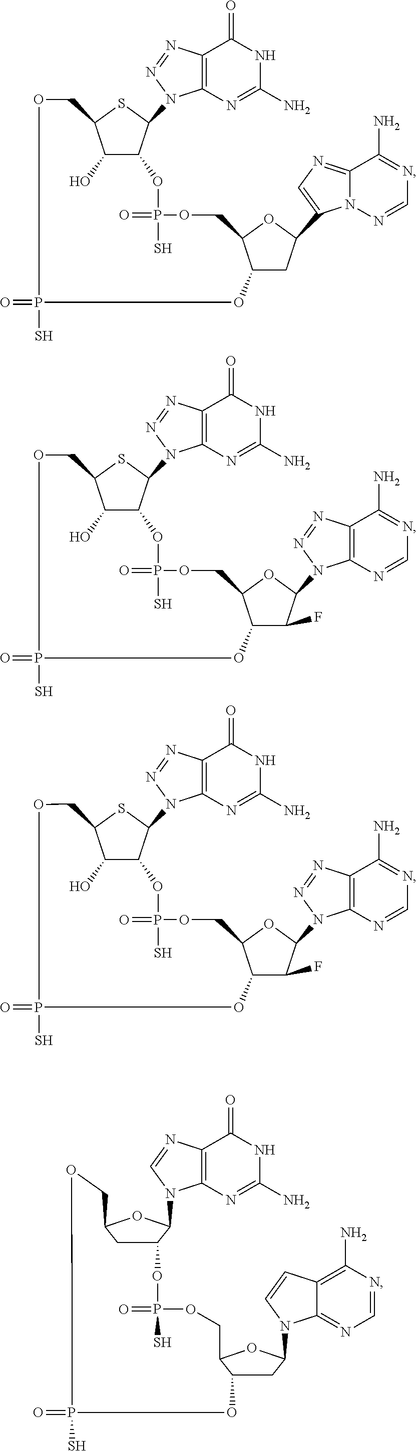 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1]

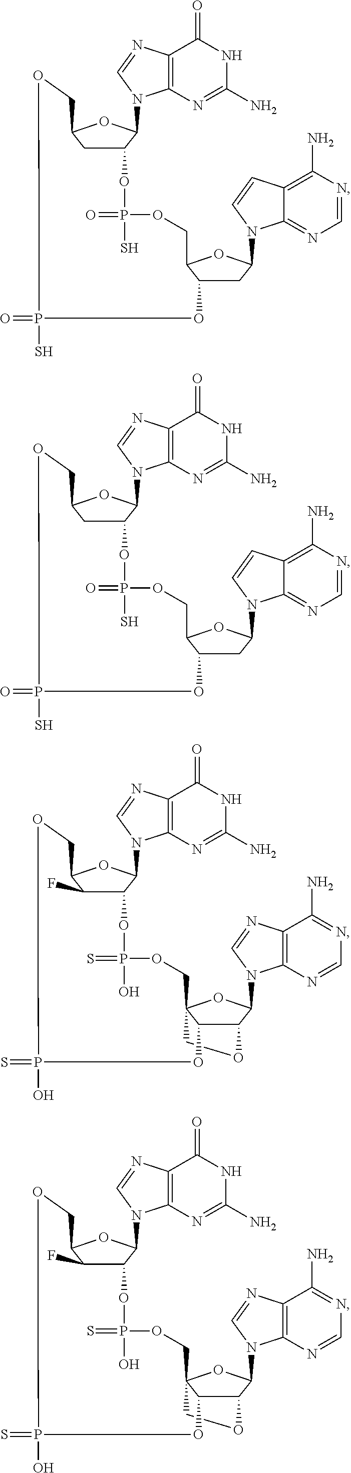 [SMILES: Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1]

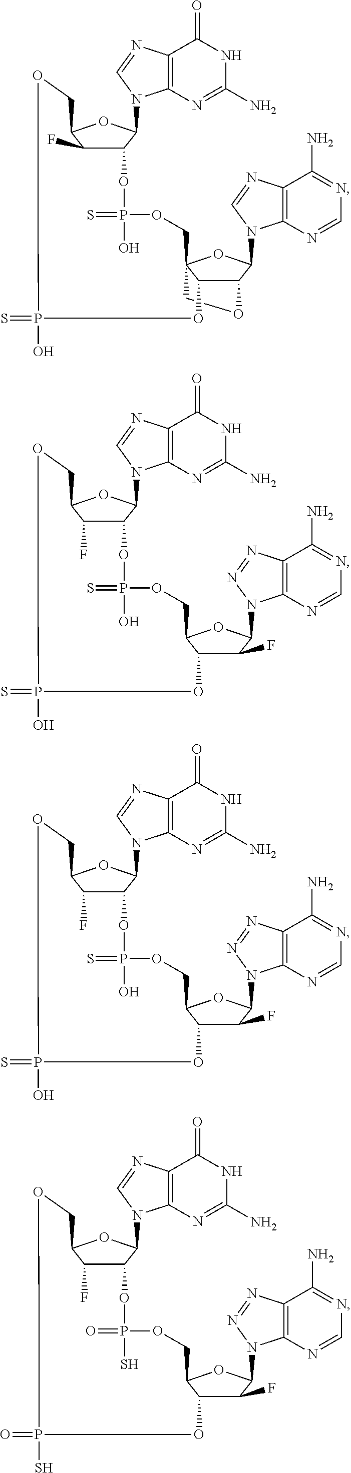 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1]

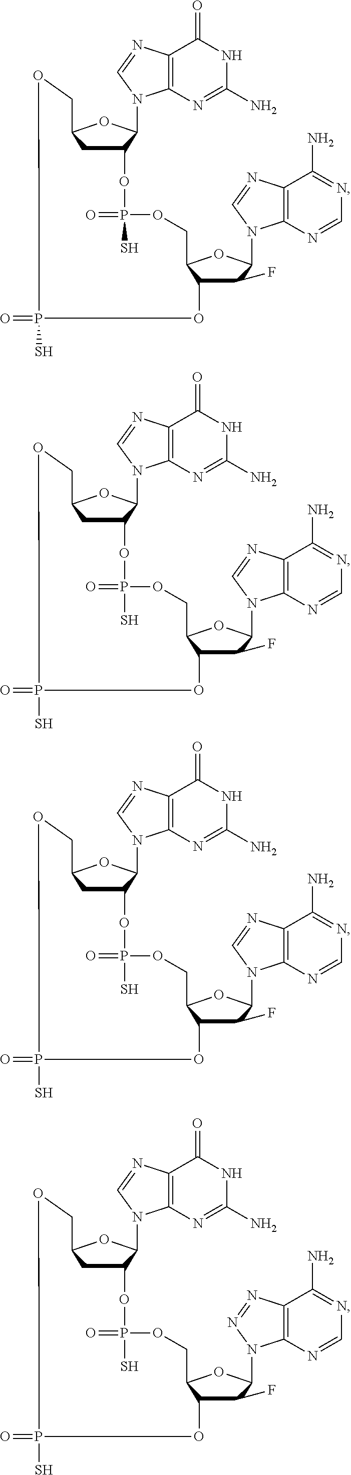

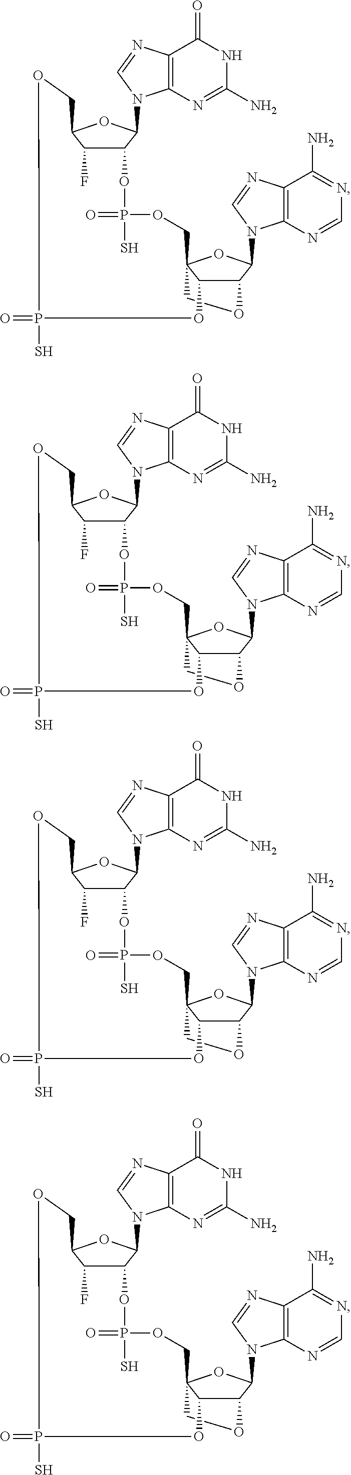 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1]

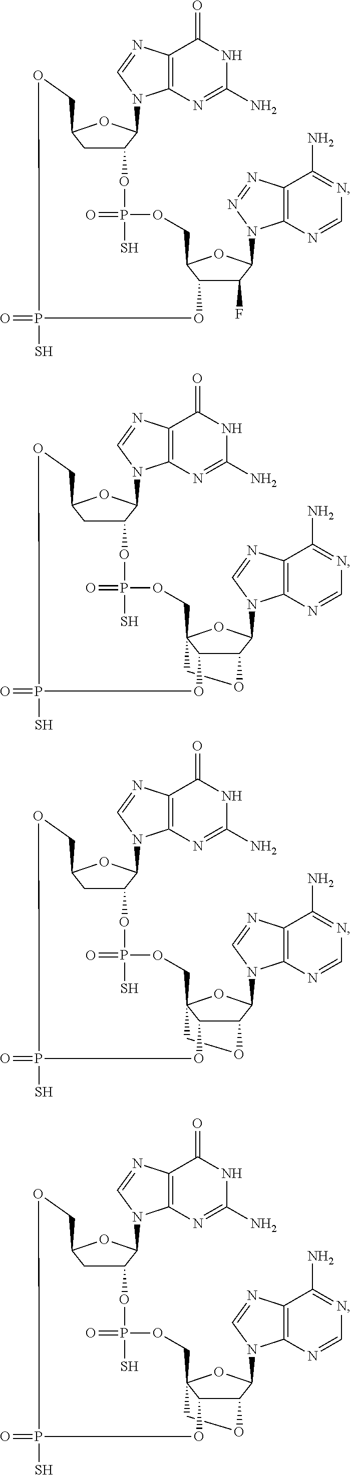 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1]

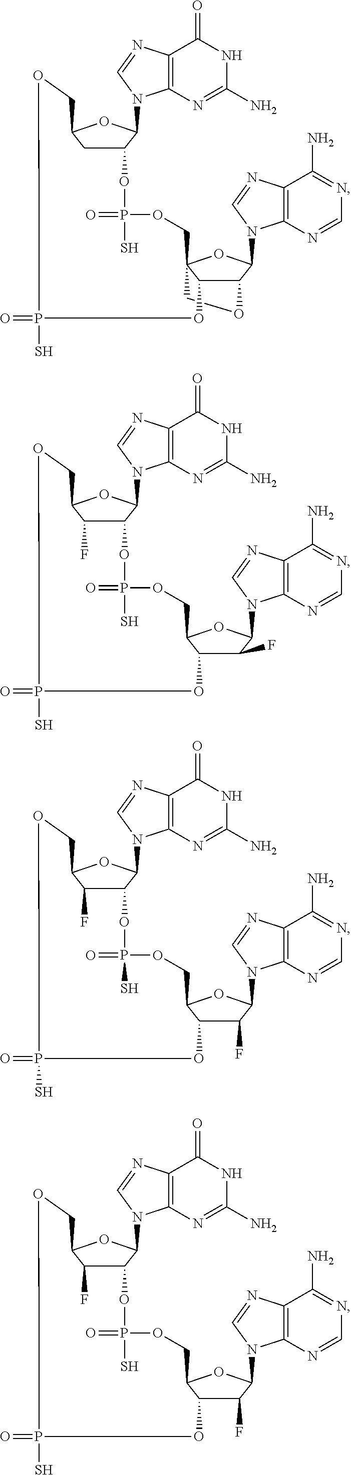 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1]

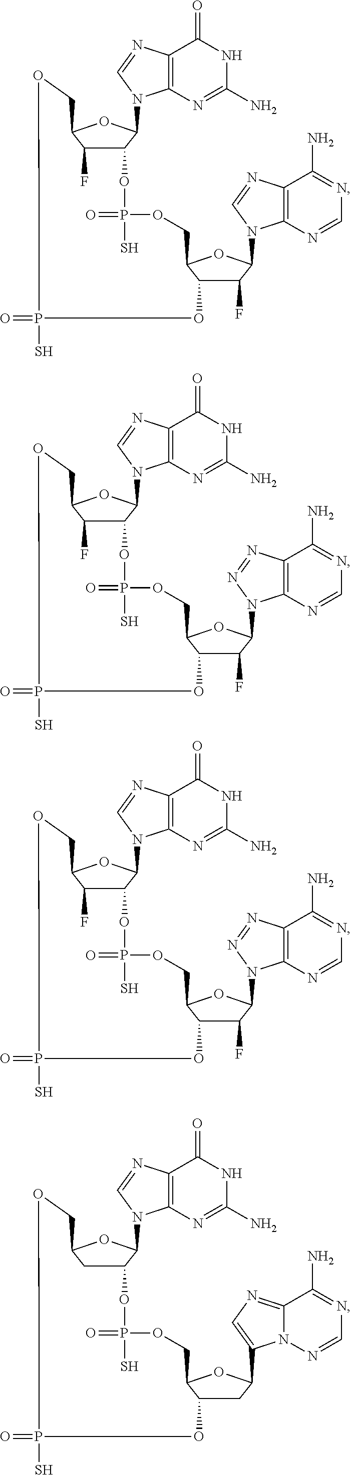 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1]

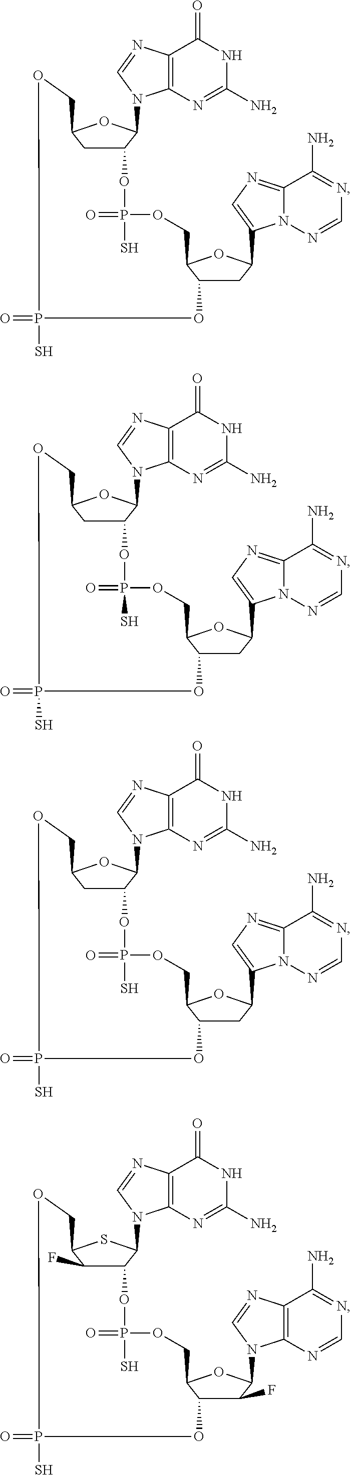 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2O[C@@H]3CO[P@@](=O)(S)O[C@H]4C[C@H](c5cnc6c(N)ncnn56)O[C@@H]4CO[P@@](=O)(S)O[C@@H]2C3)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1]

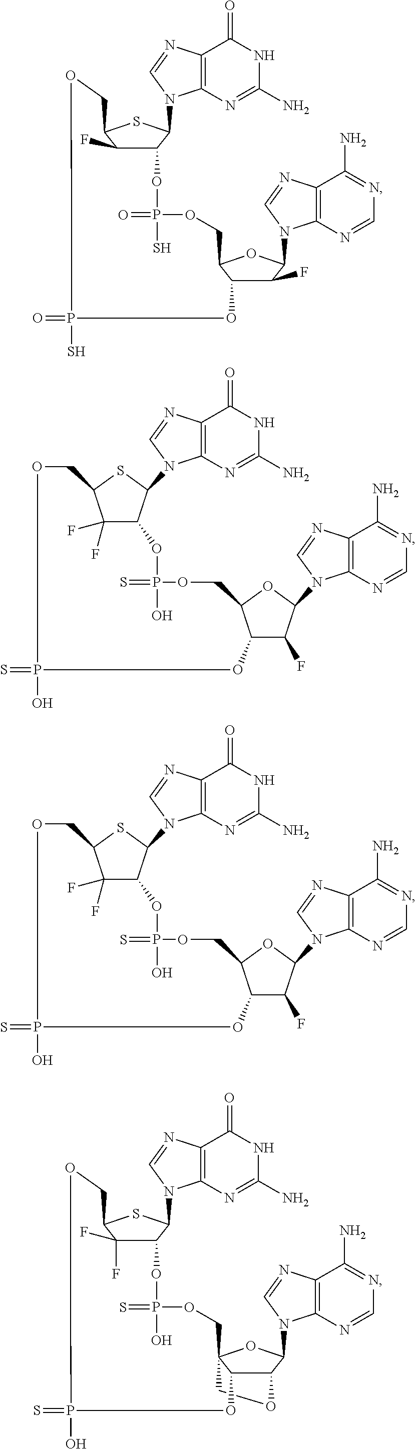 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2S[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2S[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1]

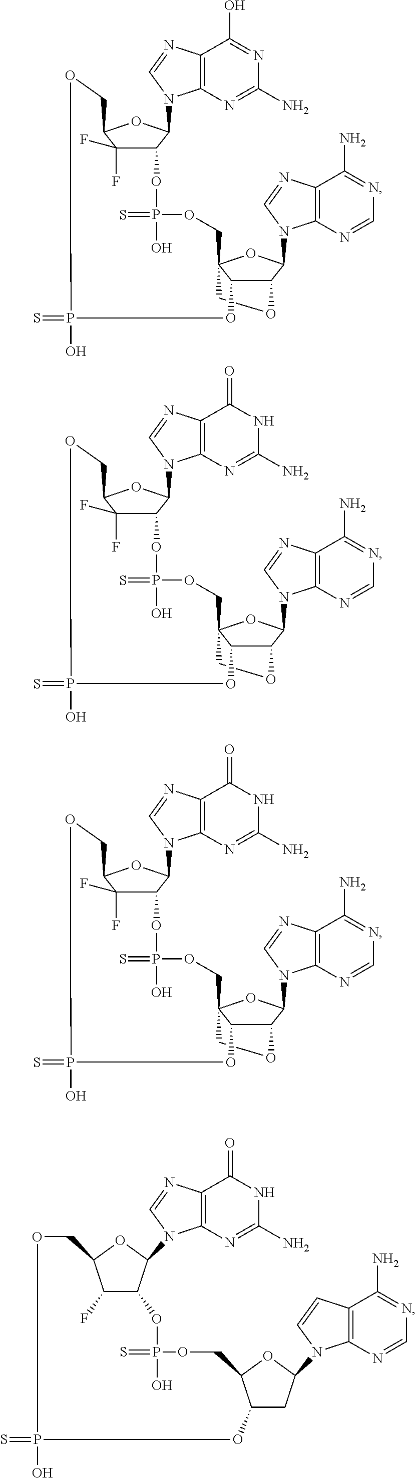 [SMILES: Nc1nc(O)c2ncn([C@@H]3O[C@@H]4COP(O)(=S)O[C@H]5[C@H]6OC[C@]5(COP(O)(=S)O[C@@H]3C4(F)F)O[C@H]6n3cnc4c(N)ncnc43)c2n1 ; Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1]

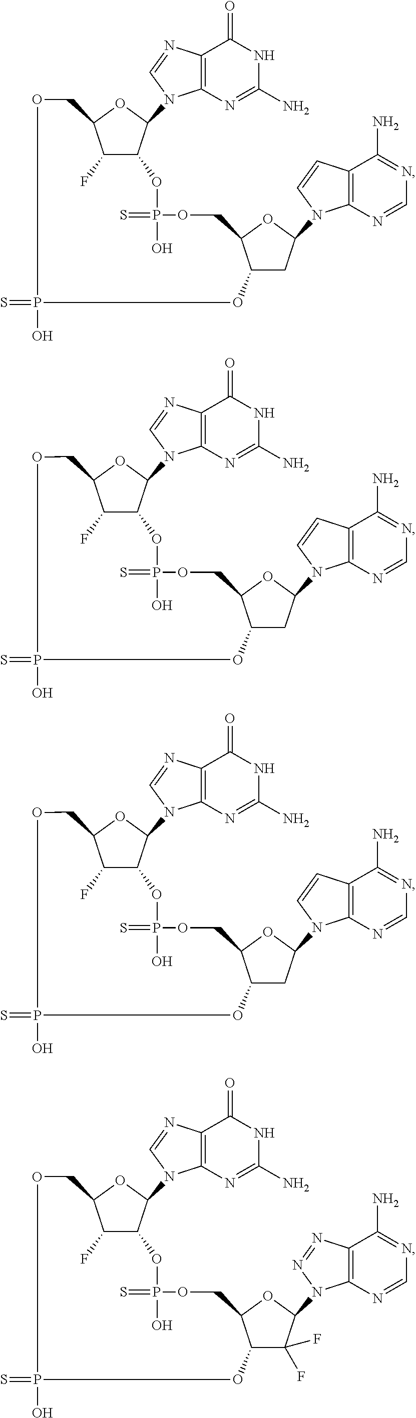 [SMILES: Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1]

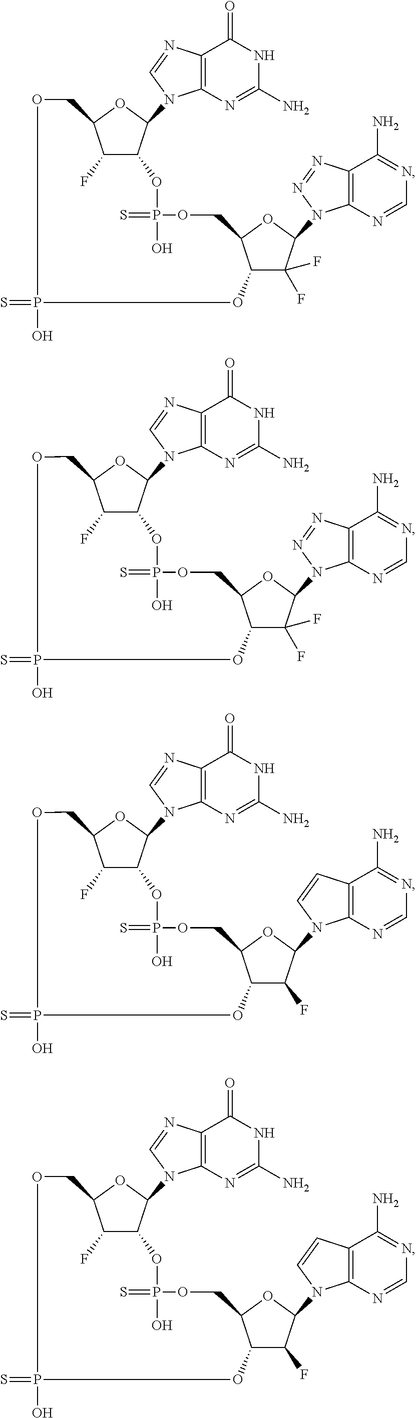 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1]

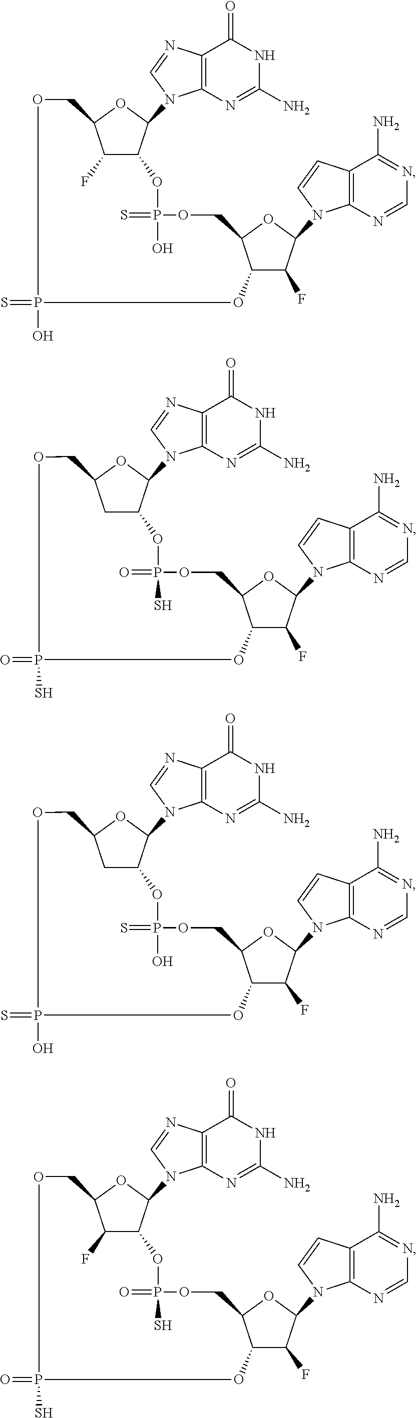 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(S)O[C@@H]2C3)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(S)O[C@@H]2[C@H]3F)c(=O)[nH]1]

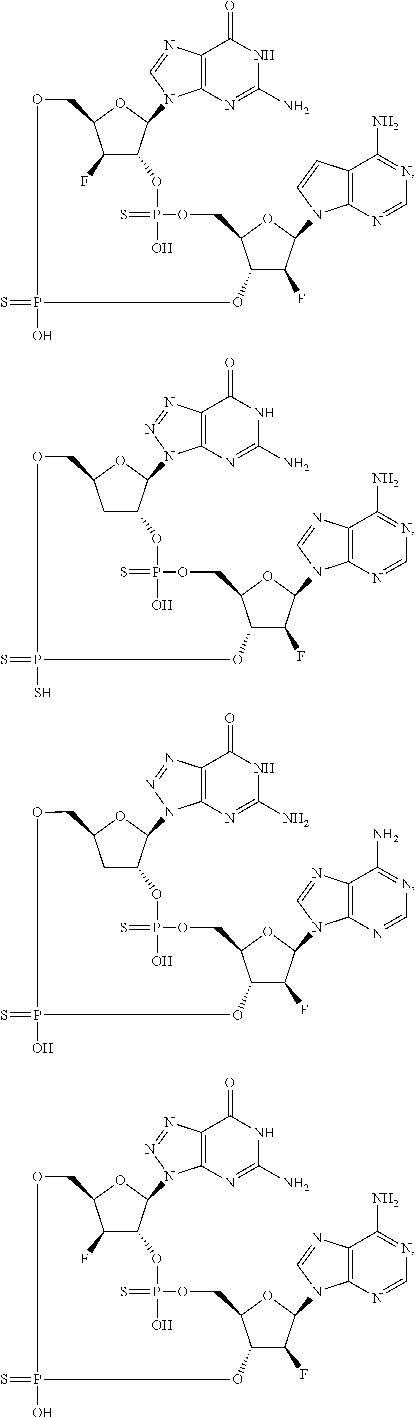 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@H]3F)c(=O)[nH]1 ; Nc1nc2c(nnn2[C@@H]2O[C@@H]3COP(=S)(S)O[C@H]4[C@H](F)[C@H](n5cnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1]

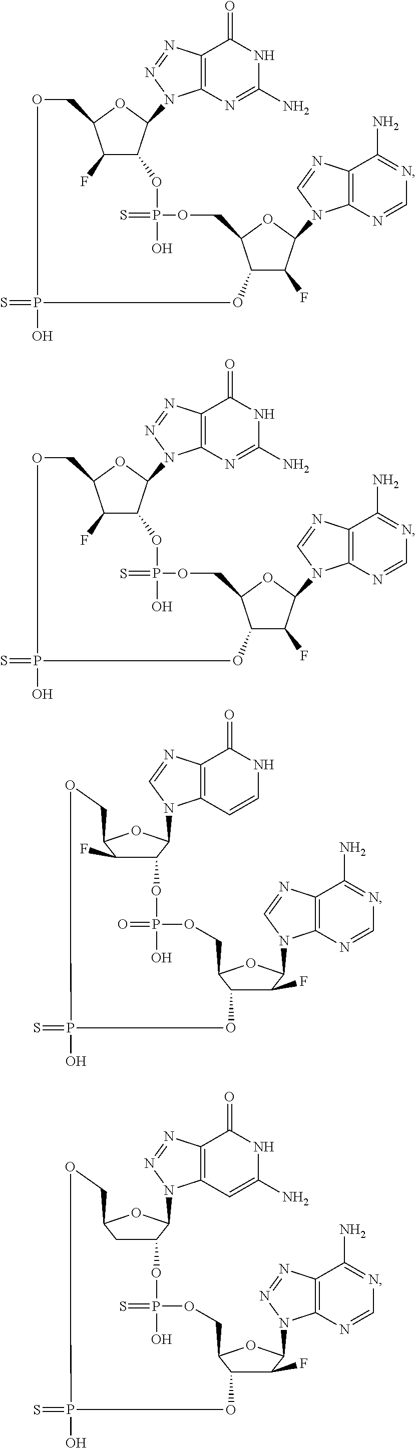 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1ncnc2c1ncn2[C@@H]1O[C@@H]2COP(=O)(O)O[C@@H]3[C@@H](F)[C@@H](COP(O)(=S)O[C@H]2[C@@H]1F)O[C@H]3n1cnc2c(=O)[nH]ccc21 ; Nc1cc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1]

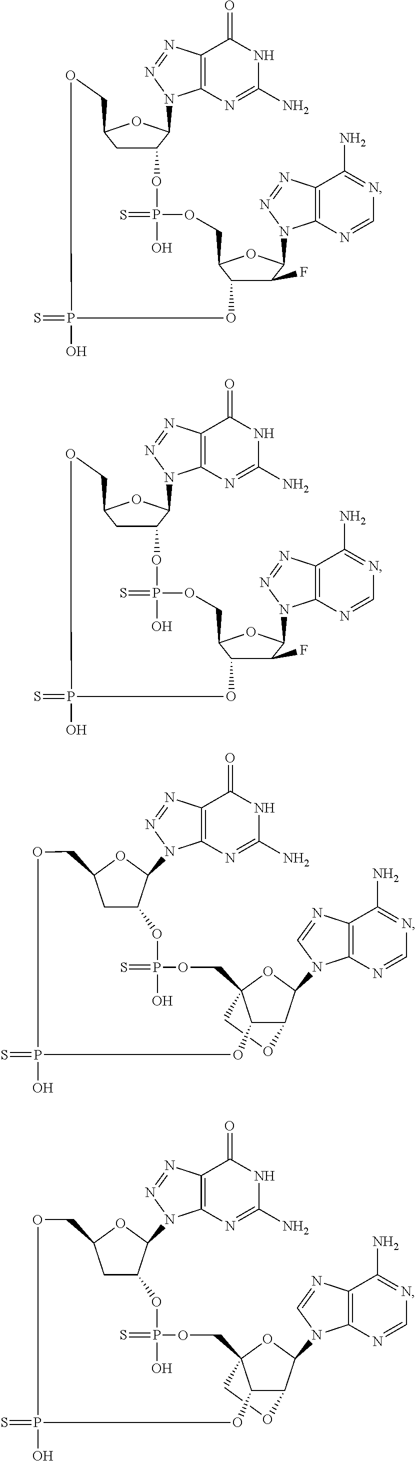 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1]

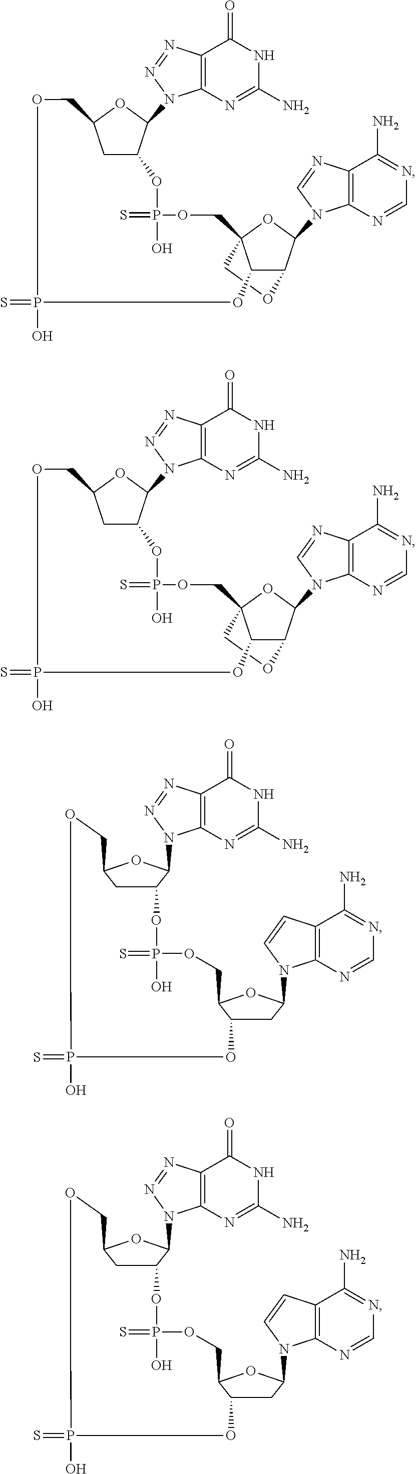 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1]

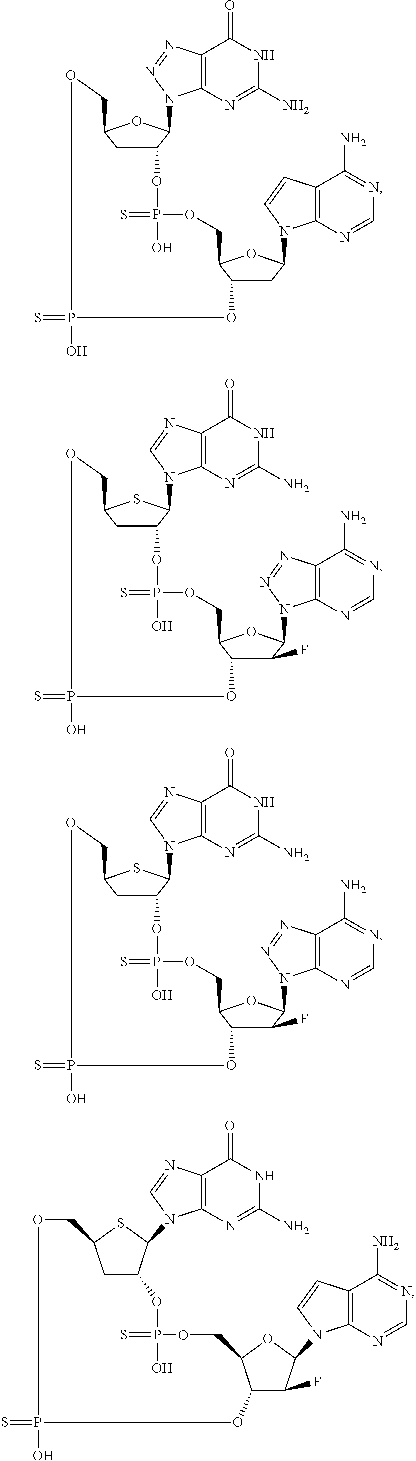 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5ccc6c(N)ncnc65)O[C@@H]4COP(O)(=S)O[C@@H]2C3)c(=O)[nH]1]

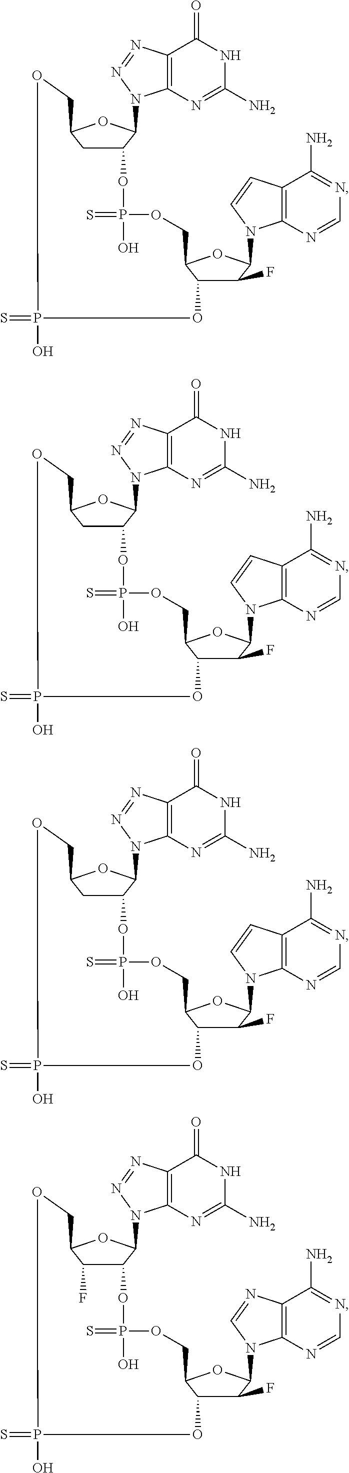 [SMILES: Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

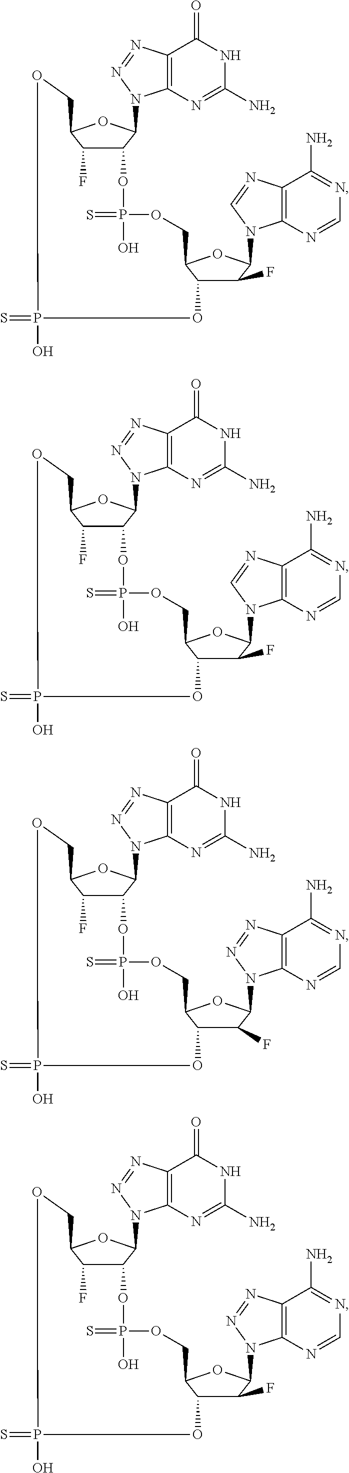 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1]

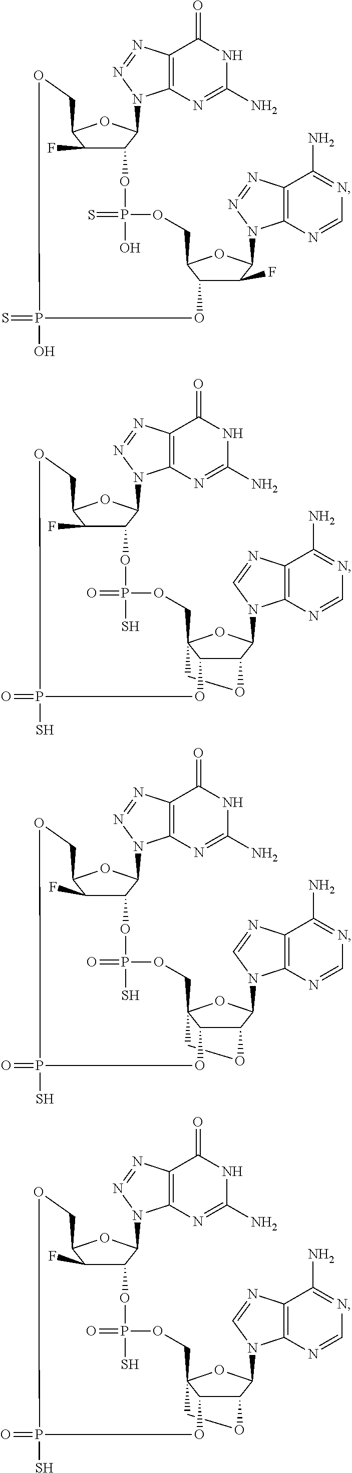 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1]

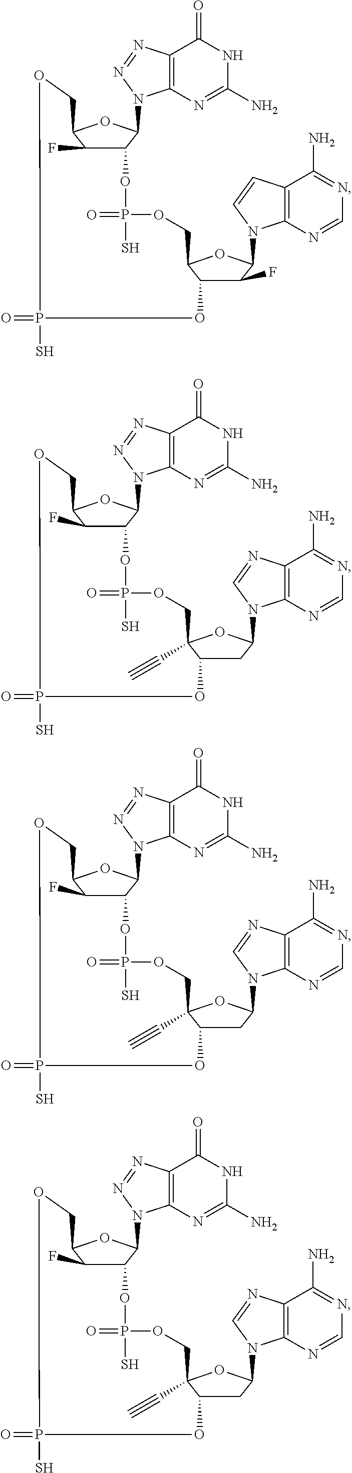 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21]

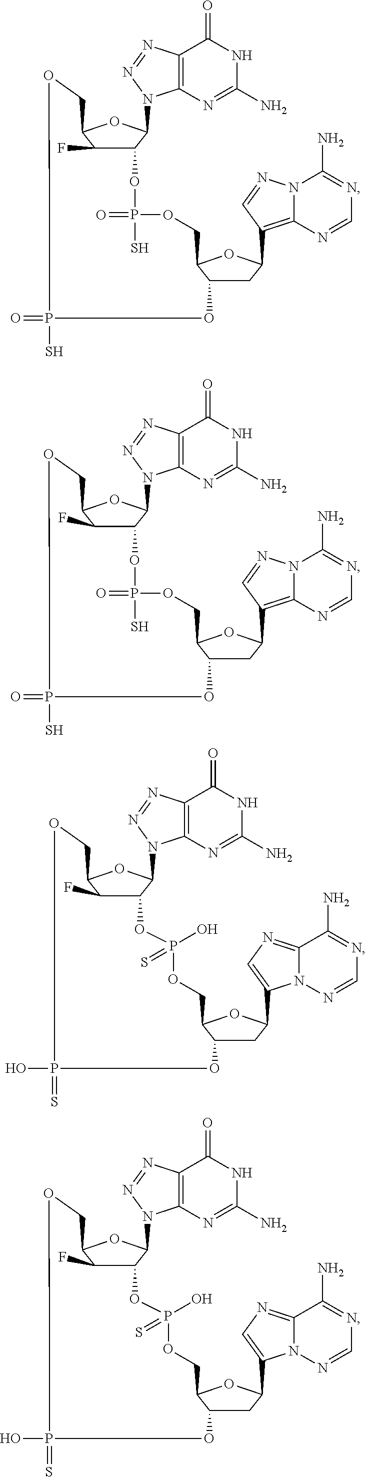 [SMILES: Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1 ; Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1 ; Nc1nc2c(nnn2[C@@H]2O[C@@H]3COP(O)(=S)O[C@H]4C[C@H](c5cnc6c(N)ncnn56)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4C[C@H](c5cnc6c(N)ncnn56)O[C@@H]4COP(O)(=S)O[C@@H]2[C@H]3F)c(=O)[nH]1]

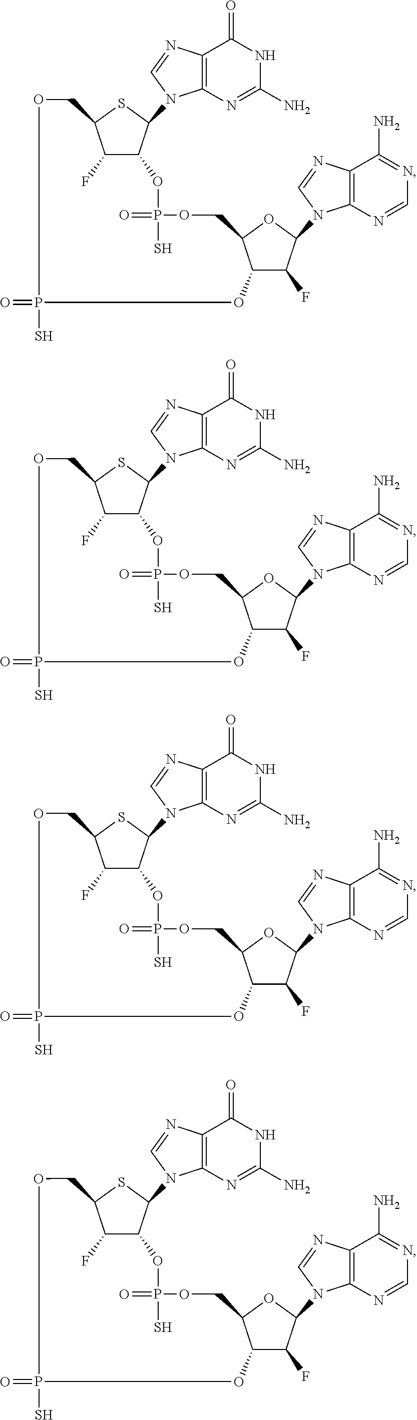 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

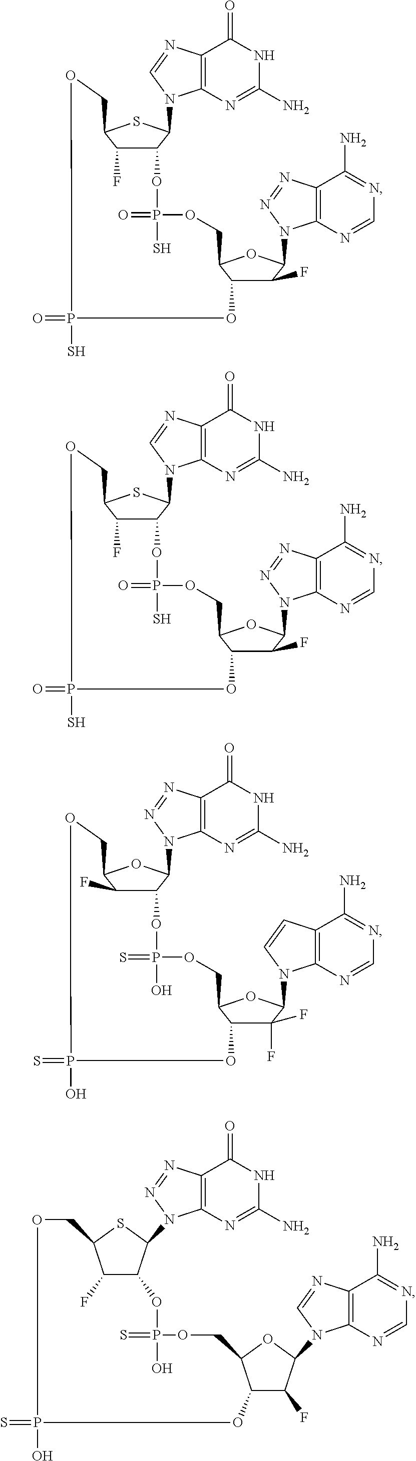 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2O[C@@H]3COP(O)(=S)O[C@@H]4[C@@H](COP(O)(=S)O[C@@H]2[C@H]3F)O[C@@H](n2ccc3c(N)ncnc32)C4(F)F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

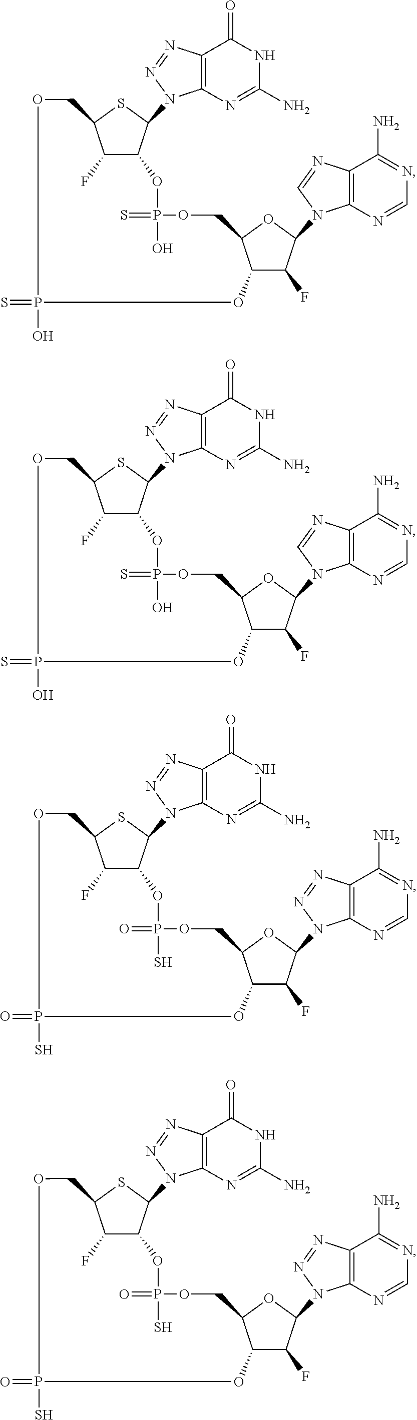 [SMILES: Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1]

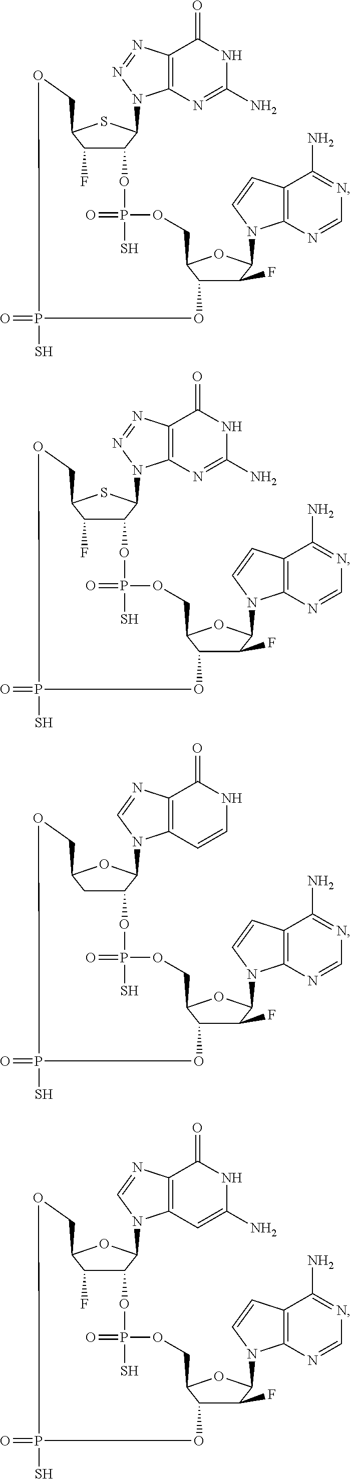 [SMILES: Nc1nc2c(nnn2[C@@H]2S[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1ncnc2c1ccn2[C@@H]1O[C@@H]2COP(=O)(S)O[C@@H]3C[C@@H](COP(=O)(S)O[C@H]2[C@@H]1F)O[C@H]3n1cnc2c(=O)[nH]ccc21 ; Nc1cc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1]

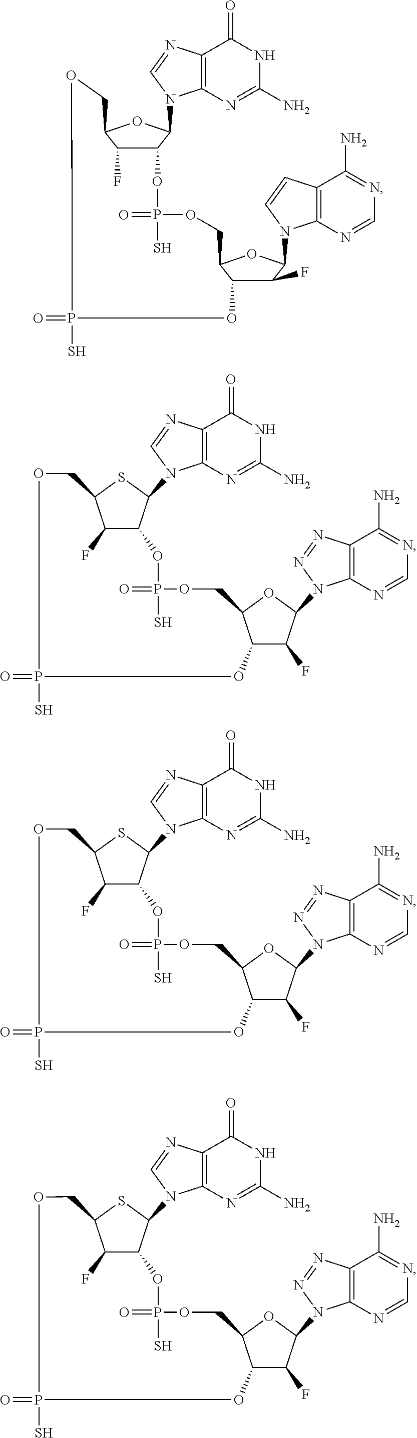 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1]

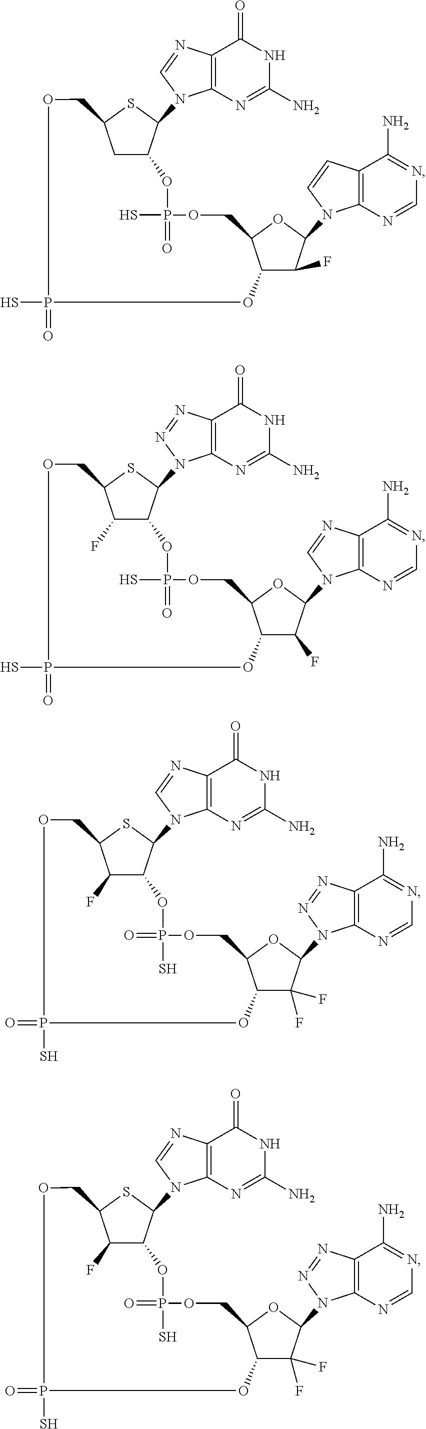 [SMILES: Nc1nc2c(ncn2[C@@H]2S[C@@H]3COP(=O)(S)O[C@H]4[C@H](F)[C@H](n5ccc6c(N)ncnc65)O[C@@H]4COP(=O)(S)O[C@@H]2C3)c(=O)[nH]1 ; Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1]

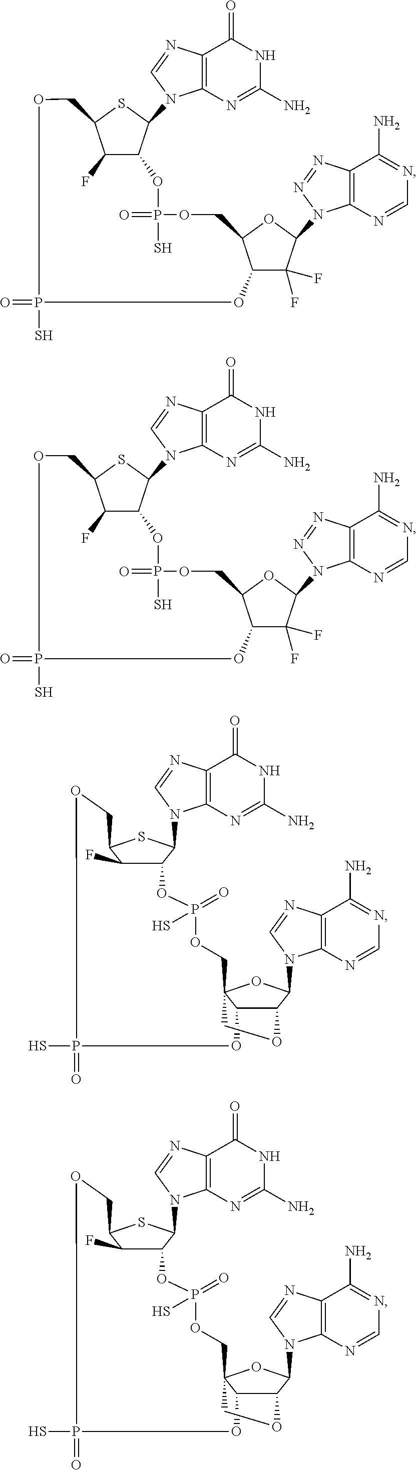 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1]

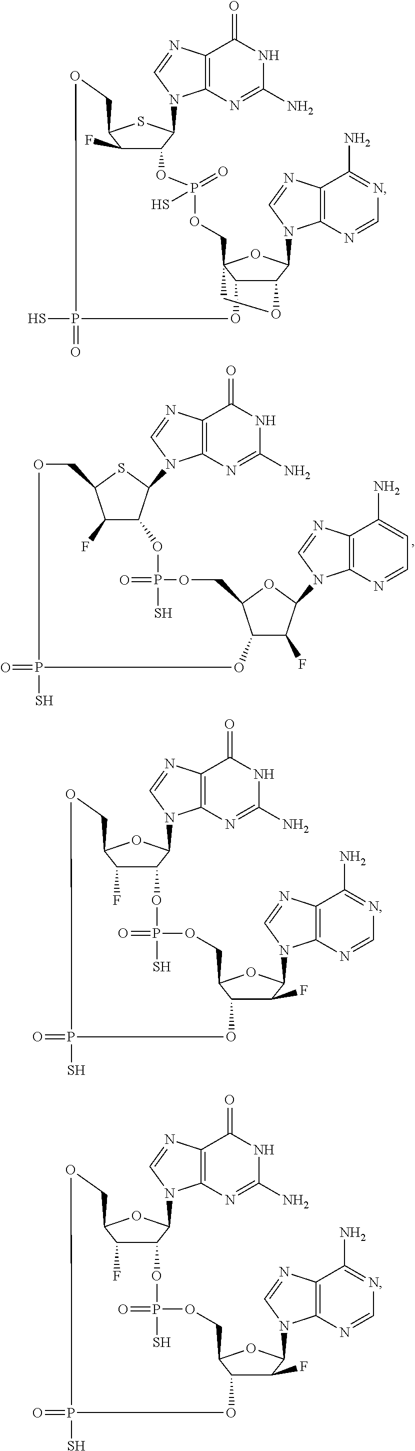 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

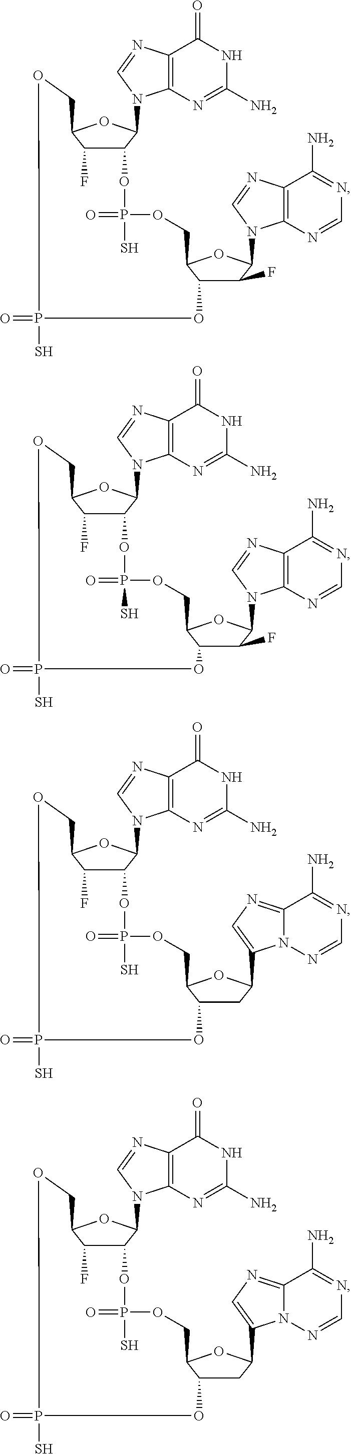 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4CO[P@@](=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1]

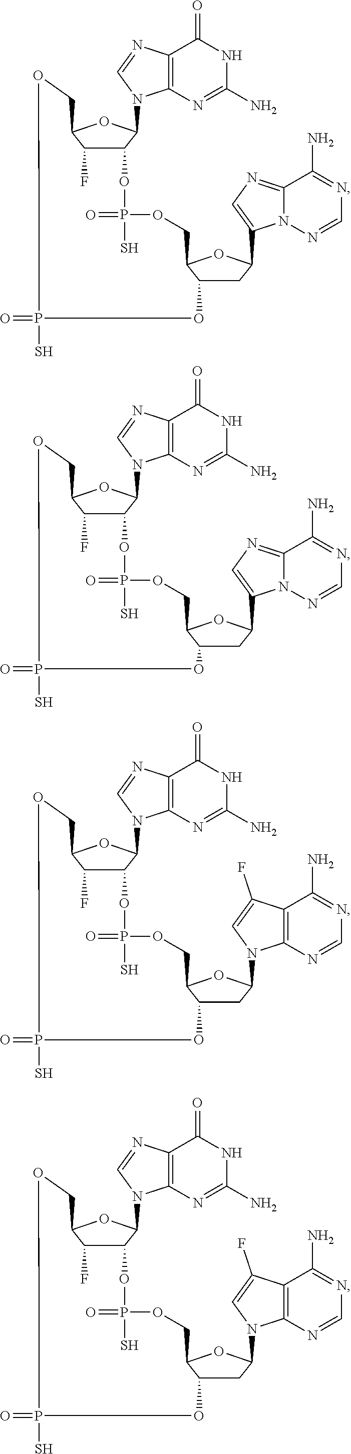 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1]

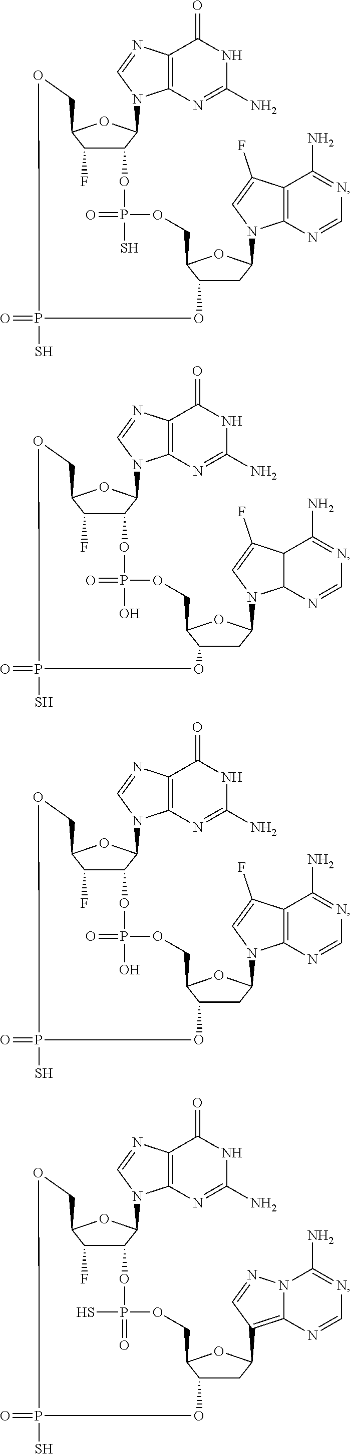 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1 ; NC1=NC=NC2C1C(F)=CN2[C@H]1C[C@@H]2OP(=O)(S)OC[C@H]3O[C@@H](n4cnc5c(=O)[nH]c(N)nc54)[C@H](OP(=O)(O)OC[C@H]2O1)[C@@H]3F ; Nc1nc2c(ncn2[C@@H]2O[C@@H]3COP(=O)(S)O[C@H]4C[C@H](n5cc(F)c6c(N)ncnc65)O[C@@H]4COP(=O)(O)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1]

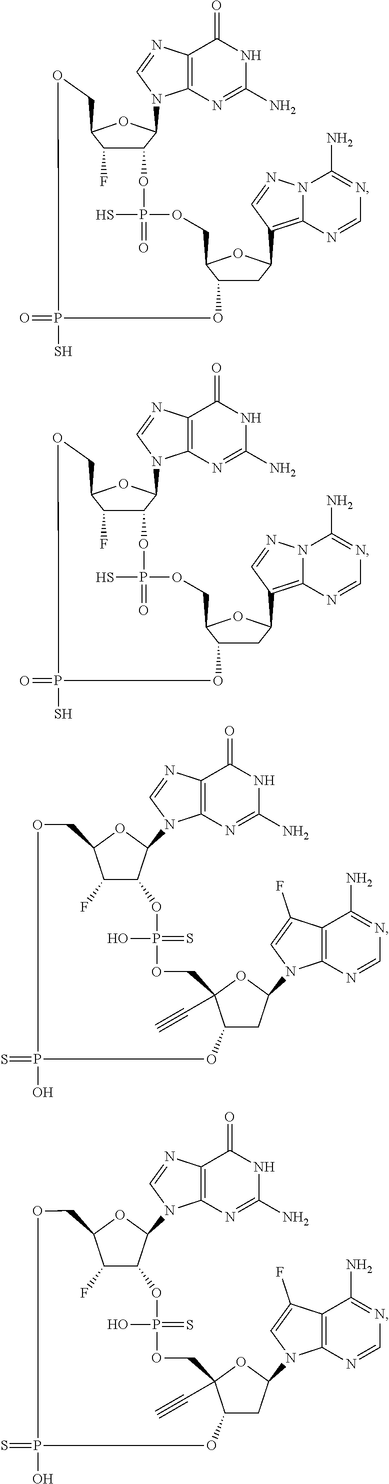 [SMILES: Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21]

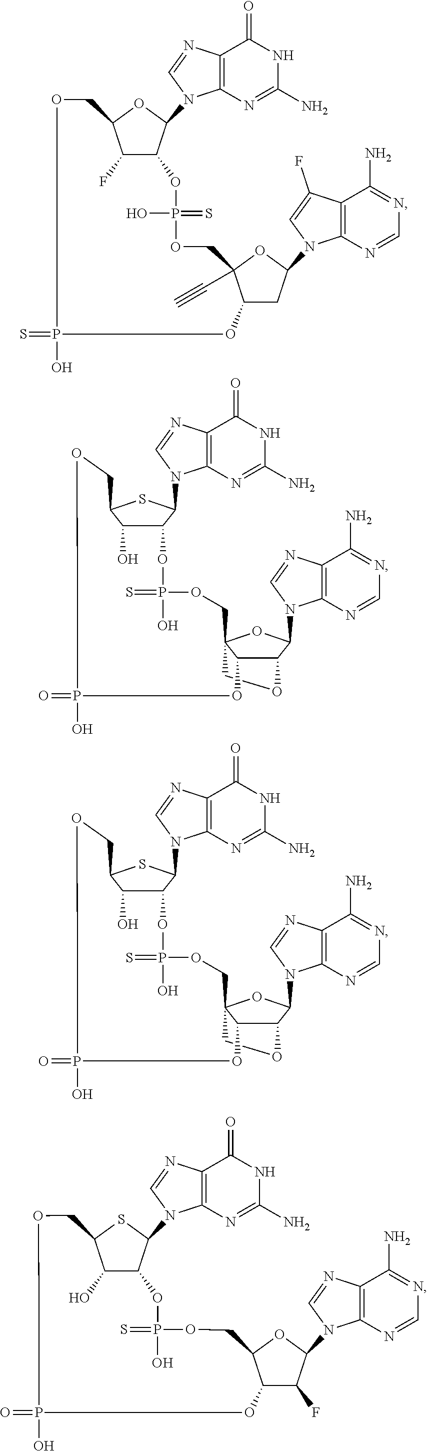 [SMILES: C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; Nc1nc2c(ncn2[C@@H]2S[C@@H]3COP(=O)(O)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(=O)(O)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(=O)(O)O[C@H]4[C@H](F)[C@H](n5cnc6c(N)ncnc65)O[C@@H]4COP(O)(=S)O[C@@H]2[C@@H]3O)c(=O)[nH]1]

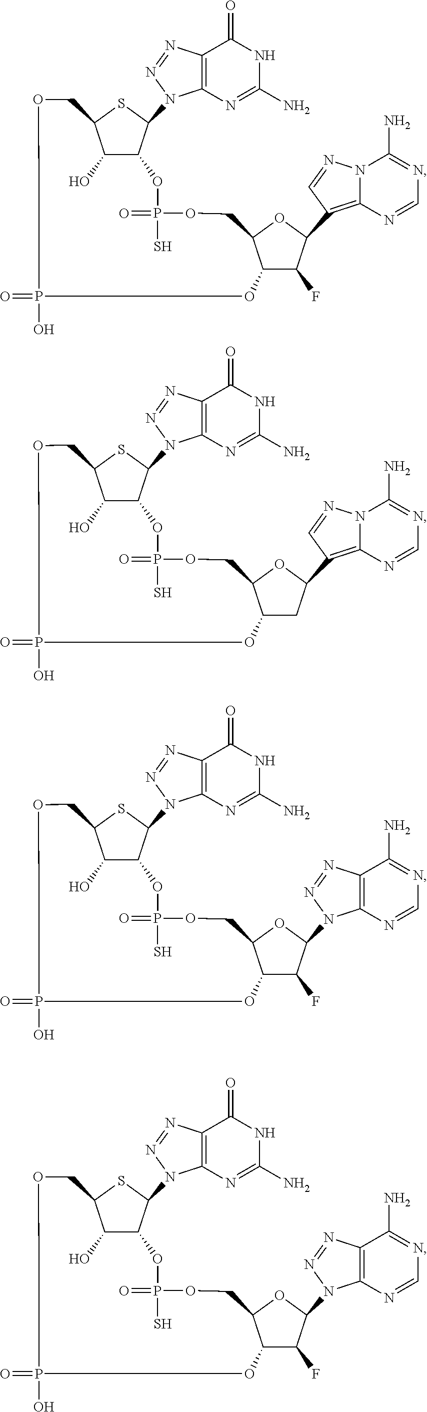 [SMILES: Nc1nc2c(nnn2[C@@H]2S[C@@H]3COP(=O)(O)O[C@H]4[C@H](F)[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1]

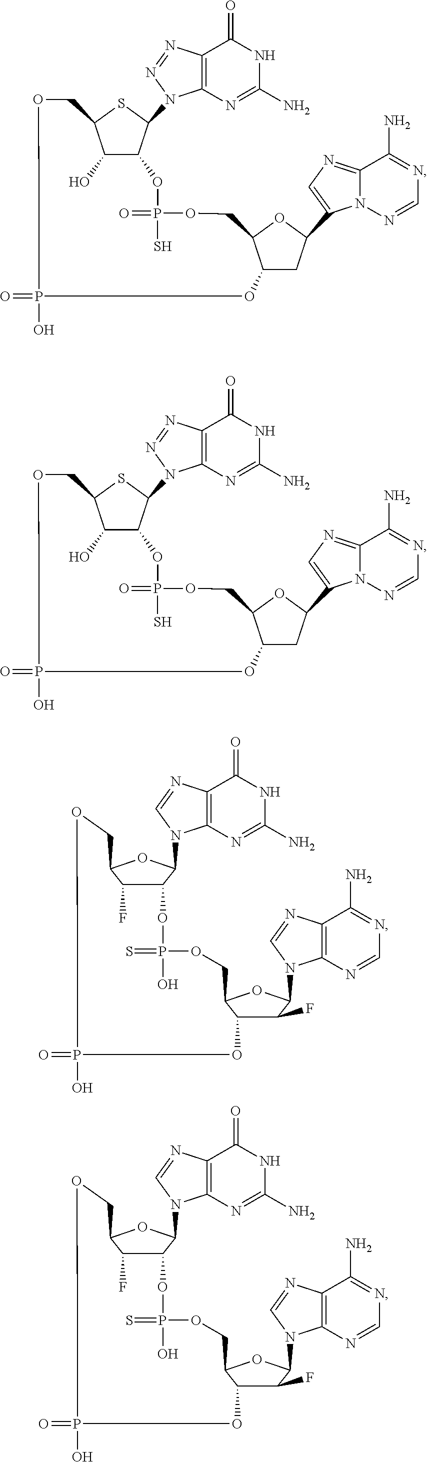 [SMILES: Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1]

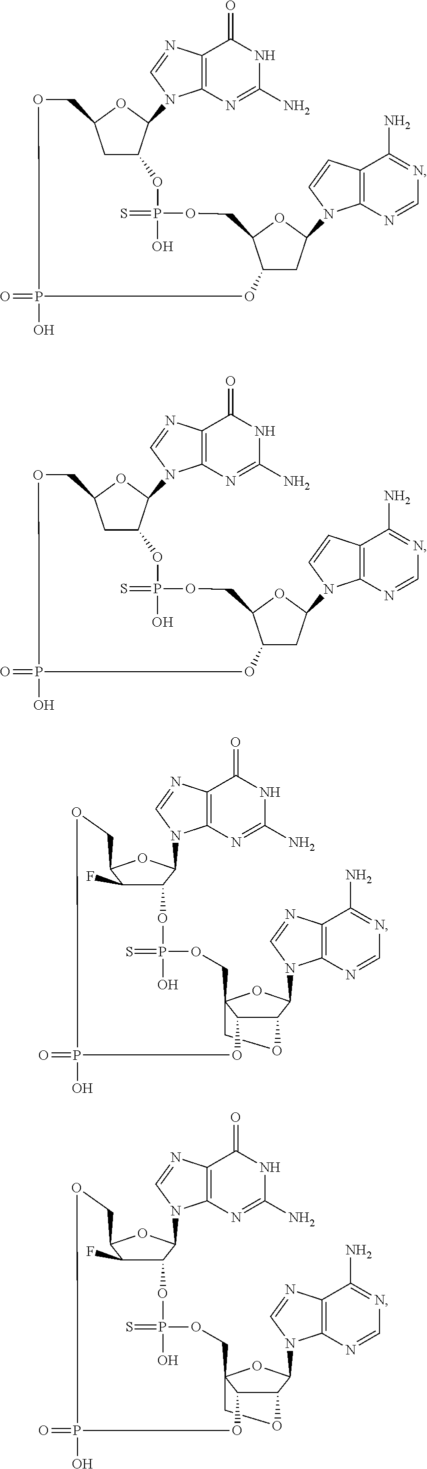 [SMILES: Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1]

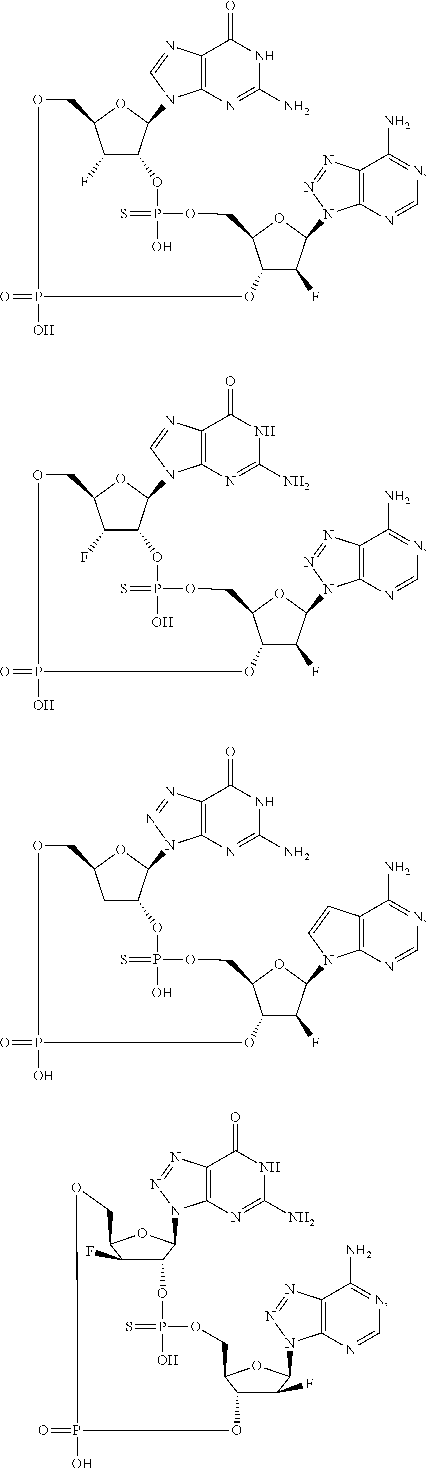 [SMILES: Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(=O)(O)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(=O)(O)O[C@H]4[C@H](F)[C@H](n5nnc6c(N)ncnc65)O[C@@H]4COP(O)(=S)O[C@@H]2[C@H]3F)c(=O)[nH]1]

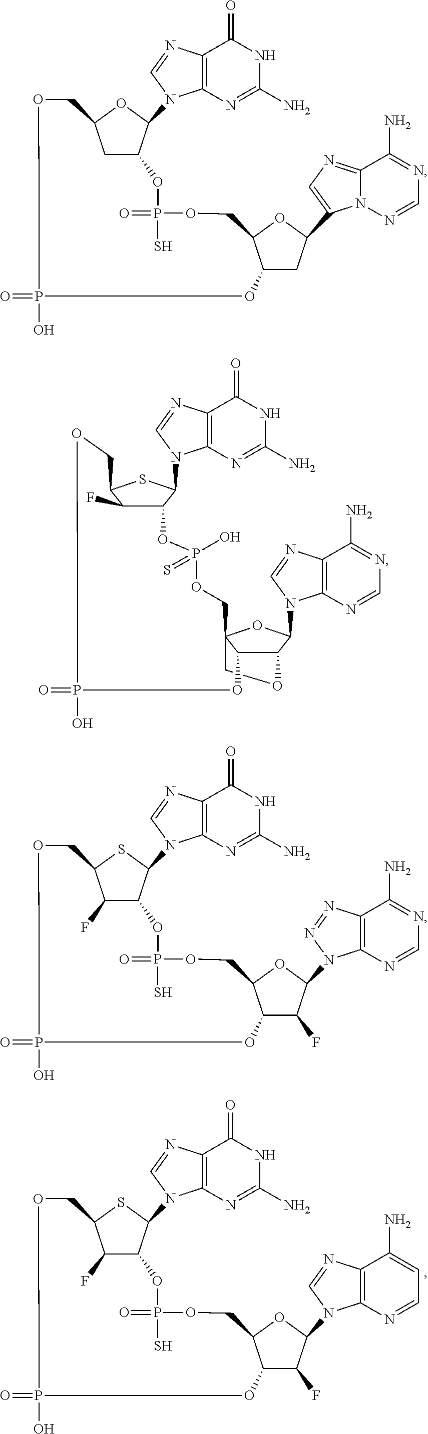 [SMILES: Nc1nc2c(ncn2[C@@H]2O[C@@H]3COP(=O)(O)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2S[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2S[C@@H]3COP(=O)(O)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1]

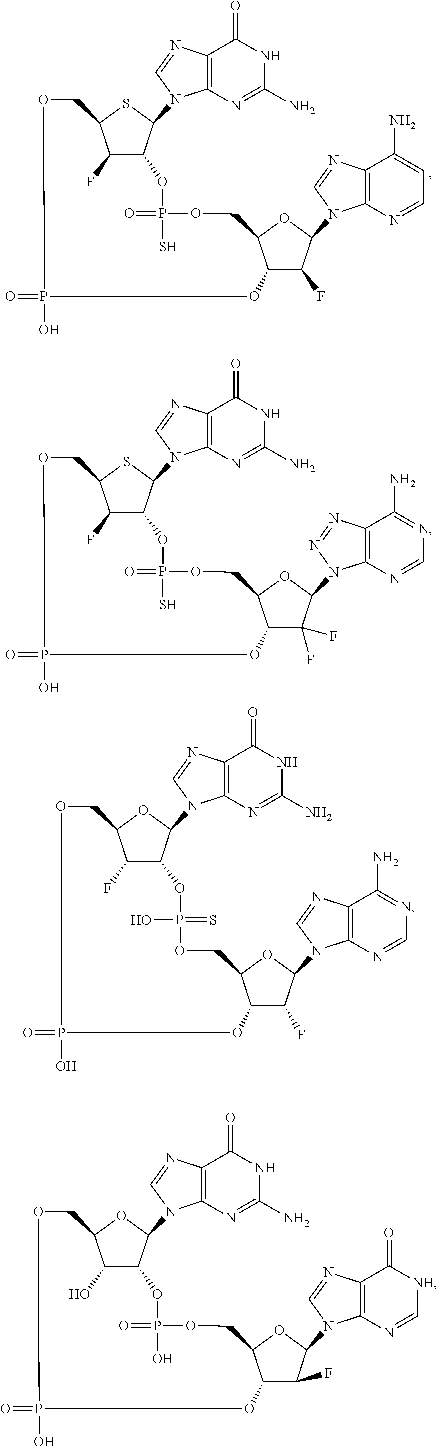 [SMILES: Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2O[C@@H]3COP(=O)(O)O[C@H]4[C@@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2[C@@H]3O)c(=O)[nH]1]

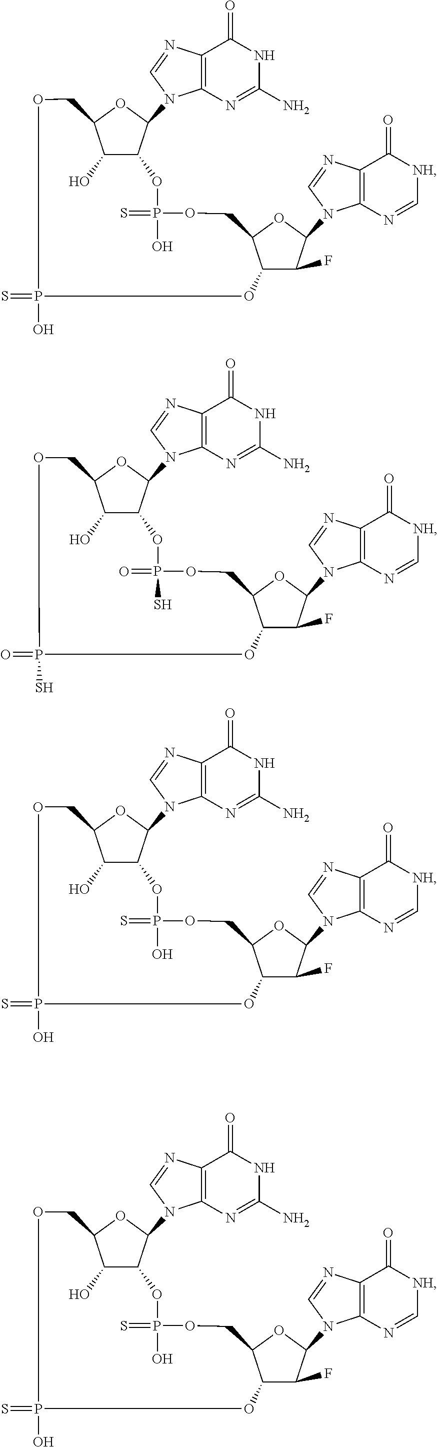

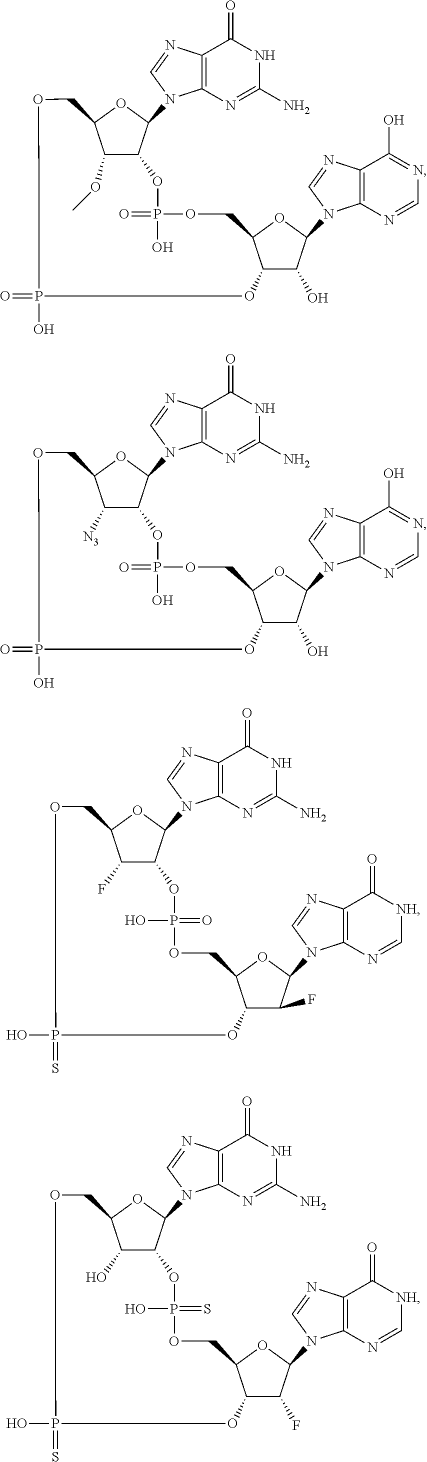

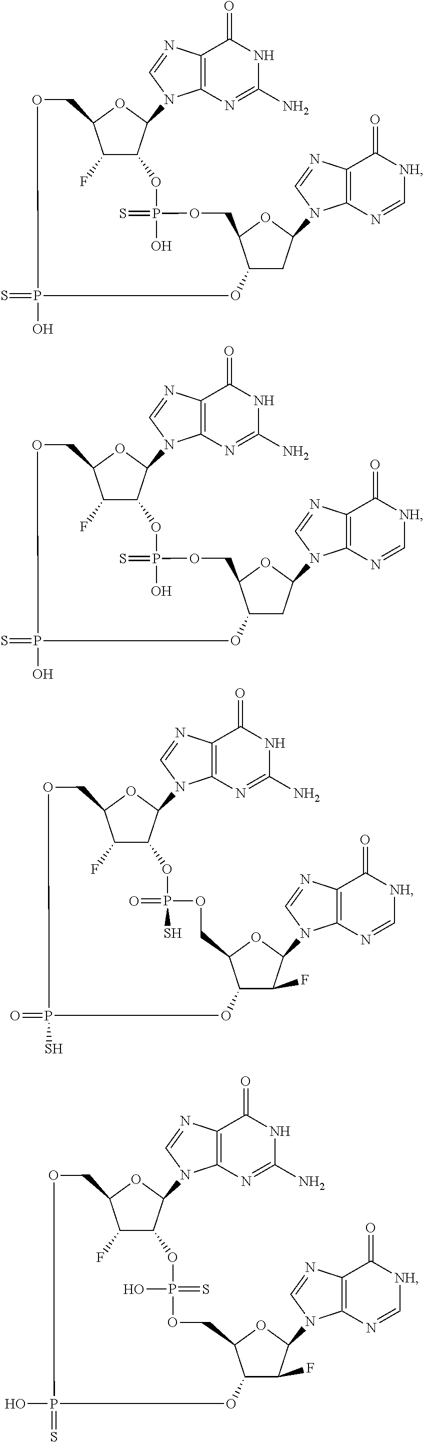 [SMILES: Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

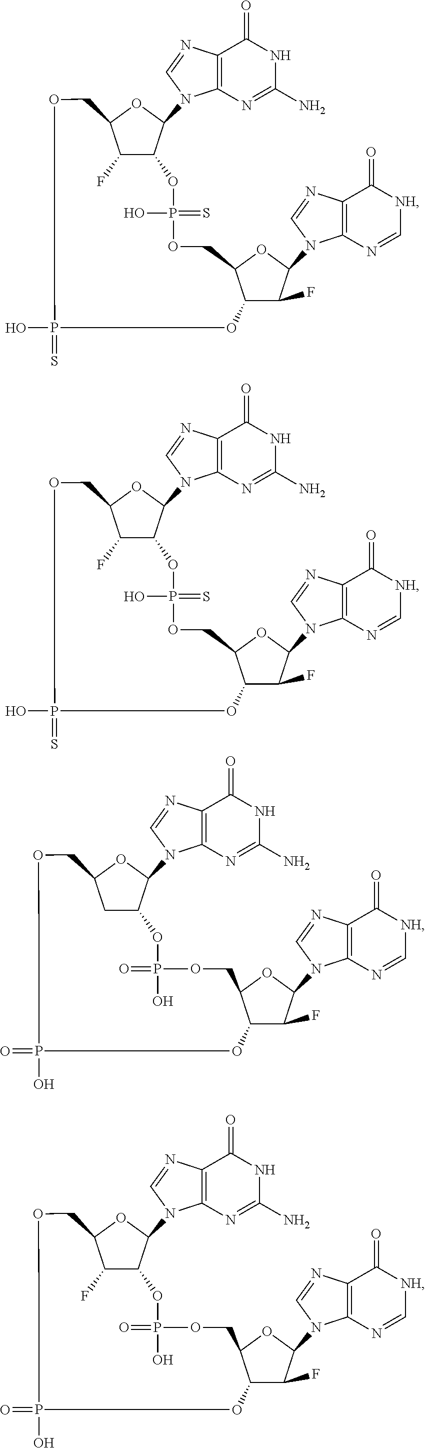 [SMILES: Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2C3)c(=O)[nH]1 ; Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2[C@@H]3F)c(=O)[nH]1]

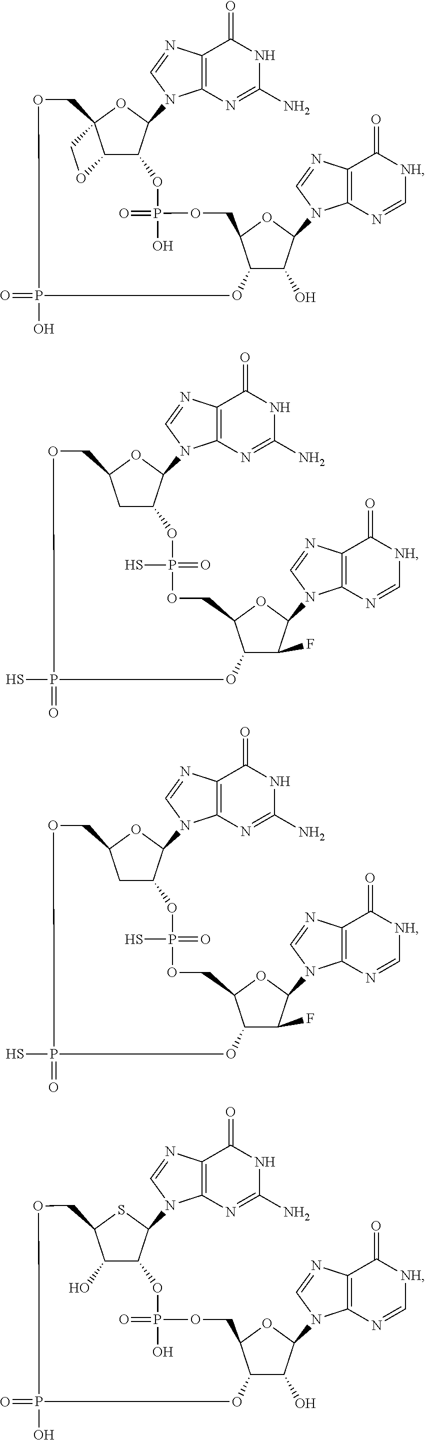 [SMILES: Nc1nc2c(ncn2[C@@H]2O[C@@]34CO[C@H]3[C@H]2OP(=O)(O)OC[C@H]2O[C@@H](n3cnc5c(=O)[nH]cnc53)[C@H](O)[C@@H]2OP(=O)(O)OC4)c(=O)[nH]1 ; Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2C3)c(=O)[nH]1 ; Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2C3)c(=O)[nH]1 ; Nc1nc2c(ncn2[C@@H]2S[C@@H]3COP(=O)(O)O[C@H]4[C@@H](O)[C@H](n5cnc6c(=O)[nH]cnc65)O[C@@H]4COP(=O)(O)O[C@@H]2[C@@H]3O)c(=O)[nH]1]

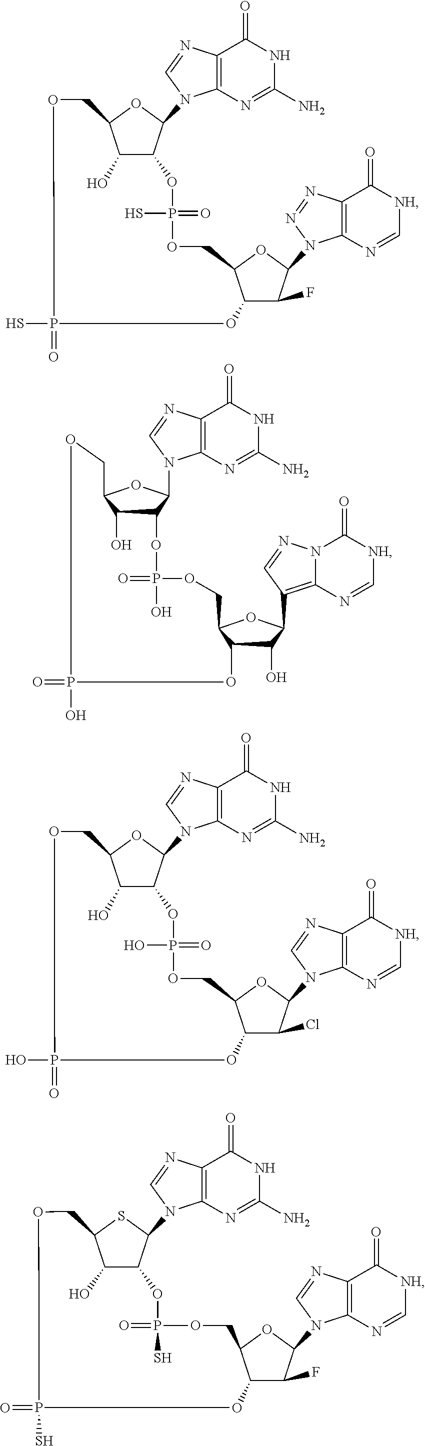

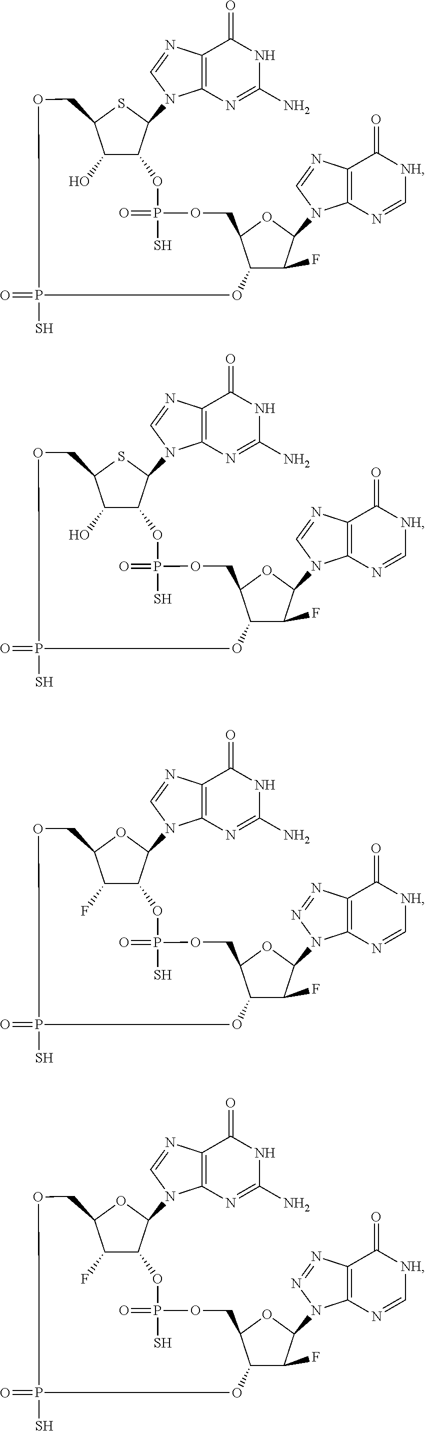

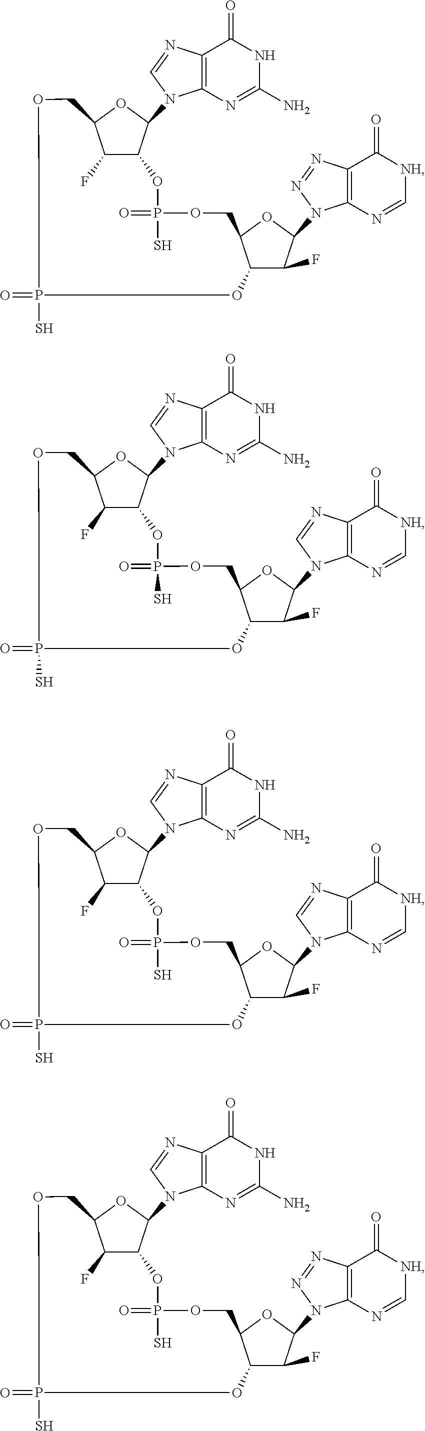

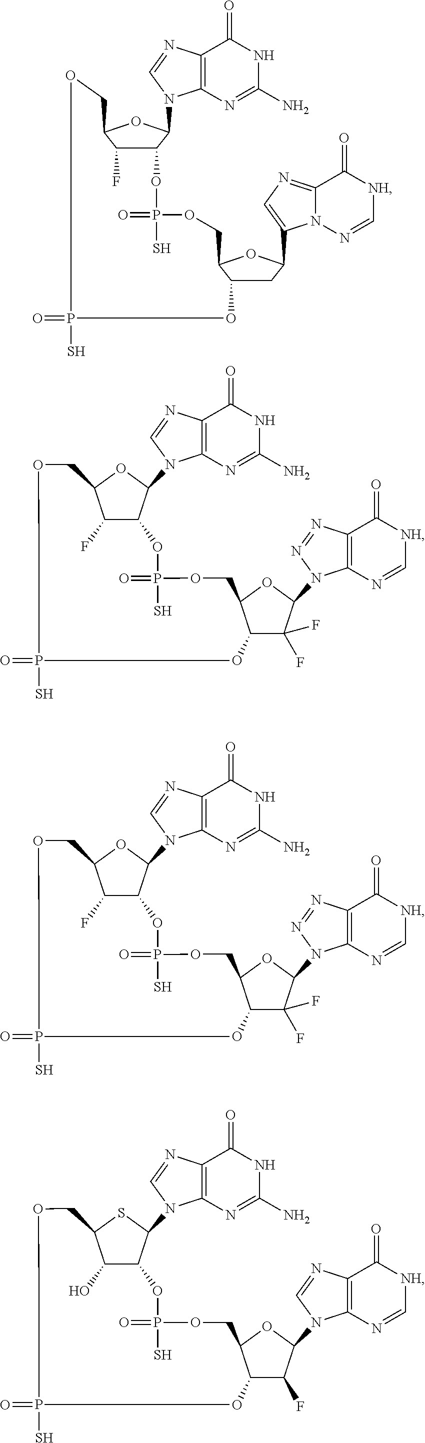 [SMILES: Nc1nc2c(ncn2[C@@H]2O[C@@H]3COP(=O)(S)O[C@H]4C[C@H](c5cnc6c(=O)[nH]cnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1 ; Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1 ; Nc1nc2c(ncn2[C@@H]2S[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1]

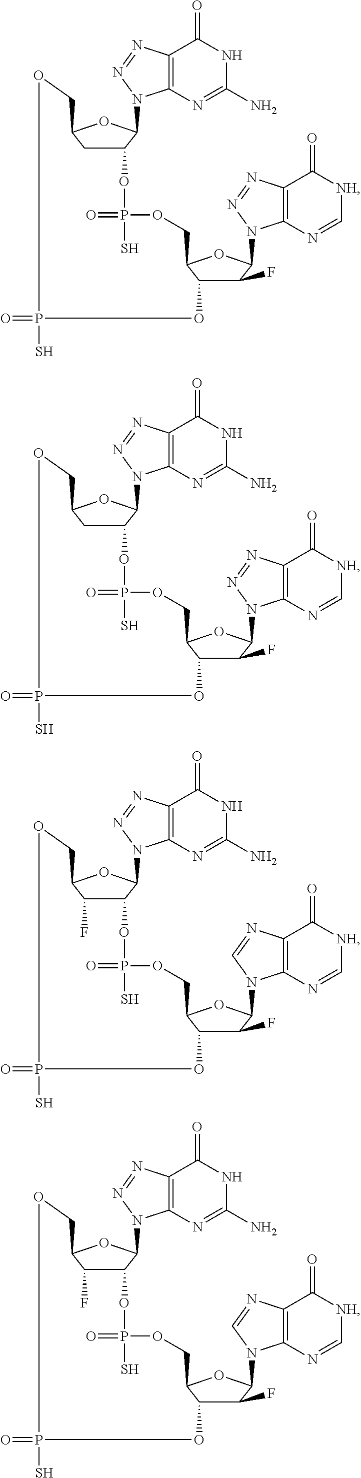 [SMILES: Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1 ; Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1 ; Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1 ; Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1]

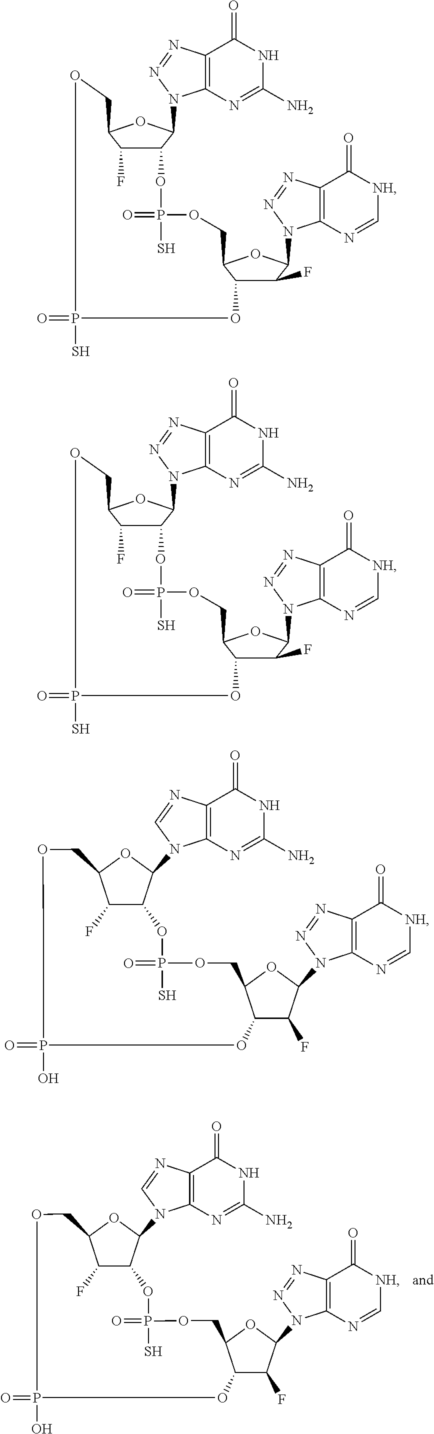

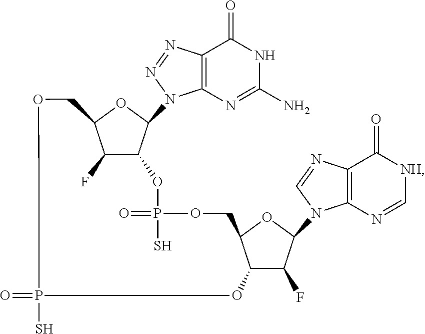 [SMILES: Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1]

and pharmaceutically acceptable salts thereof.

In particular embodiments, the cyclic dinucleotide STING agonist compound is selected from the group consisting of

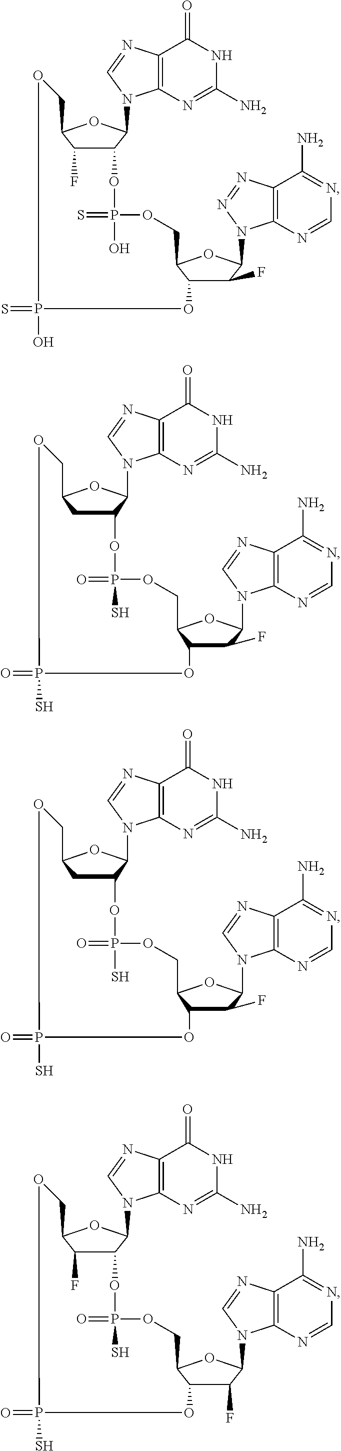

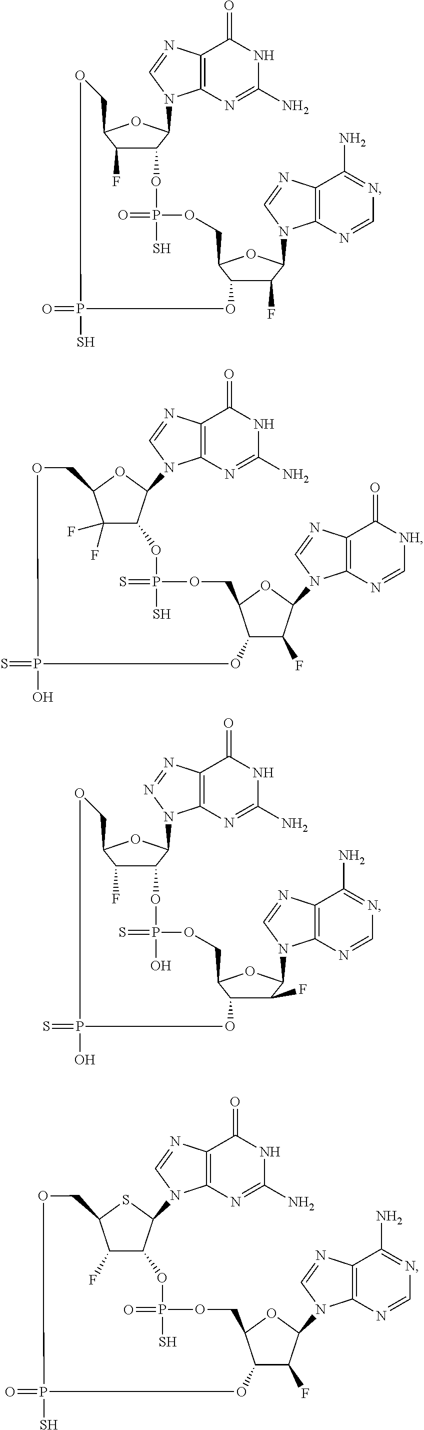 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(=S)(S)O[C@@H]2C3(F)F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

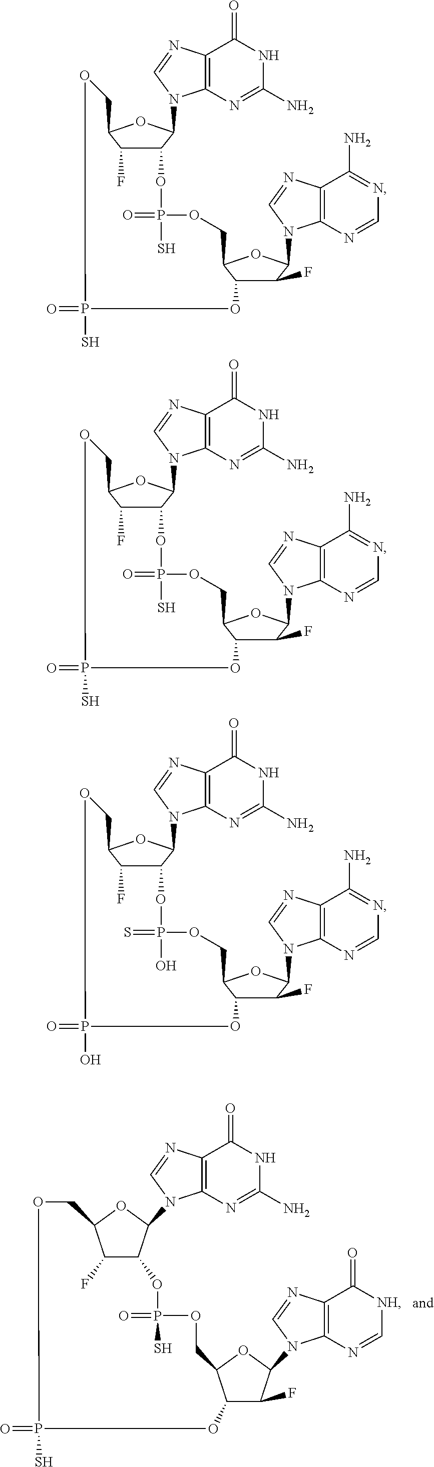

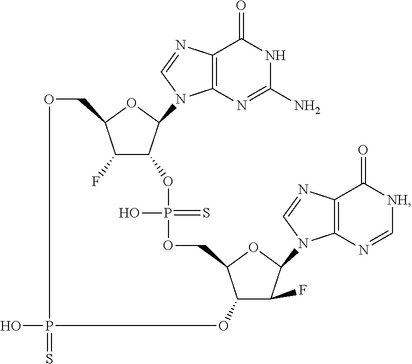 [SMILES: Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

and pharmaceutically acceptable salts thereof. In more particular embodiments, the cyclic dinucleotide STING agonist compound is selected from the group consisting of

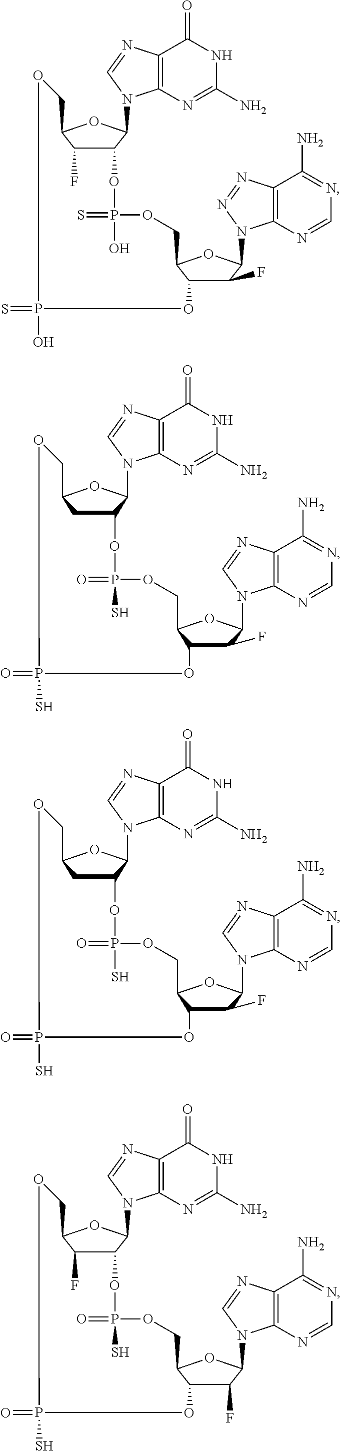

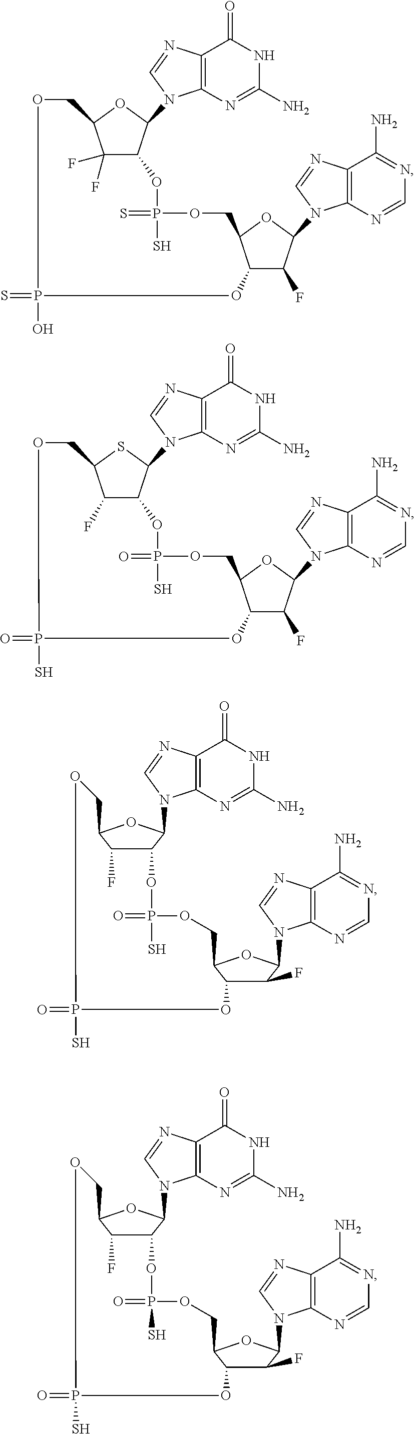 [SMILES: Nc1nc2c(ncn2[C@@H]2O[C@@H]3COP(O)(=S)O[C@H]4[C@H](F)[C@H](n5cnc6c(N)ncnc65)O[C@@H]4COP(=S)(S)O[C@@H]2C3(F)F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

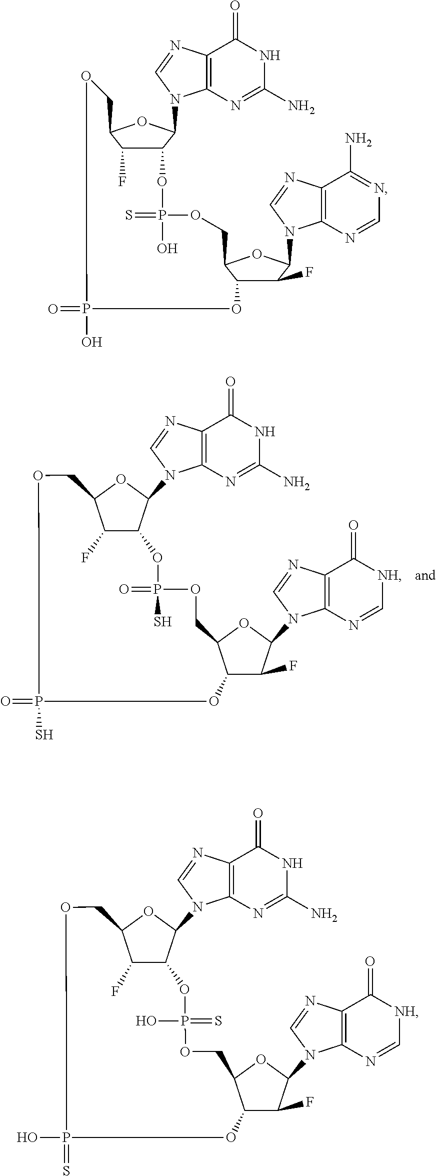 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2[C@@H]3F)c(=O)[nH]1 ; "and" ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

and pharmaceutically acceptable salts thereof. In still more particular embodiments, the cyclic dinucleotide STING agonist compound is selected from the group consisting of

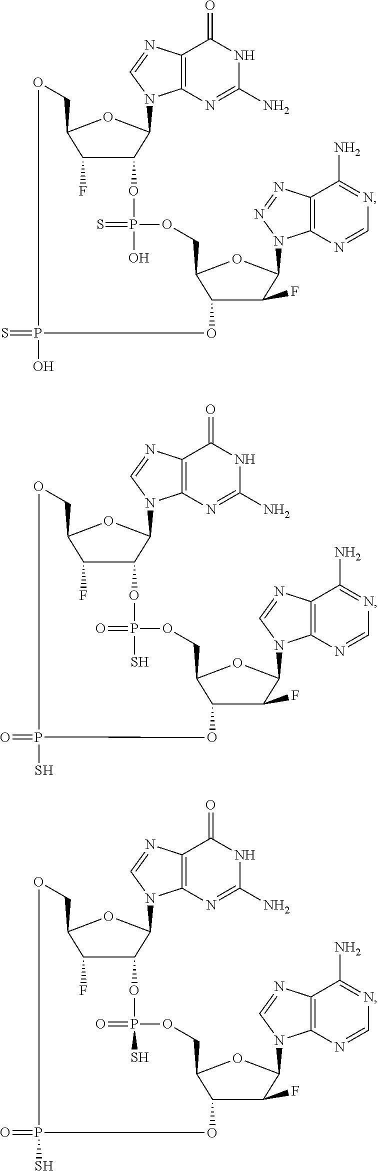 [SMILES: Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

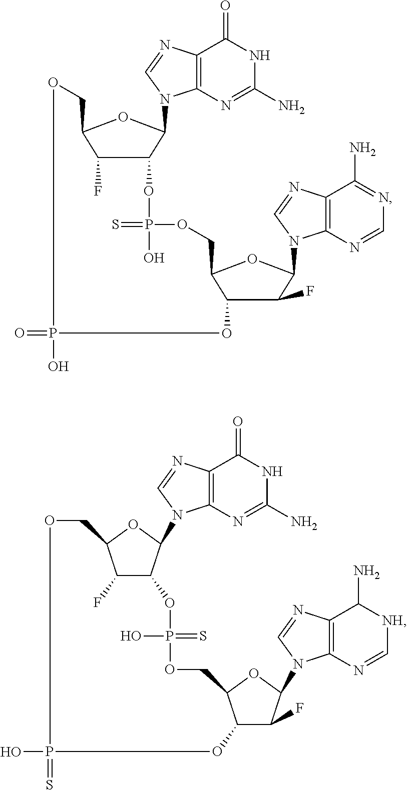 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c5N=CNC6N)O[C@@H]4COP(O)(=S)O[C@@H]2[C@@H]3F)c(=O)[nH]1]

and pharmaceutically acceptable salts thereof. In specific embodiments, the cyclic dinucleotide STING agonist compound is selected from the group consisting of

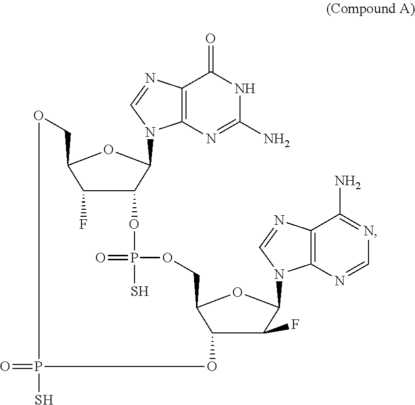 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

and pharmaceutically acceptable salts thereof. In more specific embodiments, the cyclic dinucleotide STING agonist compound is a pharmaceutically acceptable salt of

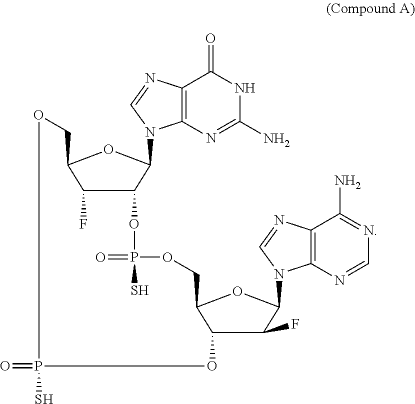 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

In further embodiments, the cyclic dinucleotide STING agonist compound is selected from the group consisting of

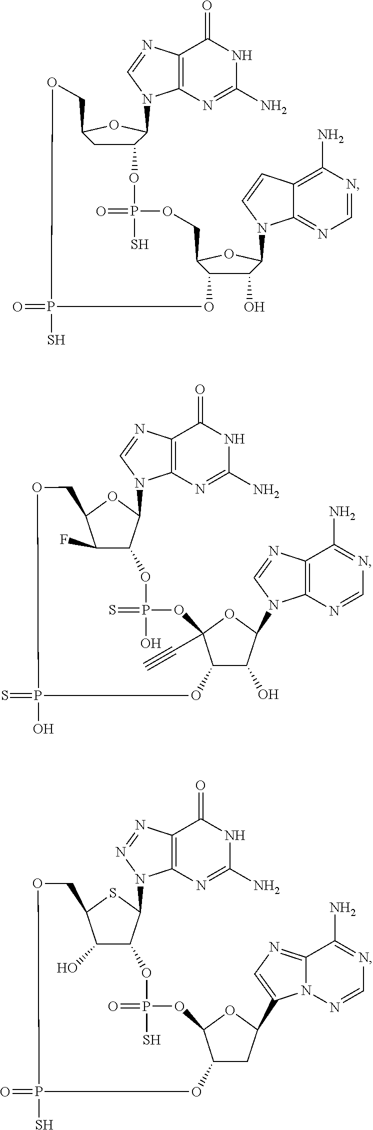 [SMILES: Nc1nc2c(ncn2[C@@H]2O[C@@H]3COP(=O)(S)O[C@H]4[C@@H](O)[C@H](n5ccc6c(N)ncnc65)O[C@@H]4COP(=O)(S)O[C@@H]2C3)c(=O)[nH]1 ; C#C[C@]12O[C@@H](n3cnc4c(N)ncnc43)[C@H](O)[C@@H]1OP(O)(=S)OC[C@H]1O[C@@H](n3cnc4c(=O)[nH]c(N)nc43)[C@H](OP(O)(=S)O2)[C@H]1F ; Nc1nc2c(nnn2[C@@H]2S[C@@H]3COP(=O)(S)O[C@H]4C[C@H](c5cnc6c(N)ncnn56)O[C@@H]4OP(=O)(S)O[C@@H]2[C@@H]3O)c(=O)[nH]1]

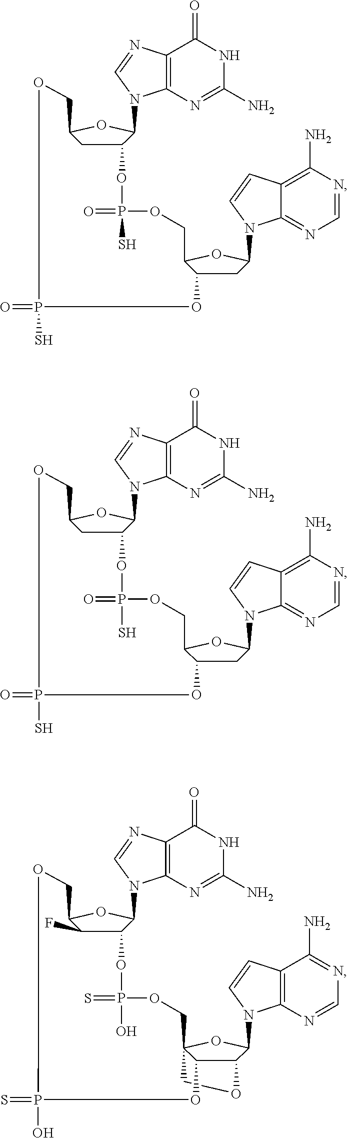 [SMILES: Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2ccc3c(N)ncnc32)c(=O)[nH]1]

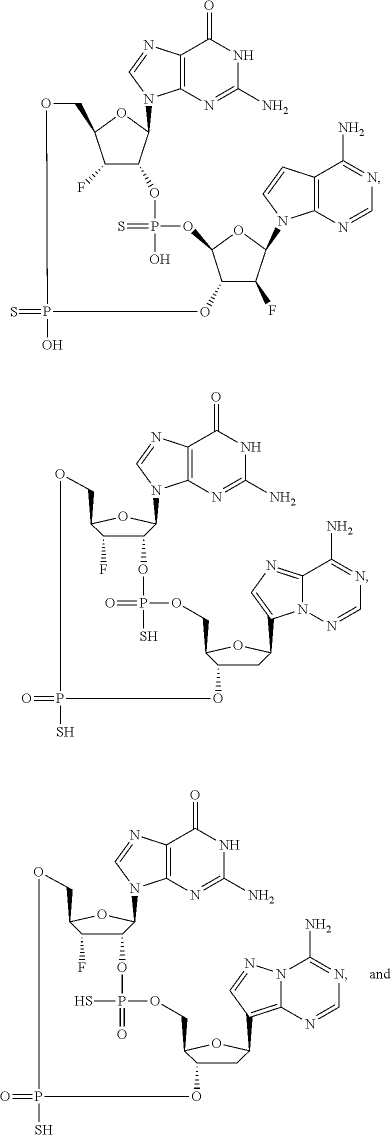 [SMILES: Nc1nc2c(ncn2[C@@H]2O[C@@H]3COP(O)(=S)O[C@@H]4[C@H](O[C@@H](n5ccc6c(N)ncnc65)[C@H]4F)OP(O)(=S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; "and"]

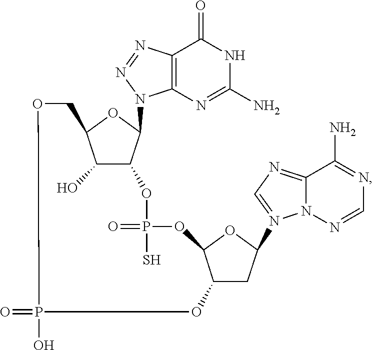

and pharmaceutically acceptable salts thereof. In more particular embodiments, the compound is selected from the group consisting of

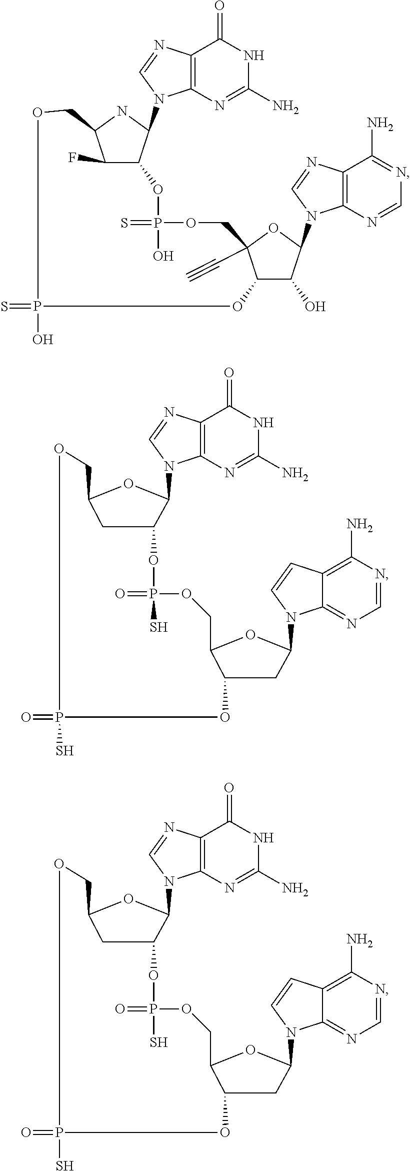 [SMILES: C#C[C@@]12COP(O)(=S)O[C@@H]3[C@@H](F)[C@@H](COP(O)(=S)O[C@H]1[C@@H](O)[C@H](n1cnc4c(N)ncnc41)O2)N[C@H]3n1cnc2c(=O)[nH]c(N)nc21 ; Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1]

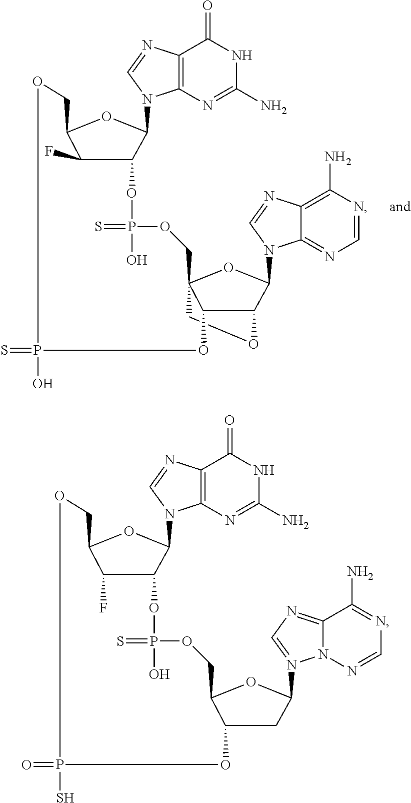

and pharmaceutically acceptable salts thereof. In still more particular embodiments, the cyclic dinucleotide STING agonist compound is selected from the group consisting of

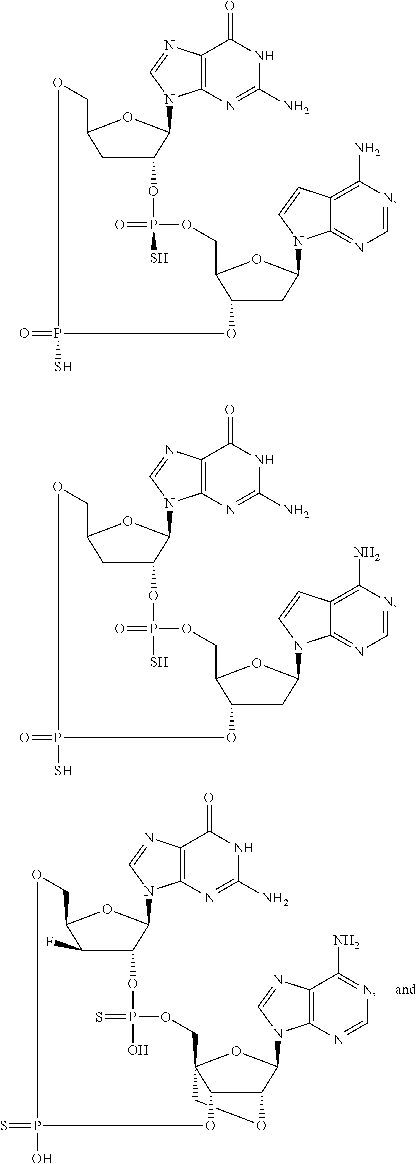 [SMILES: Nc1nc2c(ncn2[C@@H]2OC3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2OC3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1]

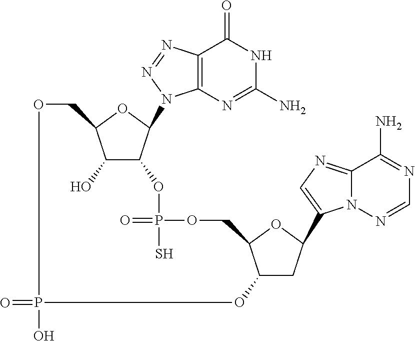 [SMILES: Nc1nc2c(nnn2[C@@H]2O[C@@H]3COP(=O)(O)O[C@H]4C[C@H](c5cnc6c(N)ncnn56)O[C@@H]4COP(=O)(S)O[C@@H]2[C@@H]3O)c(=O)[nH]1]

and pharmaceutically acceptable salts thereof. In specific embodiments, the cyclic dinucleotide STING agonist compound is selected from the group consisting of

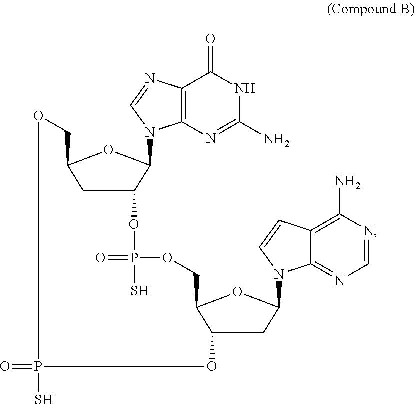 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1]

and pharmaceutically acceptable salts thereof.

In some embodiments, the cyclic dinucleotide STING agonist compound is present in the formulations in an amount of about 0.1 mg/ml to about 6.0 mg/ml. In further embodiments, the cyclic dinucleotide STING agonist compound is present in an amount of about 0.25 mg/ml to about 6.0 mg/ml, about 0.1 mg/ml to about 4.0 mg/ml, about 0.25 mg/ml to about 4.0 mg/ml, or about 0.54 mg/ml to about 4.0 mg/ml, or about 0.54 mg/ml.

Recitation or depiction of a specific compound in the claims (i.e., a species) without a specific stereoconfiguration designation, or with such a designation for less than all chiral centers, is intended to encompass the racemate, racemic mixtures, each individual enantiomer, a diastereoisomeric mixture and each individual diastereomer of the compound where such forms are possible due to the presence of one or more asymmetric centers.

### Methods of Preparing Compounds

The cyclic dinucleotide STING agonist compound useful in formulations of the present disclosure may be prepared according to the methods disclosed in PCT International Patent Application No. PCT/US2016/046444, which published as PCT International Patent Application Publication No. WO2017/027646, and U.S. patent application Ser. No. 15/234,182, which published as U.S. Patent Application Publication No. US2017/0044206, which are incorporated herein by reference in their entirety. In particular, several methods for preparing the compounds of general formula (I′), or pharmaceutically acceptable salts, hydrates, solvates, or prodrugs thereof, are described in the following Schemes. Starting materials and intermediates are purchased from commercial sources, made from known procedures, or are otherwise illustrated.

In some cases, the order of carrying out the steps of the reaction schemes may be varied to facilitate the reaction or to avoid unwanted reaction products.

### Method 1

One method for the preparation of the cyclic dinucleotide STING agonist compounds is detailed in Scheme 1. This procedure was adequately modified from the previously reported procedure for cyclic dinucleotide synthesis (Barbara L. Gaffney et al., One-Flask Syntheses of c-di-GMP and the [Rp,Rp] and [Rp,Sp] Thiophosphate Analogues, 12 ORG. LETT. 3269-3271 (2010)). The sequence starts with modified ribo-nucleoside with a nucleobase of which amino group was appropriately protected with an alkyl or phenyl carbonyl group, a phosphoramidite functionality at 2′-O position, and DMTr ether at 5′-O position. It was treated with aqueous TFA/pyridine and subsequently t-butylamine to convert the 2′-phosphoramidite moiety to an H-phosphonate. Then, the DMTr ether was removed under acidic conditions. The resulting 5′-hydroxyl group was reacted with 3′-phosphoramidites of a fully protected modified ribo-nucleoside to give a cyclized compound. It was immediately oxidized with t-butyl hydroperoxide. Then, the 5′-hydroxyl group of the second ribo-nucleoside was deprotected with dichloroacetic acid. Using 2-chloro-5,5-dimethyl-1,3,2-dioxaphosphinane 2-oxide as a coupling reagent, the H-phosphonate at 2′-O of the first ribo-nucleoside was reacted with 5′-OH of the second ribo-nucleoside to give a cyclic product. It was immediately oxidized with aqueous iodine. Treatment with t-butylamine and methylamine plus fluoride anion in case silyl protection was used provided the desired cyclic dinucleotide 1G.

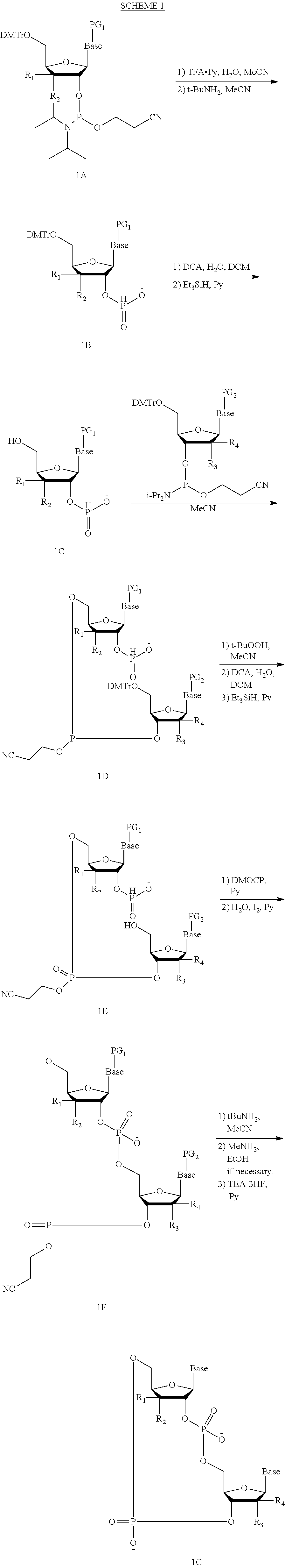

### Method 2

Another method for the preparation of the cyclic dinucleotide STING agonist compounds useful in formulations of this disclosure is detailed in Scheme 2. This procedure was modified from Scheme 1. The sequence starts with modified ribo-nucleoside with a nucleobase of which amino group was appropriately protected with an alkyl or phenyl carbonyl group, a phosphoramidite functionality at 2′-O position, and DMTr ether at 5′-O position. It was treated with aqueous TFA/pyridine condition and subsequently t-butylamine to convert the 2′-phosphoramidite moiety to an H-phosphonate. Then, the DMTr ether was removed under acidic conditions. The resulting 5′-hydroxyl group was reacted with 3′-phosphoramidites of fully protected second modified ribo-nucleoside to give a cyclized compound. It was immediately thioated with (E)-N,N-dimethyl-N′-(3-thioxo-3H-1,2,4-dithiazol-5-yl) formimidamide. Then, the 5′-hydroxyl group of the second ribo-nucleoside was deprotected with dichloroacetic acid. Using 2-chloro-5,5-dimethyl-1,3,2-dioxaphosphinane 2-oxide as a coupling reagent, the H-phosphonate at 2′-O of the first ribo-nucleoside was reacted with 5′-OH of the second ribo-nucleoside to give a cyclic product. It was immediately thioated with 3H-benzo[c][1,2]dithiol-3-one. Treatment with t-butylamine and methylamine plus fluoride anion in case silyl protection was used provided the desired cyclic dinucleotide diphosphorothioate 2G.

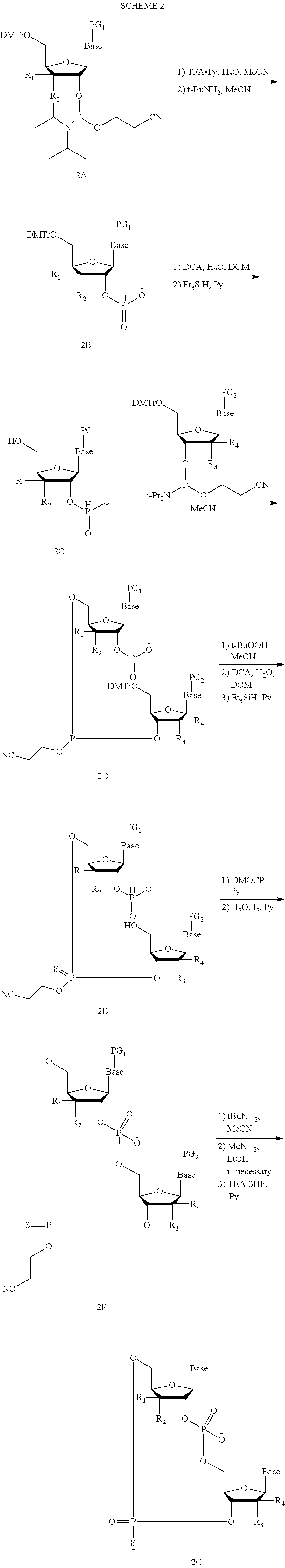

### Compound A

A method for preparing Compound A, as well as its diastereomers, is disclosed in PCT International Patent Application No. PCT/US2016/046444, which published as PCT International Patent Application Publication No. WO2017/027646, and U.S. patent application Ser. No. 15/234,182, which published as U.S. Patent Application Publication No. US2017/0044206, which are incorporated herein by reference in their entirety, as Examples 244, 245, 246, and 247, 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one (Diastereomers 1-3) and 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one (Diastereomer 4), respectfully.

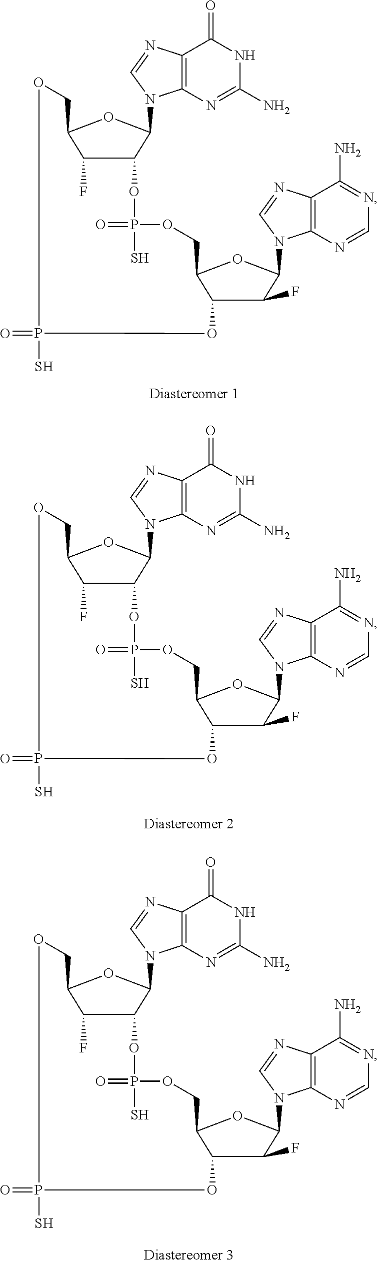 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

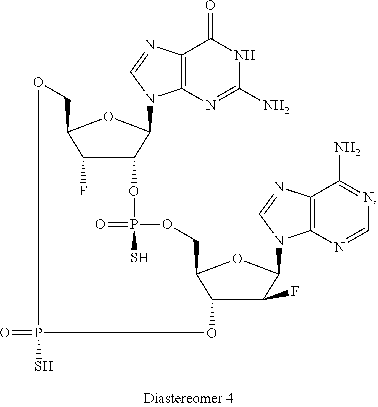 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

The compounds were prepared by the following process, as set forth in WO2017/027646 and US2017/0044206.

### Step 1: (2R,3S,4R,5R)-5-((((((2R,3R,4S,5R)-5-(6-benzamido-9H-purin-9-yl)-2-((bis(4-methoxyphenyl)(phenyl)methoxy)methyl)-4-fluorotetrahydrofuran-3-yl)oxy)(2-cyanoethoxy)phosphanyl)oxy)methyl)-4-fluoro-2-(2-isobutyramido-6-oxo-1,6-dihydro-9H-purin-9-yl)tetrahydrofuran-3-yl hydrogen phosphonate

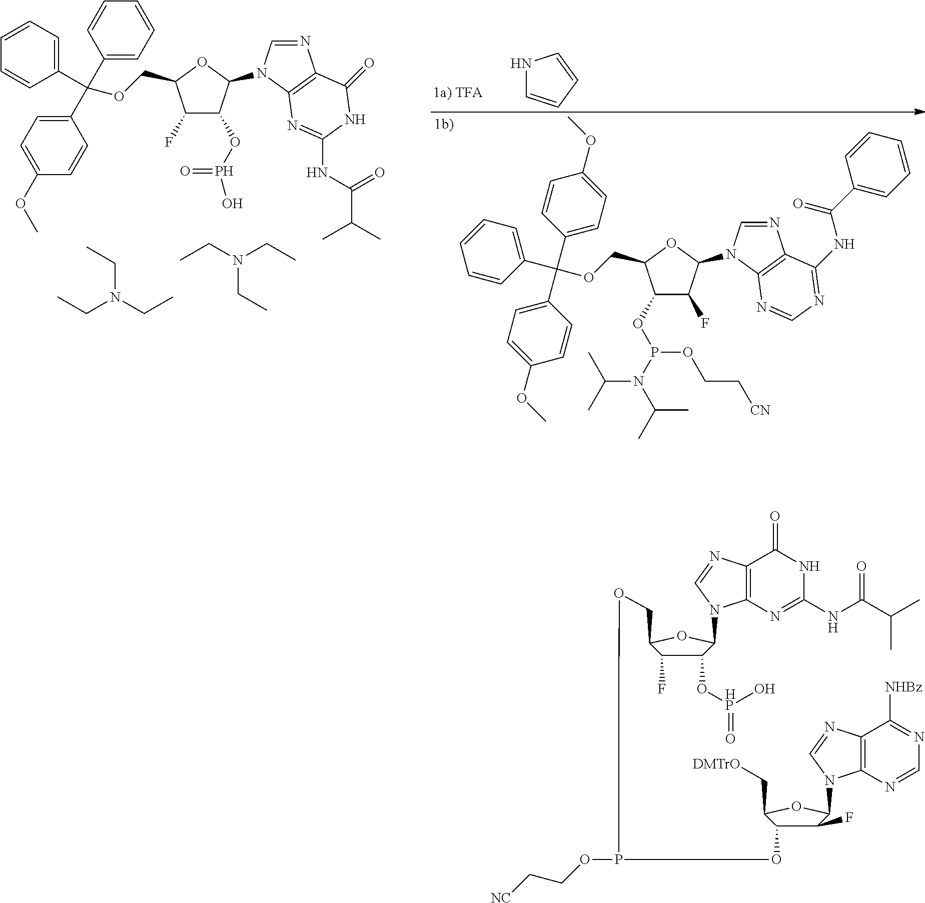

Pyrrole (0.087 mL, 1.2 mmol) was added to a solution of (2R,3S,4R,5R)-5-((bis(4-methoxyphenyl)(phenyl)methoxy)methyl)-4-fluoro-2-(2-isobutyramido-6-oxo-1,6-dihydro-9H-purin-9-yl)tetrahydrofuran-3-yl hydrogen phosphonate triethylamine salt (1:2) (0.34 g, 0.41 mmol) in acetonitrile (3.0 mL) under an argon atmosphere at 0° C. After 5 min, TFA (0.096 mL, 0.14 mmol) was added, and the reaction mixture was stirred at 0° C. for 30 min. Pyridine (0.13 mL, 1.7 mmol) was added drop wise at 0° C. The reaction mixture was then stirred for 10 min at 0° C. At that time, a mixture of (2R,3R,4S,5R)-5-(6-benzamido-9H-purin-9-yl)-2-((bis(4-methoxyphenyl)(phenyl)methoxy)methyl)-4-fluorotetrahydrofuran-3-yl (2-cyanoethyl) diisopropylphosphoramidite (0.48 g, 0.55 mmol) in acetonitrile (3.0 mL) was added drop wise over 5 min to the reaction mixture under an argon atmosphere at 0° C. The reaction mixture was stirred at 0° C. for 20 min and immediately used in the next step without further manipulation.

### Step 2: (2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy)(2-cyanoethoxy)phosphorothioyl)oxy)methyl)-4-fluoro-2-(2-isobutyramido-6-oxo-1,6-dihydro-9H-purin-9-yl)tetrahydrofuran-3-yl hydrogen phosphonate

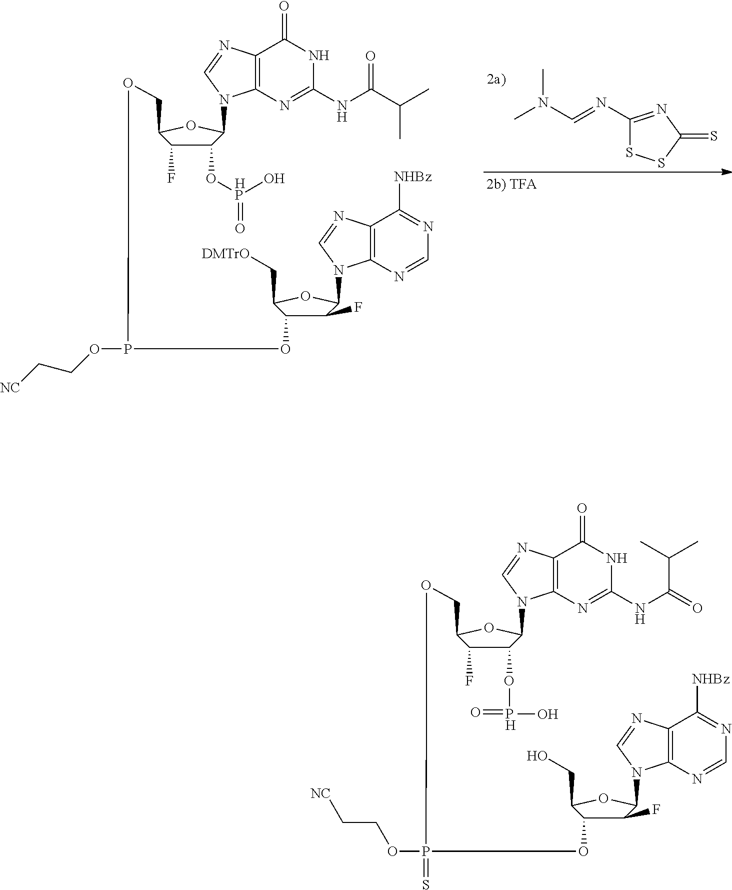

To the crude reaction mixture from Step 1 was added (E)-N,N-dimethyl-N′-(3-thioxo-3H-1,2,4-dithiazol-5-yl)formimidamide (0.10 g, 0.50 mmol) under an argon atmosphere at 0° C. The reaction mixture was stirred for 45 minutes at 0° C. At that time, 1-propanol (0.31 mL, 4.13 mmol) was added to the reaction mixture under an argon atmosphere at 0° C. The reaction mixture was then allowed to warm to ambient temperature and stirred for 10 min. TFA (0.32 mL, 4.1 mmol) was added to the reaction mixture, and the reaction mixture was stirred for 30 min at ambient temperature. Pyridine (0.37 mL, 4.6 mmol) was added at ambient temperature, and the reaction mixture was stirred for 10 min. The reaction mixture was concentrated under reduced pressure to approximately one-half volume. The mixture was then diluted with isopropyl acetate (20 mL) and stirred for 30 min at ambient temperature. The resulting suspension was filtered. The collected solids were dried overnight under high vacuum to afford (2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy)(2-cyanoethoxy)phosphorothioyl)oxy)methyl)-4-fluoro-2-(2-isobutyramido-6-oxo-1,6-dihydro-9H-purin-9-yl)tetrahydrofuran-3-yl hydrogen phosphonate. LCMS (ES, m/z): 922 [M−H]⁻.

### Step 3: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one

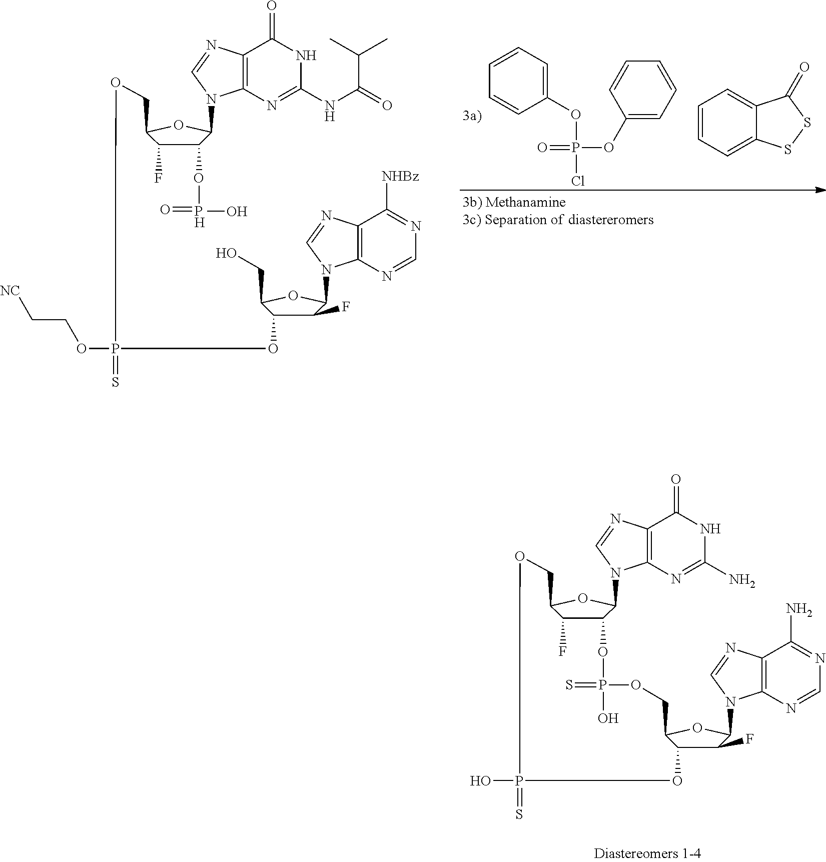

(2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy)(2-cyanoethoxy)phosphorothioyl)oxy)methyl)-4-fluoro-2-(2-isobutyramido-6-oxo-1,6-dihydro-9H-purin-9-yl)tetrahydrofuran-3-yl hydrogen phosphonate (0.30 g, 0.33 mmol) was azeotroped with dry pyridine (2×10 mL) and then dried under high vacuum for 1 h. In a separate flask, diphenyl phosphorochloridate (0.34 mL, 1.6 mmol) was added to a mixture of acetonitrile (15 mL) and pyridine (1.0 mL). The resulting solution was then cooled to −20° C. To this mixture was added drop wise over a period of 5 min a mixture of (2R,3S,4R,5R)-5-((((((2R,3R,4S,5R)-5-(6-benzamido-9H-purin-9-yl)-4-fluoro-2-(hydroxy methyl)tetrahydrofuran-3-yl)oxy)(2-cyanoethoxy)phosphorothioyl)oxy)-methyl)-4-fluoro-2-(2-isobutyramido-6-oxo-1,6-dihydro-9H-purin-9-yl)tetrahydrofuran-3-yl hydrogen phosphonate (0.30 g, 0.33 mmol) in pyridine (4.0 mL) at −20° C. The reaction mixture was then stirred at −20° C. for 15 min post-addition. 3H-benzo[c][1,2]dithiol-3-one (0.066 g, 0.39 mmol) and water (0.12 mL, 6.5 mmol) were then added to the reaction mixture at −20° C. The reaction mixture was allowed to gradually warm to ambient temperature. The reaction mixture was stirred for 30 min at ambient temperature. The reaction mixture was then concentrated under reduced pressure to approximately one quarter volume. The reaction mixture was cooled to 0° C., and methanamine (33% in ethanol) (2.63 mL, 24 mmol) was added drop wise. After the addition was complete, the reaction mixture was allowed to warm to ambient temperature. The reaction mixture was stirred at ambient temperature for 18 h. The reaction mixture was concentrated under reduced pressure to afford the crude product residue. The crude product residue was azeotroped (3×30 mL ethanol) to afford the crude product. This material was dissolved in water (5 mL) and acetonitrile (1 mL). The resulting mixture was purified by mass-directed reverse phase HPLC (Waters Sunfire 19×250 mm, UV 215/254 nm, fraction trigger by SIM negative MS monitoring mass 709; mobile phase=100 mM triethylammonium acetate in water/acetonitrile gradient, 2-30% acetonitrile over 40 min) to afford the 4 diastereomers of 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxa-diphosphacyclotetradecin-7-yl]-1,9-dihydro-6H-purin-6-one.

Diastereomer 1: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one: T_{R}=17.7 min. LCMS (ES, m/z): 709 [M−H]⁻.

Diastereomer 2: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one: T_{R}=21.9 min. LCMS (ES, m/z): 709 [M−H]⁻. ¹H NMR (500 MHz, DMSO-d₆) δ 8.32 (s, 1H), 8.21-8.09 (m, 2H), 7.46-7.29 (m, 2H), 6.59-6.43 (m, 2H), 6.40-6.29 (m, 1H), 5.88 (d, J=8.8 Hz, 1H), 5.49-5.19 (m, 4H), 4.45-4.32 (m, 2H), 4.10-3.93 (m, 2H), 3.94-3.82 (m, 1H), 3.80-3.68 (m, 1H).

Diastereomer 3: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one: T_{R}=23.8 min. LCMS (ES, m/z): 709 [M−H]⁻. ¹H NMR (500 MHz, DMSO-d₆) δ 8.18-8.08 (m, 3H), 7.41-7.33 (m, 2H), 6.59-6.47 (m, 2H), 6.37-6.27 (m, 1H), 5.84 (d, J=8.7 Hz, 1H), 5.52-5.26 (m, 2H), 5.21-5.11 (m, 1H), 4.46-4.35 (m, 2H), 4.19-4.02 (m, 2H), 3.83-3.65 (m, 2H).

Diastereomer 4, Compound A: 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidoocta-hydro-12H-5,8-methanofuro[3,2-l][1,3,6,9,11,2,10]pentaoxadiphosphacyclotetradecin-7-yl]-1,9-dihydro-6H-purin-6-one: T_{R}=26.4 min. LCMS (ES, m/z): 709 [M−H]⁻. ¹H NMR (500 MHz, DMSO-d₆) δ 8.19-8.07 (m, 3H), 7.41-7.32 (m, 2H), 6.70-6.50 (m, 2H), 6.40-6.29 (m, 1H), 5.85 (d, J=8.7 Hz, 1H), 5.33-5.25 (m, 2H), 5.23-5.12 (m, 1H), 4.48-4.35 (m, 1H), 4.33-4.24 (m, 1H), 4.09-3.93 (m, 2H), 3.92-3.81 (m, 1H), 3.83-3.70 (m, 1H).

### Pharmaceutically Acceptable Aqueous Carriers

The pharmaceutical formulations described herein contain a pharmaceutically acceptable aqueous carrier. In embodiments, the pharmaceutically acceptable aqueous carrier is selected from the group consisting of water, about 30% captisol in water, about 30% hydroxypropyl beta-cyclodextrin in water, about 60% propylene glycol in water, about 10% polysorbate 80 in water, and about 10% dimethyl sulfoxide in water. In particular embodiments, the pharmaceutically acceptable aqueous carrier is water.

### Pharmaceutically Acceptable Tonicity Modifiers

The pharmaceutical formulations described herein contain a pharmaceutically acceptable tonicity modifier. In embodiments, the pharmaceutically acceptable tonicity modifier is selected from the group consisting of salts, sugar alcohols, polyols, and disaccharides. In specific embodiments, the pharmaceutically acceptable tonicity modifier is selected from the group consisting of mannitol, sodium chloride, glycerol, sucrose, and trehalose. In more specific embodiments, the the pharmaceutically acceptable tonicity modifier is selected from the group consisting of mannitol, sodium chloride, and sucrose. In even more specific embodiments, the pharmaceutically acceptable tonicity modifier is mannitol.

In some embodiments, the pharmaceutically acceptable tonicity modifier is present in the formulations in an amount of about 30 mg/ml to about 70 mg/ml. In further embodiments, the pharmaceutically acceptable tonicity modifier is present in an amount of about 20 mg/ml to about 60 mg/ml, or about 30 mg/ml to about 50 mg/ml, or about 30 mg/ml to about 40 mg/ml, or about 40 mg/ml, or about 34 mg/ml. In some embodiments, the pharmaceutically acceptable tonicity modifier is present in the formulations in a concentration of about 165 mM to about 385 mM. In further embodiments, the pharmaceutically acceptable tonicity modifier is present in a concentration of about 165 mM to about 274 mM, or 165 mM to about 220 mM, or about 220 mM, or about 187 mM.

### Pharmaceutically Acceptable Buffers

The pharmaceutical formulations described herein contain a buffer. In embodiments, pharmaceutically acceptable buffer has a pKa of between about 5.5 and about 8.5. In embodiments, the pharmaceutically acceptable buffer is selected from the group consisting of histidine, tris(hydroxymethyl)aminomethane (TRIS), sodium citrate, and sodium phosphate. In specific embodiments, the pharmaceutically acceptable buffer is histidine. In further specific embodiments, the pharmaceutically acceptable buffer is L-histidine.

In some embodiments, the pharmaceutically acceptable buffer is present in the formulations in an amount of about 5 mg/ml to about 10 mg/ml. In further embodiments, the pharmaceutically acceptable buffer is present in an amount of about 6 mg/ml to about 8 mg/ml, or about 7.75 mg/ml or about 7.5 mg/ml. In some embodiments, the pharmaceutically acceptable buffer is present in the formulations in a concentration of about 10 mM to about 65 mM. In further embodiments, the pharmaceutically acceptable buffer is present in a concentration of about 25 mM to about 65 mM, about 30 mM to about 50 mM, or about 50 mM.

In embodiments, the formulations described herein have a pH of from about 6 to about 7.5. In particular embodiments, the pharmaceutical formulation has a pH of from about 6 to about 7. In specific such embodiments, the pharmaceutical formulation has a pH of from about 6.3 to about 6.7, such as 6.5.

When a range of pH values is recited, such as “a pH between pH 5.5 and 6.0,” the range is intended to be inclusive of the recited values. The pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

### Pharmaceutically Acceptable Antioxidants

The pharmaceutical formulations described herein contain a pharmaceutically acceptable antioxidant. In embodiments, the pharmaceutically acceptable antioxidant is selected from the group consisting of L-methionine, sodium metabisulfite, thiogylcerol, cysteine, and glutathione. In specific embodiments, the pharmaceutically acceptable antioxidant is methionine. In embodiments, the pharmaceutically acceptable antioxidant is methionine, or a pharmaceutically acceptable salt thereof. In further embodiments, the pharmaceutically acceptable antioxidant is methionine. In still further embodiments, the pharmaceutically acceptable antioxidant is L-methionine. In other embodiments, the pharmaceutically acceptable antioxidant is L-methionine HCl.

In some embodiments, the pharmaceutically acceptable antioxidant is present in the formulations in an amount of about 0.15 mg/ml to about 1.0 mg/ml, such as about 0.15 mg/ml to about 1.0 mg/ml, or about 0.373 mg/ml, or about 0.500 mg/ml, or about 0.750 mg/ml. In some embodiments, the pharmaceutically acceptable antioxidant is present in the formulations in a concentration of about 3 mM to about 7 mM. In further embodiments, the pharmaceutically acceptable antioxidant is present in a concentration of about 1 mM to about 6.8 mM, or about 1 mM to about 6.8 mM, or about 1 mM, or about 2.5 mM, or about 5 mM.

### Pharmaceutically Acceptable Metal Chelators

In embodiments, the formulations contain a pharmaceutically acceptable metal chelator, which may be diethylenetriaminepentaacetic acid (DTPA) or edetate disodium dehydrate (EDTA) or any other suitable metal chelator. In specific embodiments, the metal chelator is EDTA.

In some embodiments, the pharmaceutically acceptable metal chelator is present in the formulations in an amount of about 0.01 mg/ml to about 0.04 mg/ml. In further embodiments, the pharmaceutically acceptable metal chelator is present in an amount of about 0.01 mg/ml to about 0.03 mg/ml, such as about 0.0175 mg/ml. In some embodiments, the pharmaceutically acceptable metal chelator is present in the formulations in a concentration of about 0.03 mM to about 0.11 mM. In further embodiments, the pharmaceutically acceptable metal chelator is present in a concentration of about 0.03 mM to about 0.08 mM, such as about 0.05 mM.

### Formulations

Embodiments of the formulations as disclosed herein are directed to pharmaceutical formulations comprising (a) a compound selected from the group consisting of compounds of formula (I′):

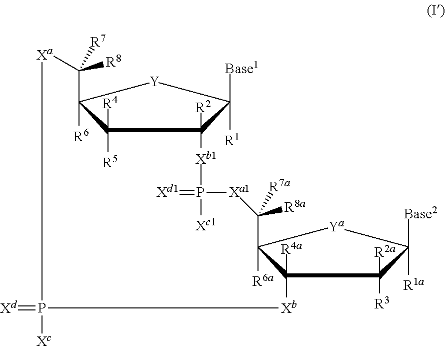

or a pharmaceutically acceptable salt, hydrate, solvate, or prodrug thereof, wherein Base¹ and Base² are each independently selected from the group consisting of

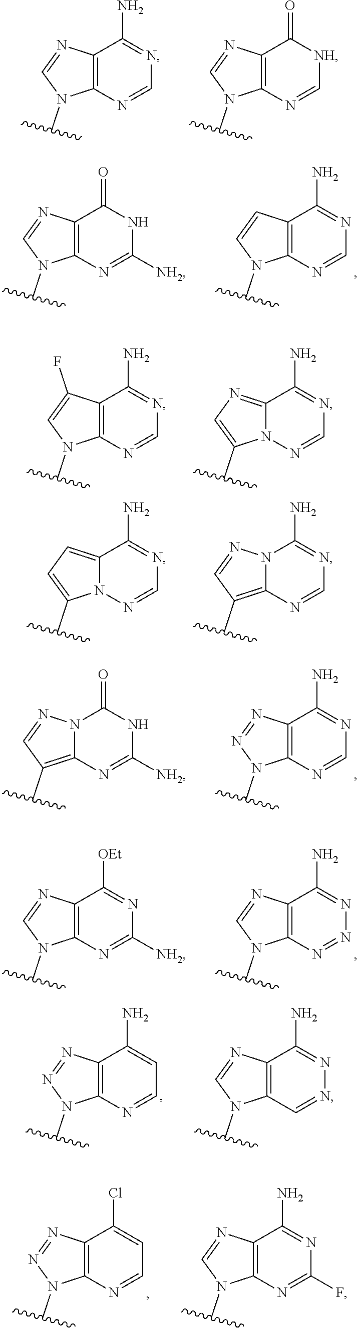

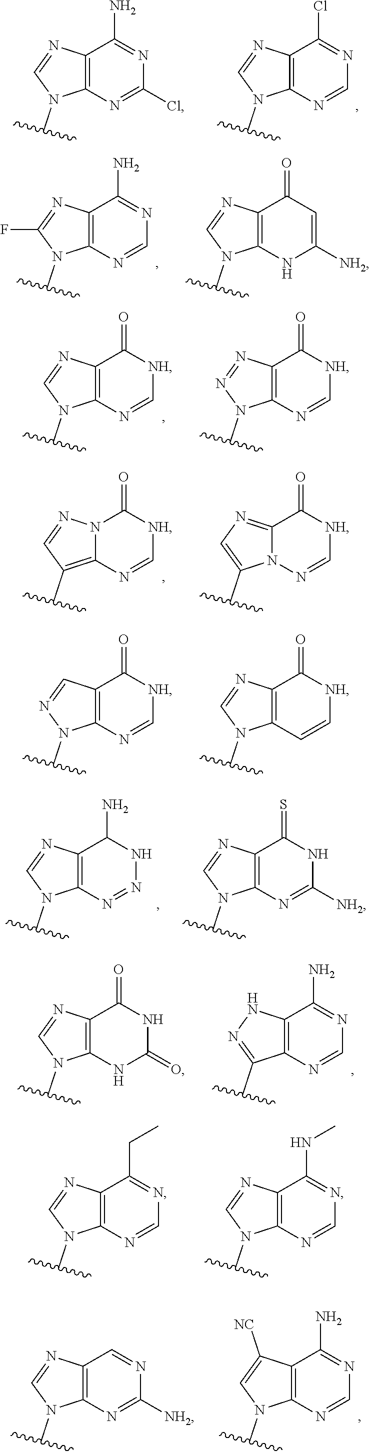

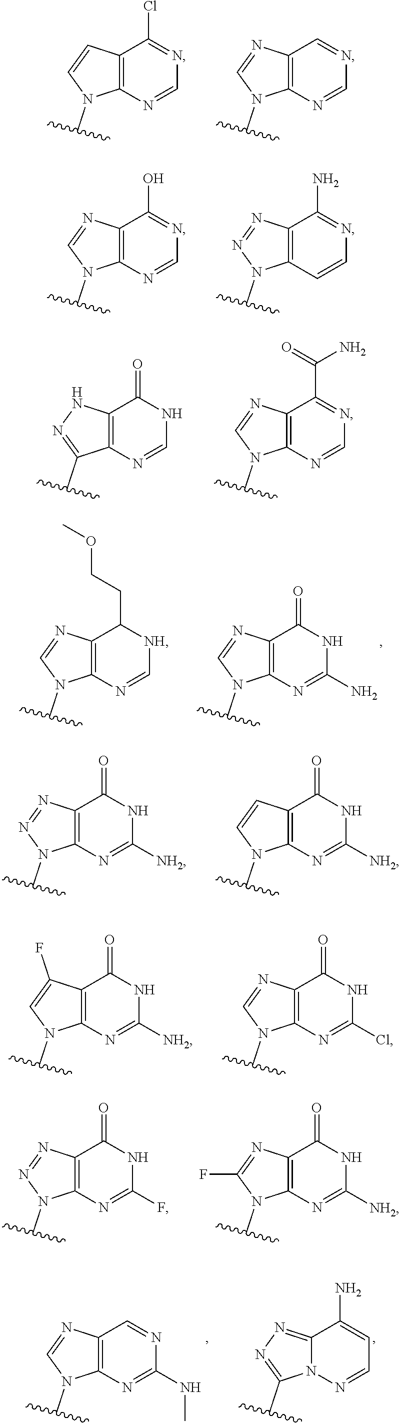

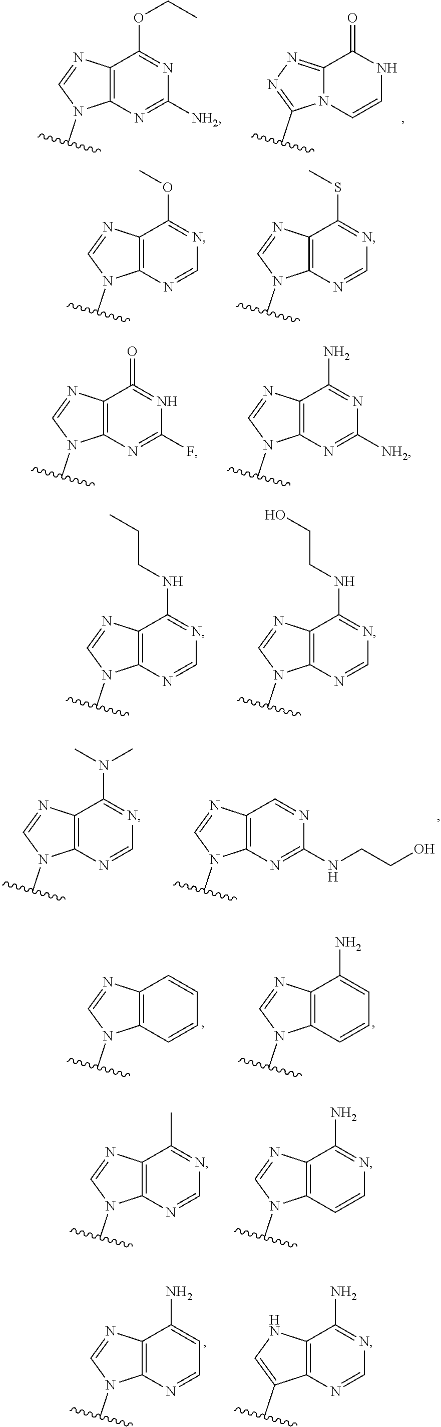

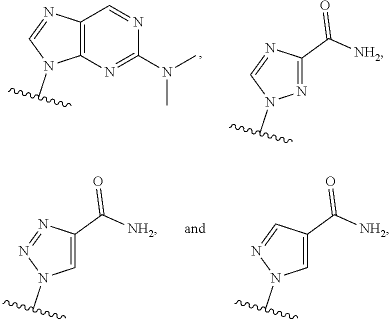 [SMILES: *n1cnc2cnc(N(C)C)nc21 ; *n1cnc(C(N)=O)n1 ; *n1cc(C(N)=O)nn1 ; "and" ; *n1cc(C(N)=O)cn1]

where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each independently selected from the group consisting of O, and S; X^{b} and X^{b1} are each independently selected from the group consisting of O, and S; X^{c} and X^{c1} are each independently selected from the group consisting of OR⁹, SR⁹, and NR⁹R⁹; X^{d} and X^{d1} are each independently selected from the group consisting of O and S; R¹ and R^{1a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R¹ and R^{1a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R² and R^{2a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R² and R^{2a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R³ is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁴ and R^{4a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁴ and R^{4a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁵ is selected from the group consisting of H, F, Cl, Br, I, OH, CN, NH₂, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁵ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, NR⁹R⁹, and N₃; R⁶ and R^{6a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁶ and R^{6a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁷ and R^{7a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁷ and R^{7a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁸ and R^{8a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁸ and R^{8a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; each R⁹ is independently selected from the group consisting of H, C₁-C₂₀ alkyl,

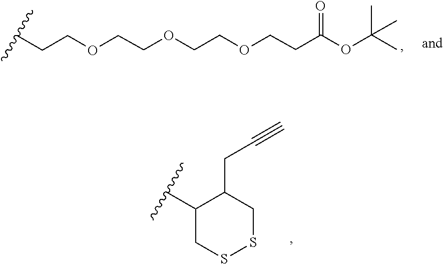

where each R⁹ C₁-C₂₀ alkyl is optionally substituted by 0 to 3 substituents independently selected from the group consisting of OH, —O—C₁-C₂₀ alkyl, —S—C(O)C₁-C₆ alkyl, and C(O)OC₁-C₆ alkyl; optionally R^{1a} and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{1a} and R³ are connected to form —O—C₁-C₆ alkylene, —O—C₂-C₆ alkenylene, or —O—C₂-C₆ alkynylene, said O is bound at the R³ position; optionally R^{2a} and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{2a} and R³ are connected to form —O—C₁-C₆ alkylene, —O—C₂-C₆ alkenylene, or —O—C₂-C₆ alkynylene, said O is bound at the R³ position; optionally R³ and R^{6a} are connected to form —O—C₁-C₆ alkylene, —O—C₂-C₆ alkenylene, or —O—C₂-C₆ alkynylene, such that where R³ and R^{6a} are connected to form —O—C₁-C₆ alkylene, —O—C₂-C₆ alkenylene, or —O—C₂-C₆ alkynylene, said O is bound at the R³ position; optionally R⁴ and R⁵ are connected to form are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R⁴ and R⁵ are connected to form —O—C₁-C₆ alkylene, —O—C₂-C₆ alkenylene, or —O—C₂-C₆ alkynylene, said O is bound at the R⁵ position; optionally R⁵ and R⁶ are connected to form —O—C₁-C₆ alkylene, —O—C₂-C₆ alkenylene, or —O—C₂-C₆ alkynylene, such that where R⁵ and R⁶ are connected to form —O—C₁-C₆ alkylene, —O—C₂-C₆ alkenylene, or —O—C₂-C₆ alkynylene, said O is bound at the R⁵ position; optionally R⁷ and R⁸ are connected to form C₁-C₆ alkylene, C₂-C₆ alkenylene, or C₂-C₆ alkynylene; and optionally R^{7a} and R^{8a} are connected to form C₁-C₆ alkylene, C₂-C₆ alkenylene, or C₂-C₆ alkynylene; and providing that when Y and Y^{a} are each O, X^{a} and X^{a1} are each O, X^{b} and X^{b1} are each O, and X^{b} and X^{b1} are each OH or SH, X^{d} and X^{d1} are each O, R¹ and R^{1a} are each H, R² is H, R⁶ and R^{6a} are each H, R⁷ and R^{7a} are each H, R⁸ and R^{8a} are each H, and Base¹ and Base² are each selected from the group consisting of

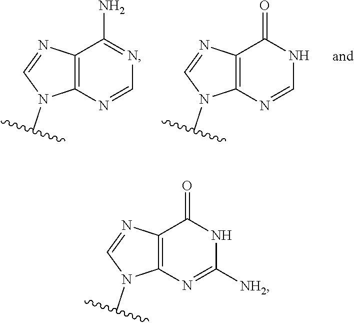 [SMILES: *n1cnc2c(N)ncnc21 ; *n1cnc2c(=O)[nH]cnc21 ; *n1cnc2c(=O)[nH]c(N)nc21]

R⁵ and R³ are not both selected from the group consisting of H, F and OH; (b) a pharmaceutically acceptable aqueous carrier; (c) one or more pharmaceutically acceptable tonicity modifier, (d) one or more pharmaceutically acceptable buffering agent, (e) one or more pharmaceutically acceptable antioxidant, and (f) one or more pharmaceutically acceptable metal chelator.

In aspects of these embodiments, the compound is selected from the group consisting of

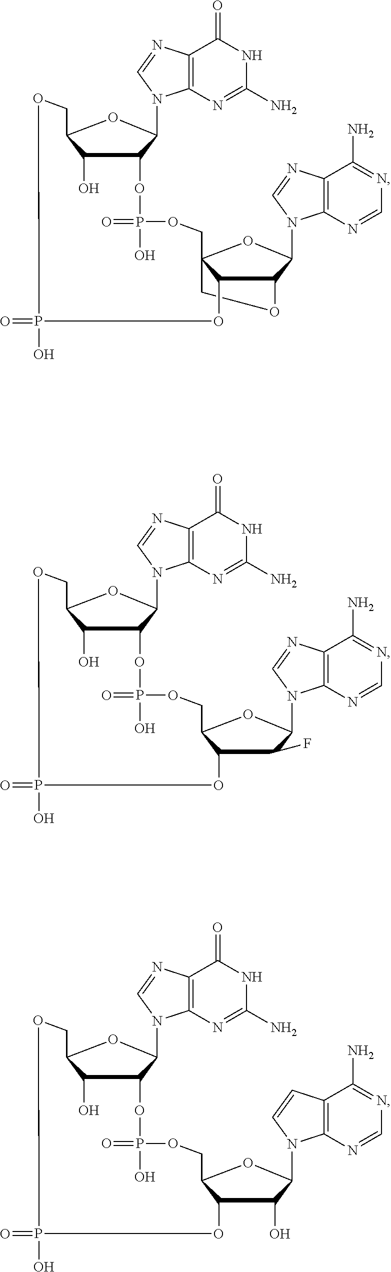

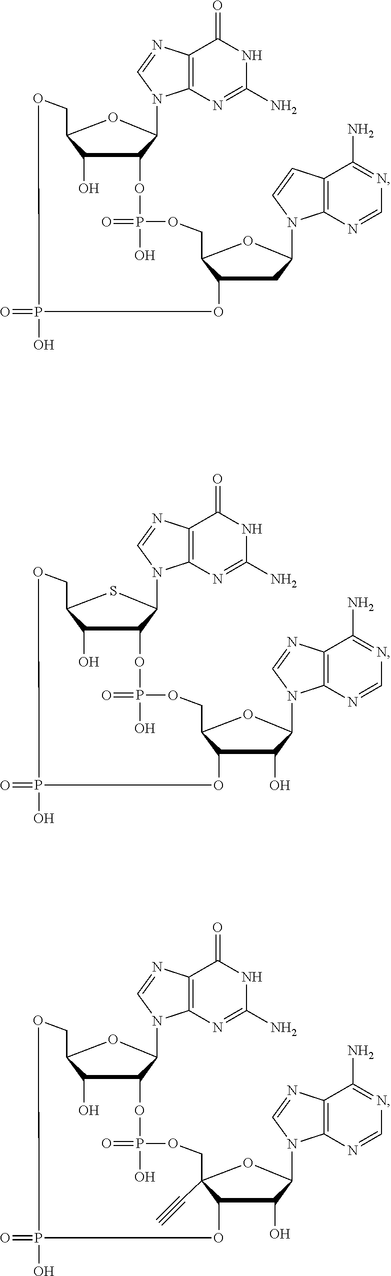

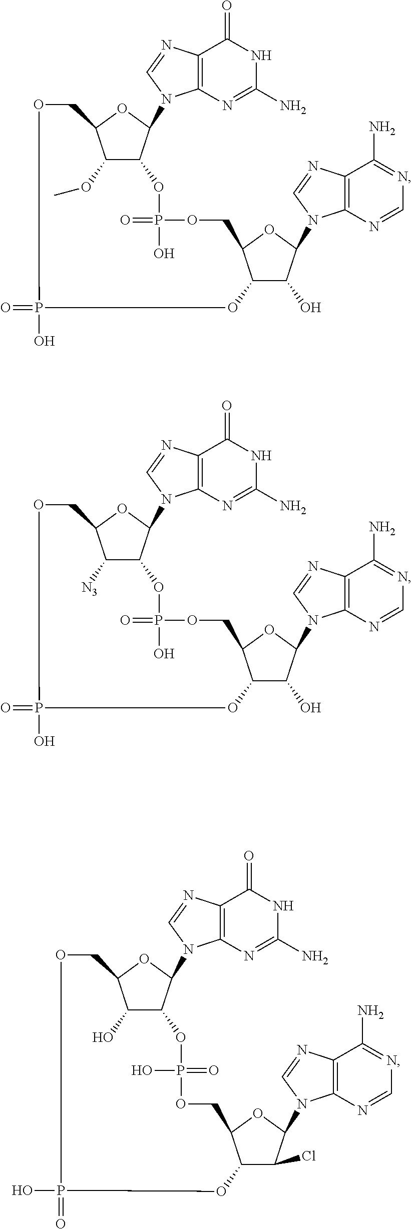 [SMILES: CO[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; [N-]=[N+]=N[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1nc2c(ncn2[C@@H]2O[C@@H]3COP(=O)(O)O[C@H]4[C@H](Cl)[C@H](n5cnc6c(N)ncnc65)O[C@@H]4COP(=O)(O)O[C@@H]2[C@@H]3O)c(=O)[nH]1]

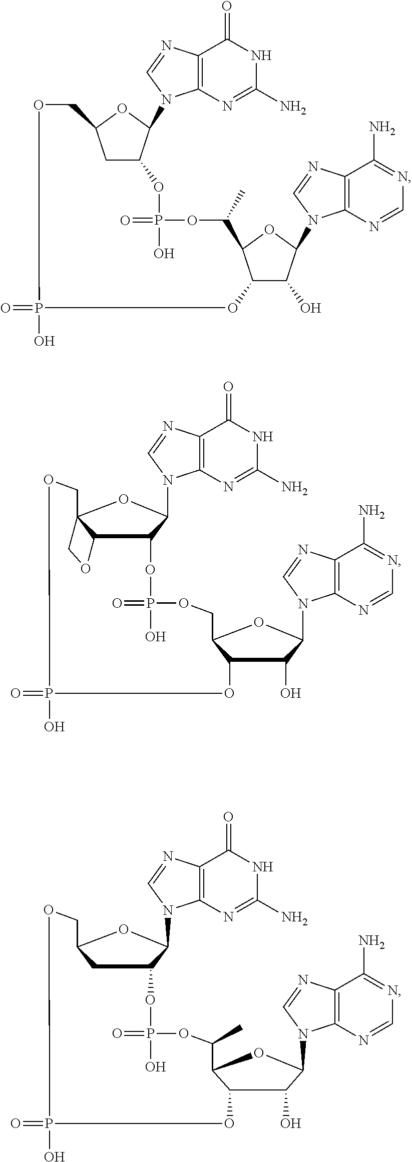

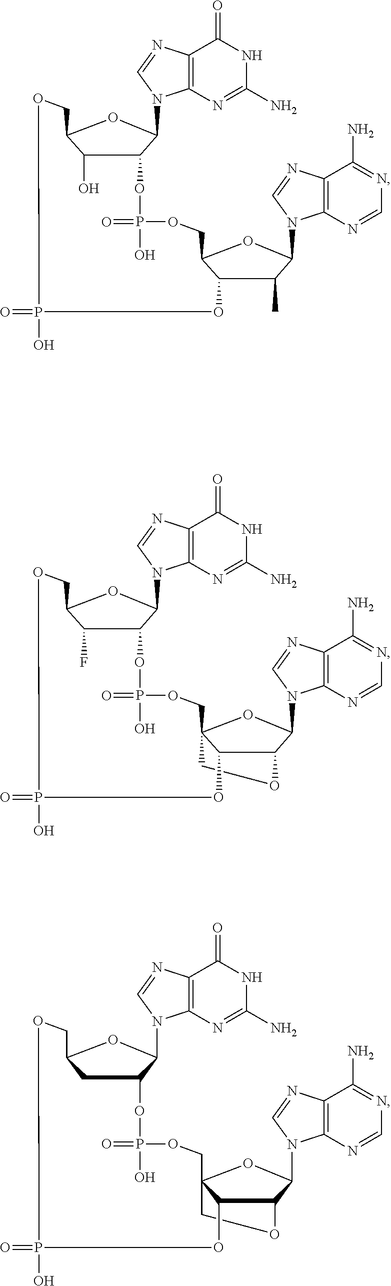

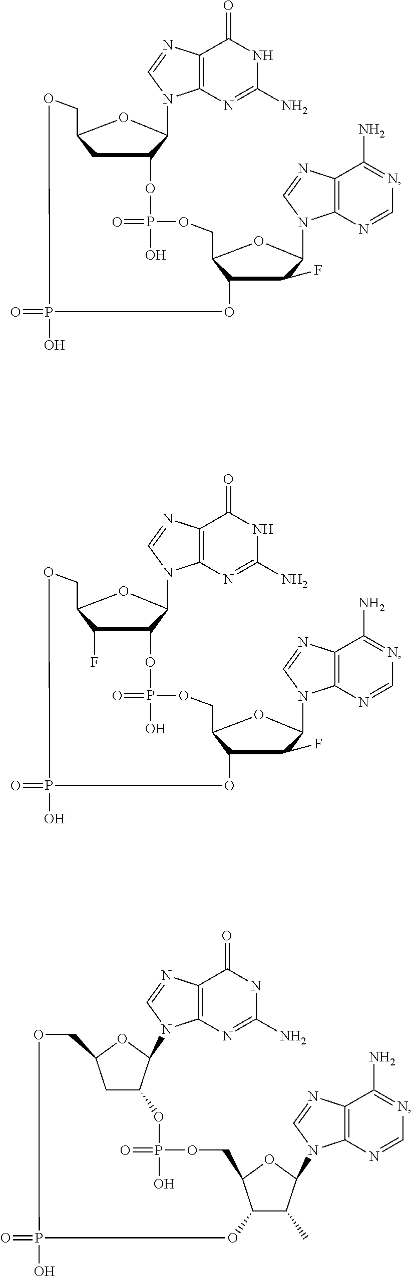

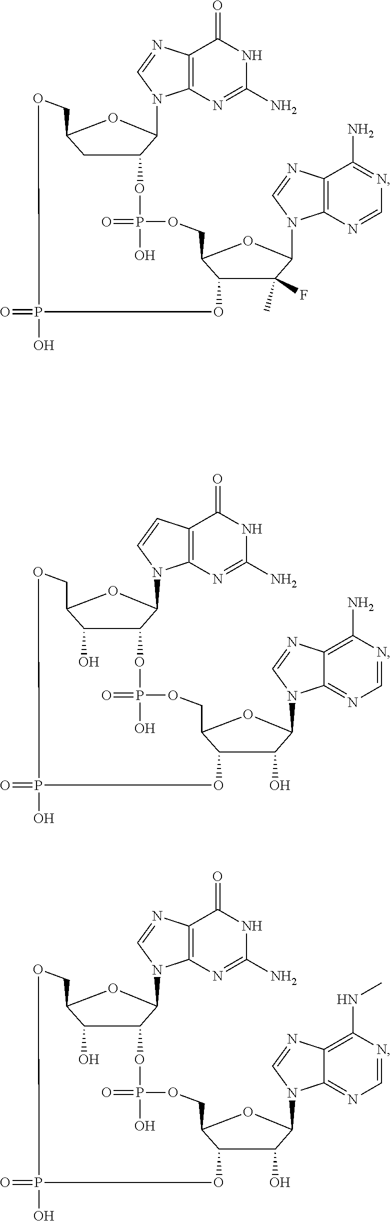 [SMILES: C[C@@]1(F)C(n2cnc3c(N)ncnc32)O[C@@H]2COP(=O)(O)O[C@@H]3C[C@@H](COP(=O)(O)O[C@H]21)OC3n1cnc2c(=O)[nH]c(N)nc21 ; Nc1nc2c(ccn2[C@@H]2OC3COP(=O)(O)O[C@@H]4C(COP(=O)(O)O[C@@H]2[C@@H]3O)O[C@@H](n2cnc3c(N)ncnc32)[C@@H]4O)c(=O)[nH]1 ; CNc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21]

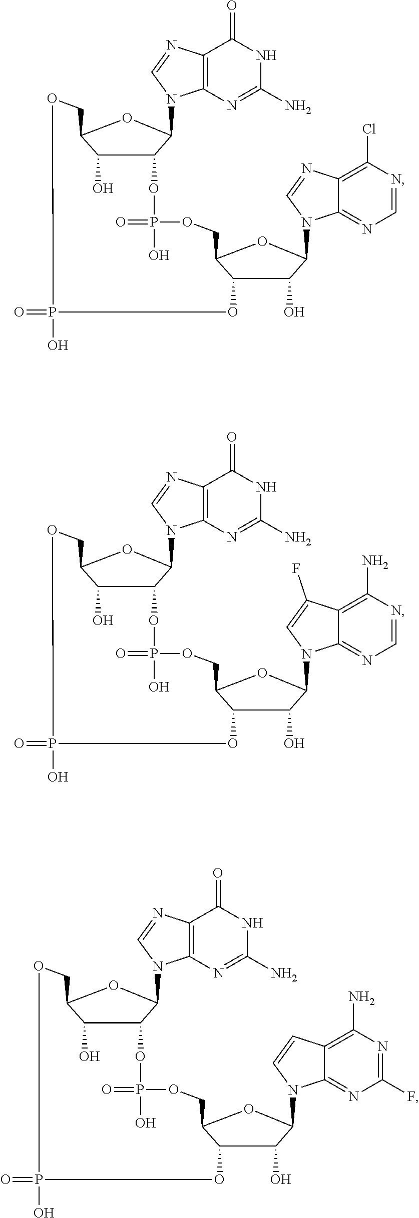 [SMILES: Nc1nc2c(ncn2[C@@H]2O[C@@H]3COP(=O)(O)O[C@H]4[C@@H](O)[C@H](n5cnc6c(Cl)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cc(F)c6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5ccc6c(N)nc(F)nc65)O[C@@H]4COP(=O)(O)O[C@@H]2[C@@H]3O)c(=O)[nH]1]

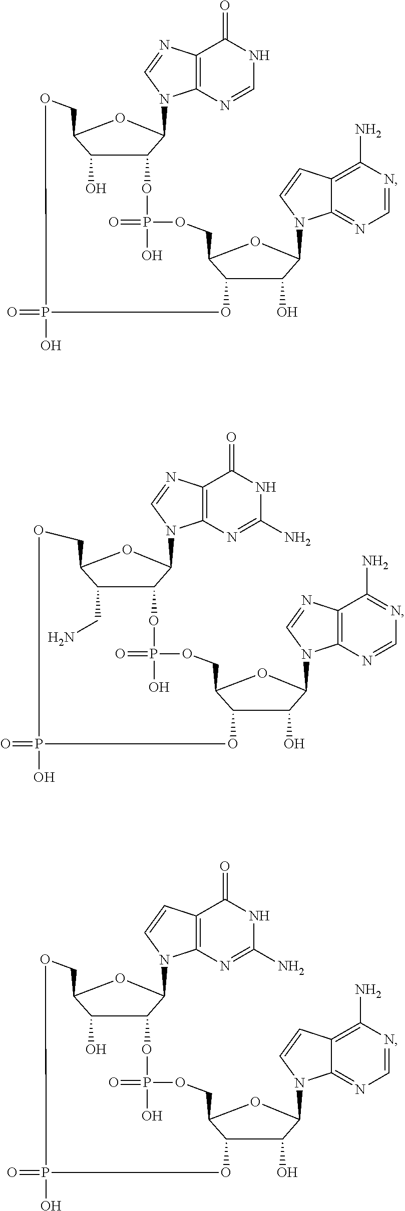 [SMILES: Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21 ; NC[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1nc2c(ccn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1]

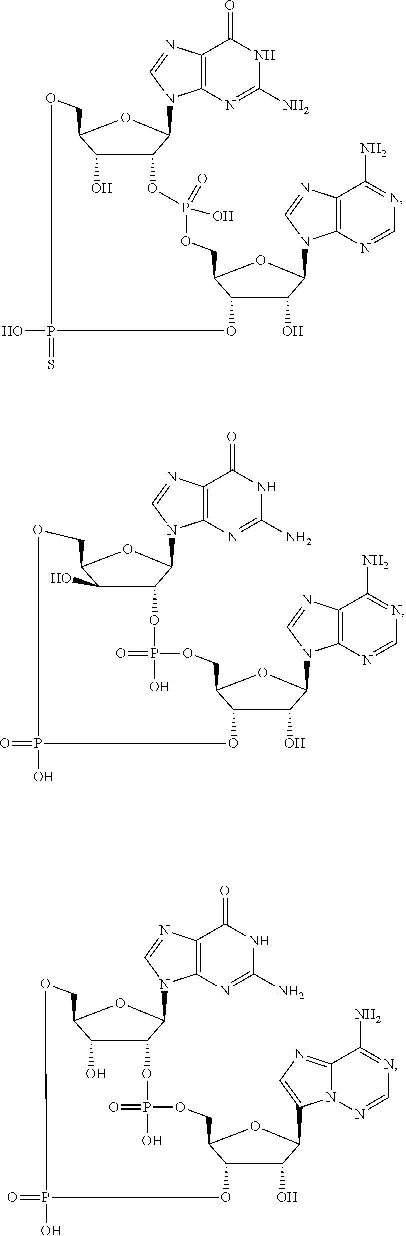 [SMILES: Nc1nc2c(ncn2[C@@H]2O[C@@H]3COP(O)(=S)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3O)c(=O)[nH]1 ; Nc1nc2c(ncn2[C@@H]2O[C@@H]3COP(=O)(O)O[C@H]4[C@@H](O)[C@H](c5cnc6c(N)ncnn56)O[C@@H]4COP(=O)(O)O[C@@H]2[C@@H]3O)c(=O)[nH]1]

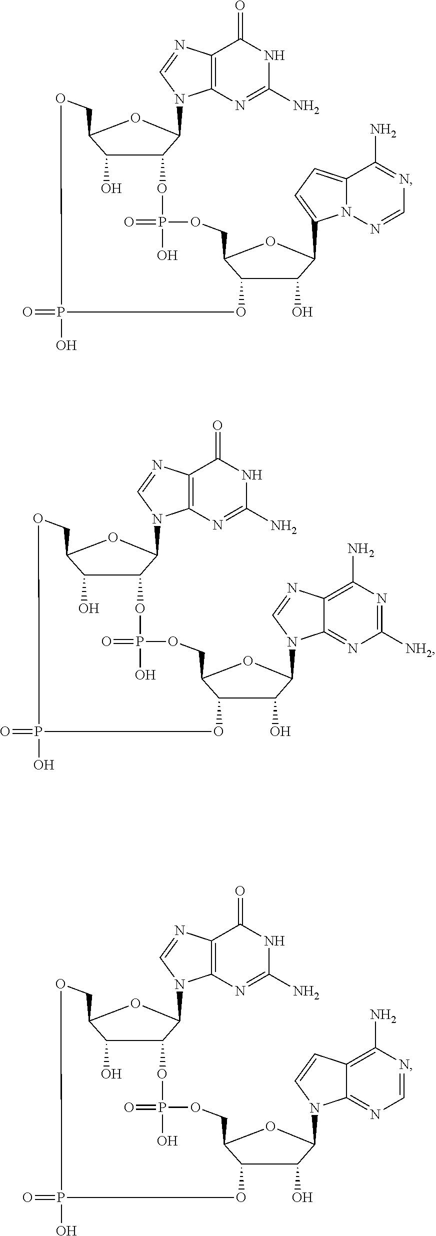 [SMILES: Nc1nc2c(ncn2[C@@H]2O[C@@H]3COP(=O)(O)O[C@H]4[C@@H](O)[C@H](c5ccc6c(N)ncnn56)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1]

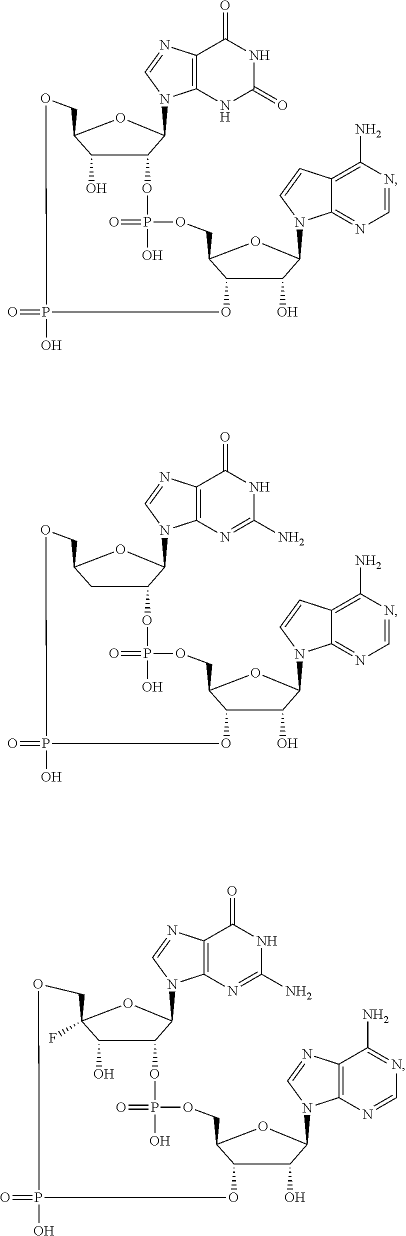 [SMILES: Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(=O)[nH]c21 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2C3)c(=O)[nH]1 ; Nc1nc2c(ncn2[C@@H]2O[C@]3(F)COP(=O)(O)O[C@H]4[C@@H](O)[C@H](n5cnc6c(N)ncnc65)O[C@@H]4COP(=O)(O)O[C@@H]2[C@@H]3O)c(=O)[nH]1]

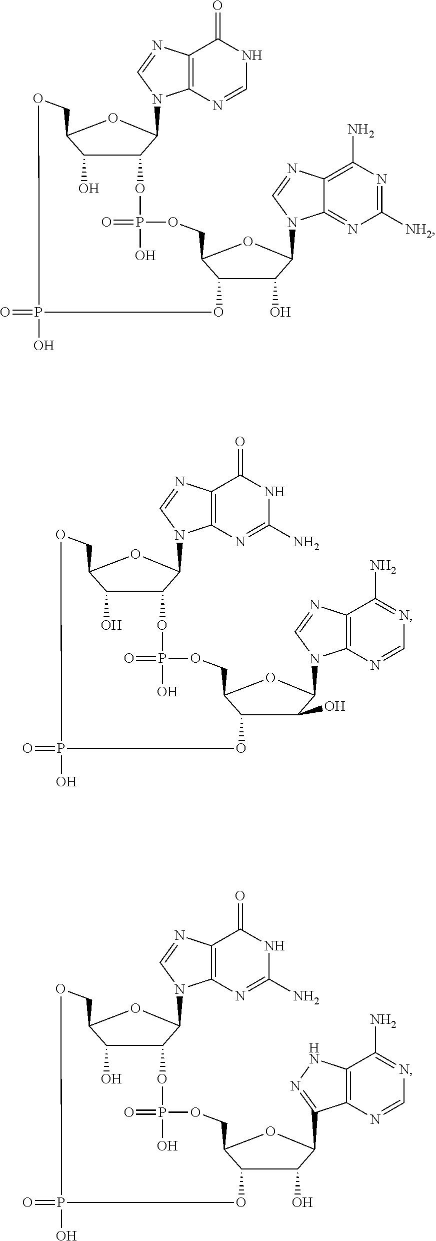 [SMILES: Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]cnc43)c2n1 ; Nc1nc2c(ncn2[C@@H]2O[C@@H]3COP(=O)(O)O[C@H]4[C@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](c5n[nH]c6c(N)ncnc56)O[C@@H]4COP(=O)(O)O[C@@H]2[C@@H]3O)c(=O)[nH]1]

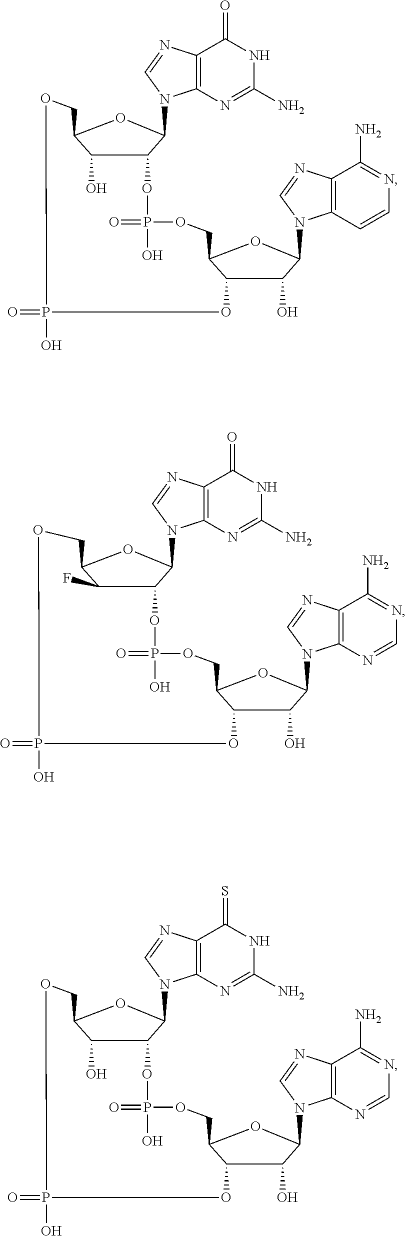 [SMILES: Nc1nc2c(ncn2[C@@H]2O[C@@H]3COP(=O)(O)O[C@H]4[C@@H](O)[C@H](n5cnc6c(N)nccc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3F)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=S)[nH]1]

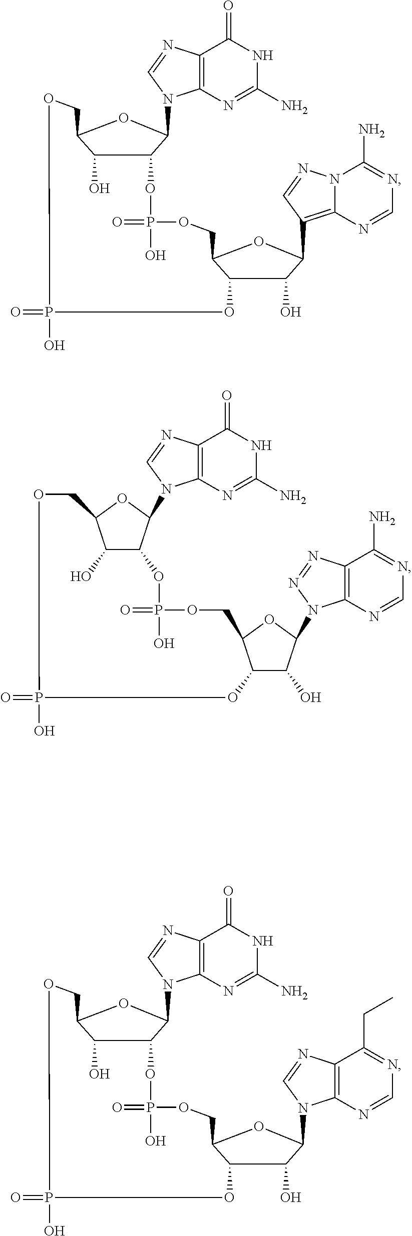 [SMILES: Nc1nc2c(ncn2[C@@H]2O[C@@H]3COP(=O)(O)O[C@H]4[C@@H](O)[C@H](c5cnn6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)ncnc65)O[C@@H]4COP(=O)(O)O[C@@H]2[C@@H]3O)c(=O)[nH]1 ; CCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21]

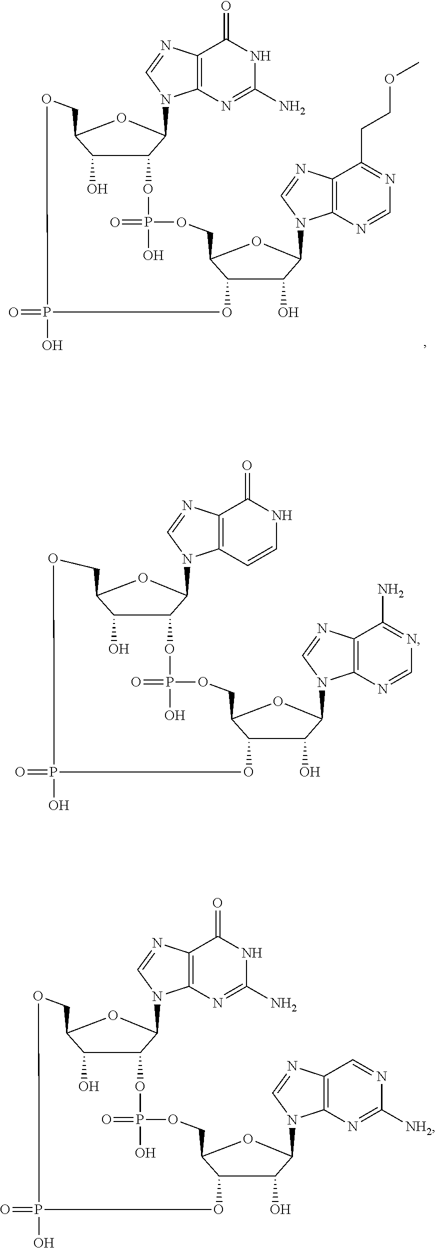 [SMILES: COCCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; Nc1ncc2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1]

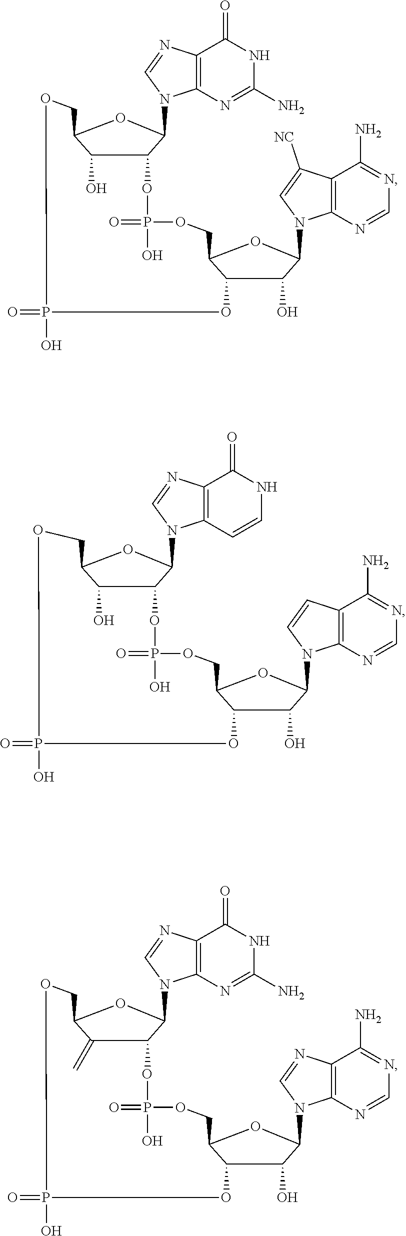 [SMILES: N#Cc1cn([C@@H]2O[C@@H]3COP(=O)(O)O[C@@H]4[C@H](O)[C@@H](COP(=O)(O)O[C@H]3[C@H]2O)O[C@H]4n2cnc3c(=O)[nH]c(N)nc32)c2ncnc(N)c12 ; Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; C=C1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21]

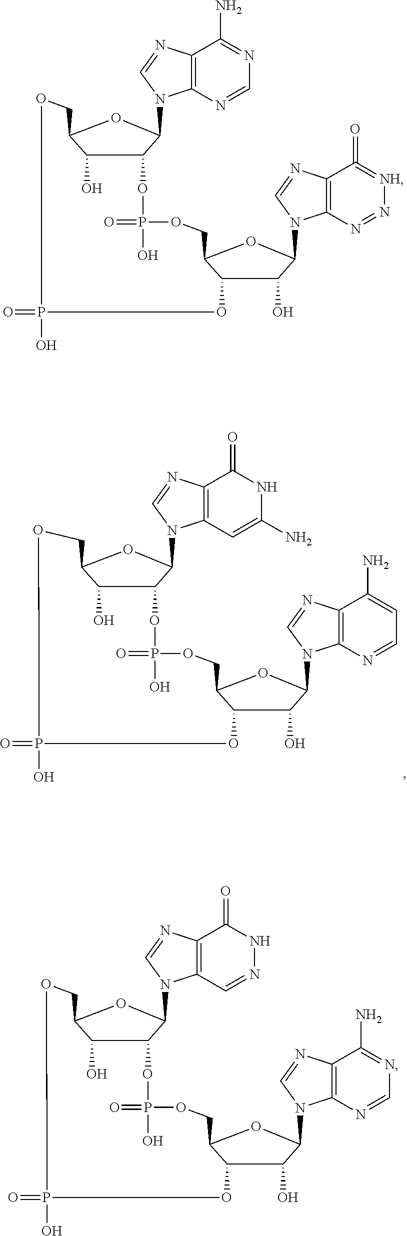

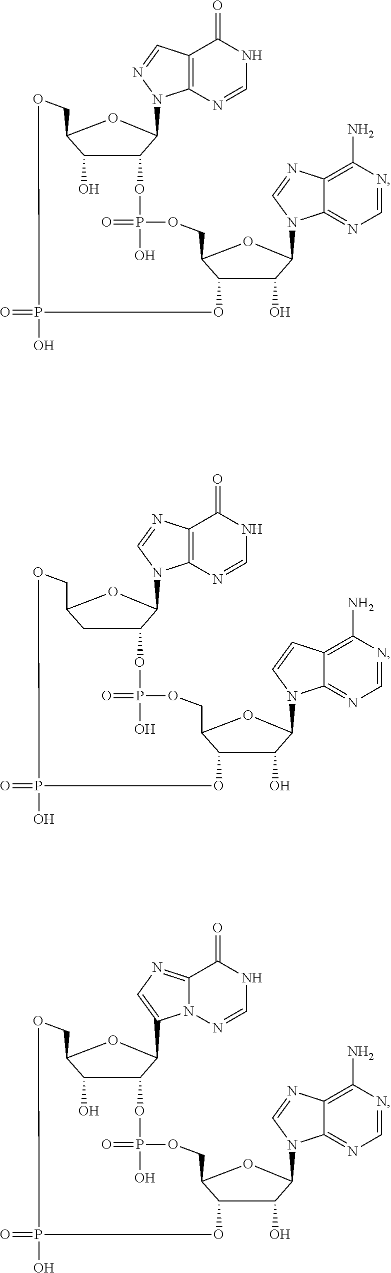 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1ncc2c(=O)[nH]cnc21 ; Nc1ncnc2c1ccn2[C@@H]1OC2COP(=O)(O)O[C@@H]3CC(COP(=O)(O)O[C@H]2[C@H]1O)O[C@H]3n1cnc2c(=O)[nH]cnc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1cnc2c(=O)[nH]cnn12]

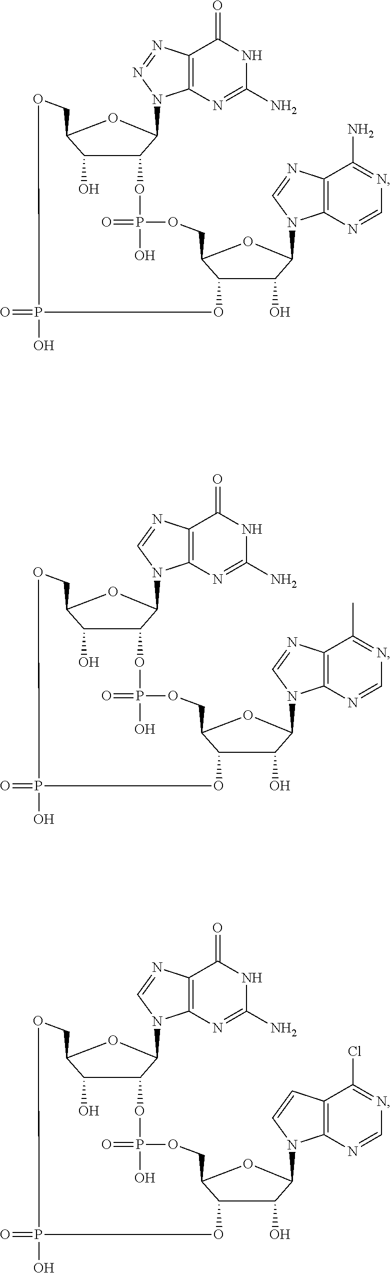 [SMILES: Nc1nc2c(nnn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Cc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2O[C@@H]3COP(=O)(O)O[C@H]4[C@@H](O)[C@H](n5ccc6c(Cl)ncnc65)O[C@@H]4COP(=O)(O)O[C@@H]2[C@@H]3O)c(=O)[nH]1]

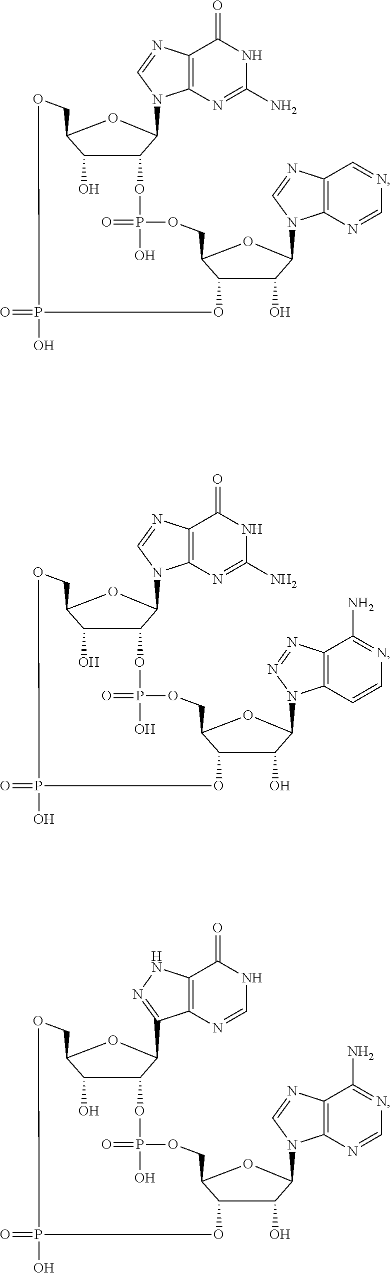 [SMILES: Nc1nc2c(ncn2[C@@H]2O[C@@H]3COP(=O)(O)O[C@H]4[C@@H](O)[C@H](n5cnc6cncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)nccc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1n[nH]c2c(=O)[nH]cnc12]

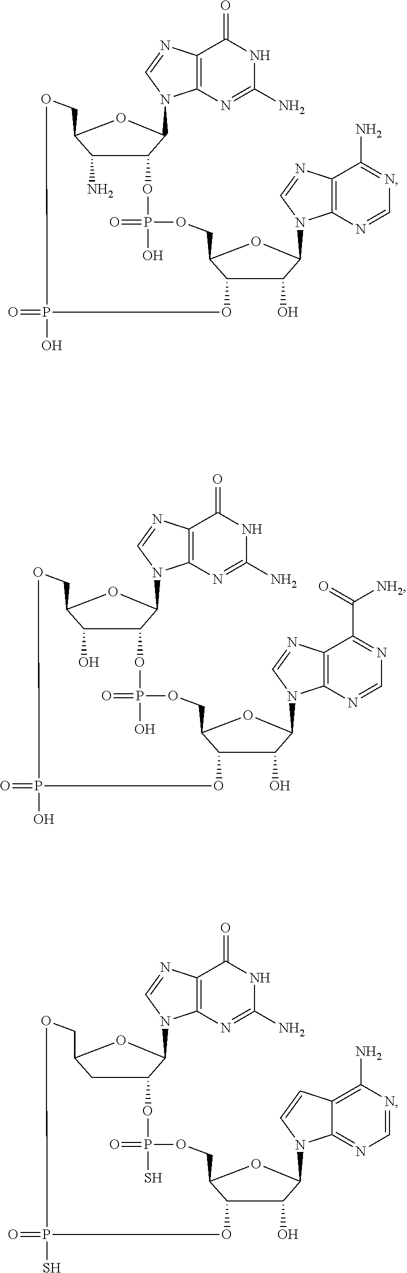

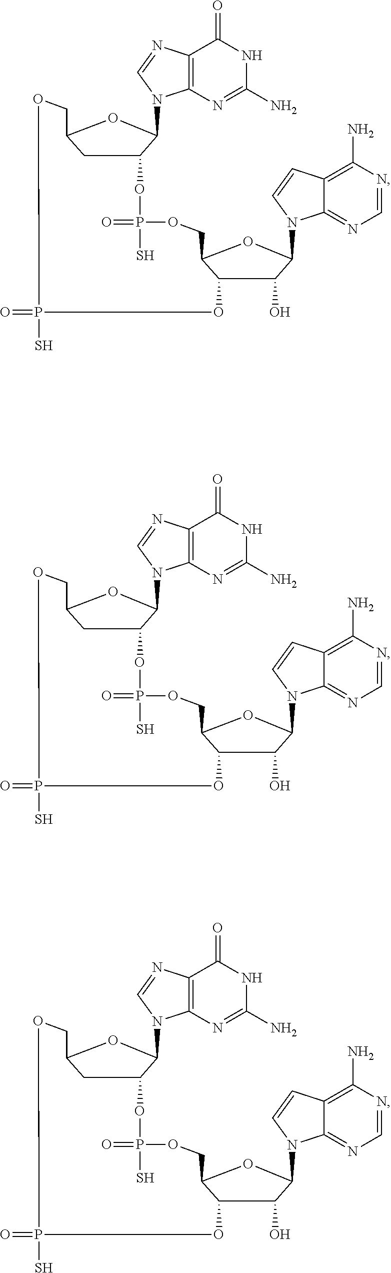 [SMILES: Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1]

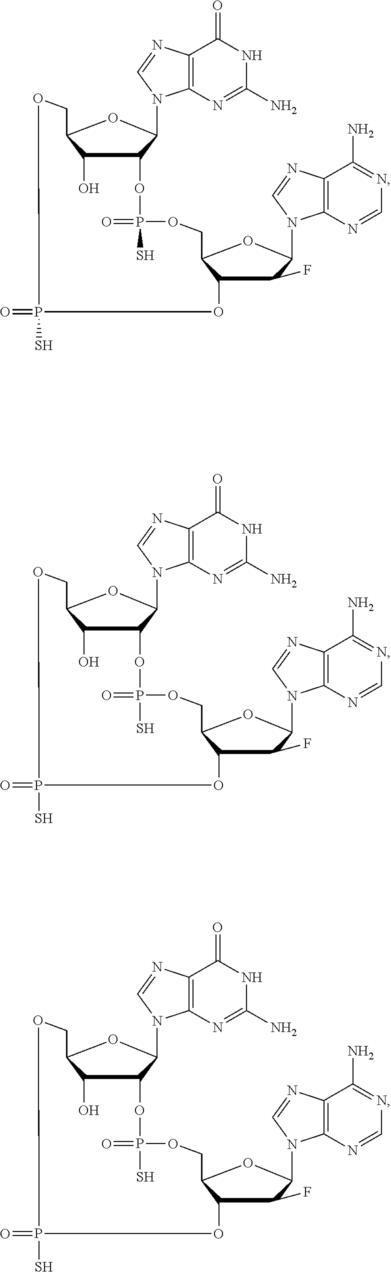

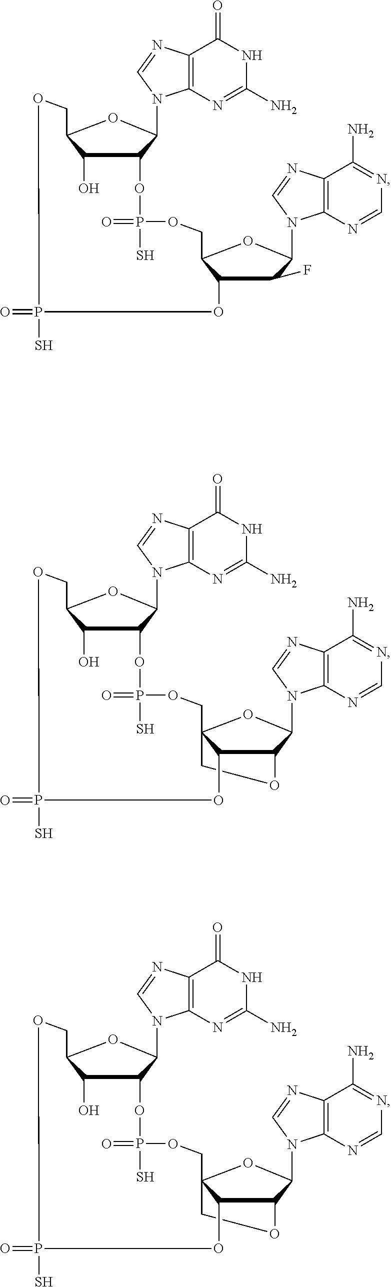

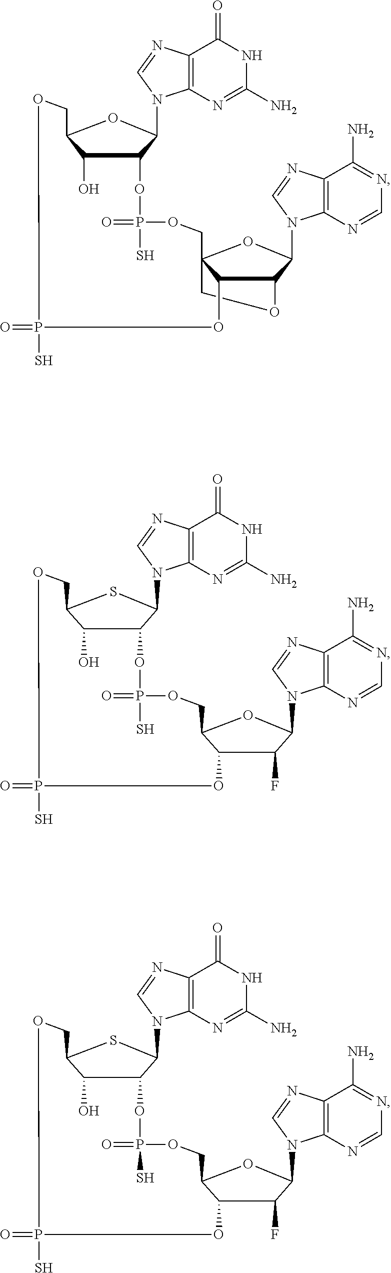 [SMILES: Nc1nc2c(ncn2[C@@H]2O[C@@H]3COP(=O)(S)O[C@@H]4C5OC[C@]4(COP(=O)(S)OC2[C@H]3O)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4CO[P@@](=O)(S)O[C@@H]2[C@@H]3O)c(=O)[nH]1]

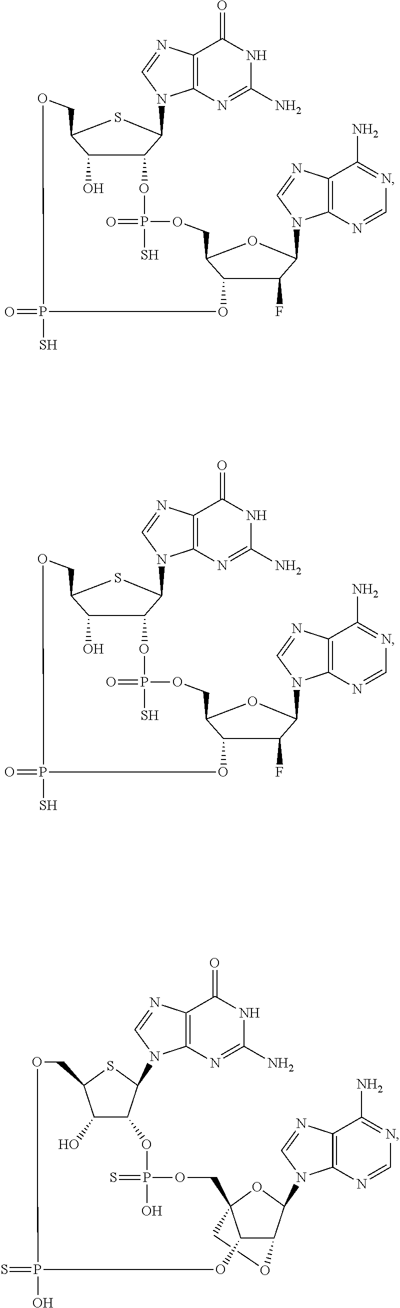 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1]

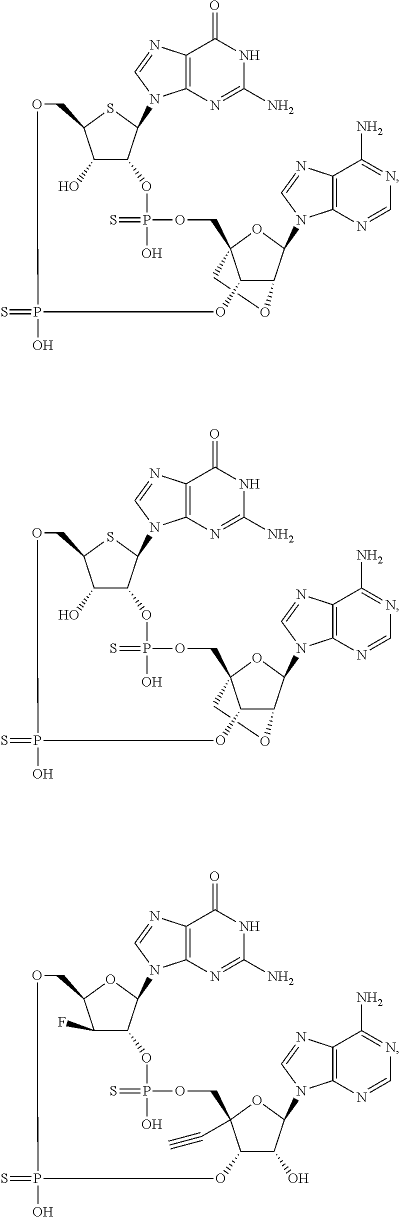 [SMILES: Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21]

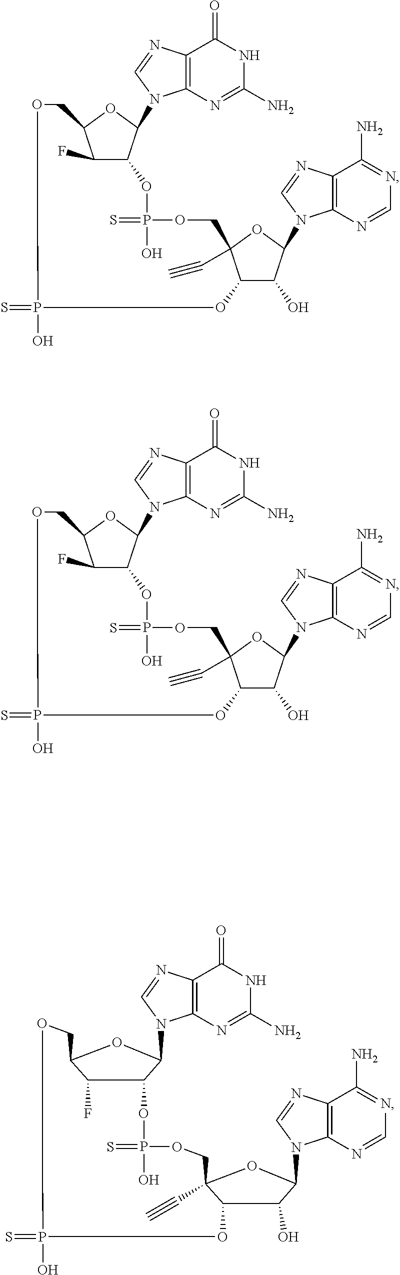 [SMILES: C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21]

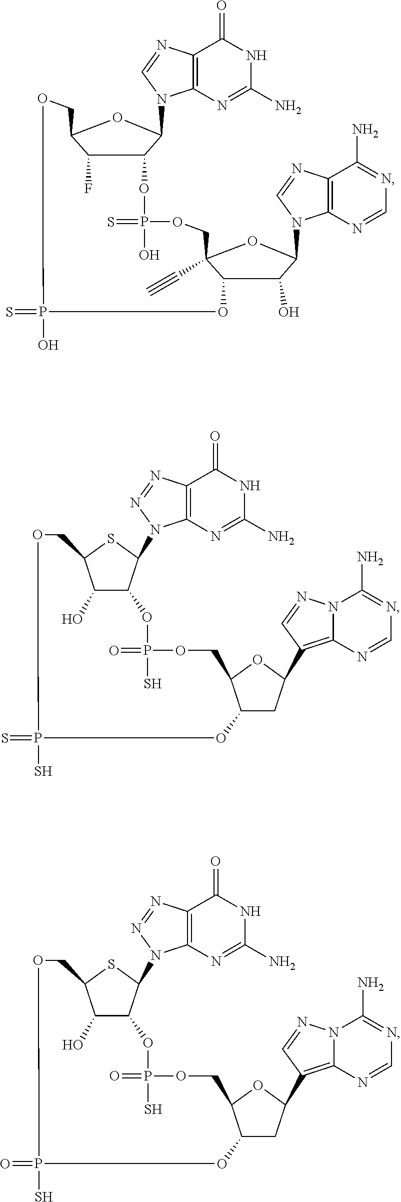 [SMILES: C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; Nc1nc2c(nnn2[C@@H]2S[C@@H]3COP(=S)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1]

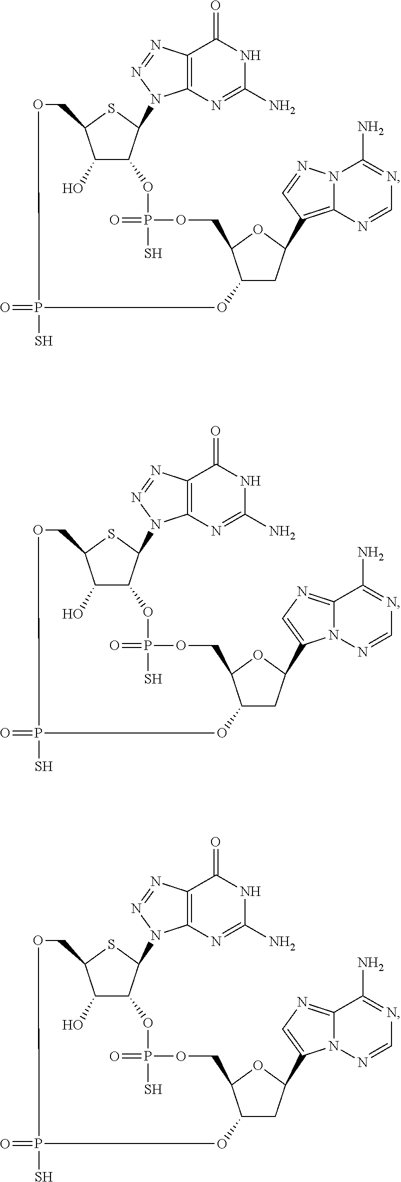 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1]

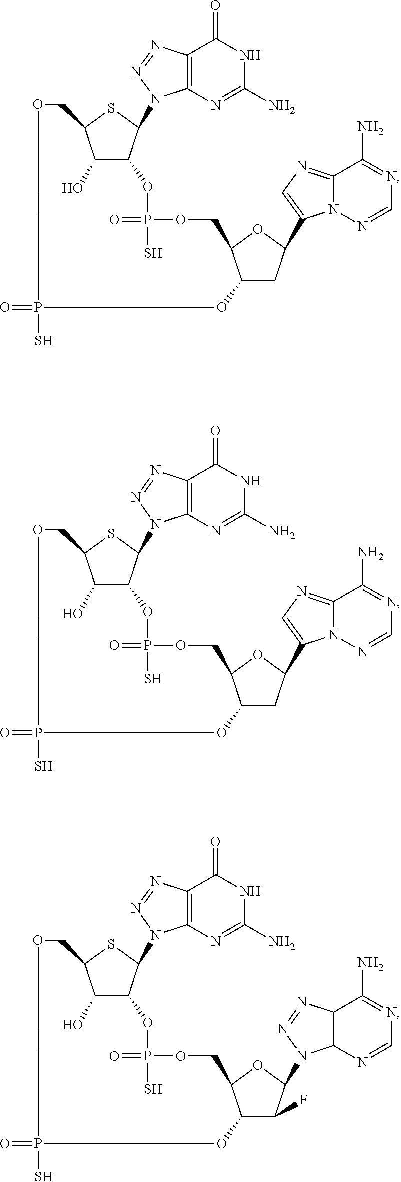 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; NC1=NC=NC2C1N=NN2[C@@H]1O[C@@H]2COP(=O)(S)O[C@@H]3[C@H](O)[C@@H](COP(=O)(S)O[C@H]2[C@@H]1F)S[C@H]3n1nnc2c(=O)[nH]c(N)nc21]

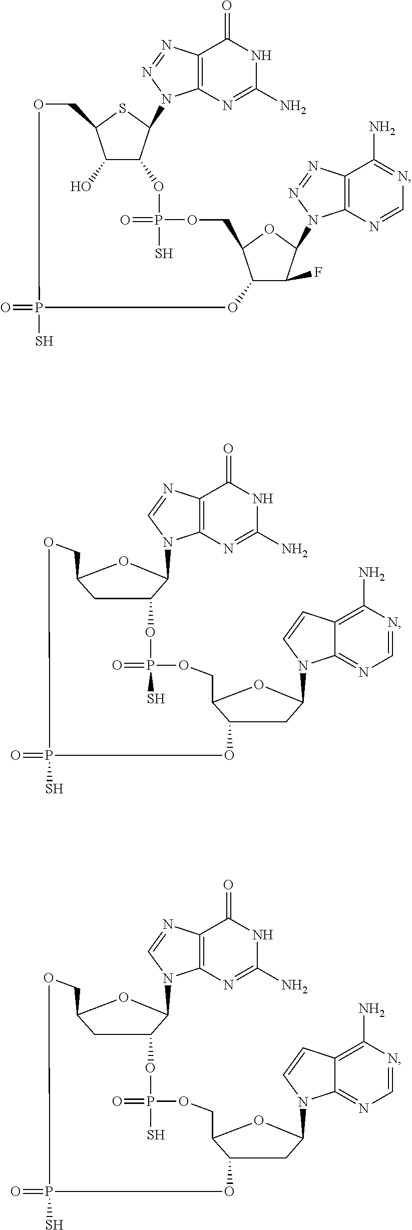 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3CO[P@@](=O)(S)O[C@H]4C[C@H](n5ccc6c(N)ncnc65)OC4COP(=O)(S)O[C@@H]2C3)c(=O)[nH]1]

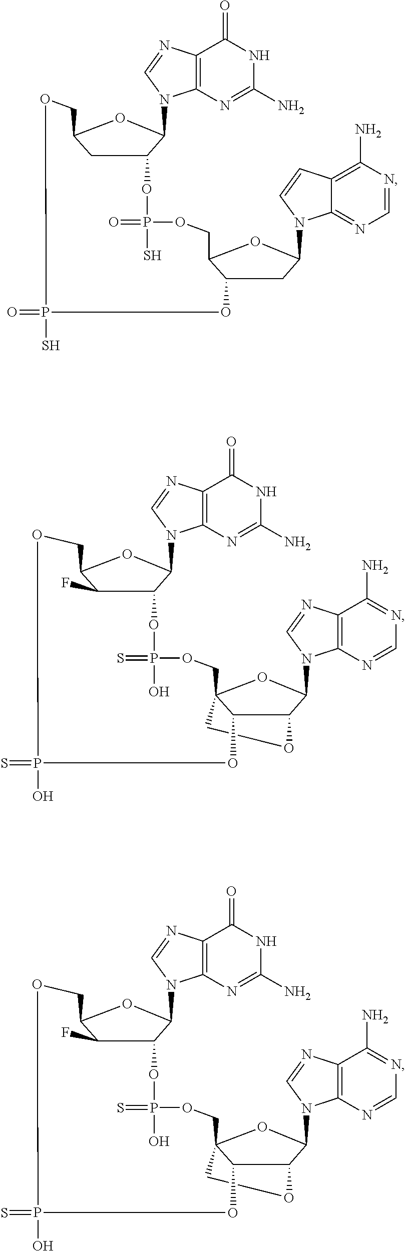 [SMILES: Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1]

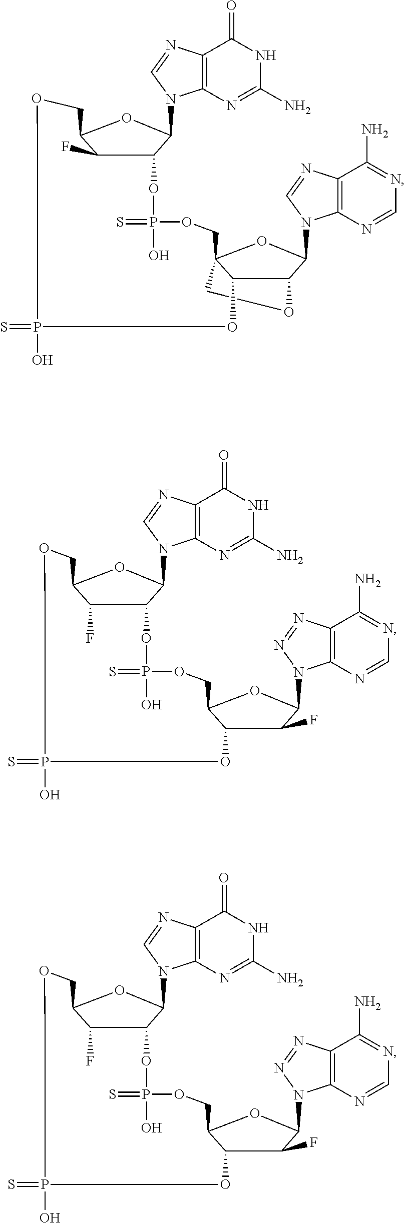 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1]

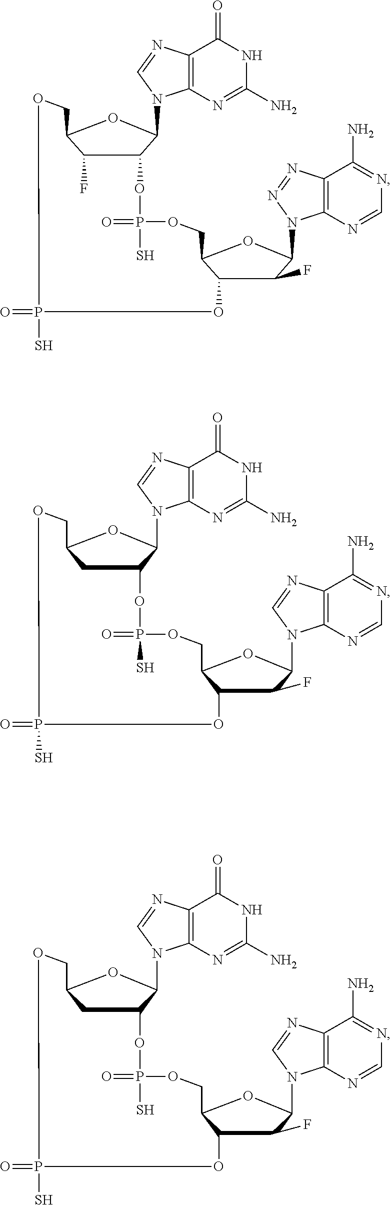

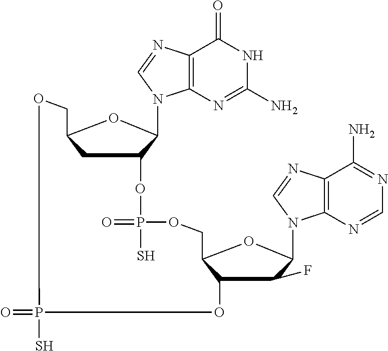

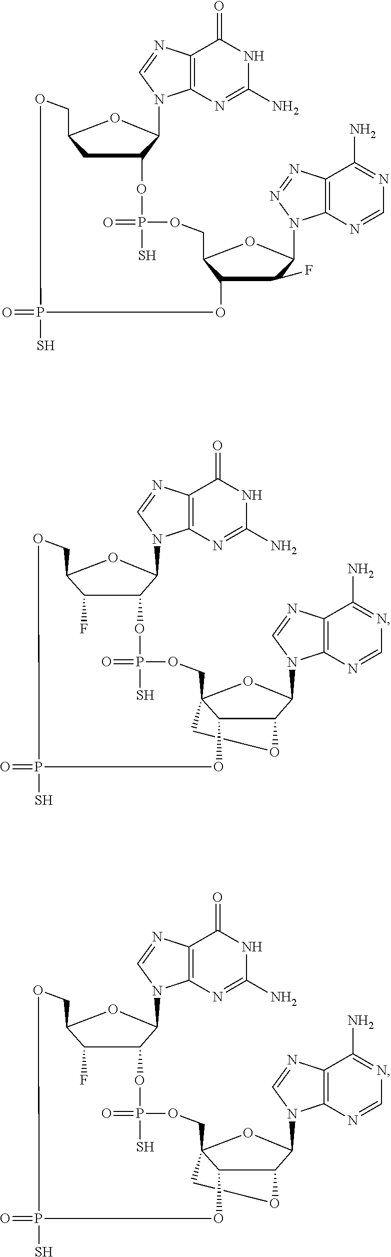

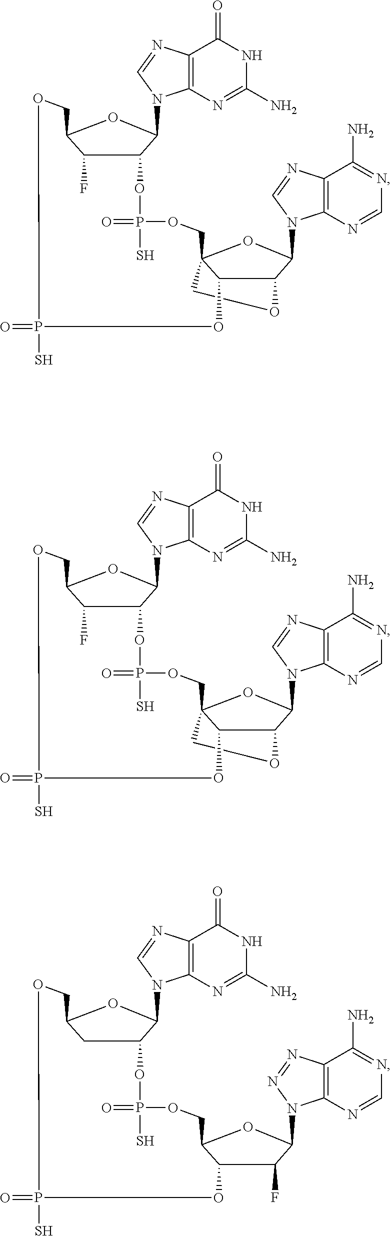 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1]

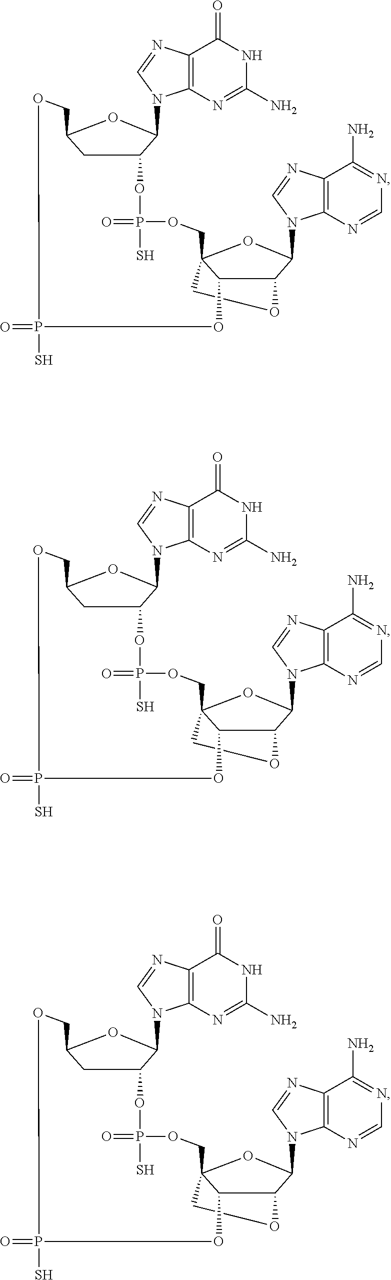 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1]

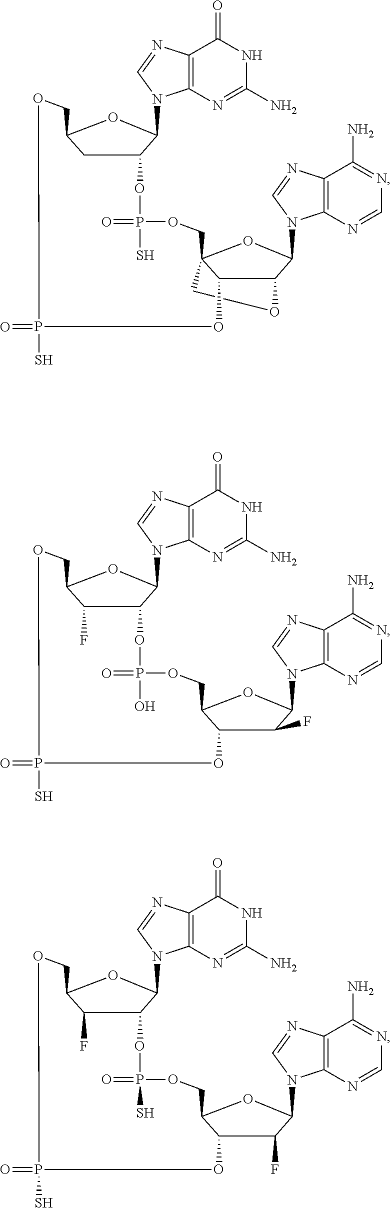 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(O)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1]

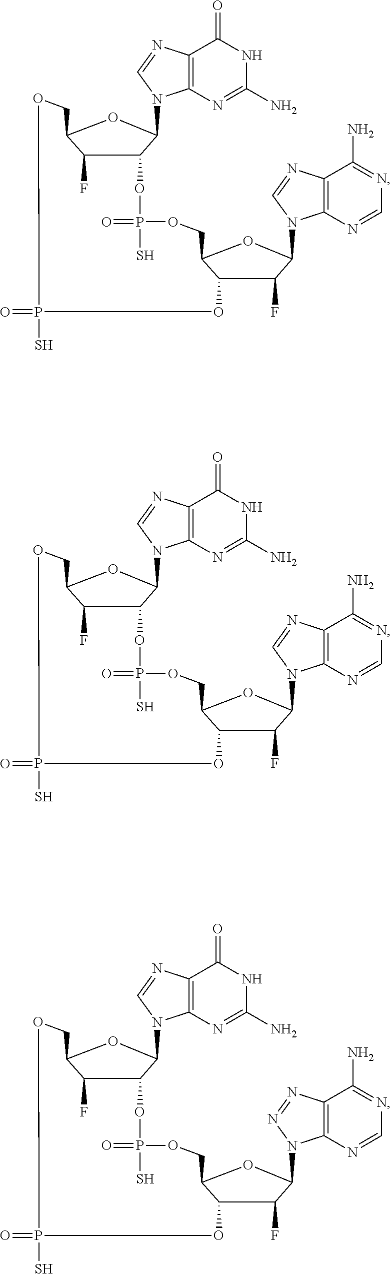 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1]

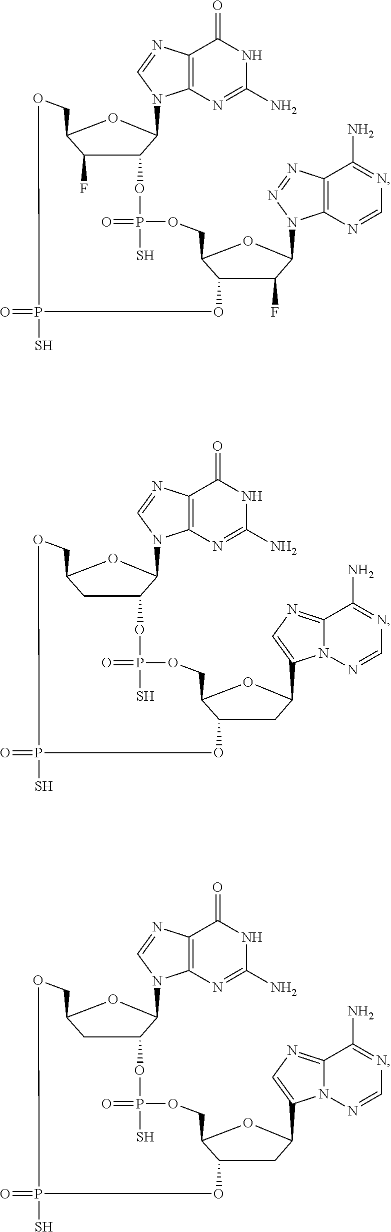 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1]

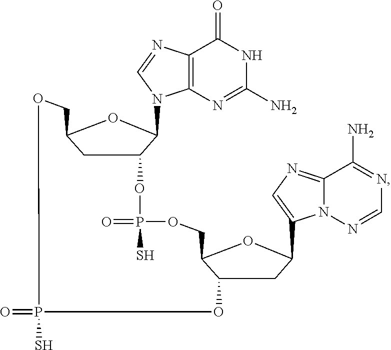 [SMILES: Nc1nc2c(ncn2[C@@H]2O[C@@H]3CO[P@@](=O)(S)O[C@H]4C[C@H](c5cnc6c(N)ncnn56)O[C@@H]4CO[P@@](=O)(S)O[C@@H]2C3)c(=O)[nH]1]

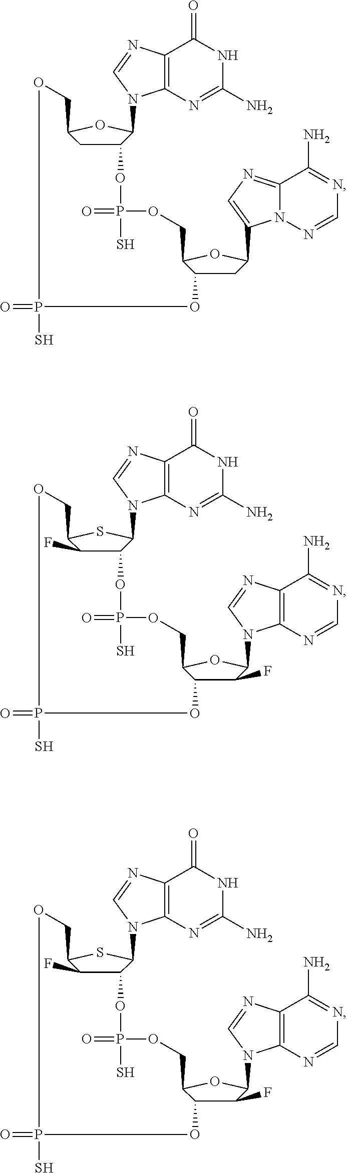 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1]

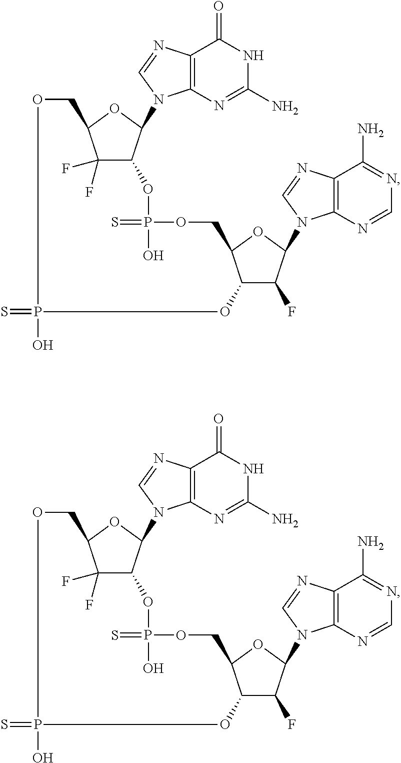 [SMILES: Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1]

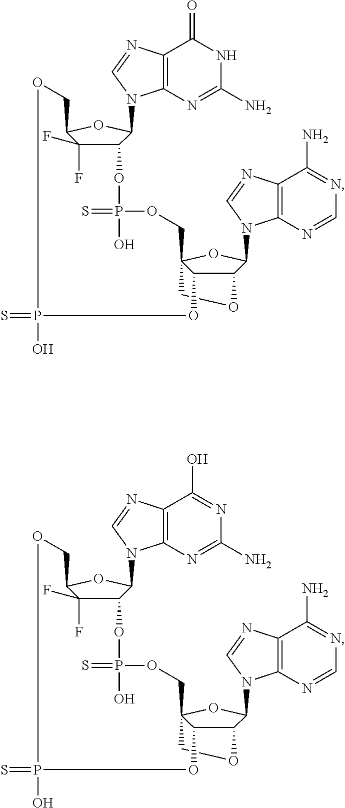 [SMILES: Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; Nc1nc(O)c2ncn([C@@H]3O[C@@H]4COP(O)(=S)O[C@H]5[C@H]6OC[C@]5(COP(O)(=S)O[C@@H]3C4(F)F)O[C@H]6n3cnc4c(N)ncnc43)c2n1]

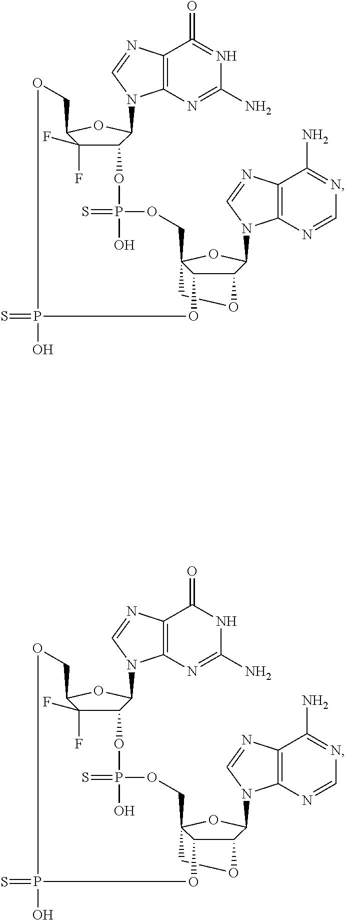

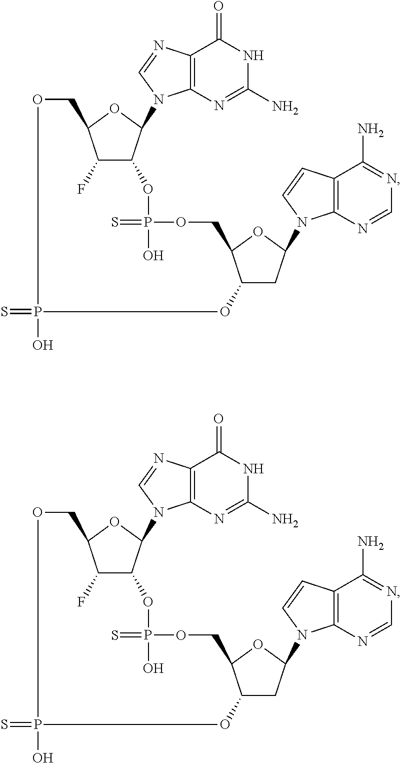 [SMILES: Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1]

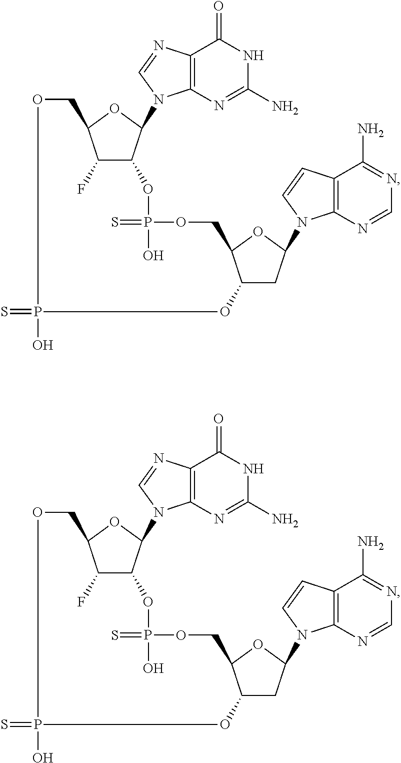 [SMILES: Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1]

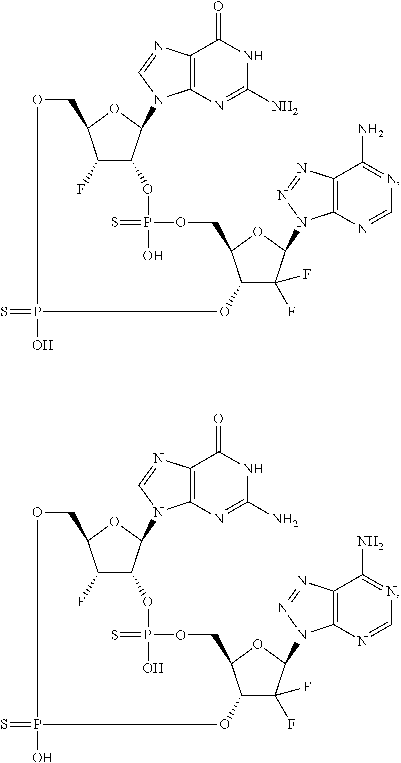 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1]

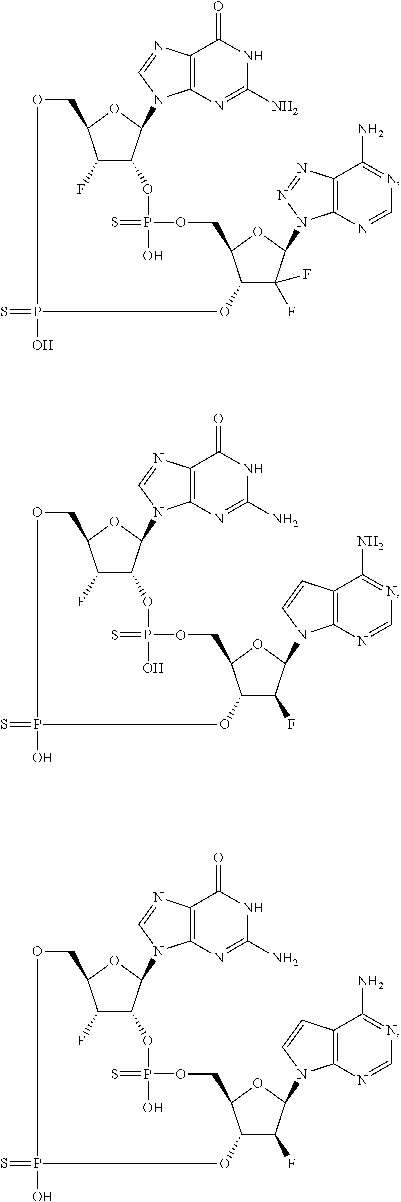 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1]

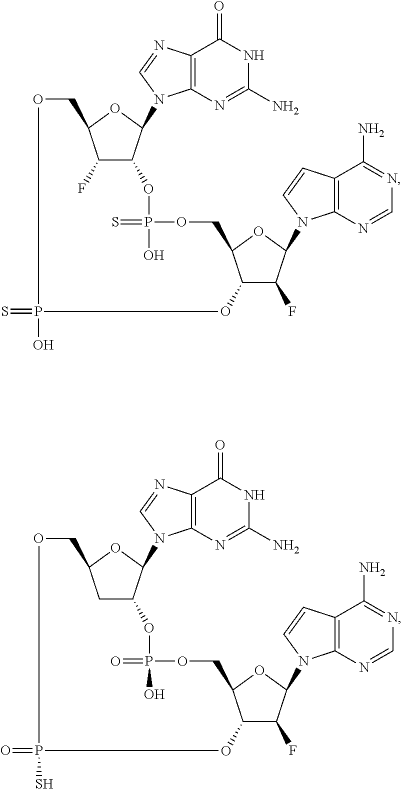 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(O)O[C@@H]2C3)c(=O)[nH]1]

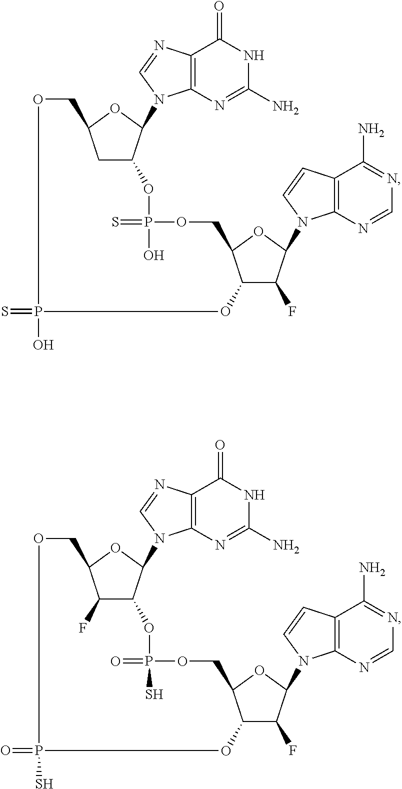 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(S)O[C@@H]2[C@H]3F)c(=O)[nH]1]

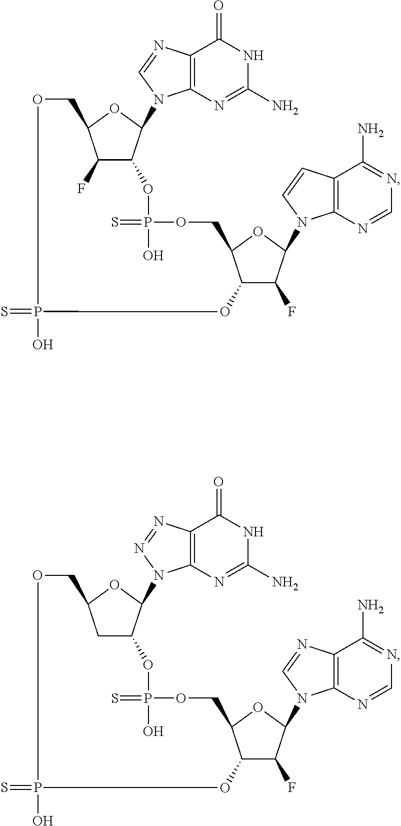 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1]

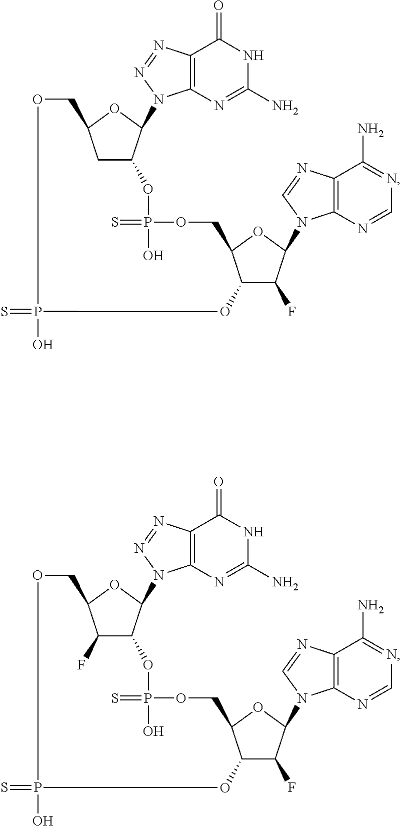 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1]

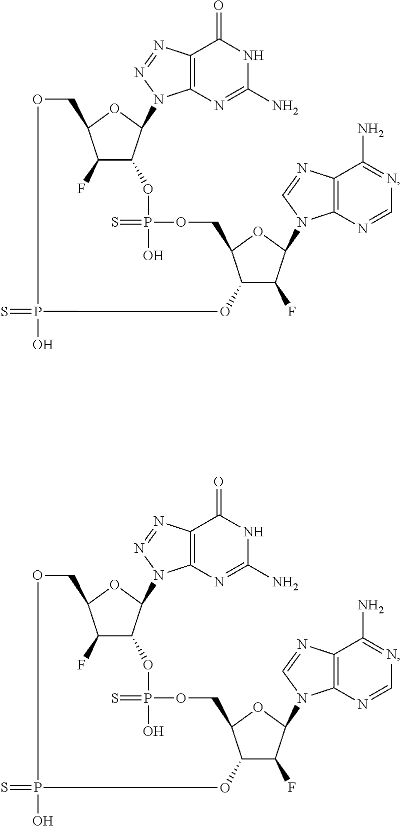 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1]

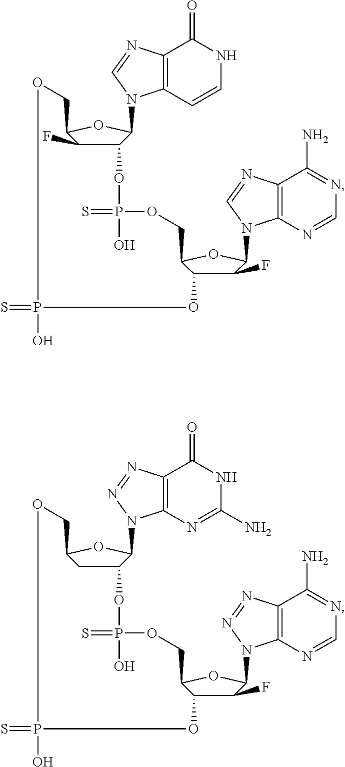 [SMILES: Nc1ncnc2c1ncn2[C@@H]1O[C@@H]2COP(O)(=S)O[C@@H]3[C@@H](F)[C@@H](COP(O)(=S)O[C@H]2[C@@H]1F)O[C@H]3n1cnc2c(=O)[nH]ccc21 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1]

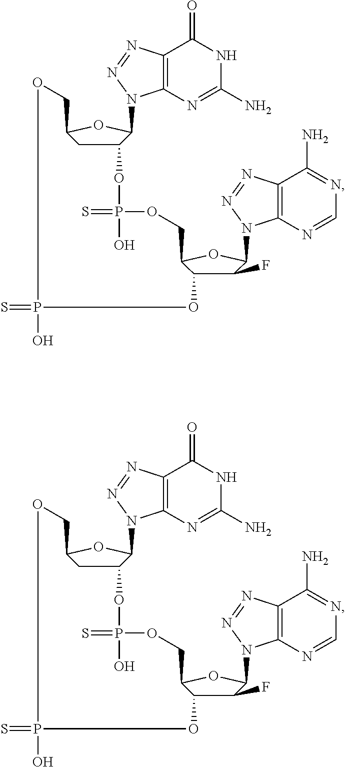 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1]

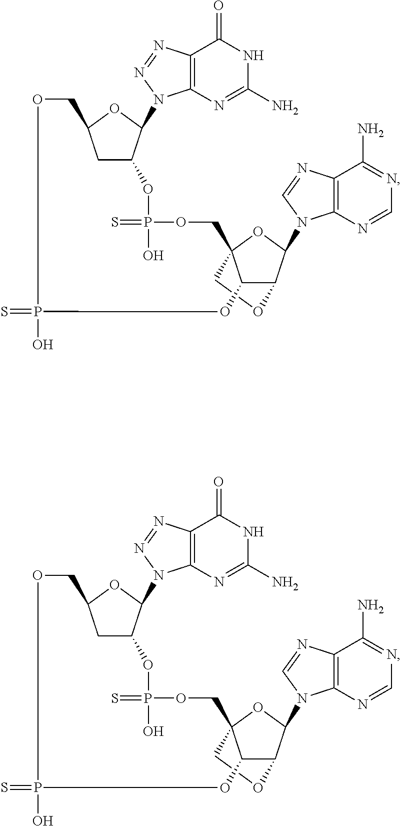 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1]

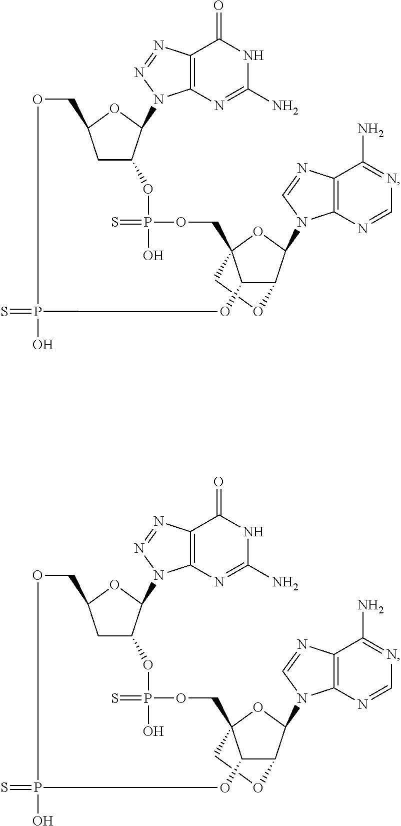 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1]

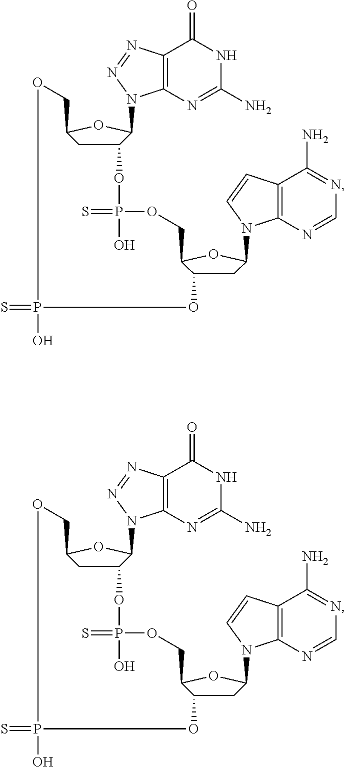 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1]

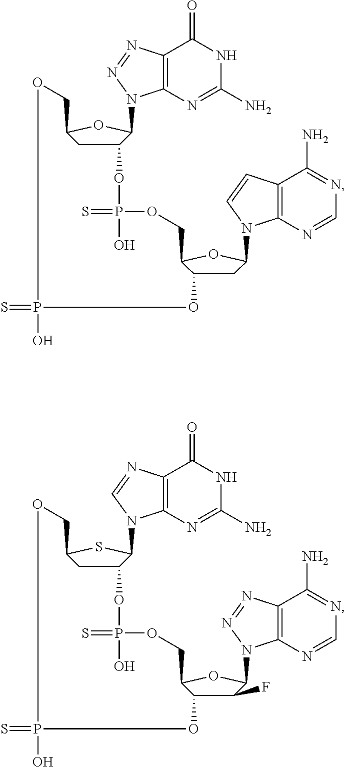 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1]

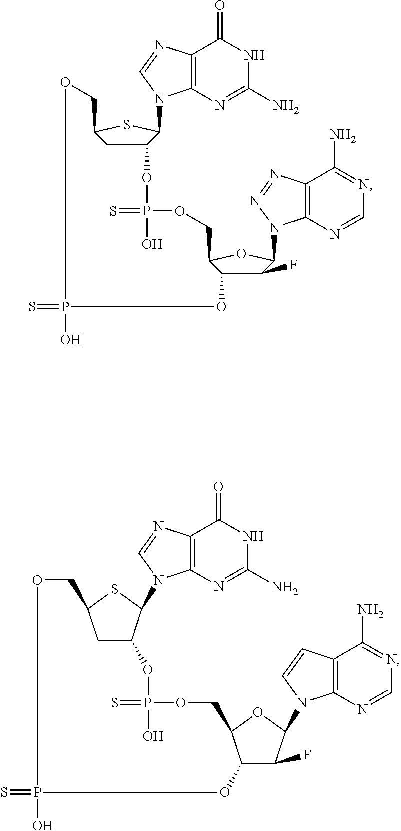 [SMILES: Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5ccc6c(N)ncnc65)O[C@@H]4COP(O)(=S)O[C@@H]2C3)c(=O)[nH]1]

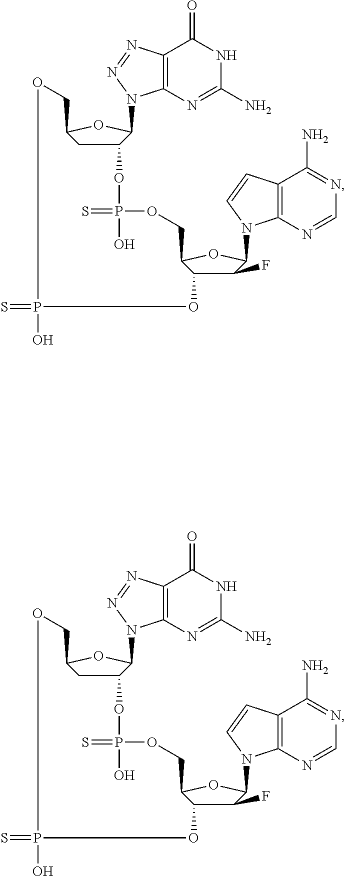 [SMILES: Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1]

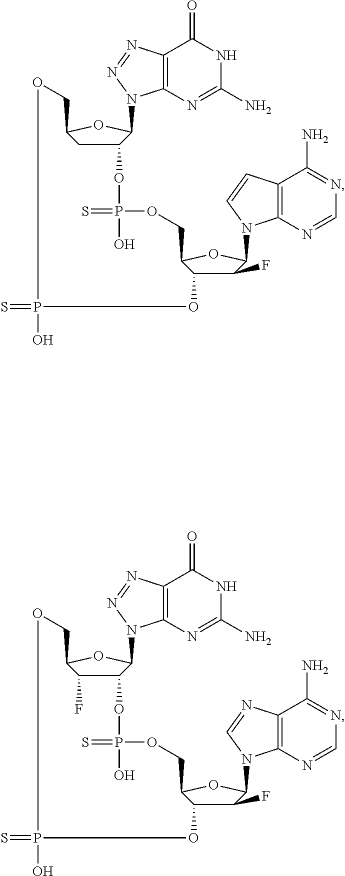 [SMILES: Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

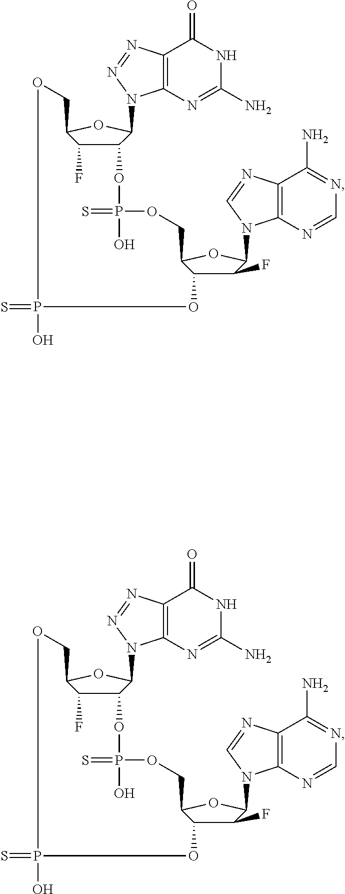 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

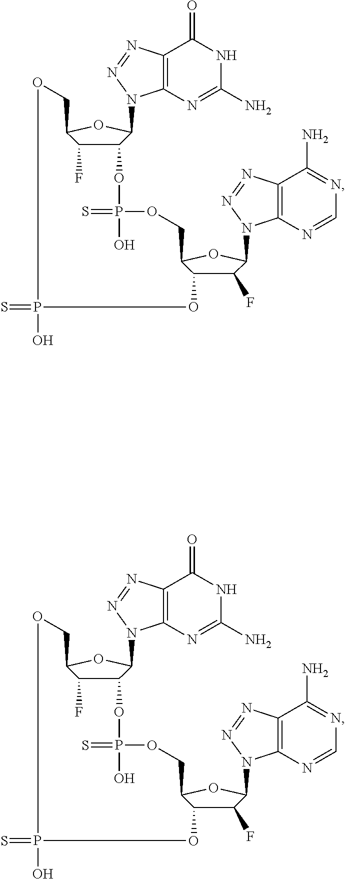 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1]

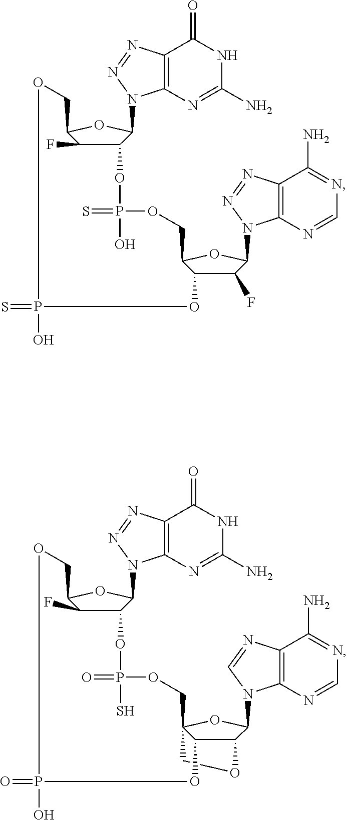 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(=O)(O)O[C@H]4[C@H]5OC[C@]4(COP(=O)(S)O[C@@H]2[C@H]3F)O[C@H]5n2cnc3c(N)ncnc32)c(=O)[nH]1]

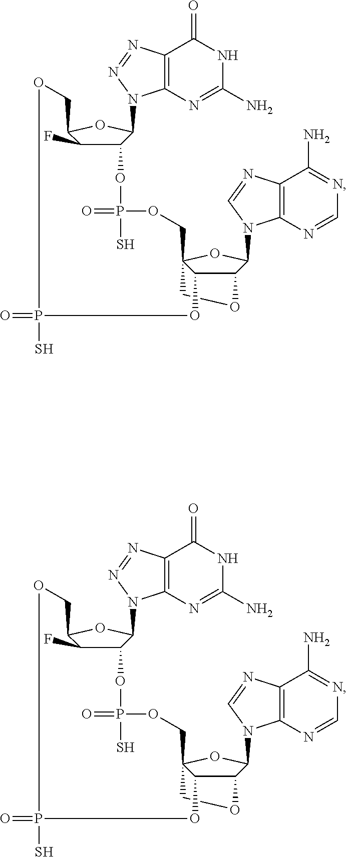 [SMILES: Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1]

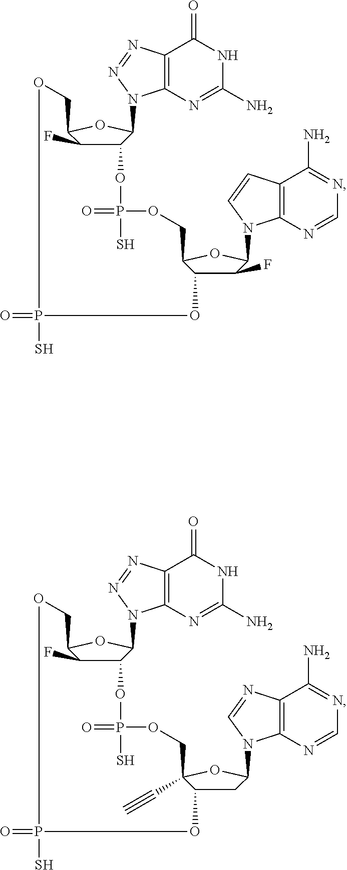 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21]

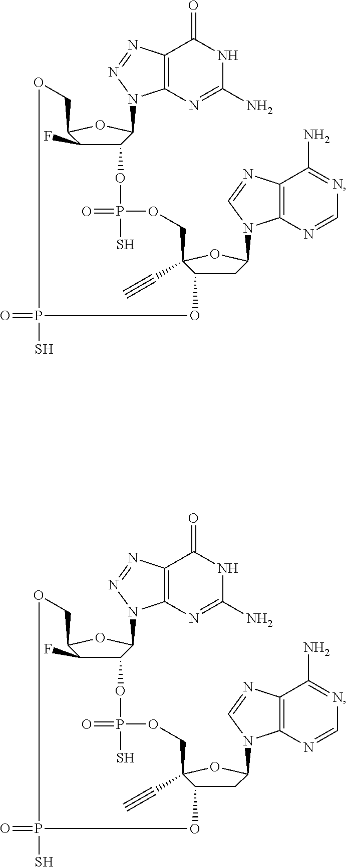 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21]

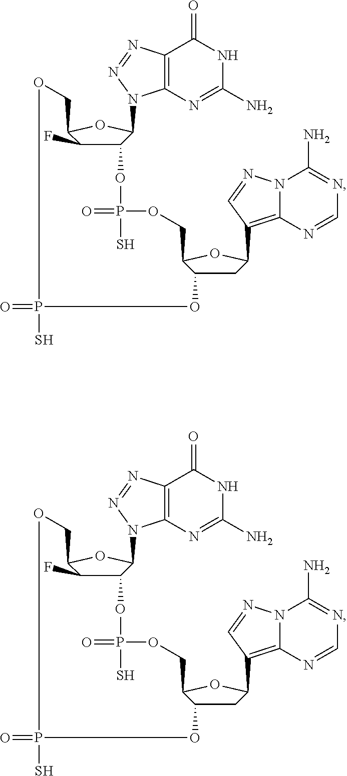 [SMILES: Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1 ; Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1]

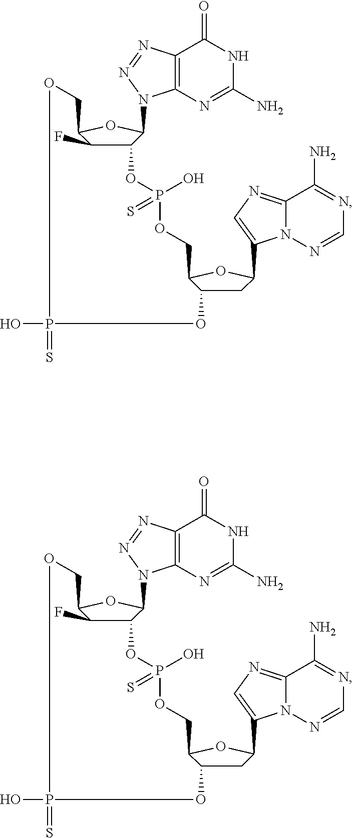 [SMILES: Nc1nc2c(nnn2[C@@H]2O[C@@H]3COP(O)(=S)O[C@H]4C[C@H](c5cnc6c(N)ncnn56)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4C[C@H](c5cnc6c(N)ncnn56)O[C@@H]4COP(O)(=S)O[C@@H]2[C@H]3F)c(=O)[nH]1]

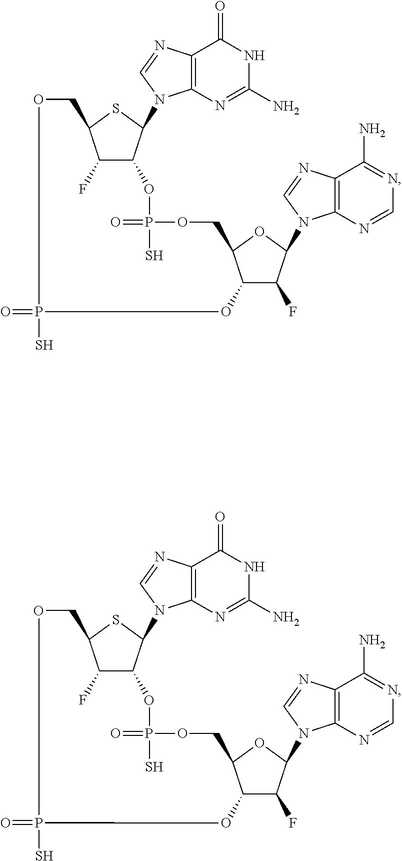 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

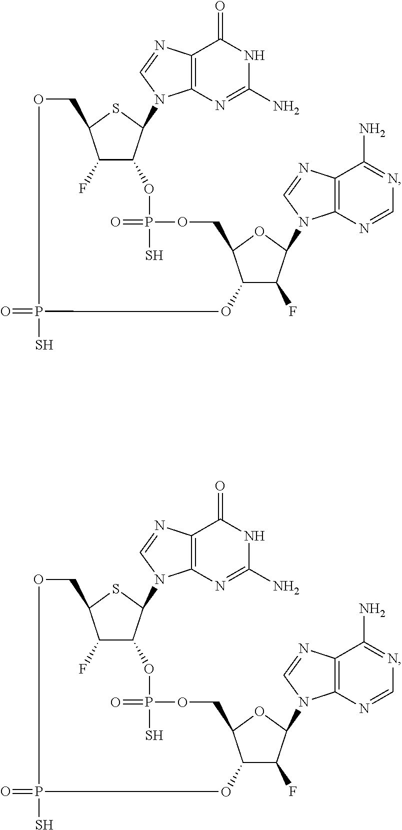 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

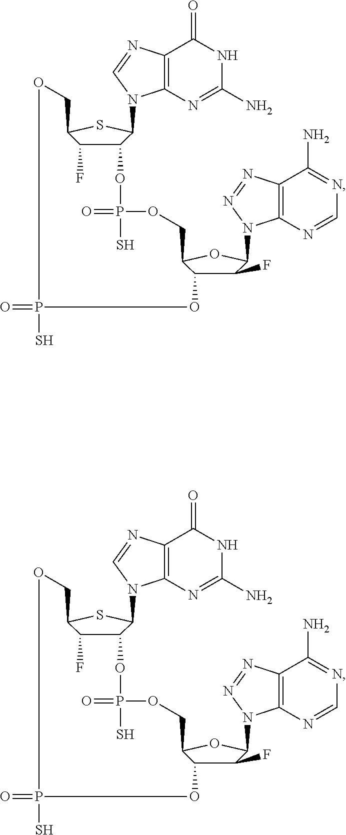 [SMILES: Nc1nc2c(ncn2[C@@H]2S[C@@H]3COP(=O)(S)CO[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)CO[C@H]4[C@H](F)[C@H](n5nnc6c(N)ncnc65)O[C@@H]4COP(=O)(S)O[C@@H]2[C@@H]3F)c(=O)[nH]1]

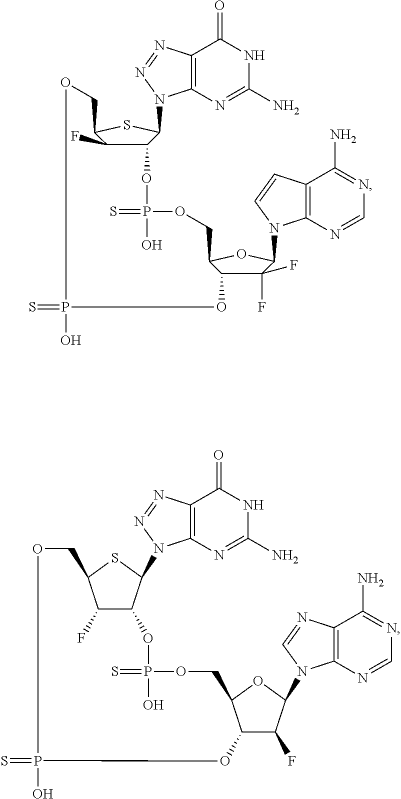 [SMILES: Nc1nc2c(nnn2[C@@H]2S[C@@H]3COP(O)(=S)CO[C@@H]4[C@@H](COP(O)(=S)O[C@@H]2[C@H]3F)O[C@@H](n2ccc3c(N)ncnc32)C4(F)F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

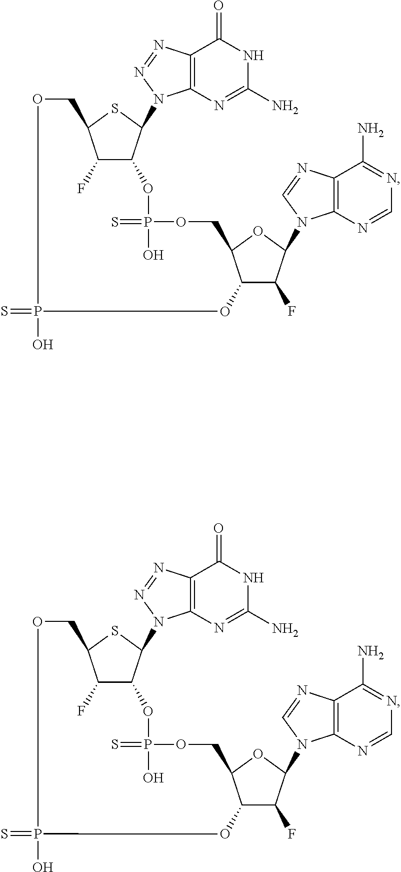 [SMILES: Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1]

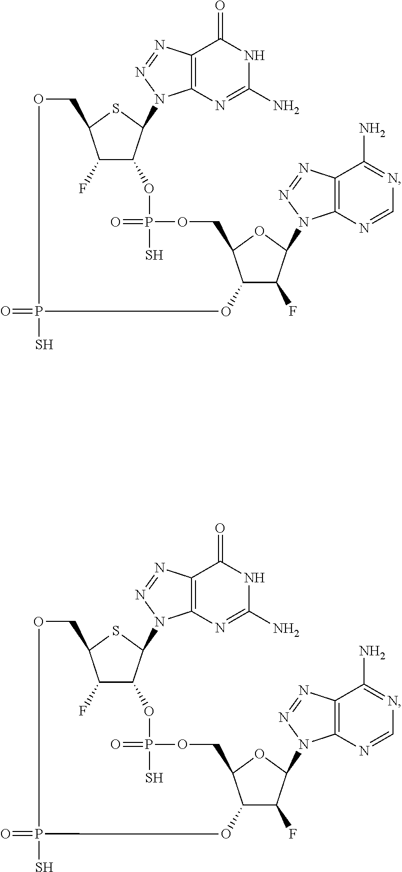 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1]

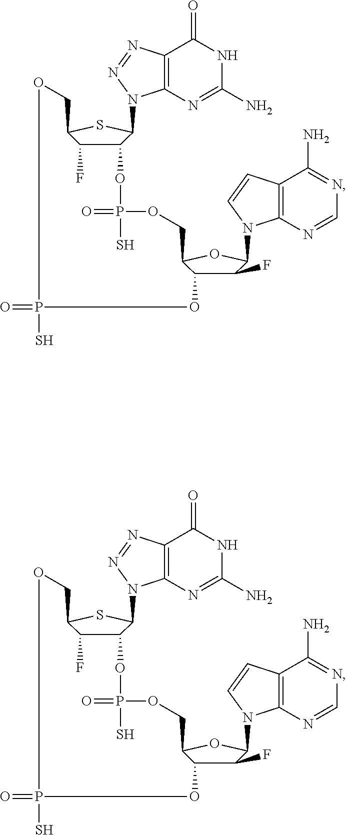 [SMILES: Nc1nc2c(nnn2[C@@H]2S[C@@H]3COP(=O)(S)CO[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)CO[C@H]4[C@H](F)[C@H](n5ccc6c(N)ncnc65)O[C@@H]4COP(=O)(S)O[C@@H]2[C@@H]3F)c(=O)[nH]1]

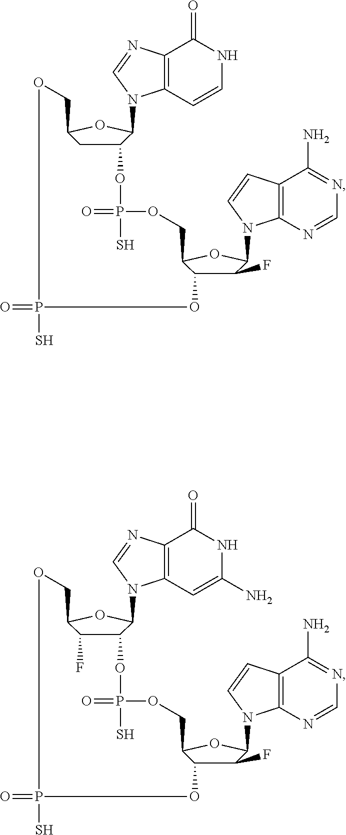 [SMILES: Nc1ncnc2c1ccn2[C@@H]1O[C@@H]2COP(=O)(S)O[C@@H]3C[C@@H](COP(=O)(S)CO[C@H]2[C@@H]1F)O[C@H]3n1cnc2c(=O)[nH]ccc21 ; Nc1cc2c(ncn2[C@@H]2O[C@@H]3COP(=O)(S)CO[C@H]4[C@H](F)[C@H](n5ccc6c(N)ncnc65)O[C@@H]4COP(=O)(S)O[C@@H]2[C@@H]3F)c(=O)[nH]1]

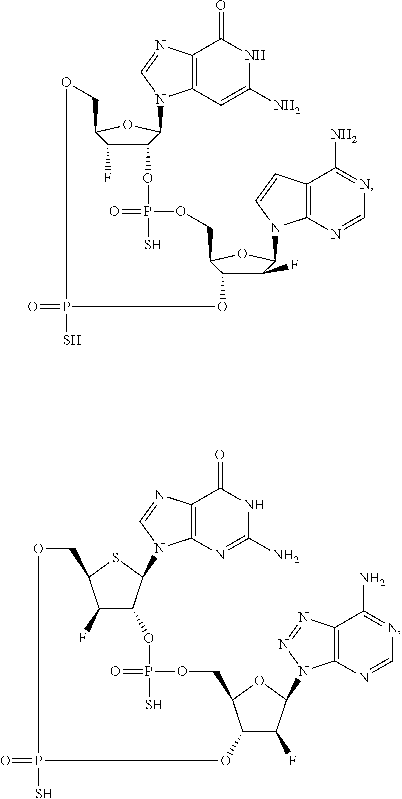 [SMILES: Nc1cc2c(ncn2[C@@H]2O[C@@H]3COP(=O)(S)CO[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1]

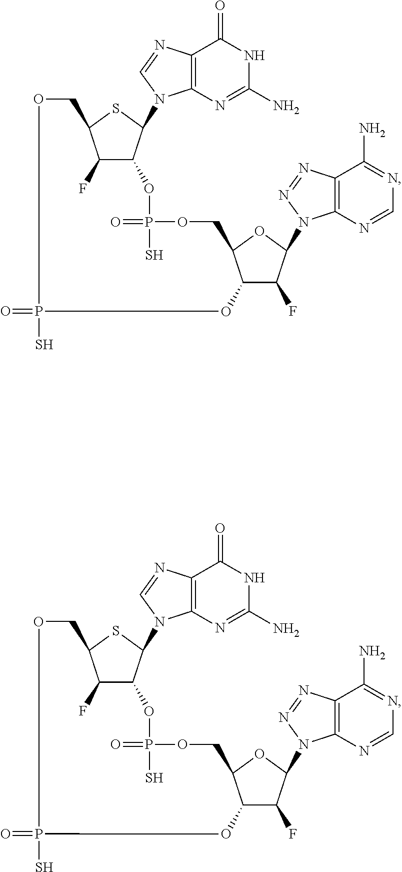 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1]

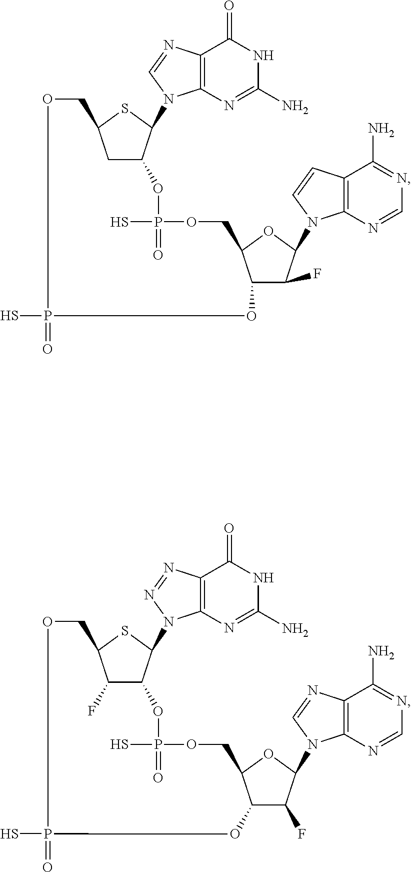 [SMILES: Nc1nc2c(ncn2[C@@H]2S[C@@H]3COP(=O)(S)O[C@H]4[C@H](F)[C@H](n5ccc6c(N)ncnc65)O[C@@H]4COP(=O)(S)O[C@@H]2C3)c(=O)[nH]1 ; Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

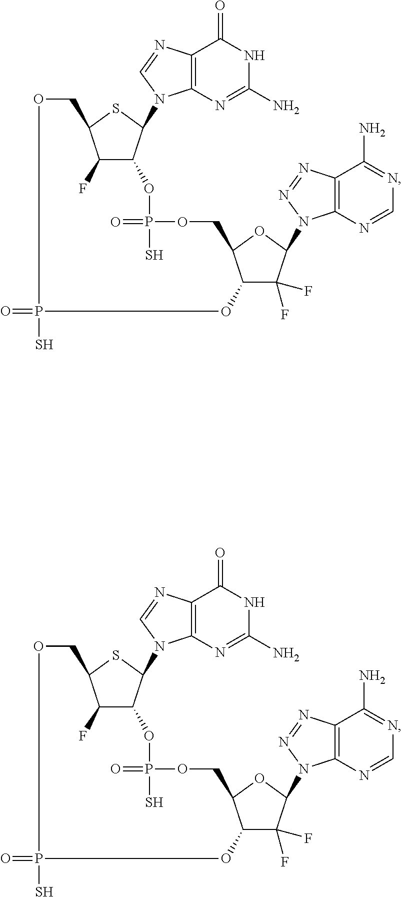 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1]

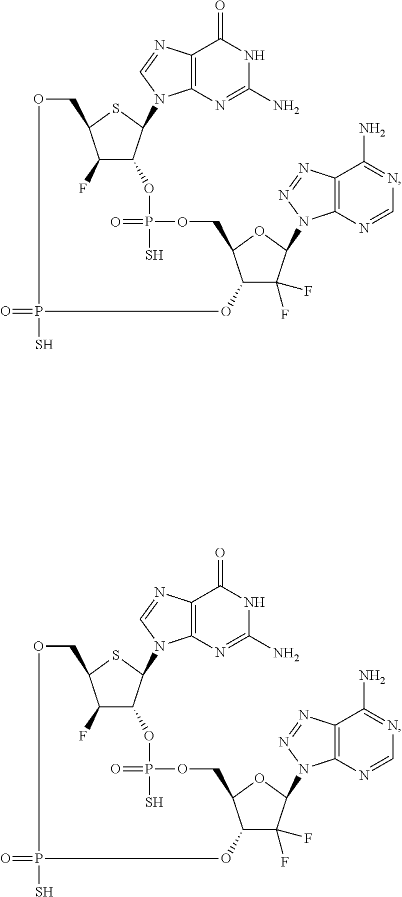 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1]

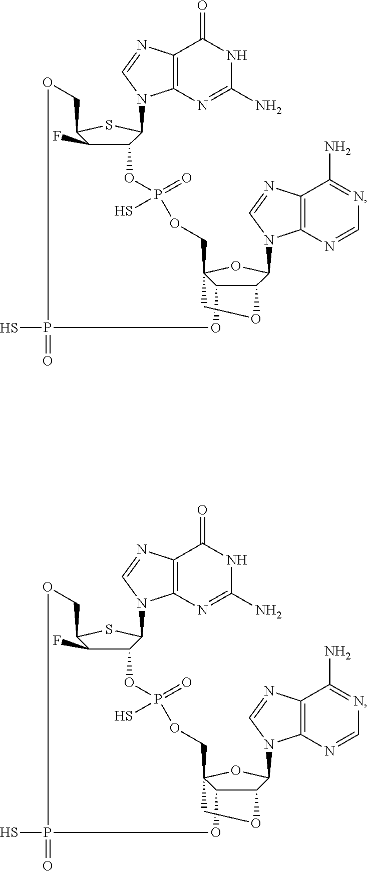 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1]

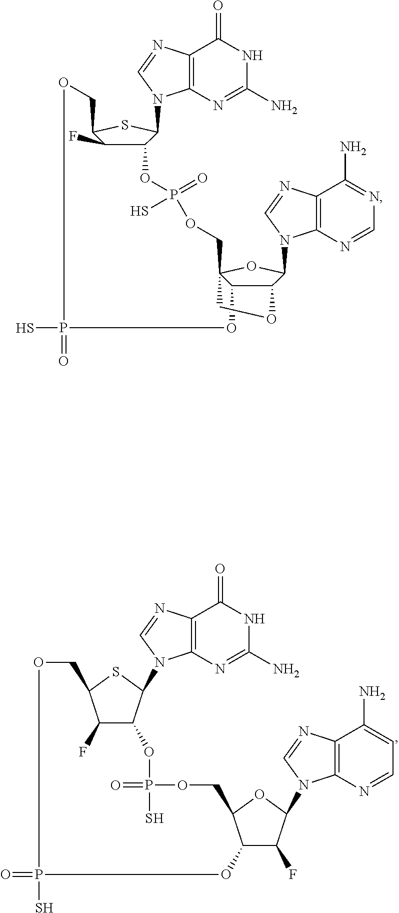 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ccnc65)O[C@@H]4COP(=O)(S)O[C@@H]2[C@H]3F)c(=O)[nH]1]

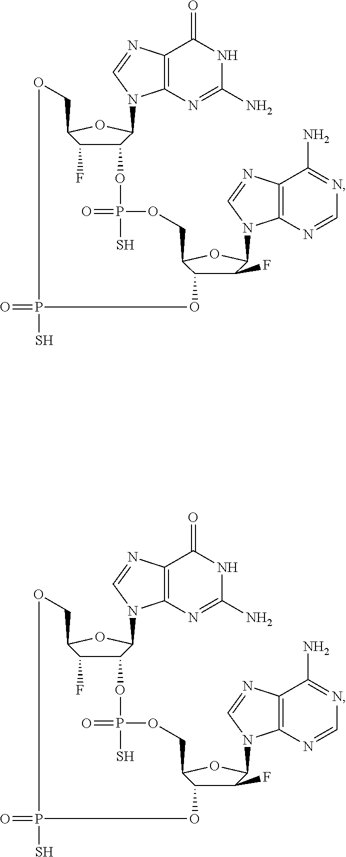 [SMILES: Nc1nc2c(ncn2[C@@H]2O[C@@H]3COP(=O)(S)CO[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4[C@H](F)[C@H](n5cnc6c(N)ncnc65)O[C@@H]4COP(=O)(S)O[C@@H]2[C@@H]3F)c(=O)[nH]1]

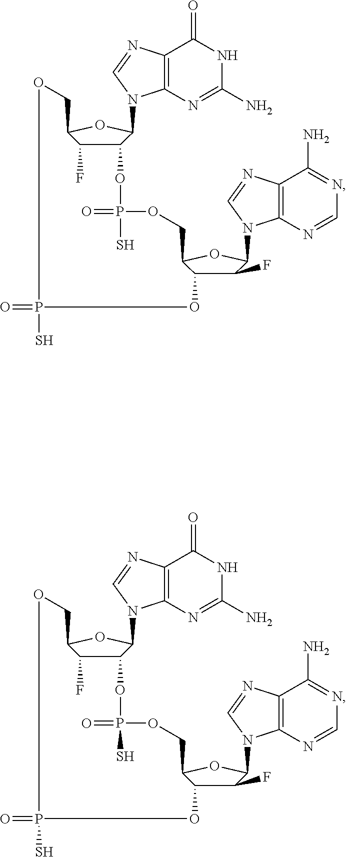 [SMILES: Nc1nc2c(ncn2[C@@H]2O[C@@H]3COP(=O)(S)CO[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)CO[C@H]4[C@H](F)[C@H](n5cnc6c(N)ncnc65)O[C@@H]4CO[P@@](=O)(S)O[C@@H]2[C@@H]3F)c(=O)[nH]1]

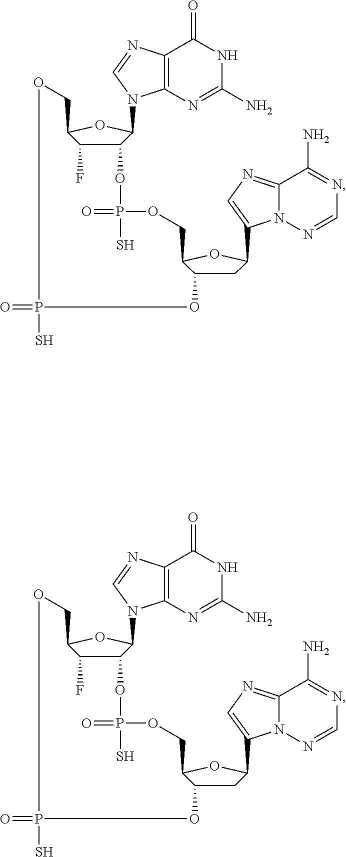 [SMILES: Nc1nc2c(ncn2[C@@H]2O[C@@H]3COP(=O)(S)CO[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4C[C@H](c5cnc6c(N)ncnn56)O[C@@H]4COP(=O)(S)O[C@@H]2[C@@H]3F)c(=O)[nH]1]

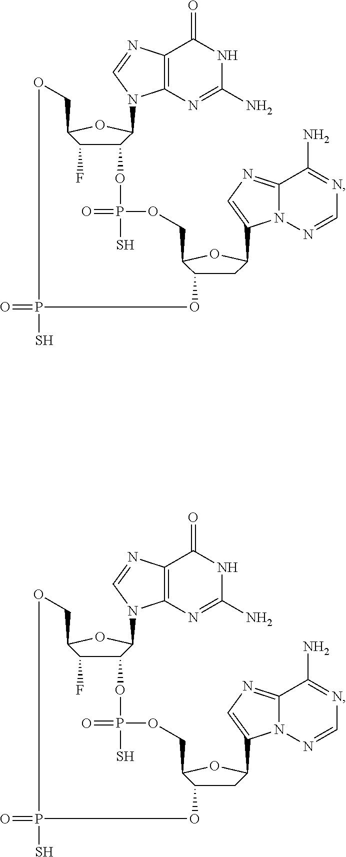 [SMILES: Nc1nc2c(ncn2[C@@H]2O[C@@H]3COP(=O)(S)CO[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4C[C@H](c5cnc6c(N)ncnn56)O[C@@H]4COP(=O)(S)O[C@@H]2[C@@H]3F)c(=O)[nH]1]

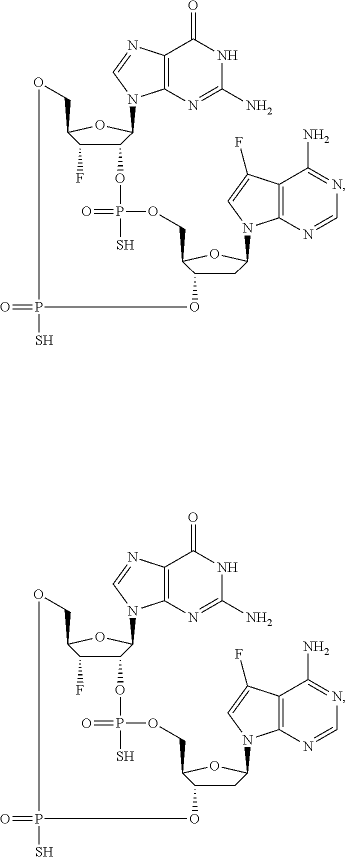 [SMILES: Nc1nc2c(ncn2[C@@H]2O[C@@H]3COP(=O)(S)CO[C@H]4C[C@H](n5cc(F)c6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4C[C@H](n5cc(F)c6c(N)ncnc65)O[C@@H]4COP(=O)(S)O[C@@H]2[C@@H]3F)c(=O)[nH]1]

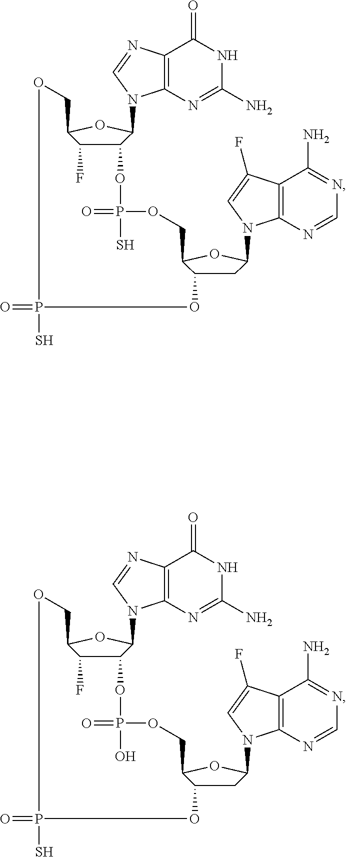 [SMILES: Nc1nc2c(ncn2[C@@H]2O[C@@H]3COP(=O)(S)CO[C@H]4C[C@H](n5cc(F)c6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4C[C@H](n5cc(F)c6c(N)ncnc65)O[C@@H]4COP(=O)(O)O[C@@H]2[C@@H]3F)c(=O)[nH]1]

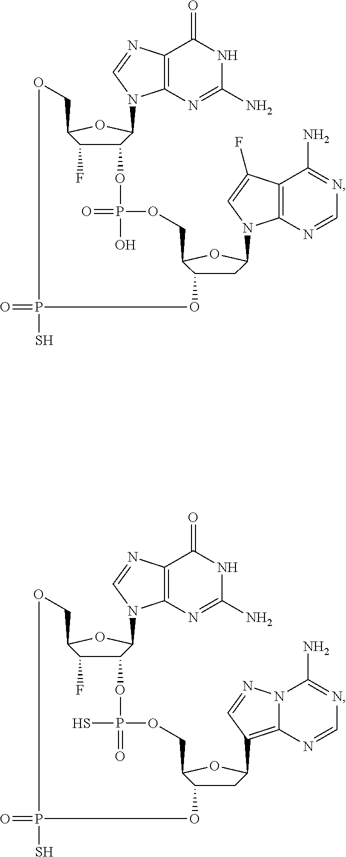 [SMILES: Nc1nc2c(ncn2[C@@H]2O[C@@H]3COP(=O)(S)CO[C@H]4C[C@H](n5cc(F)c6c(N)ncnc65)O[C@@H]4COP(=O)(O)O[C@@H]2[C@@H]3F)c(=O)[nH]1 ; Nc1nc2c(ncn2[C@@H]2O[C@@H]3COP(=O)(S)CO[C@H]4C[C@H](c5cnn6c(N)ncnc56)O[C@@H]4COP(=O)(S)O[C@@H]2[C@@H]3F)c(=O)[nH]1]

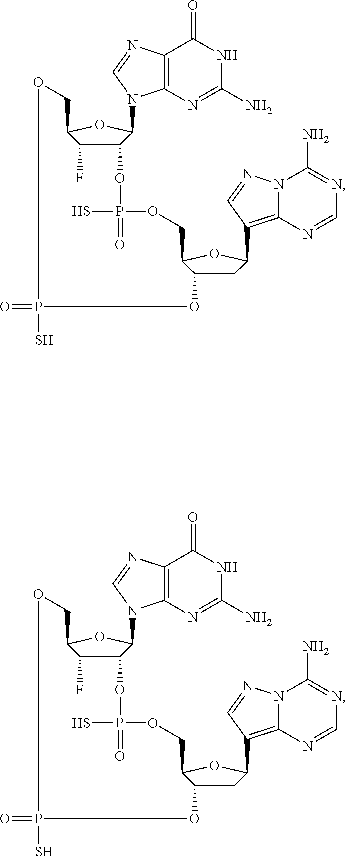 [SMILES: Nc1nc2c(ncn2[C@@H]2O[C@@H]3COP(=O)(S)CO[C@H]4C[C@H](c5cnn6c(N)ncnc56)O[C@@H]4COP(=O)(S)O[C@@H]2[C@@H]3F)c(=O)[nH]1 ; Nc1nc2c(ncn2[C@@H]2O[C@@H]3COP(=O)(S)CO[C@H]4C[C@H](c5cnn6c(N)ncnc56)O[C@@H]4COP(=O)(S)O[C@@H]2[C@@H]3F)c(=O)[nH]1]

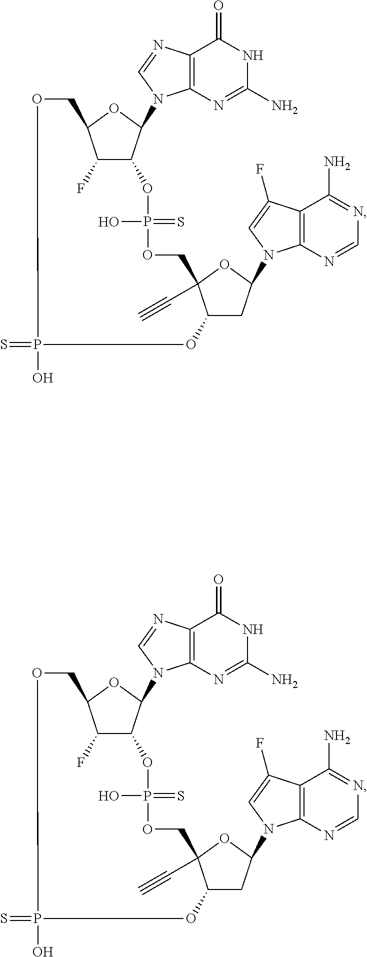 [SMILES: C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21]

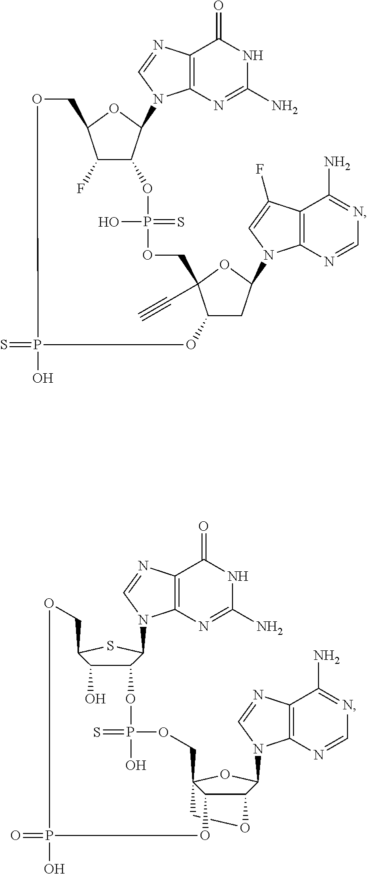 [SMILES: C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; Nc1nc2c(ncn2[C@@H]2S[C@@H]3COP(=O)(O)CO[C@H]4[C@H]5OC[C@]4(COP(O)(=S)O[C@@H]2[C@@H]3O)O[C@H]5n2cnc3c(N)ncnc32)c(=O)[nH]1]

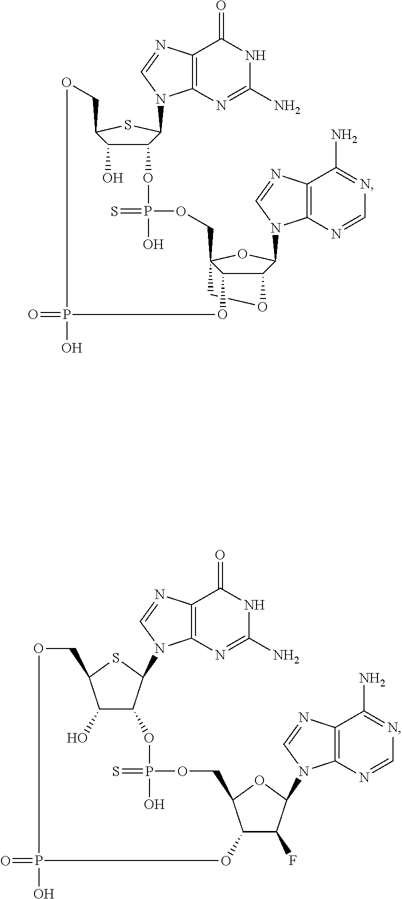 [SMILES: Nc1nc2c(ncn2[C@@H]2S[C@@H]3COP(=O)(O)CO[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(=O)(O)O[C@H]4[C@H](F)[C@H](n5cnc6c(N)ncnc65)O[C@@H]4COP(O)(=S)O[C@@H]2[C@@H]3O)c(=O)[nH]1]

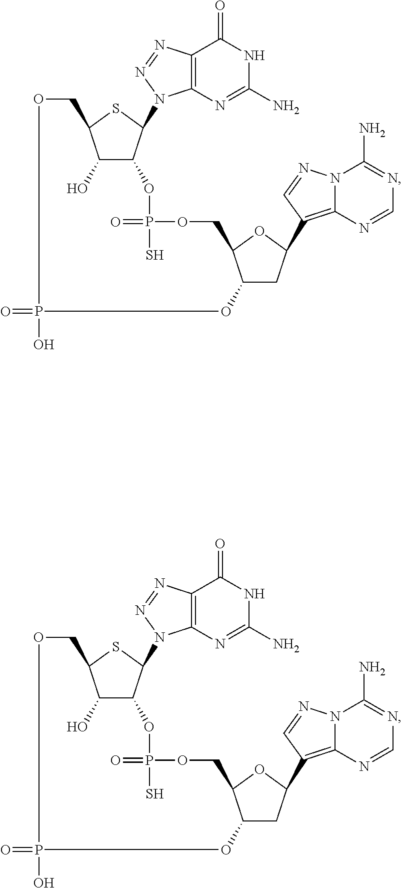 [SMILES: Nc1nc2c(nnn2[C@@H]2S[C@@H]3COP(=O)(O)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnn6c(N)ncnc56)O[C@@H]4COP(=O)(S)O[C@@H]2[C@@H]3O)c(=O)[nH]1]

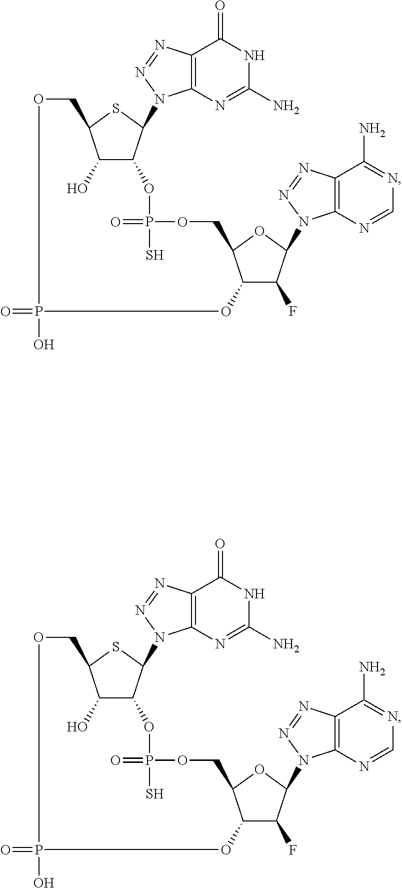 [SMILES: Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1]

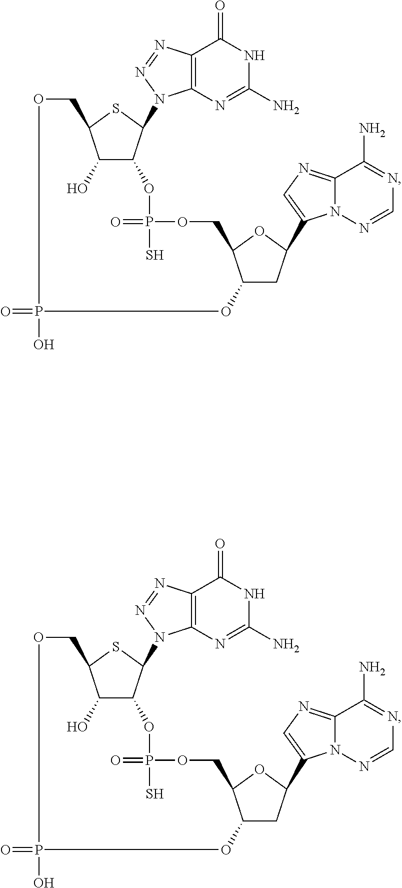 [SMILES: Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1]

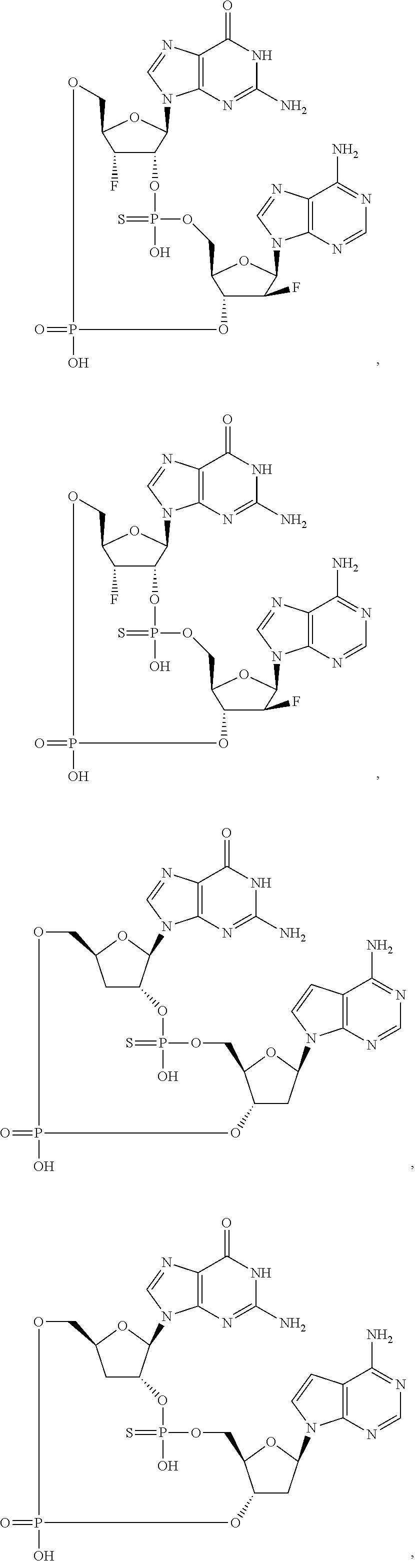 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1]

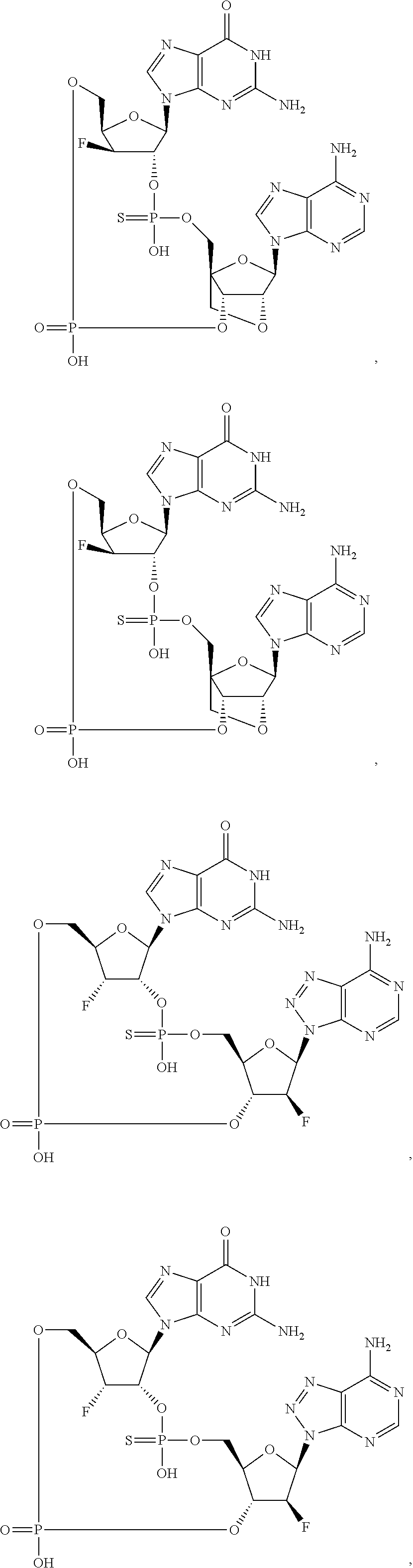 [SMILES: Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1]

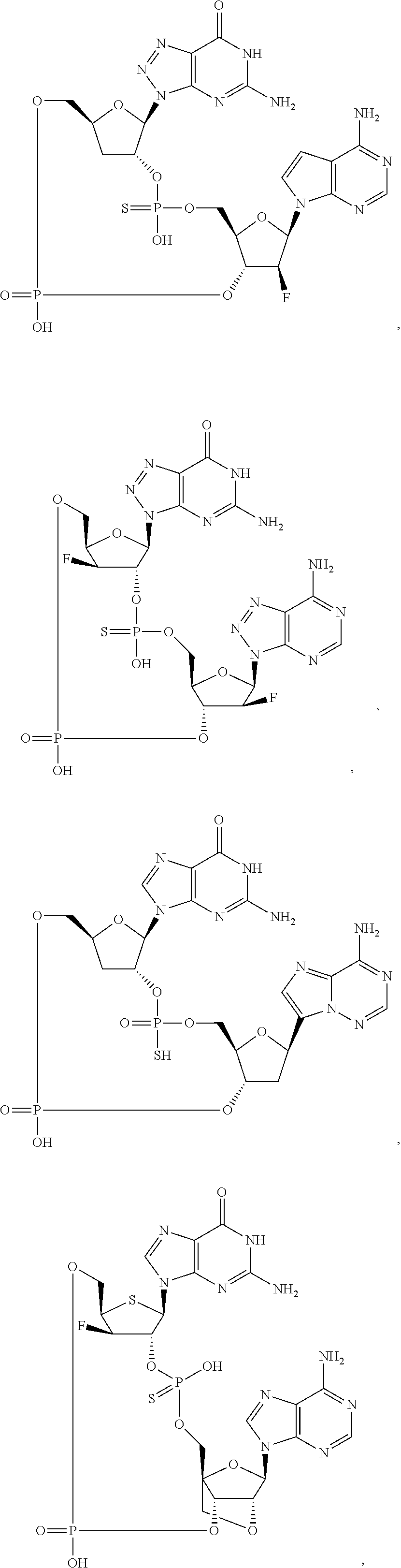 [SMILES: Nc1nc2c(nnn2[C@@H]2O[C@@H]3COP(=O)(O)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(=O)(O)O[C@H]4[C@H](F)[C@H](n5nnc6c(N)ncnc65)O[C@@H]4COP(O)(=S)O[C@@H]2[C@H]3F)c(=O)[nH]1 ; Nc1nc2c(ncn2[C@@H]2O[C@@H]3COP(=O)(O)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2S[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1]

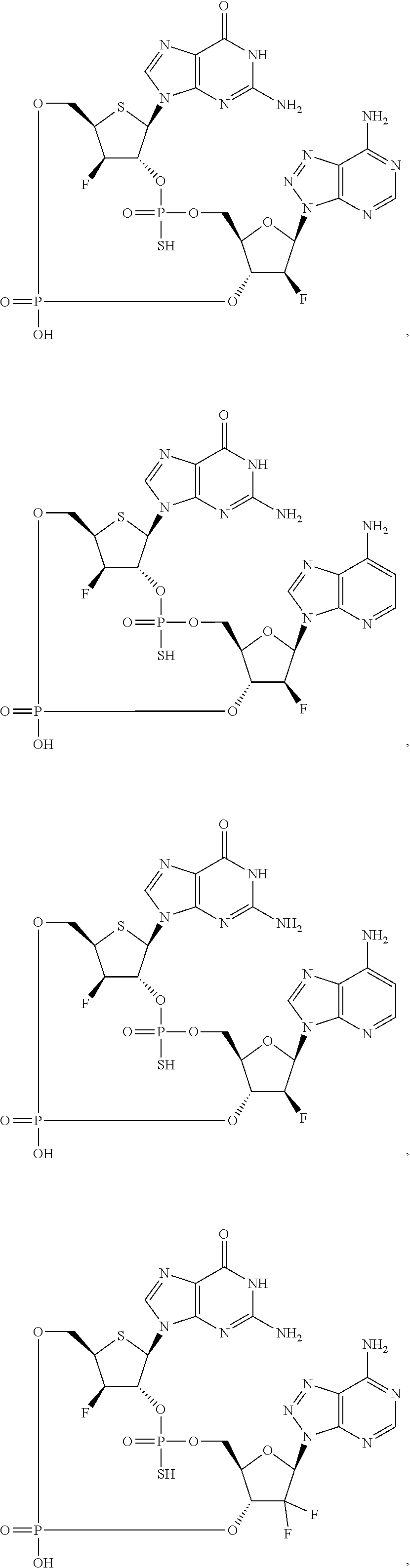 [SMILES: Nc1nc2c(ncn2[C@@H]2S[C@@H]3COP(=O)(O)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@@H]4[C@@H](COP(=O)(S)O[C@@H]2[C@H]3F)O[C@@H](n2nnc3c(N)ncnc32)C4(F)F)c(=O)[nH]1]

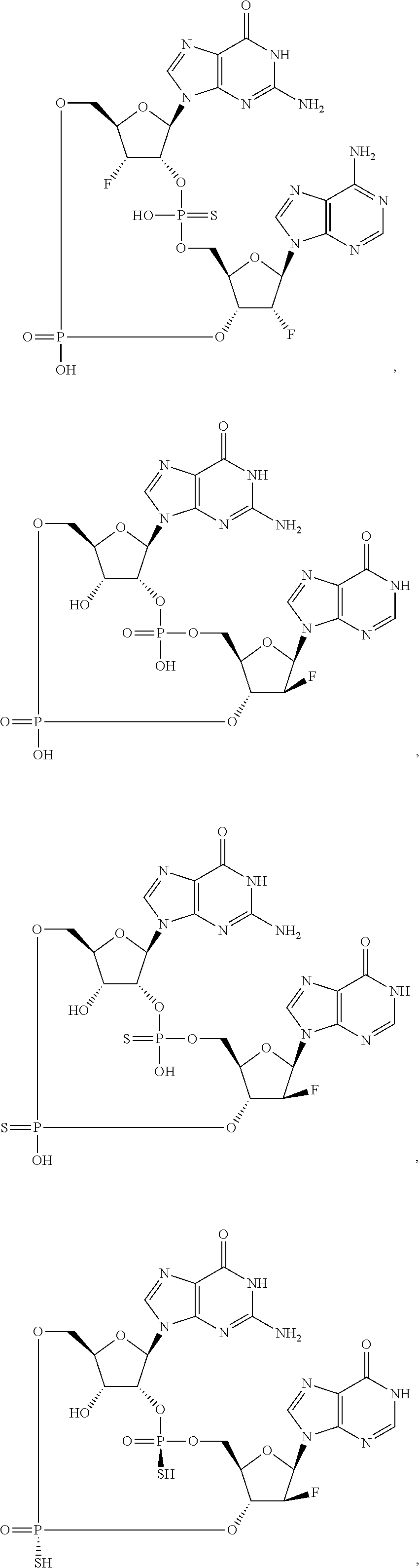

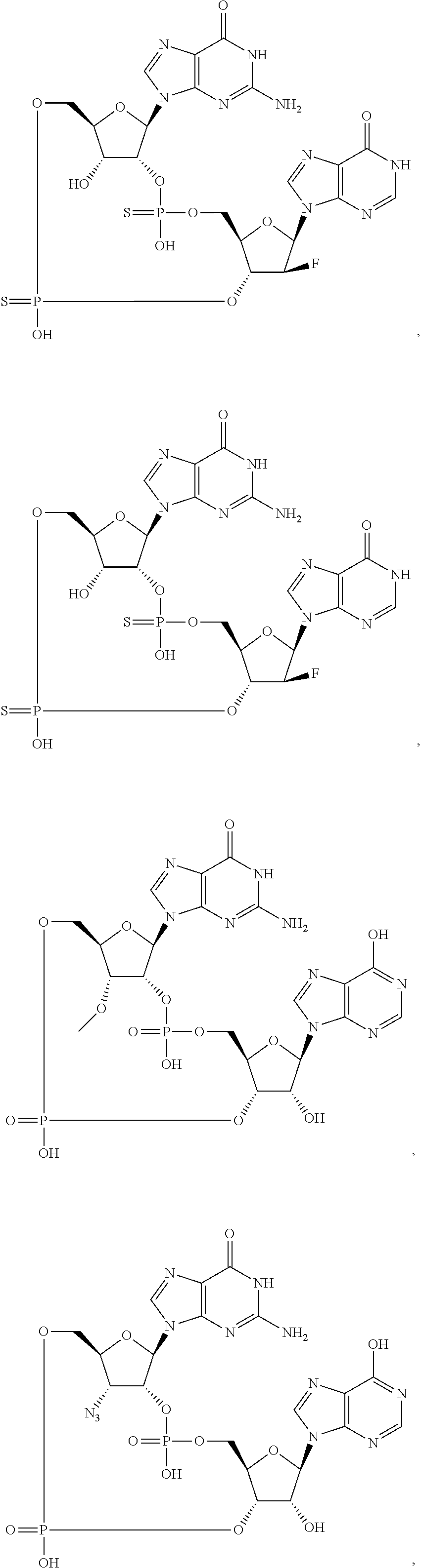

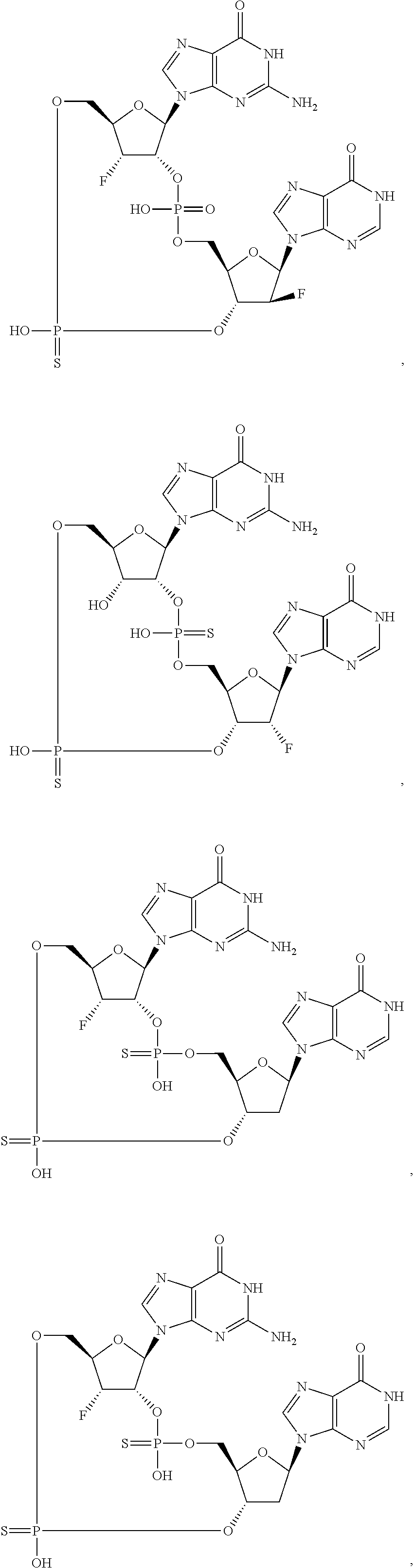 [SMILES: Nc1nc2c(ncn2[C@@H]2O[C@@H]3COP(O)(=S)O[C@H]4[C@H](F)[C@H](n5cnc6c(=O)[nH]cnc65)O[C@@H]4COP(=O)(O)O[C@@H]2[C@@H]3F)c(=O)[nH]1 ; Nc1nc2c(ncn2[C@@H]2O[C@@H]3COP(O)(=S)O[C@H]4[C@@H](F)[C@H](n5cnc6c(=O)[nH]cnc65)O[C@@H]4COP(O)(=S)O[C@@H]2[C@@H]3O)c(=O)[nH]1 ; Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1]

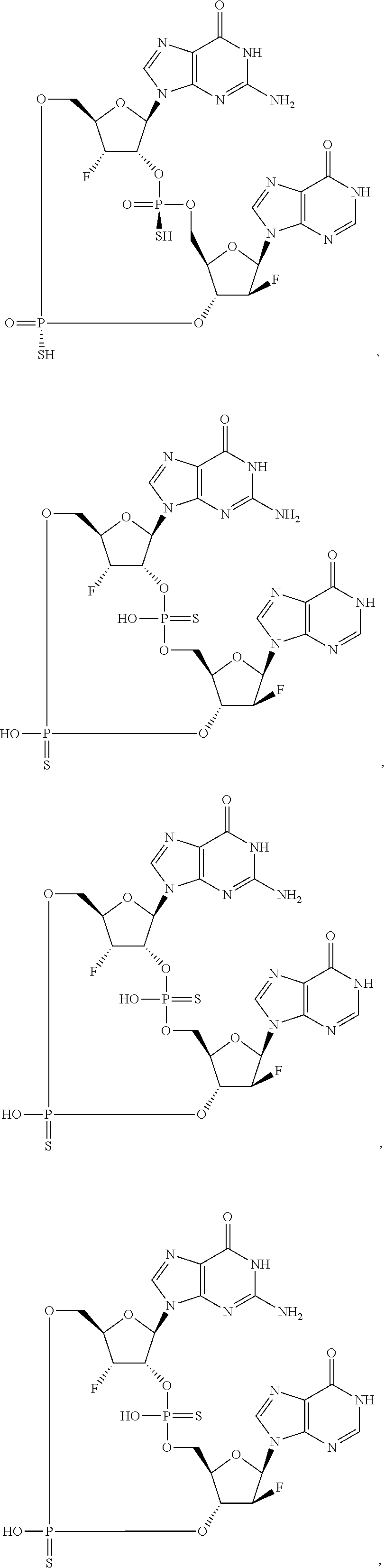

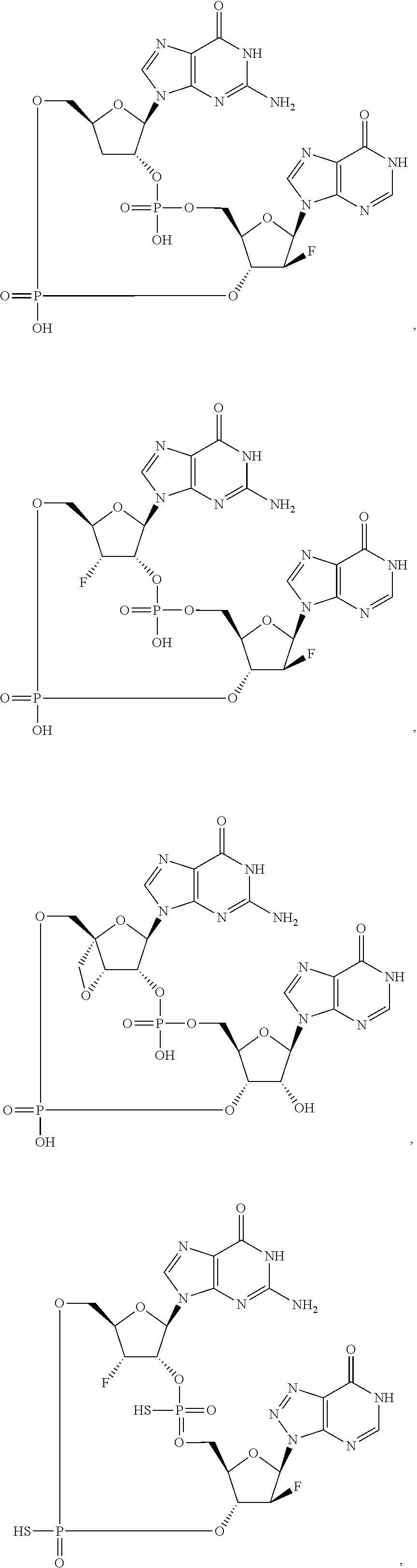

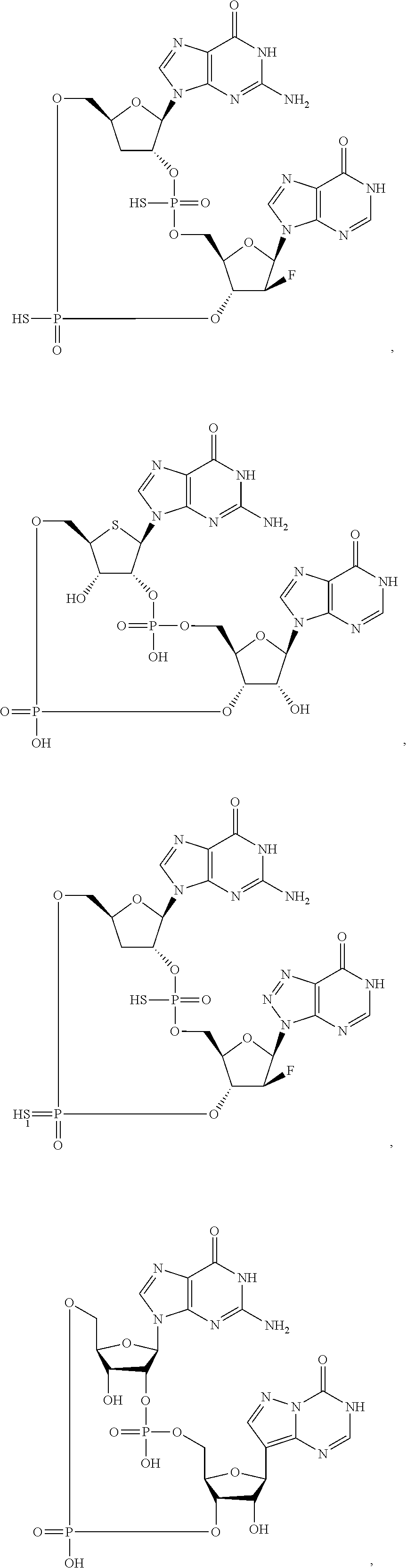

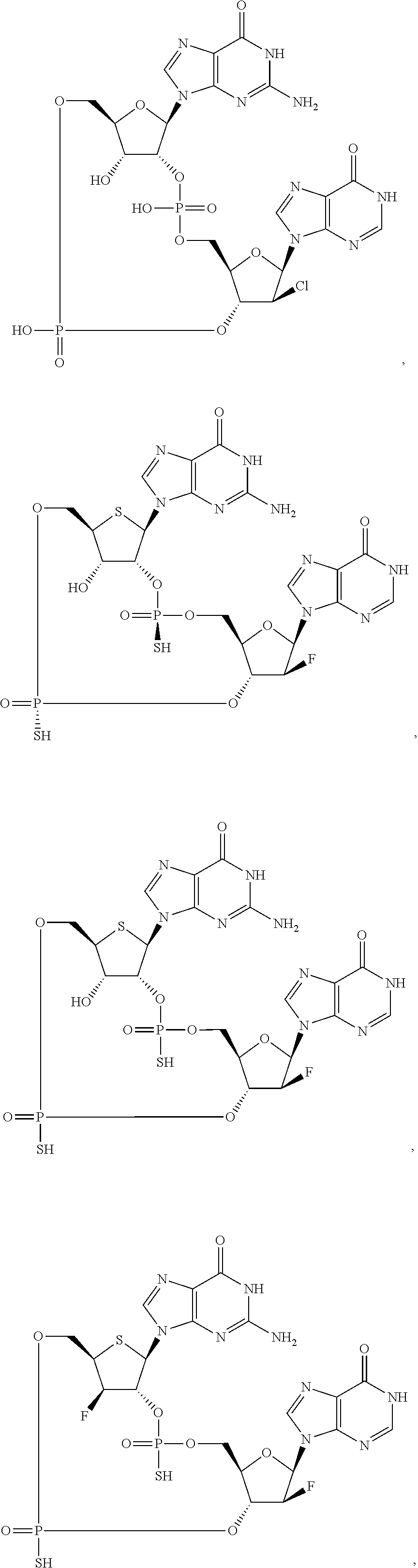

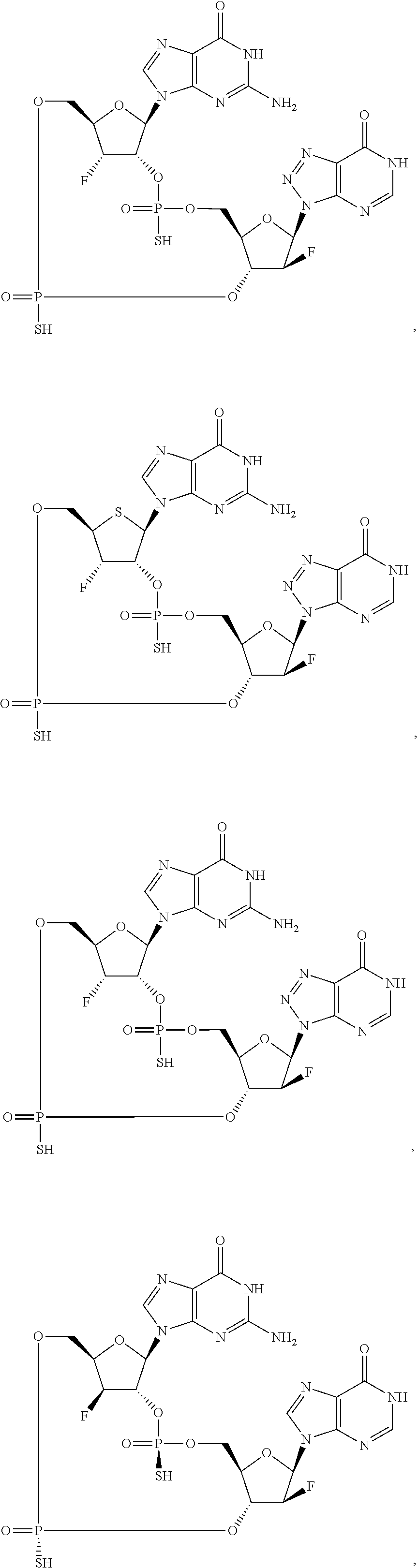

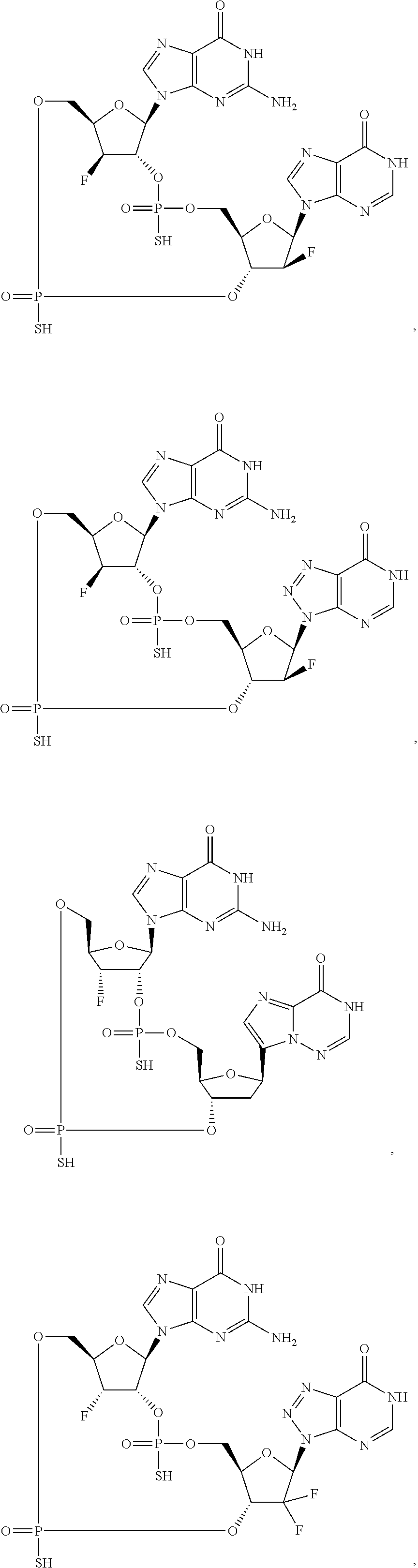 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4C[C@H](c5cnc6c(=O)[nH]cnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1]

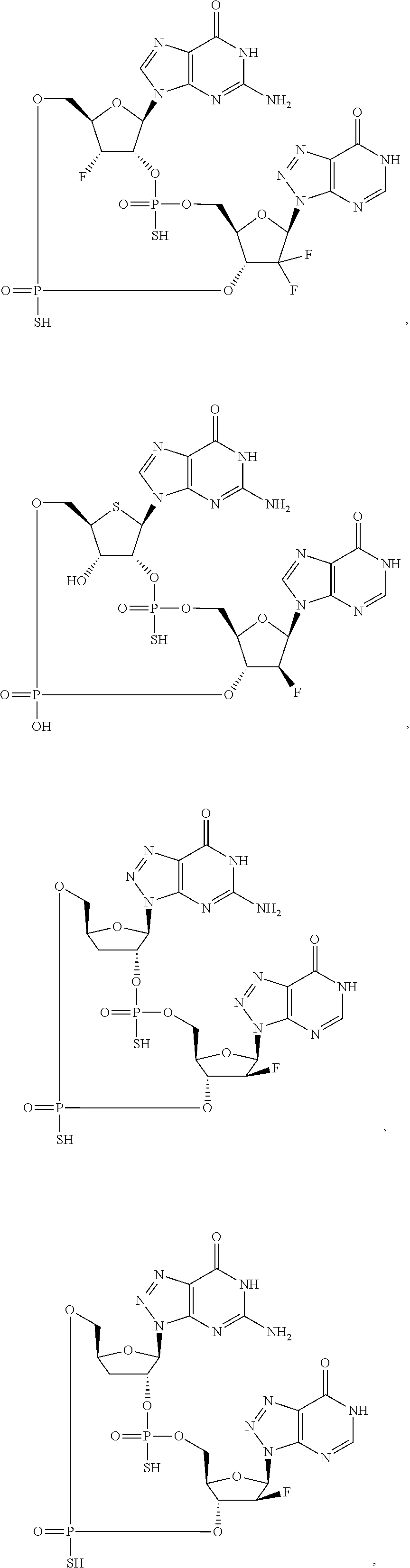 [SMILES: Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1 ; Nc1nc2c(ncn2[C@@H]2S[C@@H]3COP(=O)(O)O[C@H]4[C@H](F)[C@H](n5cnc6c(=O)[nH]cnc65)O[C@@H]4COP(=O)(S)O[C@@H]2[C@@H]3O)c(=O)[nH]1 ; Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1 ; Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1]

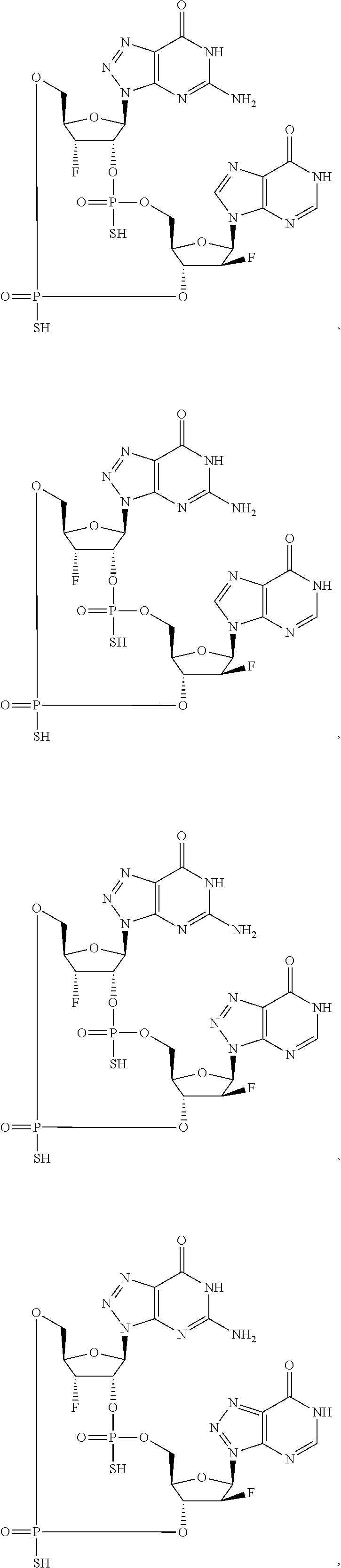

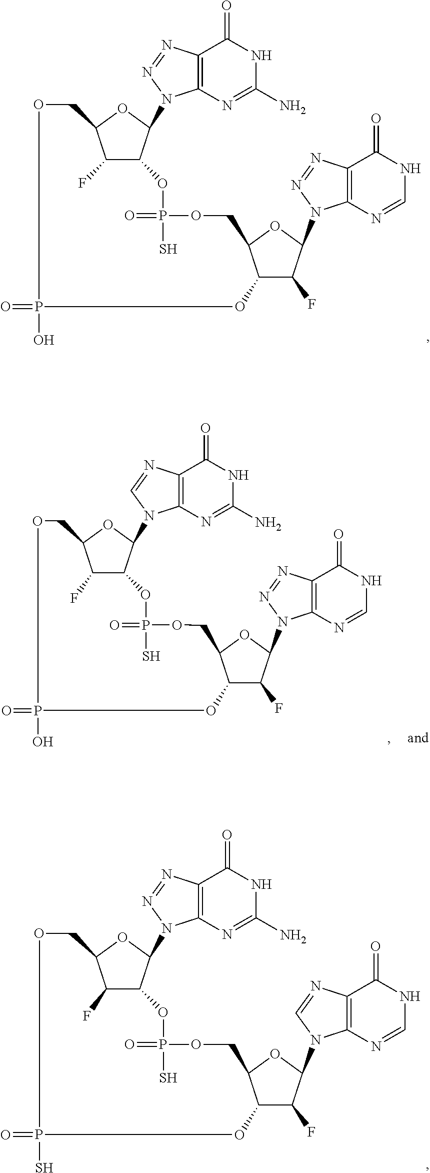 [SMILES: Nc1nc2c(nnn2[C@@H]2O[C@@H]3COP(=O)(O)O[C@H]4[C@H](F)[C@H](n5nnc6c(=O)[nH]cnc65)O[C@@H]4COP(=O)(S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5nnc6c(=O)[nH]cnc65)O[C@@H]4COP(=O)(S)O[C@@H]2[C@@H]3F)c(=O)[nH]1 ; Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1]

and
pharmaceutically acceptable salts thereof.

In particular aspects, the compound is selected from the group consisting of

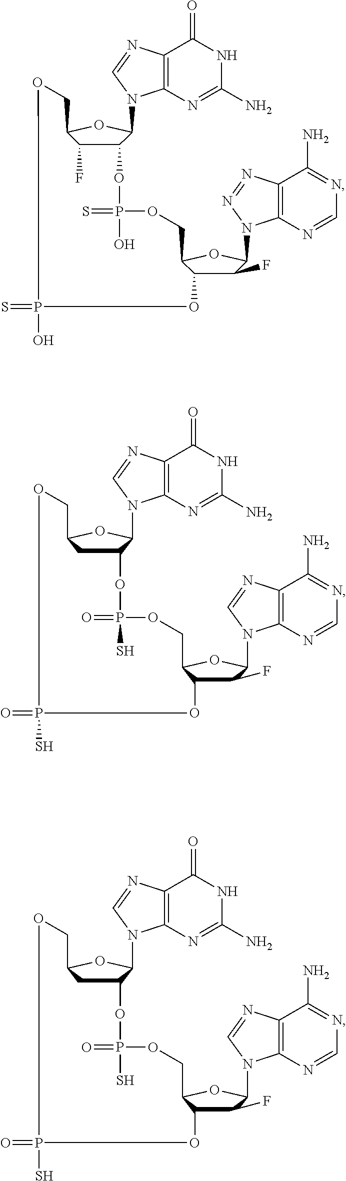

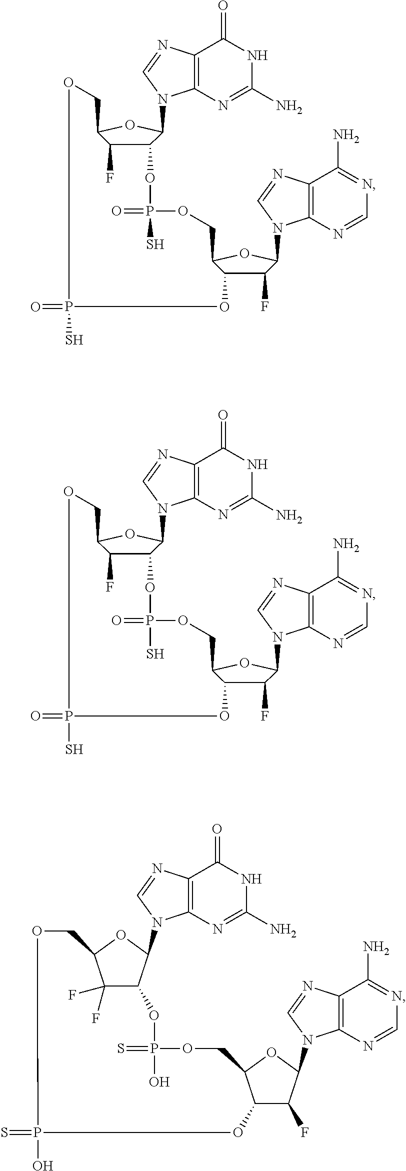 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1]

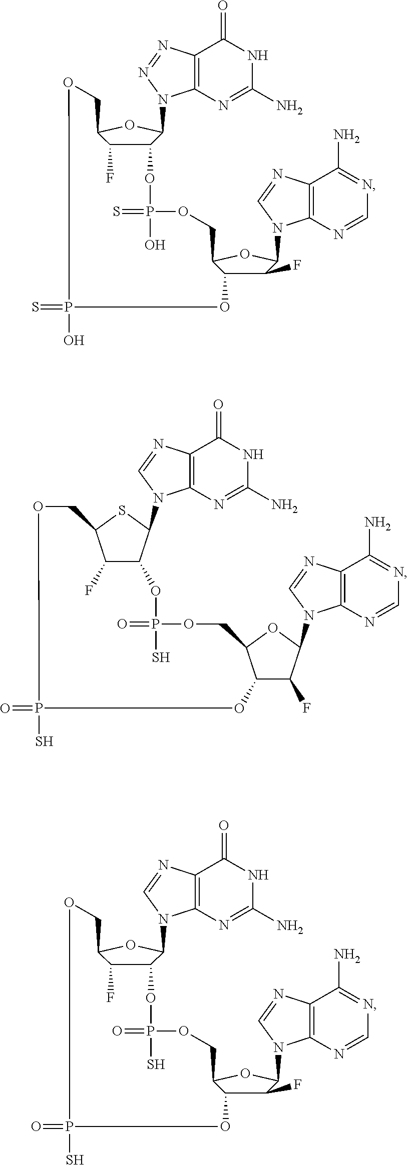 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

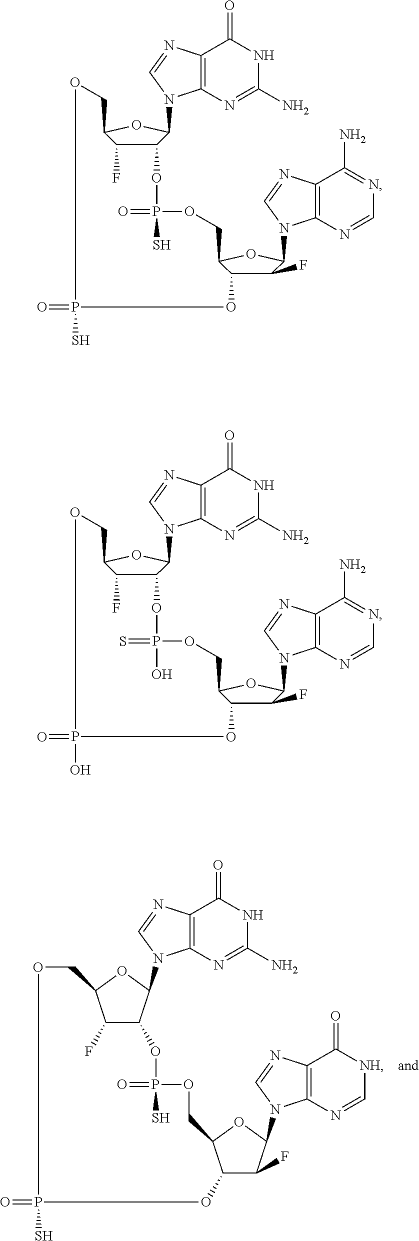 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2[C@@H]3F)c(=O)[nH]1]

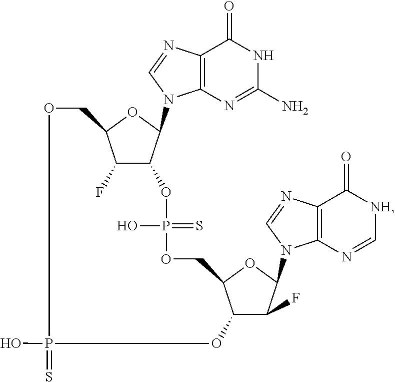 [SMILES: Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

and pharmaceutically acceptable salts thereof. In more particular aspects, the compound is selected from the group consisting of

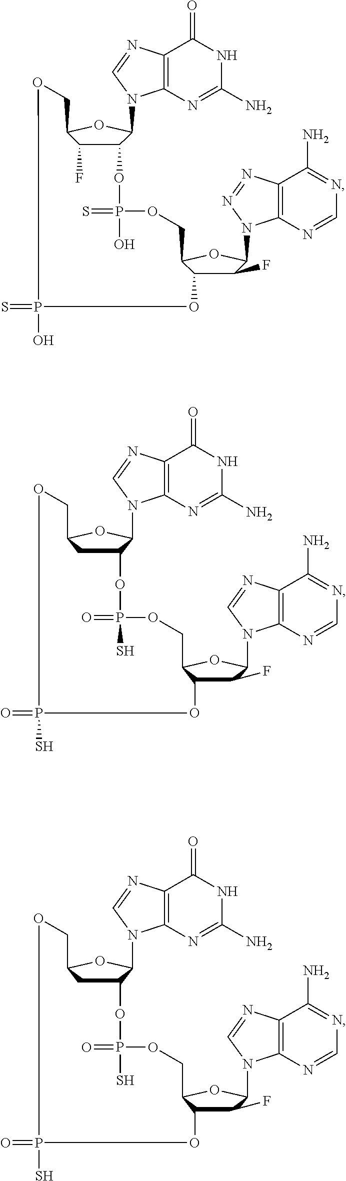

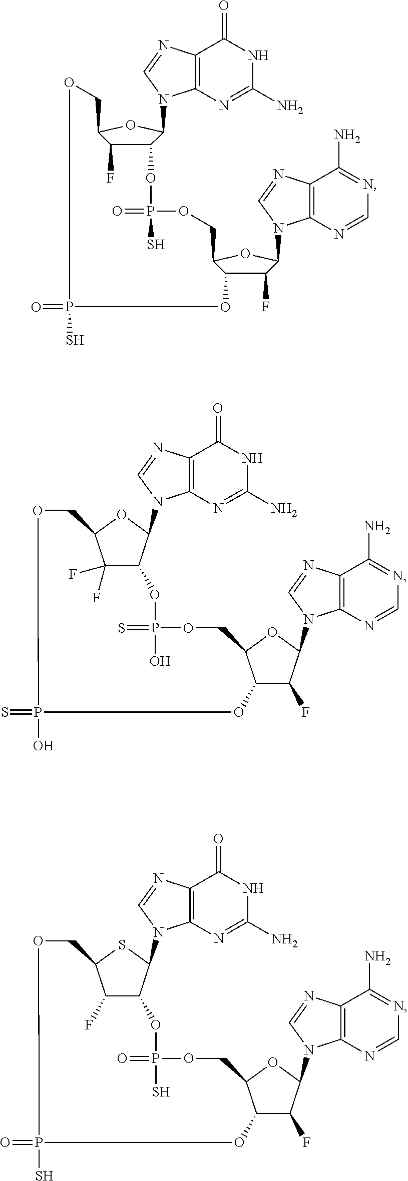 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1 ; Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

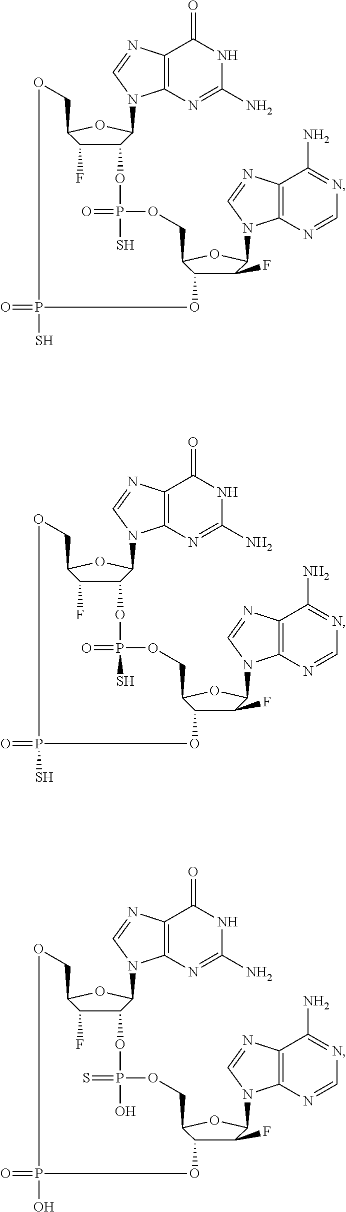 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1]

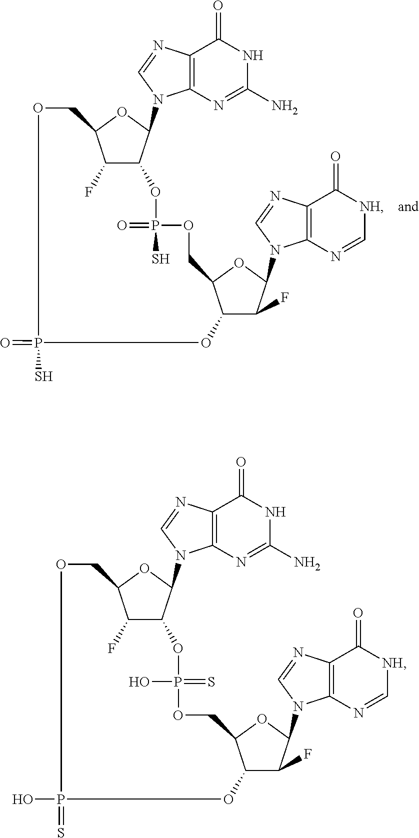 [SMILES: Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

and pharmaceutically acceptable salts thereof.

In still more particular aspects, the compound is selected from the group consisting of

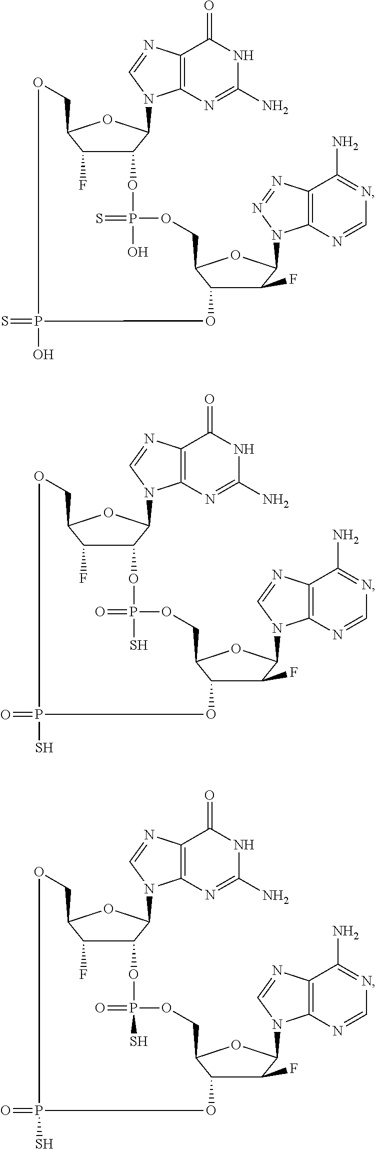 [SMILES: Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

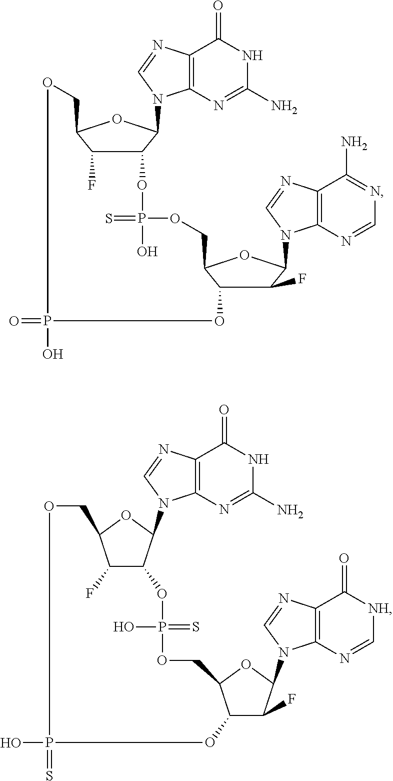 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1]

and pharmaceutically acceptable salts thereof. In specific aspects, the compound is

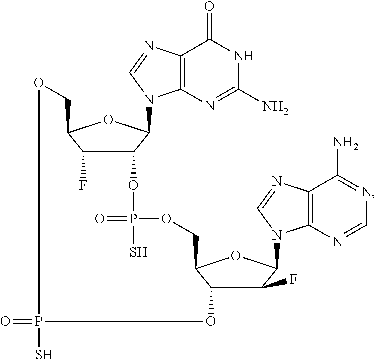 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

or a pharmaceutically acceptable salt thereof. In more specific aspects, the compound is

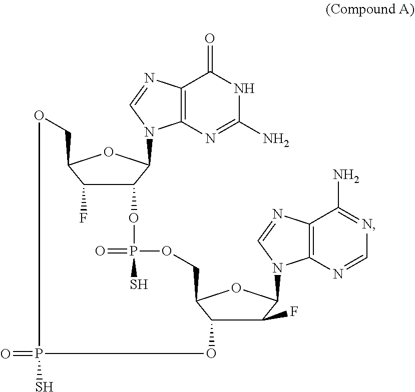 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

or a pharmaceutically acceptable salt thereof.

In further aspects, the cyclic dinucleotide STING agonist compound is selected from the group consisting of

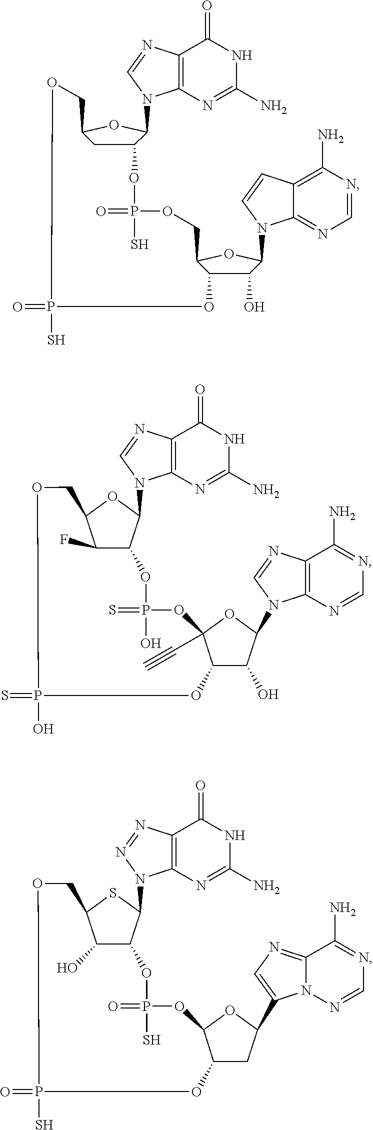 [SMILES: Nc1nc2c(ncn2[C@@H]2O[C@@H]3COP(=O)(S)O[C@H]4[C@@H](O)[C@H](n5ccc6c(N)ncnc65)O[C@@H]4COP(=O)(S)O[C@@H]2C3)c(=O)[nH]1 ; C#C[C@]12O[C@@H](n3cnc4c(N)ncnc43)[C@H](O)[C@@H]1OP(O)(=S)OC[C@H]1O[C@@H](n3cnc4c(=O)[nH]c(N)nc43)[C@H](OP(O)(=S)O2)[C@H]1F ; Nc1nc2c(nnn2[C@@H]2S[C@@H]3COP(=O)(S)O[C@H]4C[C@H](c5cnc6c(N)ncnn56)O[C@@H]4OP(=O)(S)O[C@@H]2[C@@H]3O)c(=O)[nH]1]

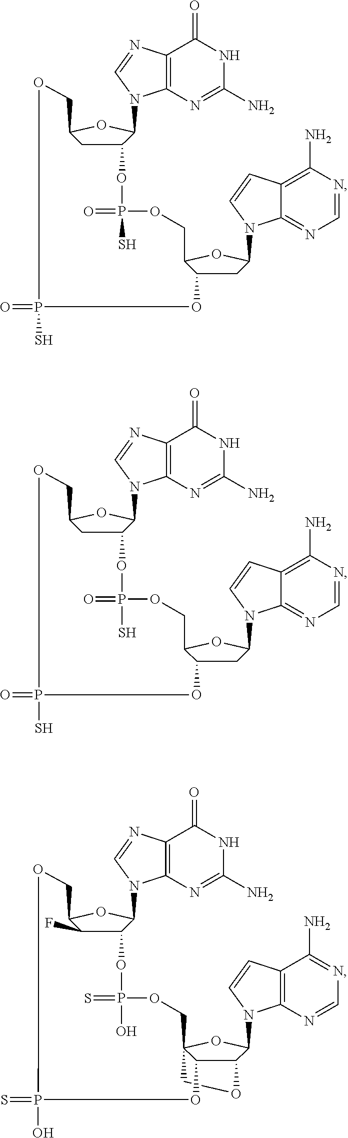 [SMILES: Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2ccc3c(N)ncnc32)c(=O)[nH]1]

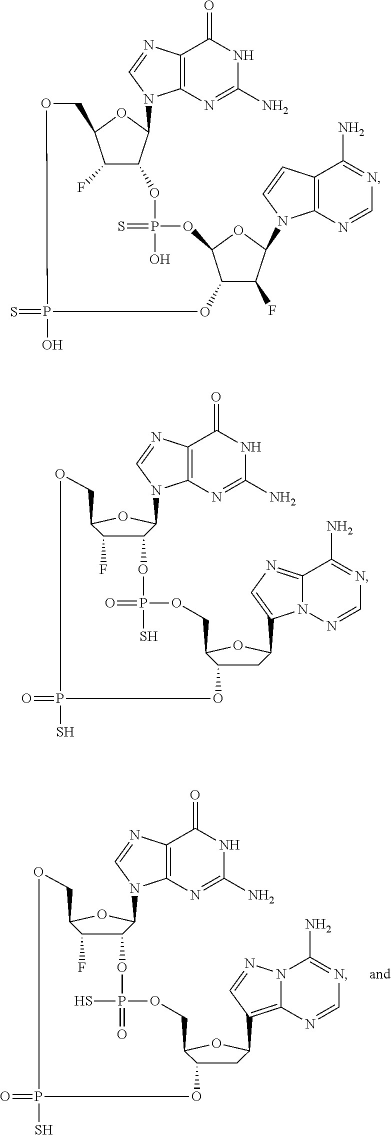 [SMILES: Nc1nc2c(ncn2[C@@H]2O[C@@H]3COP(O)(=S)O[C@@H]4[C@H](O[C@@H](n5ccc6c(N)ncnc65)[C@H]4F)OP(O)(=S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; "and"]

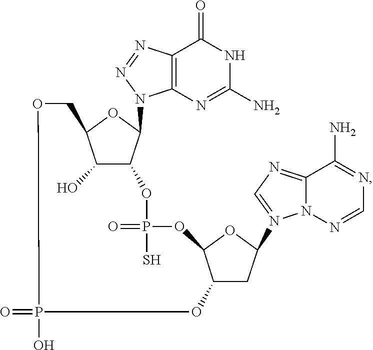

and pharmaceutically acceptable salts thereof. In more particular aspects, the compound is selected from the group consisting of

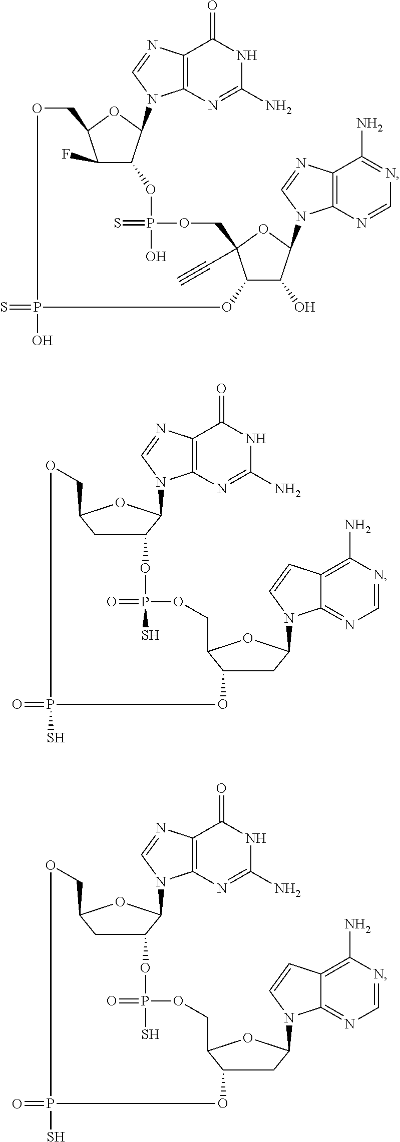 [SMILES: C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1]

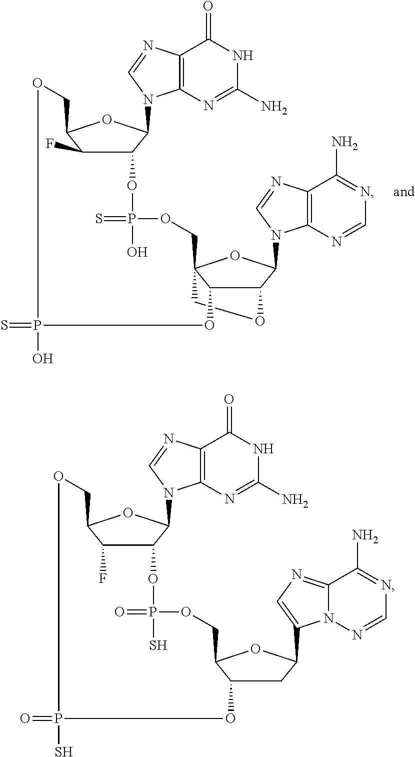 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1]

and pharmaceutically acceptable salts thereof. In still more particular aspects, the compound is selected from the group consisting of

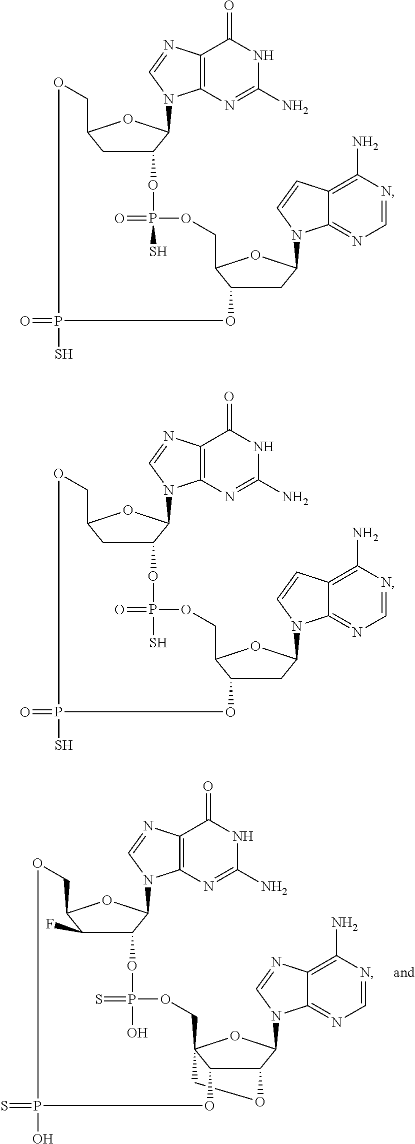 [SMILES: Nc1nc2c(ncn2[C@@H]2OC3COP(=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2OC3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1]

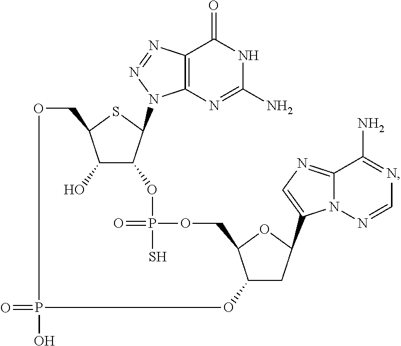 [SMILES: Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1]

and pharmaceutically acceptable salts thereof. In specific aspects, the compound is

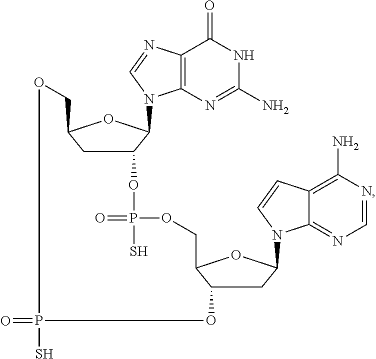 [SMILES: Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1]

or a pharmaceutically acceptable salt thereof. In more specific embodiments, the compound is

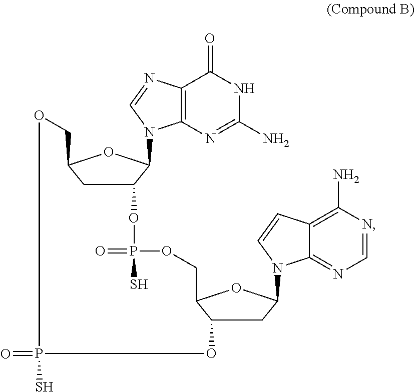 [SMILES: Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1]

or a pharmaceutically acceptable salt thereof.

In aspects of embodiments as described above, the pharmaceutically acceptable aqueous carrier is water.

In aspects of the embodiments described above, the pharmaceutically acceptable tonicity modifier is selected from the group consisting of mannitol, sodium chloride, glycerol, sucrose, and trehalose. In particular aspects, the pharmaceutically acceptable tonicity modifier is selected from the group consisting of mannitol, sodium chloride, and sucrose. In still more particular aspects, the pharmaceutically acceptable tonicity modifier is selected from the group consisting of mannitol.

In aspects of the embodiments described above, the pharmaceutically acceptable buffer is selected from histidine, tris(hydroxymethyl)aminomethane (TRIS), sodium citrate, and sodium phosphate. In particular aspects, the pharmaceutically acceptable buffer is histidine. In more particular aspects, the pharmaceutically acceptable buffer is L-histidine. In specific aspects, the pharmaceutical formulation has a pH of from about 6 to about 7.5, of from about 6 to about 7, of from about 6.3 to about 6.7, or of about 6.5.

In aspects of the embodiments described above, the pharmaceutically acceptable antioxidant is selected from the group consisting of methionine, sodium metabisulfite, thiogylcerol, cysteine, and glutathione. In particular aspects, the pharmaceutically acceptable antioxidant is methionine. In more particular aspects, the pharmaceutically acceptable antioxidant is L-methionine. In even more particular aspects, the pharmaceutically acceptable antioxidant is L-methionine HCl salt.

In aspects of the embodiments described above, the pharmaceutically acceptable metal chelator is selected from the group consisting of diethylenetriaminepentaacetic acid (DTPA) or edetate disodium dehydrate (EDTA). In specific aspects, the pharmaceutically acceptable metal chelator is EDTA.

Another additional embodiment relates to a pharmaceutical formulation comprising (a) one or more cyclic dinucleotide STING agonist compound, present in a total amount of from about 0.25 mg/ml to about 6.0 mg/mL; (b) a pharmaceutically acceptable aqueous carrier, which is water; (c) one or more pharmaceutically acceptable tonicity modifier, present in a total amount of from about 30 mg/ml to about 70 mg/ml; (d) one or more pharmaceutically acceptable buffer, present in a total amount of from about 5 mg/ml to about 10 mg/ml; (e) one or more pharmaceutically acceptable antioxidant, present in a total amount of from about 0.5 mg/ml to about 1.0 mg/ml; and (0 one or more pharmaceutically acceptable metal chelator, present in a total amount of from about 0.01 mg/ml to about 0.04 mg/ml; and wherein said pharmaceutical composition has a pH from about 6 to about 7. A further embodiment relates to a pharmaceutical formulation comprising (a) one or more cyclic dinucleotide STING agonist compound, present in a total amount of from about 0.1 mg/ml to about 4.0 mg/mL; (b) a pharmaceutically acceptable aqueous carrier, which is water; (c) one or more pharmaceutically acceptable tonicity modifier, present in a total amount of from about 30 mg/ml to about 40 mg/ml; (d) one or more pharmaceutically acceptable buffer, present in a total amount of from about 6 mg/ml to about 8 mg/ml; (e) one or more pharmaceutically acceptable antioxidant, present in a total amount of from about 0.5 mg/ml to about 1.0 mg/ml; and (0 one or more pharmaceutically acceptable metal chelator, present in a total amount of from about 0.01 mg/ml to about 0.03 mg/ml; and wherein said pharmaceutical composition has a pH from about 6 to about 7.

Another additional embodiment relates to a pharmaceutical formulation comprising (a) one or more cyclic dinucleotide STING agonist compound, present in a total amount of from about 0.25 mg/ml to about 6.0 mg/mL; (b) a pharmaceutically acceptable aqueous carrier, which is water; (c) one or more pharmaceutically acceptable tonicity modifier, present in a total amount of from about 20 mg/ml to about 60 mg/ml; (d) one or more pharmaceutically acceptable buffer, present in a total amount of from about 6 mg/ml to about 8 mg/ml; (e) one or more pharmaceutically acceptable antioxidant, present in a total amount of from about 0.15 mg/ml to about 1.0 mg/ml; and (0 one or more pharmaceutically acceptable metal chelator, present in a total amount of from about 0.01 mg/ml to about 0.04 mg/ml; and wherein said pharmaceutical composition has a pH from about 6 to about 7. A further embodiment relates to a pharmaceutical formulation comprising (a) one or more cyclic dinucleotide STING agonist compound, present in a total amount of from about 0.1 mg/ml to about 4.0 mg/mL; (b) a pharmaceutically acceptable aqueous carrier, which is water; (c) one or more pharmaceutically acceptable tonicity modifier, present in a total amount of from about 30 mg/ml to about 50 mg/ml; (d) one or more pharmaceutically acceptable buffer, present in a total amount of from about 6 mg/ml to about 8 mg/ml; (e) one or more pharmaceutically acceptable antioxidant, present in a total amount of from about 0.15 mg/ml to about 1.0 mg/ml; and (0 one or more pharmaceutically acceptable metal chelator, present in a total amount of from about 0.01 mg/ml to about 0.03 mg/ml; and wherein said pharmaceutical composition has a pH from about 6 to about 7.

Another additional embodiment relates to a pharmaceutical formulation comprising (a)

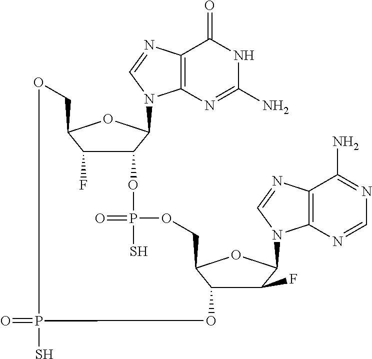 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of from about 0.25 mg/ml to about 6.0 mg/mL; (b) mannitol in an amount of from about 20 to about 60 mg/mL; (c) histidine in an amount of about 5 mg/ml to about 10 mg/ml; (d) methionine in an amount of from about 0.5 mg/ml to about 1.0 mg/ml; and (e) EDTA in an amount of about 0.01 mg/ml to about 0.04 mg/ml; and wherein said pharmaceutical composition has a pH about 6.5.

Another additional embodiment relates to a pharmaceutical formulation comprising (a)

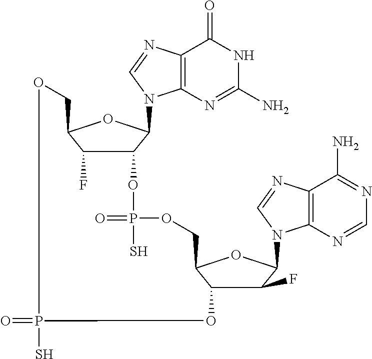 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of from about 0.25 mg/ml to about 6.0 mg/mL; (b) mannitol in an amount of from about 30 to about 40 mg/mL; (c) histidine in an amount of about 6 mg/ml to about 8 mg/ml; (d) methionine in an amount of from about 0.15 mg/ml to about 1.0 mg/ml; and (e) EDTA in an amount of about 0.01 mg/ml to about 0.04 mg/ml; and wherein said pharmaceutical composition has a pH about 6.5.

Another additional embodiment relates to a pharmaceutical formulation comprising (a)

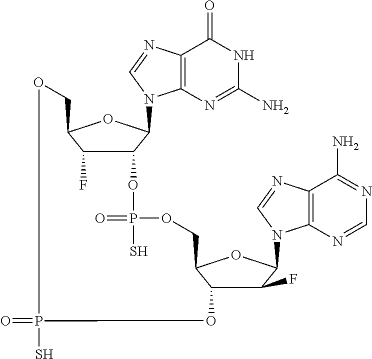 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of from about 0.25 mg/ml to about 6.0 mg/mL; (b) mannitol in an amount of about 34 mg/mL; (c) histidine in an amount of about 7.75 mg/ml; (d) methionine in an amount of from about 0.750 mg/ml; and (e) EDTA in an amount of about 0.0175 mg/ml; and wherein said pharmaceutical composition has a pH about 6.5.

Another additional embodiment relates to a pharmaceutical formulation comprising (a)

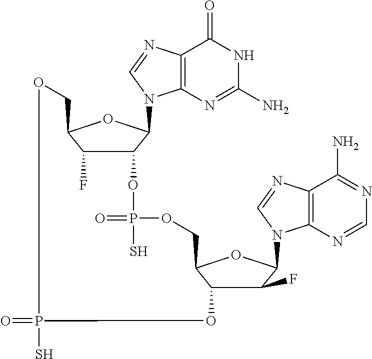 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of from about 0.25 mg/ml to about 6.0 mg/mL; (b) mannitol in an amount of about 40 mg/mL; (c) histidine in an amount of about 7.75 mg/ml; (d) methionine in an amount of from about 0.373 mg/ml; and (e) EDTA in an amount of about 0.0175 mg/ml; and wherein said pharmaceutical composition has a pH about 6.5.

Another additional embodiment relates to a pharmaceutical formulation comprising (a)

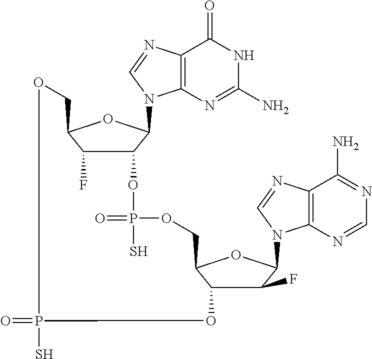 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of from 0.25 mg/ml to about 6.0 mg/mL; (b) mannitol in an amount of about 34 mg/mL; (c) L-histidine in an amount of about 7.75 mg/ml; (d) L-methionine in an amount of from about 0.750 mg/ml; and (e) EDTA in an amount of about 0.0175 mg/ml; and wherein said pharmaceutical composition has a pH about 6.5.

Another additional embodiment relates to a pharmaceutical formulation comprising (a)

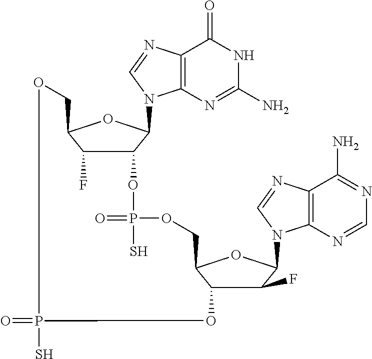 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

in an amount of about 0.54 mg/mL; (b) mannitol in an amount of about 40 mg/mL; (c) L-histidine in an amount of about 7.5 mg/ml; (d) L-methionine in an amount of from about 0.373 mg/ml; and (e) EDTA in an amount of about 0.0175 mg/ml; and wherein said pharmaceutical composition has a pH about 6.5.

In some embodiments, the formulations described herein is in aqueous solution.

In the embodiments as provided above, it is to be understood that each embodiment may be combined with one or more other embodiments, to the extent that such a combination provides a stable formulation and is consistent with the description of the embodiments.

The disclosure also provides a formulation as described herein, wherein the formulation is contained in a glass vial or injection device (e.g. a syringe).

In embodiments of the formulations of the invention, the formulation has one or more of the following attributes after storage at from about 23° C. to about 27° C. for:

a) stability of solution following terminal sterilization,

b) storage at room temperature (about 23° C. to about 27° C., about 25° C.), for at least about three months, and

c) that the formulation is essentially free of visible particles and has a sub-visible particle count below the acceptable United States Pharmacopeia (USP 788) limits: maximum 6000 particles≥10 μm per vial, maximum 600 particles≥25 μm per vial.

### Administration

The cyclic dinucleotide STING agonist formulations described herein will typically be formulated into a dosage form adapted for administration to a subject by a desired route of administration, such as intratumoral or parenteral administration, such as sterile solutions, suspensions, and powders for reconstitution.

The cyclic dinucleotide STING agonist formulations described herein are administered once every 1 to 30 days. In embodiments, the cyclic dinucleotide STING agonist formulations described herein are administered once every 3 to 28 days. In particular embodiments, the cyclic dinucleotide STING agonist formulations described herein are administered once every 3, 7, 14, 21, or 28 days.

In embodiments of such methods, the cyclic dinucleotide STING agonist formulations described herein are administered for from 2 to 36 months. In specific embodiments, the cyclic dinucleotide STING agonist formulations described herein are administered for up to 3 months.

In additional embodiments of such methods, the cyclic dinucleotide STING agonist formulations described herein are administered once every 3, 7, 14, 21, or 28 days for from 2 to 36 months. In further embodiments, the cyclic dinucleotide STING agonist formulations described herein are administered once every 3, 7, 14, 21, or 28 days for up to 3 months. In specific embodiments, the cyclic dinucleotide STING agonist formulations described herein are administered once every 3, 7, 14, 21, or 28 days for up to 3 months, followed by a period, lasting at least 2 months, in which the time interval between doses is increased by at least two-fold. In more specific embodiments, the cyclic dinucleotide STING agonist formulations described herein are administered once every 3, 7, 14, 21, or 28 days for up to 3 months, followed by a period, lasting at least 2 months, in which the time interval between doses is increased by at least three-fold. For example, if the cyclic dinucleotide STING agonist formulations described herein are administered once every 7 days for up to 3 months, it may be followed by a period in which the cyclic dinucleotide STING agonist formulations described herein are administered once every 14 or 21 days for up to two years.

The cyclic dinucleotide STING agonist formulations described herein may be administered prior to or following surgery to remove a tumor and may be used prior to, during, or after radiation treatment.

In some embodiments, the cyclic dinucleotide STING agonist formulations described herein are administered to a patient who has not previously been treated with a biotherapeutic or chemotherapeutic agent, targeted therapy, or hormonal therapy, i.e., is treatment-naïve. In other embodiments, the cyclic dinucleotide STING agonist formulations described herein are administered to a patient who failed to achieve a sustained response after prior therapy with the biotherapeutic or chemotherapeutic agent, i.e., is treatment-experienced.

In specific embodiments, the cyclic dinucleotide STING agonist formulation is administered once every 3 to 30 days for 9 to 90 days, then optionally once every 3 to 30 days for up to 1050 days. In specific embodiments, the cyclic dinucleotide STING agonist formulation is administered once every 3 to 21 days for 9 to 63 days, then optionally once every 3 to 21 days for up to 735 days. In further specific embodiments, the cyclic dinucleotide STING agonist formulation is administered once every 7 to 21 days for 21 to 63 days, then optionally once every 7 to 21 days for up to 735 days. In still further embodiments, the cyclic dinucleotide STING agonist formulation is administered once every 7 to 10 days for 21 to 30 days, then optionally once every 21 days for up to 735 days. In still further embodiments, the cyclic dinucleotide STING agonist formulation is administered once every 7 days for 21 days, then optionally once every 21 days for up to 735 days. In additional embodiments, the cyclic dinucleotide STING agonist formulation is administered once every 21 days for 63 days, then optionally once every 21 days for up to 735 days. In specific embodiments of the foregoing, the cyclic dinucleotide STING agonist formulation is administered at least three times.

In some embodiments, one or more optional “rest” periods, during which the CDN STING agonist formulation is not administered, may be included in the treatment period. In specific embodiments, the optional rest period may be for from 3 to 30 days, from 7 to 21 days, or from 7 to 14 days. Following the rest period, dosing of the CDN STING agonist formulation may be resumed as described above.

### Cell-Proliferation Disorders

The therapies disclosed herein are potentially useful in treating diseases or disorders including, but not limited to, cell-proliferation disorders. Cell-proliferation disorders include, but are not limited to, cancers, benign papillomatosis, gestational trophoblastic diseases, and benign neoplastic diseases, such as skin papilloma (warts) and genital papilloma. The terms “cancer”, “cancerous”, or “malignant” refer to or describe the physiological condition in mammals that is typically characterized by unregulated cell growth.

In specific embodiments, the disease or disorder to be treated is a cell-proliferation disorder. In certain embodiments, the cell-proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In specific embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In specific embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In more specific instances of these embodiments, the breast cancer is triple-negative breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In embodiments, the cell-proliferation disorder is a cancer that has metastasized, for example, liver metastases from colorectal cancer.

In embodiments, the cell-proliferation disorder is selected from the group consisting of solid tumors. In particular embodiments, the cell-proliferation disorder is selected from the group consisting of advanced or metastatic solid tumors. In more particular embodiments, the cell-proliferation disorder is selected from the group consisting of malignant melanoma, head and neck squamous cell carcminoma, and breast adenocarcinoma.

In particular embodiments, the cell-proliferation disorder is classified as stage III cancer or stage IV cancer. In instances of these embodiments, the cancer is not surgically resectable.

### Methods and Uses

Therapy including treatment with the formulations described herein may also comprise one or more additional therapeutic agents. The additional therapeutic agent may be, e.g., a chemotherapeutic, a biotherapeutic agent (including but not limited to antibodies to VEGF, VEGFR, EGFR, Her2/neu, other growth factor receptors, CD20, CD40, CD-40L, CTLA-4, OX-40, 4-1BB, and ICOS), an immunogenic agent (for example, attenuated cancerous cells, tumor antigens, antigen presenting cells, such as dendritic cells pulsed with tumor derived antigen or nucleic acids, immune stimulating cytokines (for example, IL-2, IFNα2, GM-CSF), and cells transfected with genes encoding immune stimulating cytokines, such as but not limited to GM-CSF).

The therapies disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefore, contemporaneously or sequentially with a compound of the present disclosure.

The additional active agent(s) may be one or more agents selected from the group consisting of STING agonists, anti-viral compounds, antigens, adjuvants, anti-cancer agents, CTLA-4, LAG-3, and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines. It will be understood the descriptions of the above additional active agents may be overlapping. It will also be understood that the treatment combinations are subject to optimization, and it is understood that the best combination to use of the CDN STING agonist, and one or more additional active agents will be determined based on the individual patient needs.

When the therapies disclosed herein are used contemporaneously with one or more other active agents, the CDN STING agonist formulation described herein may be administered either simultaneously with, or before or after, one or more other active agent(s). The CDN STING agonist formulation may be administered separately, by the same or different route of administration, or together in the same pharmaceutical composition as the other agent(s).

The dosage amount of the CDN STING agonist formulation may be varied and will depend upon the therapeutically effective dose of each agent. Generally, a therapeutically effective dose of each will be used. Combinations including at least one CDN STING agonist, and other active agents will generally include a therapeutically effective dose of each active agent. In such combinations, the CDN STING agonist formulation and other active agents may be administered separately or in conjunction. In addition, the administration of one element may be prior to, concurrent with, or subsequent to the administration of other agent(s).

In one embodiment, the disclosure provides a kit comprising two or more separate pharmaceutical formulations, one of which is a CDN STING agonist formulation. In one embodiment, the kit comprises means for separately retaining said compositions, such as a container, divided bottle, or divided foil packet. A kit of this disclosure may be used for administration of different dosage forms, for example, oral and parenteral, for administration of the separate formulations at different dosage intervals, or for titration of the separate compositions against one another. To assist with compliance, a kit of the disclosure typically comprises directions for administration.

The disclosure also provides the use of a CDN STING agonist formulation for treating a cell-proliferation disorder, where the patient has previously (e.g., within 24 hours) been treated with another agent.

Anti-viral compounds that may be used in combination with the therapies disclosed herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

Antigens and adjuvants that may be used in combination with the therapies disclosed herein include B7 costimulatory molecule, interleukin-2, interferon-γ, GM-CSF, CTLA-4 antagonists, OX-40/0X-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, lipopolysaccharide (LPS), monophosphoryllipid A, lipoteichoic acid, imiquimod, resiquimod, and in addition retinoic acid-inducible gene I (RIG-I) agonists such as poly I:C, used separately or in combination are also potential adjuvants.

Examples of cytotoxic agents that may be used in combination with the therapies disclosed herein include, but are not limited to, arsenic trioxide (sold under the tradename TRISENOX®), asparaginase (also known as L-asparaginase, and Erwinia L-asparaginase, sold under the tradenames ELSPAR® and KIDROLASE®).

Chemotherapeutic agents that may be used in combination with the therapies disclosed herein include abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea and taxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine, and pharmaceutically acceptable salts thereof.

Examples of vascular endothelial growth factor (VEGF) receptor inhibitors include, but are not limited to, bevacizumab (sold under the trademark AVASTIN by Genentech/Roche), axitinib (described in PCT International Patent Publication No. WO01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinylmethy)amino]-3-pyridinecarboxamide. and described in PCT International Patent Application Publication No. WO02/068470), pasireotide (also known as SO 230, and described in PCT International Patent Publication No. WO02/010192), and sorafenib (sold under the tradename NEXAVAR).

Examples of topoisomerase II inhibitors, include but are not limited to, etoposide (also known as VP-16 and Etoposide phosphate, sold under the tradenames TOPOSAR, VEPESID, and ETOPOPHOS), and teniposide (also known as VM-26, sold under the tradename VUMON).

Examples of hypomethylating agents and alkylating agents, include but are not limited to, 5-azacytidine (sold under the trade name VIDAZA), decitabine (sold under the trade name of DECOGEN), temozolomide (sold under the trade names TEMODAR and TEMODAL), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX® and MYLERAN), carboplatin (sold under the tradename PARAPLATIN), lomustine (also known as CCNU, sold under the tradename CEENU®, cisplatin (also known as CDDP, sold under the tradenames PLATINOL® and PLATINOL®-AQ), chlorambucil (sold under the tradename LEUKERAN®), cyclophosphamide (sold under the tradenames CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (sold under the tradename IFEX®, procarbazine (sold under the tradename MATULANE®), mechlorethamine (also known as nitrogen mustard, mustine and mechloroethamine hydrochloride, sold under the tradename MUSTARGEN®), streptozocin (sold under the tradename ZANOSAR®), thiotepa (also known as thiophosphoamide, TESPA and TSPA, and sold under the tradename THIOPLEX®, and pharmaceutically acceptable salts thereof.

Examples of anti-tumor antibiotics include, but are not limited to, doxorubicin (sold under the tradenames ADRIAMYCIN® and RUBEX®, bleomycin (sold under the tradename LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME®), mitoxantrone (also known as DHAD, sold under the tradename NOVANTRONE®), epirubicin (sold under the tradename ELLENCE™), idarubicin (sold under the tradenames IDAMYCIN®, IDAMYCIN PFS®), and mitomycin C (sold under the tradename MUTAMYCIN®).

Examples of anti-metabolites include, but are not limited to, claribine (2-chlorodeoxyadenosine, sold under the tradename LEUSTATIN®), 5-fluorouracil (sold under the tradename ADRUCIL®), 6-thioguanine (sold under the tradename PURINETHOL®), pemetrexed (sold under the tradename ALIMTA®), cytarabine (also known as arabinosylcytosine (Ara-C), sold under the tradename CYTOSAR-U®), cytarabine liposomal (also known as Liposomal Ara-C, sold under the tradename DEPOCYT™), decitabine (sold under the tradename DACOGEN®), hydroxyurea and (sold under the tradenames HYDREA®, DROXIA™ and MYLOCEL™), fludarabine (sold under the tradename FLUDARA®), floxuridine (sold under the tradename FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (sold under the tradename NIPENT®).

Examples of retinoids include, but are not limited to, alitretinoin (sold under the tradename PANRETIN®), tretinoin (all-trans retinoic acid, also known as ATRA, sold under the tradename VESANOID®), Isotretinoin (13-c/s-retinoic acid, sold under the tradenames ACCUTANE®, AMNESTEEM®, CLARAVIS®, CLARUS®, DECUTAN®, ISOTANE®, IZOTECH®, ORATANE®, ISOTRET®, and SOTRET®), and bexarotene (sold under the tradename TARGRETIN®).

The invention further relates to a method of treating cancer in a human patient comprising administration of a cyclic dinucleotide STING agonist compound and a PD-1 antagonist to the patient. The cyclic dinucleotide STING agonist compound and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck & Co., Inc., Kenilworth, N.J., USA), nivolumab (OPDIVO™, Bristol-Myers Squibb Company, Princeton, N.J., USA), cemiplimab (LIBTAYQ™, Regeneron Pharmaceuticals, Inc., Tarrytown, N.Y., USA), atezolizumab (TECENTRIQ™, Genentech, San Francisco, Calif., USA), durvalumab (IMFINZI™, AstraZeneca Pharmaceuticals LP, Wilmington, Del.), or avelumab (BAVENCIO™, Merck KGaA, Darmstadt, Germany).

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

### ADDITIONAL EMBODIMENTS

The present disclosure further relates to methods of treating a cell-proliferation disorder, said method comprising administering to a subject in need thereof a therapy that comprises a cyclic dinucleotide STING agonist compound formulation; wherein the cyclic dinucleotide STING agonist formulation is administered once every 1 to 30 days. In embodiments, the cyclic dinucleotide STING agonist formulation is administered once every 3 to 28 days. In particular embodiments, the cyclic dinucleotide STING agonist formulation is administered once every 3, 7, 14, 21, or 28 days.

In embodiments of such methods, the cyclic dinucleotide STING agonist formulation is administered for from 2 to 36 months. In specific embodiments, the cyclic dinucleotide STING agonist formulation is administered for up to 3 months.

In additional embodiments of such methods, the cyclic dinucleotide STING agonist formulation is administered once every 3, 7, 14, 21, or 28 days for from 2 to 36 months. In further embodiments, the cyclic dinucleotide STING agonist formulation is administered once every 3, 7, 14, 21, or 28 days for up to 3 months. In specific embodiments, the cyclic dinucleotide STING agonist formulation is administered once every 3, 7, 14, 21, or 28 days for up to 3 months, followed by a period, lasting at least 2 months, in which the time interval between doses is increased by at least two-fold. In more specific embodiments, the cyclic dinucleotide STING agonist formulation is administered once every 3, 7, 14, 21, or 28 days for up to 3 months, followed by a period, lasting at least 2 months, in which the time interval between doses is increased by at least three-fold. For example, if the cyclic dinucleotide STING agonist is administered once every 7 days for up to 3 months, it may be followed by a period in which the cyclic dinucleotide STING agonist formulation is administered once every 14 or 21 days for up to two years.

The present disclosure further relates to methods of treating a cell-proliferation disorder, said method comprising administering to a subject in need thereof a therapy that comprises a cyclic dinucleotide STING agonist compound formulation; wherein the cyclic dinucleotide STING agonist formulation is administered once every 1 to 30 days for 3 to 90 days, then optionally once every 1 to 30 days for up to 1050 days. In embodiments, the CDN STING agonist formulation is administered at least three times.

In specific embodiments, the cyclic dinucleotide STING agonist formulation is administered once every 3 to 30 days for 9 to 90 days, then optionally once every 3 to 30 days for up to 1050 days. In specific embodiments, the cyclic dinucleotide STING agonist formulation is administered once every 3 to 21 days for 9 to 63 days, then optionally once every 3 to 21 days for up to 735 days. In further specific embodiments, the cyclic dinucleotide STING agonist is administered once every 7 to 21 days for 21 to 63 days, then optionally once every 7 to 21 days for up to 735 days. In still further embodiments, the cyclic dinucleotide STING agonist formulation is administered once every 7 to 10 days for 21 to 30 days, then optionally once every 21 days for up to 735 days. In still further embodiments, the cyclic dinucleotide STING agonist is administered once every 7 days for 21 days, then optionally once every 21 days for up to 735 days. In additional embodiments, the cyclic dinucleotide STING agonist formulation is administered once every 21 days for 63 days, then optionally once every 21 days for up to 735 days. In specific embodiments of the foregoing, the CDN STING agonist formulation is administered at least three times.

Additionally, the present disclosure relates to methods of treating a cell-proliferation disorder, said method comprising administering to a subject in need thereof a therapy that comprises a cyclic dinucleotide STING agonist formulation as described herein; wherein the cell-proliferation disorder is cancer. In specific embodiments, the cancer occurs as one or more solid tumors. In further specific embodiments, the cancer is selected from the group consisting of advanced or metastatic solid tumors. In still further specific embodiments, the cancer is selected from the group consisting of malignant melanoma, head and neck squamous cell carcinoma, and breast adenocarcinoma. In particular embodiments, the cell-proliferation disorder is a cancer that has metastasized, for example, liver metastases from colorectal cancer. In additional embodiments, the cell-proliferation disorder is a cancer is classified as stage III cancer or stage IV cancer. In embodiments, the cancer is not surgically resectable.

### EXAMPLES

### Materials and Methods

UHPLC: Ultra High Performance Liquid Chromatography was used to monitor assay and degradation products for Compound A. The gradient reverse phase UHPLC method was performed using a reversed-phase C18 column (150×2.1 mm, 1.7 μm particle size). The mobile phase consisted of a gradient mixture of 100 mM triethylammonium acetate (TEAA) in water and 100% acetonitrile or 100 mM TEAA in 90/10 acetonitrile/water. The flow rate was 0.3 mL/minute, and the column was maintained at 40° C. A UV detector monitored absorbance at 260 nm. Standard and sample solutions were prepared in 90/10 (v/v) water/methanol to a final concentration of approximately 0.06 mg/mL with an injection volume of 3-5 μL.

The pH of formulations was measured following United States Pharmacopeia procedure <791>: using a standard potentiometric pH meter with temperature adjustment, the pH meter was calibrated with buffer solutions of known pH values that span the expected pH of the test solutions. To measure pH of the test solutions, the pH probe was immersed in the solution until the pH reading stabilized. The value was read and recorded by the Analyst.

Osmolality: The osmolality of formulations was measured following United States Pharmacopeia procedure <785>: a calibration check was performed on a freezing point apparatus prior to sample testing by measuring the osmolality of two standard solutions that span the expected osmolality of the test solution. For sample measurement, the appropriate volume of test solution was transferred to a measurement cell and the test was initiated by engaging the appropriate button. The osmolality of the sample was read by the analyst and manually recorded in an electronic notebook repository.

HPLC (Methionine Assay): In certain examples, High Performance Liquid Chromatography (HPLC) was used to monitor methionine assay. The reverse phase HPLC method was performed using a hydrophilic C18 column (150×4.6 mm, 3 μm particle size). The mobile phase consisted of 0.1% phosphoric acid. The flow rate was 1.0 mL/minute, and the column was maintained at 40° C. A UV detector monitored absorbance at 214 nm. Standard and sample solutions were prepared in water to a final concentration of approximately 0.149 mg/mL.

In other examples, the methionine determination procedure for Compound A was a gradient reversed phase HPLC method using a reversed-phase C18 column (150×4.6 mm, 5 μm particle size) or equivalent. The mobile phase consisted of a gradient mixture (v/v) of 0.1% phosphoric acid in water and 80/20 acetonitrile/water. The flow rate was 1.0 mL/minute, and the column temperature is maintained at 30° C. A UV detector monitored absorbance at 205 nm. Standard and sample solutions are prepared in 90:10 (v/v) water:methanol to a final concentration of approximately 0.075 mg/mL.

HPLC (EDTA Assay): In other examples, HPLC was used to monitor EDTA assay. The gradient reverse phase HPLC method was performed using an anion exchange HPLC column (150×4.1 mm, 10 μm particle size). The mobile phase consisted of a gradient mixture of 0.25 mM copper sulfate in 89/6/4/1 water/acetonitrile/methanol/isopropanol and 100% acetonitrile. The flow rate was 1.0 mL/minute, and the column was maintained at 40° C. A UV detector monitored absorbance at 254 nm. Standard solutions were prepared in 1.25 mM copper sulfate in water to a final concentration of approximately 0.186 mg/mL.

In other examples, a gradient reversed phase HPLC method using a reversed-phase C18 column (2.5 μm, 50×3.0 mm id column) was used to monitor EDTA assay. Mobile phase A consisted of 10 mM TBA-Br+10 mM ammonium acetate in 95:5 (v/v) water:acetonitrile and mobile phase B consists of 10 mM TBA-Br+50 mM ammonium acetate in 50:50 (v/v) water:acetonitrile. A gradient elution is used to separate EDTA from other peaks in diluent and sample matrix. The flow rate was 0.5 mL/minute, and the column temperature was maintained at 30° C. EDTA standard solutions were mixed 1:1 with 0.05 mg/mL FeCl₃ in water to a final concentration of 0.01 mg/mL EDTA to allow for UV detection of the EDTA-Fe complex at 254 nm.

UHPLC, Solubility Measurement: Ultra Performance Liquid Chromatography (UHPLC) was used to measure drug solubility. The gradient reverse phase UHPLC method was performed using a reversed-phase C18 column (50×2.1 mm, 1.7 μm particle size). The mobile phase consisted of a gradient mixture of 100 mM triethylammonium acetate (TEAA) in water and 100% acetonitrile. The flow rate was 0.3 mL/minute, and the column was maintained at 40° C. A UV detector monitored absorbance at 260 nm. Standard and sample solutions were prepared in 95/5 (v/v) TEAA/acetonitrile to a final concentration of approximately 0.05 mg/mL.

Sub-Visible Particulates: Sub-visible particulates of the pharmaceutical solution formulations were monitored using a flow-imaging microscope and particle analyzer (FlowCam 8000, Fluid Imaging Technologies, Inc., Scarborough, Mass., USA). For sub-visible particle counting, about 1 mL of solution formulation is injected into the sample port for flow-imaging analysis. A 10× objective lens monitoring particle sizes from 2-100 μm was used.

### Example 1: Impact of Steam Sterilization on the Stability of Formulations

The stability of Compound A formulations upon autoclave steam sterilization was evaluated. To optimize formulation composition and understand the impact of processing variables on stability, Compound A formulations containing a buffering agent, histidine or sodium phosphate (NaPO₄), and a tonicity modifier, sucrose or sodium chloride, were evaluated with and without autoclave steam sterilization.

For this study, Compound A diluent solutions were prepared by dissolving target amounts of buffer (histidine or phosphate), tonicity modifier (sucrose or sodium chloride), L-methionine and EDTA in water (see Table 1). Diluent solutions were adjusted to pH 7.0. Each diluent formulation was filtered using 0.22 μm polyvinylidene fluoride (PVDF) membrane filter. Compound A 0.6 mg/mL drug product formulations were made by adding 190 mL Compound A diluent solution to 147 mg Compound A. Compound A drug product formulations were filtered using 0.22 μm PVDF membrane filter. Each of the formulated solutions was filled into a 6R vial (Type 1, European Blow Back) with a 1 mL formulation solution fill volume. Each vial was stoppered and sealed with an aluminum cap. At the time of preparation, the formulations were inspected for visible particulates. At the initial time point, all formulations were essentially free of visible particulates. Samples were placed in an autoclave (Tuttnauer Brinkmann 2540EK) and steam sterilized for 15 minutes at 121° C. Both autoclaved and non-autoclaved samples were protected from light and placed in a 2° C. to 8° C., 30° C., 40° C., and 60° C. environmental stability chamber for 13 weeks. These temperatures were selected to represent potential target storage conditions (5° C. and 30° C.) and accelerated storage conditions (40° C. and 60° C.). To evaluate formulation stability over the aforementioned period, the following methods were used: UHPLC (Assay and Degradation Products of Compound A), HPLC (Methionine Assay), pH, visual description, and osmolality testing.

TABLE 1
Compound A Formulations, pH 7

| | Compound A | | Tonicity | | |
|---|---|---|---|---|---|
| Formulation | Concentration | Buffer | Modifier | Stabilizer 1 | Stabilizer 2 |
| E1-F1 | 0.6 mg/mL | 50 mM | 197 mM | 10 mM | 0.5 mM |
| | | Histidine | Sucrose | Methionine | EDTA |
| E1-F2 | 0.6 mg/mL | 50mM | 99 mM | 10 mM | 0.5 mM |
| | | Histidine | NaCl | Methionine | EDTA |
| E1-F3 | 0.6 mg/mL | 50 mM | 148 mM | 10 mM | 0.5 mM |
| | | NaPO₄ | Sucrose | Methionine | EDTA |
| E1-F4 | 0.6 mg/mL | 50 mM | 74 mM | 10 mM | 0.5 mM |
| | | NaPO₄ | NaCl | Methionine | EDTA |

Table 2 summarizes the growth of degradation products monitored by UHPLC at specific time points and storage condition relative to the initial amount of Compound A. For each formulation, degradation growth was monitored for solutions that had been autoclaved as well as for solutions that had not been autoclaved. For sucrose-containing formulations (E1-F1 and E1-F3), a significant increase degradation growth was observed in autoclaved formulations versus those that had not been autoclaved. NaCl formulations (E1-F2 and E1-F4) show little differences in degradation growth between autoclaved and non-autoclaved formulations. Phosphate buffered formulations (E1-F3 and E1-F4) were not tested past four weeks due to lower solubility of Compound A in those formulations (see Example 2).

TABLE 2
Assay and Degradation Products Data

| | | Degradation Growth vs Initial of Total Related Substances | | | | |
|---|---|---|---|---|---|---|
| | | (Represented as % of Compound A) | | | | |
| | Processing | 4 Week, | 4 Week, | 13 Week, | 13 Week, | 13 Week, |
| Formulation | Condition | 40° C. | 60° C. | 5° C. | 30° C. | 40° C. |
| E1-F1 | Autoclave | 0.3 | 8.9 | 0.3 | 0.6 | 1.0 |
| | 121° C./15 min | | | | | |
| | No Autoclave | 0.0 | 2.1 | 0.4 | 0.3 | 0.4 |
| E1-F2 | Autoclave | 0.1 | 0.4 | 0.3 | 0.2 | 0.1 |
| | 121° C./15 min | | | | | |
| | No Autoclave | 0.1 | 0.3 | 0.3 | 0.3 | 0.2 |
| E1-F3 | Autoclave | 3.7 | 5.6 | NT | NT | NT |
| | 121° C./15 min | | | | | |
| | No Autoclave | 0.2 | 4.1 | NT | NT | NT |
| E1-F4 | Autoclave | 0.0 | 0.1 | NT | NT | NT |
| | 121° C./15 min | | | | | |
| | No Autoclave | 0.0 | 0.1 | NT | NT | NT |

When initially observed following autoclaving, significant degradate growth was observed in E1-F3 post autoclave, demonstrating incompatibility of phosphate/sucrose with steam sterilization. Additional Compound A degradate growth was observed after 4 weeks at 40° C. and 60° C. Greater overall degradation in formulations containing phosphate (E1-F3 and E1-F4) in comparison to histidine-containing formulations (E1-F1 and E1-F2) was observed under both autoclaved and non-autoclaved processing conditions. Formulations containing phosphate (E1-F3 and E1-F4) were not evaluated further after the 4-week time point. Significant degradate growth was observed post-autoclave after 4 weeks at 60° C. in E1-F1. Additionally, early-eluting peaks assigned to excipient degradation, also present in comparable diluent formulations, were observed post-autoclave, indicating incompatibility of sucrose with steam sterilization. No excipient-related peaks were observed in E1-F2. After 13 weeks at 5° C., 30° C., and 40° C., the rate of degradate growth in E1-F1 was higher in autoclaved formulations relative to non-autoclaved formulations, suggesting incompatibility of sucrose with steam sterilization. The rate of degradate growth in E1-F2 was similar in autoclaved and non-autoclaved formulations. Greater overall degradation was observed in sucrose-containing formulations than in sodium chloride-containing formulations.

Table 3 shows measured pH values for the formulations at the initial time point and after 13 weeks of storage at 5° C., 30° C., and 40° C. No significant changes in pH were observed for any of the formulations at any processing condition, storage condition, or time point. These data indicate that use of L-histidine buffer and sodium phosphate buffer at the indicated concentrations are sufficient for maintaining the pH of the formulation solution during the autoclave process as well as during storage of the product.

TABLE 3
pH Data

| | | | | | PH |
|---|---|---|---|---|---|
| | Processing | | 13 Week, | 13 Week, | 13 Week, |
| Formulation | Condition | Initial | 5° C. | 30° C. | 40° C. |
| E1-F1 | Autoclave | 6.9 | 6.7 | 6.8 | 6.8 |
| | 121° C./15 min | | | | |
| | No Autoclave | 6.9 | 6.9 | 6.8 | 6.7 |
| E1 -F2 | Autoclave | 7.0 | 7.0 | 7.0 | 7.1 |
| | 121° C./15 min | | | | |
| | No Autoclave | 7.0 | 7.0 | 7.0 | 7.0 |
| E1 -F3 | Autoclave | 7.0 | NT | NT | NT |
| | 121° C./15 min | | | | |
| | No Autoclave | 7.0 | NT | NT | NT |
| E1 -F4 | Autoclave | 7.0 | NT | NT | NT |
| | 121° C./15 min | | | | |
| | No Autoclave | 7.0 | NT | NT | NT |

Table 4 shows the measured osmolality values for the formulations at the initial time point and after 13 weeks of storage at 5° C., 30° C., and 40° C. No significant changes in osmolality were observed for any of the formulations at any processing condition, storage condition, or time point.

TABLE 4
Osmolality Data

| | | Osmolality (mOsmol/kg) | | | |
|---|---|---|---|---|---|
| | Processing | Initial | 13 Week, | 13 Week, | 13 Week, |
| Formulation | Condition | | 5° C. | 30° C. | 40° C. |
| E1 -F1 | Autoclave | 287 | 287 | 290 | 293 |
| | 121° C./15 min | | | | |
| | No Autoclave | 278 | 284 | 286 | 285 |
| E1-F2 | Autoclave | 253 | 253 | 256 | 259 |
| | 121° C./15 min | | | | |
| | No Autoclave | 252 | 253 | 256 | 256 |
| E1-F3 | Autoclave | 276 | NT | NT | NT |
| | 121° C./15 min | | | | |
| | No Autoclave | 273 | NT | NT | NT |
| E1-F4 | Autoclave | 258 | NT | NT | NT |
| | 121° C./15 min | | | | |
| | No Autoclave | 255 | NT | NT | NT |

The level of antioxidant in histidine-containing formulations was determined using the aforementioned HPLC methods to monitor any loss of stabilizing excipients either after autoclave processing or during storage at various temperatures for 13 weeks. There was no significant loss of methionine for any formulation, processing, and storage condition after 13 weeks, except that the E1-F1 subjected to autoclave processing that showed significant loss of methionine after 13 weeks at both 30° C. and 40° C.

TABLE 5
Methionine Assay Data

| | | % Methionine | | | |
|---|---|---|---|---|---|
| | Processing | | 13 Week, | 13 Week, | 13 Week, |
| Formulation | Condition | Initial | 5° C. | 30° C. | 40° C. |
| E1-F1 | Autoclave | 103.3 | 101.0 | 99.3 | 96.3 |
| | 121° C./15 min | | | | |
| | No Autoclave | 101.1 | 101.6 | 102.4 | 102.5 |
| E1-F2 | Autoclave | 115.6 | 112.6 | 114.0 | 114.3 |
| | 121° C./15 min | | | | |
| | No Autoclave | 112.9 | 114.2 | 114.5 | 114.2 |

### Example 2: Equilibrium Solubility of Formulations

Equilibrium solubility experiments were conducted by mixing Compound A and the diluent of interest at a concentration of about 20 mg/ml to about 30 mg/ml. If a saturated solution was not achieved, additional Compound A was added until a cloudy solution was maintained. Samples were stirred at ambient conditions of temperature, humidity, and light. Aliquots were removed, clarified by centrifugation and assayed by HPLC. When the measurements indicated that solubility had reached equilibrium, the experiment was terminated. After equilibration, the final solution pH was measured, and the terminal phase of Compound A was determined by XRPD.

TABLE 6
Equilibrium Solubility Determinations of
Compound A Crystalline Monosodium Salt in Potential Buffers

| | Crystalline | | | | |
|---|---|---|---|---|---|
| | Monosodium | | Solubility | | Terminal |
| Diluent | Compound A | Temperature | 24 hrs | 48 hrs | Phase (XRD) |
| E2-D1 | 10 mM | 5° C. | 8 | 8 | Monosodium |
| | phosphate pH 7 | RT | 9 | 10 | (starting |
| E2-D2 | 50 mM | RT | 11 | 13 | material) |
| | phosphate pH 7 | | | | |
| E2-D3 | 10 mM citrate pH 7 | RT | 8 | 8 | |

Solubility expressed in free acid equivalents (mg/ml)

TABLE 7
Equilibrium Solubility Determinations
of Compound A Crystalline Monosodium Salt in
Binary Mixtures with 10mM phosphate buffer, pH 7

| | | Solubility | | Terminal |
|---|---|---|---|---|
| Diluent | Diluent | 24 hrs | 48 hrs | Phase (XRD) |
| E2-D4 | 30% Sulfobutylether-β- | 5 | 5 | Monosodium |
| | Cyclodextrin | | | (starting |
| E2-D5 | 30% (2-Hydroxypropyl)- | | | material) |
| | beta-cyclodextrin | 9 | 9 | |
| E2-D6 | 60% propylene glycol | 10 | 9 | |
| E2-D7 | 10% Tween 80 | 10 | 10 | Indiscernible |
| E2-D8 | 10% DMSO | 9 | 9 | |
| E2-D9 | 8.2% sucrose | 8 | 9 | |

Solubility expressed in free acid equivalents (mg/ml)?

The results from Tables 6 and 7 support using a 100% aqueous buffer at neutral pH. There is no significant difference in solubility when stored at 5° C. as opposed to room temperature for the phosphate buffer.

TABLE 8
Equilibrium Solubility Determinations of
Compound A Crystalline Monosodium Salt

| | | | | | | | Approxi- |
|---|---|---|---|---|---|---|---|
| | Buffer | Tonicity | Anti- | Metal | Solubility | | mate |
| | 50 mM | Modifier | oxidant | Chelator | 24 | 48 | Terminal |
| Diluent | (pH 7) | 113 mM | 10 mM | 0.5 mM | hours | hours | pH |
| E2-D10 | phosphate | sucrose | L-meth- | EDTA | 8 | 8 | 6 |
| E2-D11 | phosphate | mannitol | ionine | | 9 | 8 | 6 |
| E2-D12 | histidine | sucrose | | | 11 | 11 | 6 |
| E2-D13 | phosphate | glycerol | | | 9 | 8 | 6 |
| E2-D14 | TRIS | sucrose | | | 5 | 5 | 5 |

Solubility expressed in free acid equivalents (mg/ml)

The results from Table 8 demonstrate an increase in Compound A solubility when histidine buffer is used, compared to the previously evaluated phosphate buffer. There is no significant effect of tonicity modifier on Compound A solubility across the phosphate buffer samples.

TABLE 9
Effect of Buffer pH and Ionic Strength on Equilibrium Solubility
of Amorphous Compound A Disodium Salt in Formulations with
Sucrose/L-methionine/EDTA (mM:113/10/0.5)

| | | | Solubility (Free | | |
|---|---|---|---|---|---|
| | Histidine | Formulation | Acid equivalents) | | Approximate |
| Formulation | (mM) | starting pH | 24 hours | 48 hours | Terminal pH |
| E2-D15 | 50 | 6.0 | 26 | 27 | 6.5 |
| E2-D12 | 50 | 6.5 | 40 | 60 | 7 |
| E2-D16 | 50 | 7.0 | 59 | 64 | 7 |
| E2-D17 | 12.5 | 7.0 | 58 | 63 | 7 |
| E2-D18 | 25 | 7.0 | 66 | 66 | 7 |
| E2-D19 | 50 | 7.0 | 59 | 64 | 7 |
| E2-D20 | 100 | 7.0 | 72 | 72 | 7 |
| E2-D21 | 200 | 7.0 | 77 | 78 | 7 |

Solubility expressed in free acid equivalents (mg/mL)

Table 9 shows the experiment used to evaluate the effect of pH and buffer concentration on the solubility of Compound A. The results from Table 9 supports using a histidine buffer, with a final pH of 6.5 (close to the pKa of histidine), which provides greater buffering capacity and maintains adequate solubility. Based on the outcomes shown in Tables 6-9, a histidine buffer was selected for safety assessment and clinical studies.

### Example 3: Stability Evaluation of Compound a Formulations

A study was performed to optimize formulation pH, buffer concentration and methionine concentration. For this study, diluent solutions were prepared by dissolving target amounts of histidine, mannitol, L-methionine, and EDTA in water (see Table 10). Solutions were adjusted to the target pH with 1N HCl and 1N NaOH. Prior to the addition of Compound A, each diluent solution was filtered using a 0.22 μm PVDF membrane filter. Compound A was added to the diluent solutions to prepare formulations having the target concentration of 0.6 mg/mL, as shown in Table 10. These formulations were filtered using a 0.22 μm PVDF membrane filter. Each of the formulated solutions was filled into a 6R vial (Type 1, European Blow Back) with a 2 mL formulation solution fill volume. Each vial was stoppered and sealed with an aluminum cap. After sealing, the formulations were autoclaved at 121° C. for 15 minutes. The formulations were then visually inspected. At the initial time point, there were no visible particulates in the formulations. Samples were staged, protected from light, and placed in a 2° C. to 8° C., 30° C., 40° C., and 60° C. environmental stability chamber for 13 weeks.

TABLE 10
Summary of Formulations Studied

| | | | | Stabi- | | |
|---|---|---|---|---|---|---|
| | | Buffer | Tonicity | lizer 1 | Stabi- | Tar- |
| Formu- | Compound A | Histi- | Modifier | Methi- | lizer 2 | get |
| lation | Concentration | dine | Mannitol | onine | EDTA | pH |
| E3-F1 | 0.6 mg/mL | 10 mM | 187 mM | 5 mM | 0.5 mM | 6 |
| E3-F2 | 0.6 mg/mL | 10 mM | 187 mM | 10 mM | 0.5 mM | 6 |
| E3-F3 | 0.6 mg/mL | 10 mM | 187 mM | 5 mM | 0.5 mM | 7.5 |
| E3-F4 | 0.6 mg/mL | 10 mM | 187 mM | 10 mM | 0.5 mM | 7.5 |
| E3-F5 | 0.6 mg/mL | 50 mM | 187 mM | 5 mM | 0.5 mM | 6 |
| E3-F6 | 0.6 mg/mL | 50 mM | 187 mM | 10 mM | 0.5 mM | 6 |
| E3-F7 | 0.6 mg/mL | 50 mM | 187 mM | 5 mM | 0.5 mM | 7.5 |
| E3-F8 | 0.6 mg/mL | 50 mM | 187 mM | 10 mM | 0.5 mM | 7.5 |

The formulations were tested to evaluate the stability of the formulations and the impact of buffer concentration, L-methionine concentration, and solution pH. Assay and degradation products of Compound A were monitored by UHPLC; pH was monitored; and methionine assay was monitored by HPLC. The formulations were also monitored for visual changes.

TABLE 11
pH Results

| | PH | | | | |
|---|---|---|---|---|---|
| | | 4 Weeks | 4 Weeks @ | 13 Weeks @ | 13 Weeks @ |
| Formulation | Initial | 5° C. | 60° C. | 5° C. | 40° C. |
| E3-F1 | 6.0 | 5.9 | 6.0 | 6.0 | 5.9 |
| E3-F2 | 6.0 | 6.0 | 6.0 | 6.0 | 5.9 |
| E3-F3 | 7.2 | 7.4 | 7.4 | 7.3 | 7.2 |
| E3-F4 | 7.2 | 7.4 | 7.4 | 7.2 | 7.3 |
| E3-F5 | 5.9 | 6.0 | 6.0 | 5.9 | 5.9 |
| E3-F6 | 5.9 | 6.0 | 6.0 | 5.8 | 5.9 |
| E3-F7 | 7.3 | 7.4 | 7.4 | 7.3 | 7.3 |
| E3-F8 | 7.4 | 7.4 | 7.4 | 7.4 | 7.3 |

For all formulations, no significant changes in pH were observed relative to the initial value, at any time point or under any storage conditions. The formulations were visually inspected for precipitate formation. After four weeks at across all conditions, no changes were observed.

TABLE 12
Degradation Product Growth

| | Total Degradation Growth (% of Compound A) vs. Initial | | | | |
|---|---|---|---|---|---|
| | 4 | 4 | 13 | 13 | 13 |
| Formu- | Weeks @ | Weeks @ | Weeks @ | Weeks @ | Weeks @ |
| lation | 40° C. | 60° C. | 5° C. | 30° C. | 40° C. |
| E3-F1 | 0.01 | 0.39 | −0.11 | −0.12 | 0.01 |
| E3-F2 | 0.04 | 0.41 | −0.09 | −0.12 | 0.00 |
| E3-F3 | −0.03 | 0.13 | −0.11 | −0.04 | −0.03 |
| E3-F4 | 0.04 | 0.22 | −0.03 | −0.03 | −0.04 |
| E3-F5 | 0.02 | 0.58 | −0.07 | −0.10 | 0.05 |
| E3-F6 | 0.05 | 0.60 | −0.07 | −0.08 | 0.03 |
| E3-F7 | 0.03 | 0.32 | −0.06 | −0.07 | −0.03 |
| E3-F8 | 0.02 | 0.29 | −0.07 | NT | −0.04 |

There was no significant loss in assay value (data not shown) or growth in Compound A degradation products for the studied formulations after storage for 4 weeks at 40° C. After 4 weeks at 60° C., some Compound A degradation growth was observed, especially in formulations E3-F1, E3-F2, E3-F5, and E3-F6, which have a solution pH of 6. No significant growth of degradation products was observed for the studied formulations at any condition after 13 weeks. All formulations studied are stable to storage at refrigerated, room temperature, and accelerated temperature conditions over the duration of the study. Storage of formulations stored for 4 weeks at 60° C. shows some differentiation based on pH.

TABLE 13
Methionine Assay

| | Total Degradation Growth (% of Methionine) vs. Initial | | |
|---|---|---|---|
| Formulation | 4 Weeks @ 40° C. | 4 Weeks @ 60° C. | 13 Weeks @ 40° C. |
| E3-F1 | ≤2.0 | ≤2.0 | ≤2.0 |
| E3-F2 | ≤2.0 | ≤2.0 | ≤2.0 |
| E3-F3 | ≤2.0 | ≤2.0 | ≤2.0 |
| E3-F4 | ≤2.0 | ≤2.0 | ≤2.0 |
| E3-F5 | ≤2.0 | ≤2.0 | ≤2.0 |
| E3-F6 | ≤2.0 | ≤2.0 | ≤2.0 |
| E3-F7 | ≤2.0 | ≤2.0 | ≤2.0 |
| E3-F8 | ≤2.0 | ≤2.0 | ≤2.0 |

Accelerated conditions were tested for the methionine assay, because these conditions are most likely to induce excipient loss in short term storage, such as 4 to 13 weeks. No significant loss of methionine (>2.0%) in methionine assay values were noted after 4 weeks at 40° C. and 60° C. After 13 weeks, no significant loss in methionine assay values was observed at 40° C.

Buffer concentrations between 10 mM and 50 mM L-histidine were not differentiated based on these results. L-methionine concentrations of 5 mM and 10 mM also were not differentiated. The formulations having pH values of 6 induce significantly more degradation growth than formulations having pH values of 7.5.

### Example 4: Evaluation of the Impact of Alternative Tonicity Modifiers on Stability in Compound a Formulations

The impact of tonicity modifiers on chemical stability and solubility of steam-sterilized formulations was studied in the following manner.

All diluent solutions were prepared by transferring appropriate amounts of L-histidine, L-methionine, EDTA, and tonicity modifier (mannitol, glycerol, or trehalose) to a 250 mL plastic Nalgene bottle (Thermo Scientific, 2019-0250) equipped with a magnetic stir bar. 200 mL HyClone™ Water for Injection (WFI) Quality Water (GE Healthcare Hyclone SH30221.10) was added to the bottle and stirred at 300 rpm until dissolved. 1N HCl was added to adjust the pH to the desired level. The remaining amount of water necessary to achieve the target batch weight was added, filtered through a 0.22 μm PVDF membrane filter and stored between 2° C. and 8° C. The weighed amounts of Compound A were transferred to glass containers, each equipped with a magnetic stir bar. The prepared solutions were added to the containers and allowed to stir at 300 rpm at room temperature until dissolved. The pH of each formulation was measured and adjusted as needed with 1N HCl or 1N NaOH and filtered through 0.22 μm PVDF membrane filters. Each of the formulations was filled at a 1 mL fill volume into 6R vials. Each vial was stoppered, sealed with an aluminum cap, and vials were autoclaved at 121° C. for 15 minutes.

TABLE 14
Summary of Formulations Tested

| | Com- | | | | | |
|---|---|---|---|---|---|---|
| | pound A | | | | | |
| | Concen- | | | | | |
| Formu- | tration | | | | | |
| lation | (mg/mL) | Solution Composition | | | | pH |
| E4-F1 | 0.6 | 50 mM | 10 mM L- | 0.5 mM | 187 mM | 7.0 |
| | | Histidine | Methionine | EDTA | Mannitol | |
| E4-F2 | 6.0 | 50 mM | 10 mM L- | 0.5 mM | 187 mM | 7.0 |
| | | Histidine | Methionine | EDTA | Mannitol | |
| E4-F3 | 0.6 | 50 mM | 10 mM L- | 0.5 mM | 185 mM | 7.0 |
| | | Histidine | Methionine | EDTA | Glycerol | |
| E4-F4 | 6.0 | 50 mM | 10 mM L- | 0.5 mM | 185 mM | 7.0 |
| | | Histidine | Methionine | EDTA | Glycerol | |
| E4-F5 | 0.6 | 50 mM | 10 mM L- | 0.5 mM | 196 mM | 7.0 |
| | | Histidine | Methionine | EDTA | Trehalose | |
| E3-F6 | 0.6 | 50 mM | 10 mM L- | 0.5 mM | 187 mM | 6.0 |
| | | Histidine | Methionine | EDTA | Mannitol | |
| E4-F7 | 6.0 | 50 mM | 10 mM L- | 0.5 mM | 187 mM | 6.0 |
| | | Histidine | Methionine | EDTA | Mannitol | |
| E4-F8 | 0.6 | 50 mM | 10 mM L- | 0.5 mM | 185 mM | 6.0 |
| | | Histidine | Methionine | EDTA | Glycerol | |
| E4-F9 | 6.0 | 50 mM | 10 mM L- | 0.5 mM | 185 mM | 6.0 |
| | | Histidine | Methionine | EDTA | Glycerol | |
| E4-F10 | 0.6 | 50 mM | 10 mM L- | 0.5 mM | 196 mM | 6.0 |
| | | Histidine | Methionine | EDTA | Trehalose | |

At the initial time point, all formulations were solutions that were free of particles.

TABLE 15
Degradation Product Growth

| | Total Degradation Growth (% of Compound A) vs Initial | | | |
|---|---|---|---|---|
| | 4 weeks @ | 4 weeks @ | 13 weeks @ | 13 weeks @ |
| Formulation | 40° C. | 60° C. | 5° C. | 40° C. |
| E4-F1 | 0.1 | 0.2 | 0.0 | 0.1 |
| E4-F2 | 0.1 | 0.3 | 0.0 | 0.1 |
| E4-F3 | 0.1 | 0.3 | NT | NT |
| E4-F4 | 0.1 | 0.4 | NT | NT |
| E4-F5 | 0.1 | 0.3 | NT | NT |
| E4-F6 | 0.0 | 0.4 | 0.1 | 0.2 |
| E4-F7 | 0.0 | 0.5 | 0.1 | 0.3 |
| E4-F8 | 0.0 | 0.6 | NT | NT |
| E4-F9 | 0.0 | 0.6 | NT | NT |
| E4-F10 | 0.1 | 0.6 | NT | NT |

Upon testing of the initial samples after autoclave processing, no significant degradation of Compound A or change in appearance were observed. Testing at the 4-week time point showed minimal degradation growth at the accelerated storage conditions (40° C. and 60° C.) for all formulations, except those formulations with a solution pH=6 (E4-F6 thru E4-F10). Testing at the 13-week time point was only conducted on the mannitol-containing formulations (E4-F1, E4-F2, E4-F6, and E4-F7), which showed minimal degradation growth at the 5° C. and 40° C. storage conditions.

### Example 5: Identification of Impact of EDTA on Compound a Formulations in the Presence of Iron

EDTA was evaluated as a metal chelator to mitigate degradation induced by the presence of metals, such as iron (III). Iron (III) can be introduced into the formulation as an impurity in Compound A, as an impurity in excipients, and from the manufacturing process train.

All solutions were prepared by transferring appropriate amounts of L-histidine, L-methionine, EDTA, and mannitol to a 125 mL plastic Nalgene bottle (Nalgene, 342020-0125) equipped with a magnetic stir bar. 80 mL HyClone™ Water for Injection (WFI) Quality Water (GE Healthcare Hyclone SH30221.10) was added to the bottle and stirred at 250-350 rpm until dissolved. 1N HCl was added to adjust the pH to the desired level. The remaining amount of water necessary to achieve the target batch weight was added, filtered through a 0.22 μm PVDF membrane filter and stored between 2° C. and 8° C. Compound A was added to the solutions by weighing appropriate amount and transferring to a 50 mL conical tube. 50 ml of the prepared solution was then added to the tube and vortexed at room temperature to mix. The pH of each formulation was measured and adjusted as needed with 1N HCl or 1N NaOH and filtered through 0.22 μm PVDF membrane filters.

TABLE 16
Summary of Diluents

| | Com- | L-Hisi- | L-Methi- | | | |
|---|---|---|---|---|---|---|
| Formu- | pound A | tine | onine | Mannitol | EDTA | |
| lation | (mg/mL) | (mM) | (mM) | (mM) | (mM) | pH |
| E5-F1 | 0.6 | 50 | 10 | 187 | 0.00 | 6.5 |
| E5-F2 | | | | | 0.05 | |
| E5-F3 | | | | | 0.25 | |
| E5-F4 | | | | | 0.50 | |

### Iron Spiking:

A 1 mg/mL solution of iron (III) chloride hexahydrate was prepared by adding 14.6 mg of FeCl₃ into a 20 mL scintillation vial and adding 14.6 mL water. This yielded a solution that was 21% iron, or 210 ug/mL iron. Active formulation samples were spiked with iron by adding 25 mL of each formulation into 100 mL plastic bottles (PN) and pipetting 119 μL FeCl₃ solution followed by vortexing to mix. This resulted in 1 ppm of Fe in each formulation sample. Control samples were also included in the formulation that were not spiked with iron (III).

Each of the formulated solutions was were filled into a 6R vial (Type 1, European Blow Back) with a 2 mL formulation solution fill volume. Each vial was stoppered and sealed with an aluminum cap. Samples were placed in autoclave and run at 121° C. for 15 minutes. Vials were brought to equilibrium at at room temperature and placed in stability chambers (protected from light) at temperatures of 5° C., 30° C., and 40° C.

TABLE 17
Assay and Degradation ProductsControl vs. 1 ppm Iron Spike

| | Total Degradation Growth (% of Compound A) vs. Control* | | | |
|---|---|---|---|---|
| | | 4 weeks @ | 4 weeks @ | 4 weeks @ |
| Formulations | Initial | 5° C. | 30° C. | 40° C. |
| E5-F1 | 0.10 | 0.12 | 0.36 | 0.53 |
| E5-F2 | −0.01 | 0.00 | 0.00 | 0.01 |
| E5-F3 | −0.01 | 0.00 | 0.04 | 0.01 |
| E5-F4 | 0.05 | 0.00 | 0.01 | −0.01 |

Degradation growth was observed in E5-F1 (0.00 mM EDTA) after 4 weeks at all storage conditions. The formulations containing EDTA (E5-F2 through E5-F4) are stable in the presence of 1 pmm Fe(III) and show no significant degradation growth at any of the time points or conditions.

### Example 6: Impact of a Double Autoclave Cycle of 121° C./15 Min in Formulations Comprised of Alternative Tonicity Modifiers at pH 6 and 7

The impact of alternative tonicity modifiers on the stability of Compound A formulations was evaluated under significant terminal sterilization (2× cycles at 121° C. for 15 min).

### Formulation Preparations

All solutions were prepared by transferring appropriate amounts of L-histidine, L-methionine, EDTA and tonicity modifier (mannitol, glycerol or trehalose) and a magnetic stir bar to a 250 mL plastic Nalgene bottle (Thermo Scientific, 2019-0250). 200 mL HyClone™ Water for Injection (WFI) Quality Water (GE Healthcare Hyclone SH30221.10) was added to the bottle and stirred at 300 rpm until dissolved. 1N HCl was added to adjust the pH to the desired level. The remaining amount of water necessary to achieve the target batch weight was added, filtered through a 0.22 μm PVDF membrane filter and stored between 2° C. and 8° C.

TABLE 18
Summary of Formulations

| Formu- | Compound | Histidine | Methionine | EDTA | Mannitol | Glycerol | Trehalose | |
|---|---|---|---|---|---|---|---|---|
| lation | A (mg/mL) | (mM) | (mM) | (mM) | (mM) | (mM) | (mM) | pH |
| E3-F6 | 0.6 | 50 | 10 | 0.5 | 187 | — | — | 6 |
| E4-F6 | 6.0 | | | | 187 | — | — | |
| E4-F7 | 0.6 | | | | — | 185 | — | |
| E4-F8 | 6.0 | | | | — | 185 | — | |
| E4-F9 | 0.6 | | | | — | — | 196 | |
| E4-F1 | 0.6 | | | | 187 | — | — | 7 |
| E4-F2 | 6.0 | | | | 187 | — | — | |
| E4-F3 | 0.6 | | | | — | 185 | — | |
| E4-F4 | 6.0 | | | | — | 185 | — | |
| E4-F5 | 0.6 | | | | — | — | 196 | |

The appropriate amount of Compound A for each formulation was added to a glass vial equipped with a magnetic stir bar. The solutions prepared above were then added to the vials and allowed to stir at 300 rpm at room temperature until Compound A was dissolved. The pH of each formulation was measured and adjusted as needed with 1N HCl or 1N NaOH and filtered through 0.22 μm PVDF membrane filters.

Each of the formulated solutions was filled into a 6R vial (Type 1, European Blow Back) with a 1 mL formulation solution fill volume. Each vial was stoppered and sealed with an aluminum cap. Samples were placed in an autoclave (Tuttnauer Brinkmann 2540EK) and steam sterilized for 15 minutes at 121° C. After the initial cycle, the samples were cooled at room temperature. The samples were then placed back in the autoclave and run on a liquids cycle at 121° C. for 15 minutes for a second time. The samples were cooled at room temperature and then stored between 2° C. and 8° C. At the time of preparation, the formulations were inspected for visible particulates. At the initial time point, all formulations were essentially free of visible particulates. Samples were then analyzed for the presence of absolute degradation products (as opposed to difference vs initial), shown in Table 19 below.

At the time of preparation, the formulations were visually inspected for changes in color or visible particulates. At the initial time point, all formulations were essentially free of visible particulates. The samples were placed in stability chambers.

TABLE 19
Double Autoclave Cycling Assay
and Degradation Summary

| | Total Degradation |
|---|---|
| Formulation | Products (% of Compound A) |
| E3-F6 | 0.95 |
| E4-F1 | 0.88 |
| E4-F7 | 1.03 |
| E4-F3 | 0.95 |
| E4-F9 | 1.03 |
| E4-F5 | 0.94 |
| E4-F6 | 0.96 |
| E4-F2 | 0.93 |
| E4-F8 | 0.96 |
| E4-F4 | 0.92 |

This study did not reveal any differentiation after a second autoclave cycle between formulations containing various tonicity modifiers or from differences in pH. All these formulations were able to withstand the worst-case terminal sterilization conditions, as the total degradates were less than 1%.

### Example 7: Evaluation of Buffer Capacity

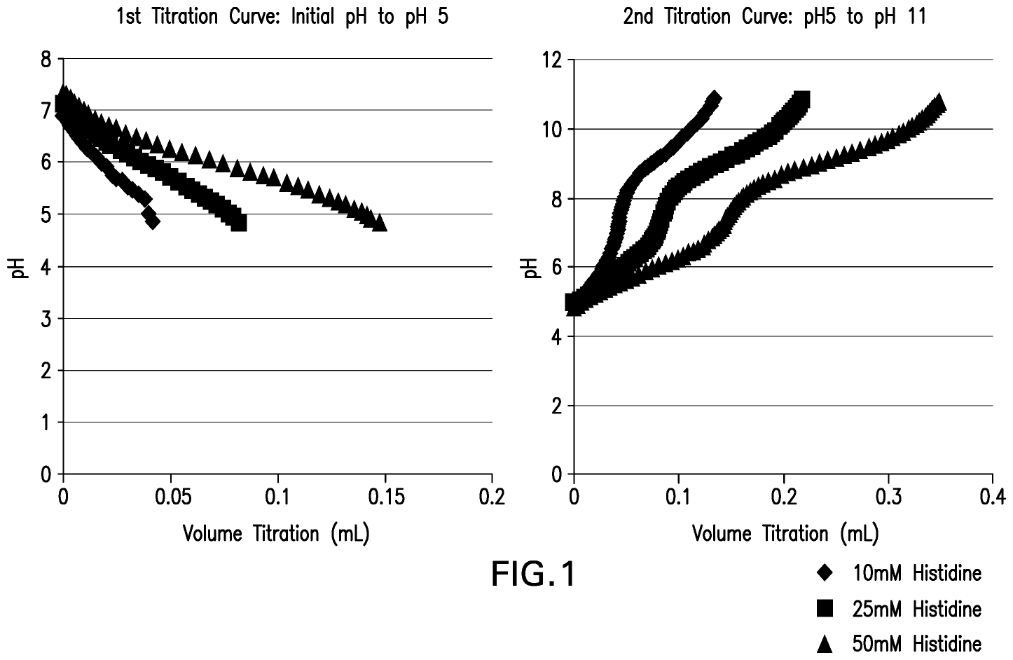

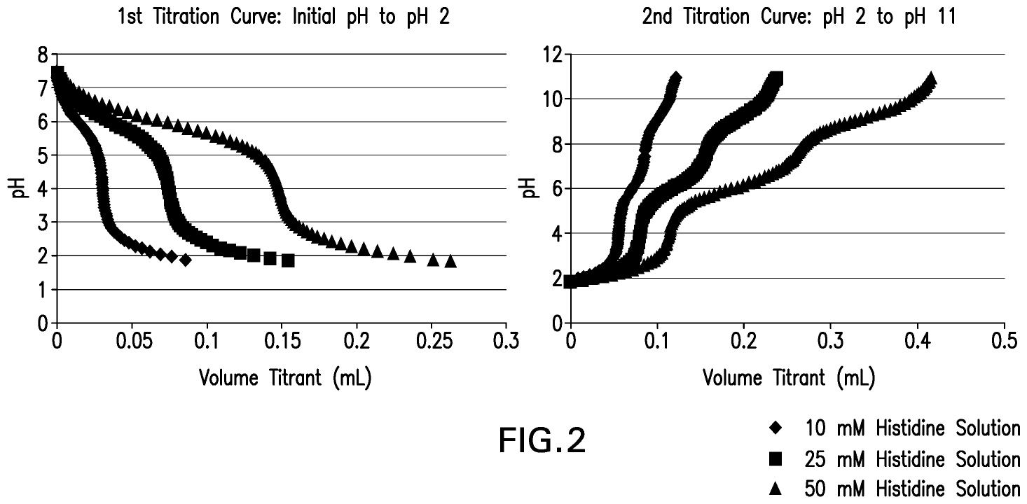

Buffer capacity of 10, 25 and 50 mM histidine-only solutions, as well as the buffer capacity of 6 mg/mL Compound A formulations was containing 10, 25, and 50 mM histidine buffers were measured using potentiometric pH methods. The titration curves for the formulations containing histidine and formulations containing histidine and Compound A are shown in FIGS. 1-3.

TABLE 20
Solution Compositions Studied for Buffer Capacity Experiments

| | Compound | Solution Composition | | | |
|---|---|---|---|---|---|
| Formulation | A (mg/mL) | Histidine | L-Methionine | EDTA | Mannitol |
| E7-F1 | — | 10 mM | — | — | — |
| E7-F2 | — | 25 mM | — | — | — |
| E7-F3 | — | 50 mM | — | — | — |
| E7-F4 | 6 | 10 mM | 10 mM | 0.5 mM | 187 mM |
| E7-F5 | 6 | 25 mM | 10 mM | 0.5 mM | 187 mM |
| E7-F6 | 6 | 50 mM | 10 mM | 0.5 mM | 187 mM |

The potentiometric titrations were performed with the Sirius T3 instrument using a double junction electrode. The electrode was standardized from pH 1.8 to 12.2 by performing a Blank standardization assay. The HCl titrant was standardized by performing a standardization assay and was approximately 0.5 M. The KOH titrant was standardized against potassium hydrogen phthalate in a KHP assay and was approximately 0.5 M.

The solutions (1.5 ml) were each manually added to the analysis vials using a digital pipette. The data collection rate was set to ΔpH=0.08.

Two titrations were performed for each solution within the same vial. For the placebo histidine buffer solutions, the starting pH of the solution was not adjusted for the 1st titration and the solution was titrated down to pH 2. After the end of the 1st titration, the 2nd titration of the same solution was upwards from pH 2 to pH 11.

For the histidine buffer solutions, the starting pH of the solution was not adjusted for the 1st titration and the solution was titrated down to pH 5. After the end of the 1st titration, the 2nd titration of the same solution was upwards from pH 5 to pH 11.

The 2nd titration with 50 mM histidine buffer had precipitate below pH 5.2.

The buffer capacity of Compound A formulations was examined at different histidine concentrations. During the first titration, the formulations were titrated from pH 7 to pH 5, followed by a second titration from pH 5 to pH 11. The buffer capacity values are shown in Table 21. The second experiment examined the buffer capacity of histidine-only solutions at various concentrations. In the initial titration, the solutions were titrated down to pH 2, followed by a second titration from pH 2 to pH 11.

A comparison of the buffer capacity of histidine only solutions and 6 mg/mL Compound A formulations are shown in Table 21.

TABLE 21
Buffer Capacity Values

| | Buffer Capacity, β | |
|---|---|---|
| Formulation | pH 6 | pH 7 |
| E7-F1 | 0.02 | 0.01 |
| E7-F2 | 0.04 | 0.02 |
| E7-F3 | 0.08 | 0.03 |
| E7-F4 | 0.02 | 0.01 |
| E7-F5 | 0.04 | 0.02 |
| E7-F6 | 0.08 | 0.03 |

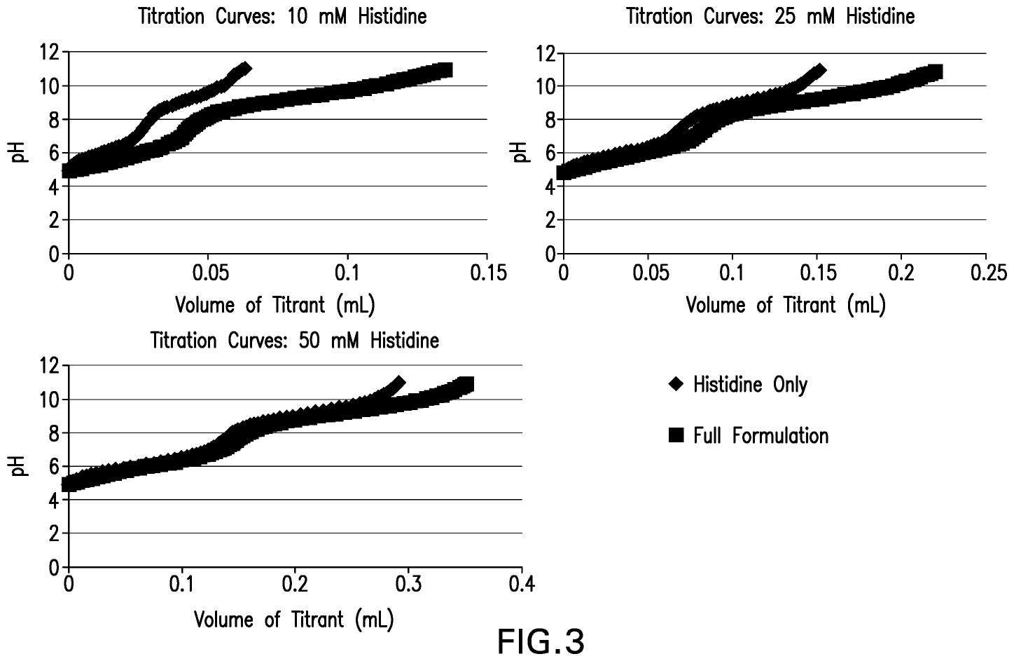

The buffer capacity of histidine was the highest at the pKa of histidine, or pH 6. In order to stabilize the pH of formulations within the pH target of 6.5±0.5, 50 mM histidine was most effective. A steep titration curve was observed for 10 mM histidine, indicating poor buffering capacity in target pH range (FIG. 3).

### Example 8: Comparison of Sucrose and Sodium Chloride Tonicity Modifier Formulations

A stability study was initiated to explore the formulation composition, probing 10-50 mM histidine, 0-10 mM methionine, 0.6-6.0 mg/mL Compound A concentration, pH range of 7-7.5, and tonicity modifier (sucrose or sodium chloride), and the effect of terminal sterilization.

For this study, Compound A diluent solutions were prepared by dissolving target amounts of histidine buffer, tonicity modifier (sucrose or sodium chloride), L-methionine and EDTA in water (see Table 22). The diluent solutions were adjusted to pH 6.0, 7.0, or 7.5. Each diluent formulation was filtered using 0.22 μm PVDF membrane filters. Compound A 0.6 mg/mL drug product formulations were made by adding the appropriate volume of Compound A diluent solution to Compound A (Table 22). Compound A formulations were filtered using 0.22 μm PVDF membrane filters. Each of the formulated solutions was filled into a 6R vial (Type 1, European Blow Back) with a 1 mL formulation solution fill volume. Each vial was stoppered and sealed with an aluminum cap. The samples were placed in an autoclave (Tuttnauer Brinkmann 2540EK) and steam sterilized for 15 minutes at 121° C. Autoclaved samples were staged, protected from light, and placed in a 2° C. to 8° C., 30° C., and 40° C. environmental stability chamber for 13 weeks.

TABLE 22
Summary of Formulations

| Formu- | Compound A | | | | | |
|---|---|---|---|---|---|---|
| lation | (mg/mL) | Solution Composition | | | | pH |
| E8-F1 | 0.6 | 50 mM | 0 mM L- | 0.5 mM | 99 mM | 7.5 |
| | | Histidine | Methionine | EDTA | NaCl | |
| E8-F2 | 0.6 | 10 mM | 0 mM L- | 0.5 mM | 184 mM | 7.5 |
| | | Histidine | Methionine | EDTA | Sucrose | |
| E8-F3 | 6 | 10 mM | 5 mM L- | 0.5 mM | 99 mM | 7 |
| | | Histidine | Methionine | EDTA | NaCl | |
| E8-F4 | 6 | 10 mM | 10 mM L- | 0.5 mM | 184 mM | 6 |
| | | Histidine | Methionine | EDTA | Sucrose | |
| E8-F5 | 6 | 50 mM | 5 mM L- | 0.5 mM | 184 mM | 6 |
| | | Histidine | Methionine | EDTA | Sucrose | |
| E8-F6 | 0.6 | 50 mM | 0 mM L- | 0.5 mM | 184 mM | 6 |
| | | Histidine | Methionine | EDTA | Sucrose | |
| E1-F2 | 0.6 | 50 mM | 10 mM L- | 0.5 mM | 99 mM | 7 |
| | | Histidine | Methionine | EDTA | NaCl | |
| E8-F7 | 0.6 | 10 mM | 5 mM L- | 0.5 mM | 184 mM | 6 |
| | | Histidine | Methionine | EDTA | Sucrose | |
| E8-F8 | 6 | 50 mM | 10 mM L- | 0.5 mM | 184 mM | 6 |
| | | Histidine | Methionine | EDTA | Sucrose | |
| E8-F9 | 6 | 10 mM | 0 mM L- | 0.5 mM | 99 mM | 7.5 |
| | | Histidine | Methionine | EDTA | NaCl | |
| E8-F10 | 0.6 | 50 mM | 5 mM L- | 0.5 mM | 99 mM | 7.5 |
| | | Histidine | Methionine | EDTA | NaCl | |
| E8-F11 | 6 | 10 mM | 0 mM L- | 0.5 mM | 184 mM | 6 |
| | | Histidine | Methionine | EDTA | Sucrose | |
| E8-F12 | 6 | 50 mM | 0 mM L- | 0.5 mM | 184 mM | 7.5 |
| | | Histidine | Methionine | EDTA | Sucrose | |
| E8-F13 | 6 | 10 mM | 5 mM L- | 0.5 mM | 184 mM | 7.5 |
| | | Histidine | Methionine | EDTA | Sucrose | |
| E8-F14 | 6 | 10 mM | 10 mM L- | 0.5 mM | 99 mM | 7 |
| | | Histidine | Methionine | EDTA | NaCl | |
| E8-F15 | 6 | 10 mM | 10 mM L- | 0.5 mM | 184 mM | 7.5 |
| | | Histidine | Methionine | EDTA | Sucrose | |
| E8-F16 | 0.6 | 50 mM | 10 mM L- | 0.5 mM | 184 mM | 7.5 |
| | | Histidine | Methionine | EDTA | Sucrose | |
| E8-F17 | 0.6 | 10 mM | 10 mM L- | 0.5 mM | 99 mM | 7.5 |
| | | Histidine | Methionine | EDTA | NaCl | |
| E8-F18 | 6 | 50 mM | 10 mM L- | 0.5 mM | 99 mM | 7.5 |
| | | Histidine | Methionine | EDTA | NaCl | |
| E8-F19 | 0.6 | 10 mM | 0 mM L- | 0.5 mM | 99 mM | 7 |
| | | Histidine | Methionine | EDTA | NaCl | |
| E8-F20 | 0.6 | 10 mM | 10 mM L- | 0.5 mM | 184 mM | 6 |
| | | Histidine | Methionine | EDTA | Sucrose | |
| E8-F21 | 6 | 50 mM | 0 mM L- | 0.5 mM | 99 mM | 7 |
| | | Histidine | Methionine | EDTA | NaCl | |

TABLE 23
Summary of Formulation Stability
Total Degradation Growth (% of Compound A) vs. Initial

| | 4 weeks, | 4 weeks, | 13 weeks, | 13 weeks, |
|---|---|---|---|---|
| Formulation | 5° C. | 40° C. | 5° C. | 40° C. |
| E8-F1 | −0.04 | 0.04 | −0.01 | 0.14 |
| E8-F2 | 0.04 | 0.66 | 0.05 | 2.46 |
| E8-F3 | −0.05 | 0.01 | −0.05 | 0.08 |
| E8-F4 | −0.12 | 0.60 | −0.14 | 1.59 |
| E8-F5 | 0.03 | 1.18 | 0.33 | 2.98 |
| E8-F6 | 0.58 | 7.83 | 1.01 | 29.06 |
| E1-F2 | −0.05 | 0.01 | −0.04 | 0.09 |
| E8-F7 | −0.09 | 1.20 | −0.04 | 2.79 |
| E8-F8 | −0.15 | 1.35 | 0.25 | 3.48 |
| E8-F9 | −0.04 | 0.04 | −0.10 | 0.06 |
| E8-F10 | −0.20 | 0.00 | −0.19 | 0.02 |
| E8-F11 | 0.02 | 0.74 | 0.02 | 2.15 |
| E8-F12 | −0.06 | 0.44 | −0.04 | 1.74 |
| E8-F13 | −0.06 | 0.15 | −0.06 | 0.37 |
| E8-F14 | −0.06 | 0.02 | −0.05 | 0.08 |
| E8-F15 | −0.04 | −0.01 | −0.05 | 0.09 |
| E8-F16 | −0.04 | 0.09 | 0.20 | 0.49 |
| E8-F17 | −0.09 | 0.00 | −0.07 | 0.01 |
| E8-F18 | −0.11 | 0.13 | −0.10 | 0.02 |
| E8-F19 | −0.00 | 0.03 | 0.02 | 0.17 |
| E8-F20 | 0.11 | 1.04 | 0.06 | 2.65 |
| E8-F21 | 0.00 | 0.05 | −0.02 | 0.15 |

These results demonstrate that sucrose formulations show significantly more degradation growth than solutions containing sodium chloride. In the formulations containing methionine in the presence of sucrose, degradation growth was mitigated as compared to those without methionine. Formulations containing sodium chloride also showed slight improvements in stability in the presence of methionine. Overall, all formulations in the presence of methionine were stable, however sodium chloride formulations were stable with and without methionine. For the sodium chloride formulation, no significant differences in degradation growth were observed for any formulation variable, including methionine level, buffer concentration, and pH. For sucrose formulations, the formulations containing 0 mM methionine were significantly less stable than the sucrose-containing formulations containing 5 mM or 10 mM methionine. Sucrose formulations containing 10 mM histidine were more chemically stable than sucrose-containing formulations containing 50 mM histidine. The sucrose-containing formulations at pH 6 were less chemically stable than sucrose formulations at pH 7.5.

### Example 9: Oxidative Degradation Mitigation Studies

The impact of a nitrogen overlay, as compared to standard ambient atmosphere, in the storage vial headspace, and the presence of methionine and EDTA on the stability of low-concentration Compound A formulations were studied.

Buffer solutions were prepared by weighing out sodium phosphate dibasic anhydrous, sodium phosphate monobasic anhydrous, and sucrose individually onto weigh paper and transferring into a 100 mL volumetric flask. To the volumetric flask, 80% of the required water was added and swirled to dissolve all solids. The pH was measured using a pH meter and recorded. 1N HCl was added to pH adjust buffer solution to pH 7.0 (+/−0.1). Additional water was added to reach the fill line on volumetric flask. The pH of solution was measured and recorded, followed by filtering the solution using a 0.22 μm PVDF membrane filter.

Formulation E9-F1 was prepared by weighing out Compound A onto weigh paper and transferring into a 100 mL beaker. The diluent solution was added by weight using a plastic syringe and stirred at room temperature for 5 minutes. The pH of the solution was checked and recorded using a pH meter. In a laminar flow hood, the formulation was filtered using a sterile plastic syringe fitted with a sterile syringe filter into a 100 mL beaker. Using a sterile 10 mL multidispense pipet, 1 mL portions of the formulation solution were pipetted into 6R vials and capped. Vials were removed from laminar flow hood, and a portion of these vials were placed in a nitrogen glovebox. Once in the glovebox, the caps were removed, and solutions equilibrated for 1 hour.

Formulation E9-F2 was prepared by weighing out Compound A onto weigh paper and transferring into a 100 mL beaker. The diluent solution was added by weight using a plastic syringe and stirred at room temperature for 5 minutes. The pH of the solution was checked and recorded using a pH meter. In a laminar flow hood, the formulation was filtered using a sterile plastic syringe fitted with a sterile syringe filter into a 100 mL beaker. Using a sterile 10 mL multidispense pipet, 1 mL portions of the formulation solution were pipetted into 6R vials and capped.

Formulation E9-F3 was prepared by weighing out Compound A onto weigh paper and transferring into a 100 mL beaker. The diluent solution was added by weight using a plastic syringe and stirred at room temperature for 5 minutes. The pH of the solution was checked and recorded using a pH meter. In a laminar flow hood, the formulation was filtered using a sterile plastic syringe fitted with a sterile syringe filter into a 100 mL beaker. Using a sterile 10 mL multidispense pipet, 1 mL portions of the formulation solution were pipetted into 6R vials and capped. Formulation vials were stationed at 5° C. and 40° C. for up to 10 weeks.

TABLE 24
Summary of Formulations

| | Com- | Solution Composition | | | | | | |
|---|---|---|---|---|---|---|---|---|
| | pound | | L- | | | | | |
| | A | | | | | | | |
| Formu- | (mg/ | NaPO₄ | Methi- | | | | | Head- |
| lation | mL) | Buffer | onine | EDTA | | Sucrose | pH | space |
| E9-F1 | 1 | 10 mM | 0 mM | 0 | mM | 263 mM | 7 | Ambient |
| E9-F2 | 1 | 10 mM | 0 mM | 0 | mM | 263 mM | 7 | Nitrogen |
| E9-F3 | 1 | 10 mM | 10 mM | 0 | mM | 263 mM | 7 | Ambient |
| E9-F4 | 1 | 10 mM | 10 mM | 0.5 | mM | 263 mM | 7 | Ambient |

TABLE 25
Growth of Oxidative Degradation Products Compared to Initial at 5° C. and 40° C.

| | Total Oxidative Degradation Growth (% of Compound A) vs Initial | | | | | | | |
|---|---|---|---|---|---|---|---|---|
| Formu- | 2 weeks | 2 weeks | 2 weeks | 4 weeks | 4 weeks | 4 weeks | 10 weeks | 10 weeks |
| lation | @5° C. | @30° C. | @40° C. | @5° C. | @30° C. | @40° C. | @5° C. | @30° C. |
| E9-F1 | 0.00 | 0.06 | 0.14 | 0.00 | 0.06 | 0.12 | 0.00 | 0.18 |
| E9-F2 | 0.01 | NT | 0.11 | 0.01 | NT | 0.13 | 0.08 | 0.14 |
| E9-F3 | 0.01 | 0.08 | 0.10 | 0.06 | 0.09 | 0.13 | 0.07 | 0.11 |
| E9-F4 | 0.01 | 0.09 | 0.11 | 0.07 | 0.10 | 0.19 | 0.08 | 0.11 |

Formulations with nitrogen overlay as well as the inclusion of methionine and EDTA in vials with ambient headspace showed no significant degradation over 10 weeks under refrigerated storage and showed slight degradation growth over 10 weeks under accelerated storage conditions (40° C.). These data do not indicate any significant chemical stability differences between the tested oxidation mitigation strategies.

### Example 10: Formulation Stability Based on Buffer Composition and Concentration of Compound A

The stability of Compound A formulations was studied as a function of Compound A concentration and buffer composition.

The solutions were prepared by weighing out appropriate amounts of buffer (histidine or sodium phosphate), L-methionine, EDTA and sucrose into a 100 mL volumetric flask. 80 mL of water was added to the flask and swirled to dissolve all solids. The pH values of the solutions were measured and recorded using a calibrated pH meter. If required, pH was adjusted using 1N HCL. Additional water was added to reach the fill line of the volumetric flask. The final pH was measured and recorded followed by filtering the solution using a 0.22 μm PVDF membrane filter. Formulations were prepared by weighing out the appropriate amount of Compound A into a 30 mL sterile vial and adding diluent using a plastic syringe (Table 26). The formulations were stirred at room temperature for 1 hour followed by measuring and recording the pH. In a laminar flow hood, the solution was filtered using a sterile plastic syringe fated with a sterile syringe filter into a 100 mL beaker, and pipetted into 6R type 1, 20 mm neck and European Blowback vials.

TABLE 26
Summary of Formulations

| | Com- | | | | | |
|---|---|---|---|---|---|---|
| Formu- | pound A | | | | | |
| lation | (mg/mL) | Solution Composition | | | | pH |
| E10-F1 | 1.0 | 50 mM | 10 mM L- | 0.5 mM | 75 mg/mL | 7 |
| | | Histidine | Methionine | EDTA | Sucrose | |
| E10-F2 | 7.1 | 50 mM | 10 mM L- | 0.5 mM | 50 mg/mL | 7 |
| | | NaPO₄ | Methionine | EDTA | Sucrose | |
| E10-F3 | 9.3 | 50 mM | 10 mM L- | 0.5 mM | 63 mg/mL | 7 |
| | | Histidine | Methionine | EDTA | Sucrose | |

TABLE 27
Growth of Degradation Products Compared to Initial at 5° C. and 40° C.

| | Total Oxidative Degradation Growth (% of Compound A) vs Initial | | | | | | |
|---|---|---|---|---|---|---|---|
| Formu- | 2 weeks | 2 weeks | 4 weeks | 4 weeks | 4 weeks | 10 weeks | 10 weeks |
| lation | @5° C. | @40° C. | @5° C. | @30° C. | @40° C. | @5° C. | @30° C. |
| E10-F1 | 0.05 | 0.07 | 0.06 | 0.07 | 0.08 | 0.05 | 0.08 |
| E10-F2 | 0.14 | 0.20 | 0.15 | 0.18 | 0.29 | NT | NT |
| E10-F3 | 0.05 | 0.06 | 0.07 | 0.07 | 0.08 | NT | NT |

After 4 weeks of storage at accelerated stability conditions, minimal growth of the known degradates were observed. Due to these species being present as process impurities in the batch used for prototype stability, in some formulations these grew above the 0.1% reporting threshold. As such, these formulations would be classified as stable.

### Example 11: Stability of Compound a Under ForcedStressed Conditions

The stability of Compound A was studied under stressed conditions to probe the impact of peroxy radical-based oxidation, two-electron oxidation, and base-catalyzed hydrolysis.

0.05 mg/mL stock solutions of Compound A were prepared by adding 3 mg of Compound A to a 50 mL volumetric flask and diluted to volume with either 50/50 water/methanol (stock solution A), or 50/50 water/acetonitrile (stock solution B).

An azobisisobutyronitrile (AIBN) stress solution was prepared by dissolving 7.77 mg of AIBN in 10 mL of stock solution A in an amber volumetric flask and sonicating to dissolve. The AIBN control solution was prepared by dissolving 8.48 mg of AIBN in 10 mL of 50/50 methanol/water in an amber volumetric flask. Both solutions were placed in an oven at 40° C. for 24 hours.

A peroxide stress solution was prepared by adding 1.0 mL of 3% hydrogen peroxide to 9.0 mL of stock solution A in a volumetric flask. An aliquot of the solution was placed into an amber vial kept at room temperature, and another aliquot was stored at 5° C. for 24 hours. The peroxide control solution was prepared by adding 1.0 mL of 3% hydrogen peroxide to 9.0 mL 50/50 methanol/water in an amber volumetric flask. This solution was kept at room temperature for 24 hours.

A 0.1N NaOH stress solution was prepared by adding 1.0 mL of 1N sodium hydroxide to 9.0 mL of stock solution B in an amber volumetric flask. One sample was kept at room temperature, and the identical sample was placed in an oven at 60° C. for 24 hours. A 0.1N NaOH control solution was prepared by adding 1.0 mL of 1N sodium hydroxide to 9.0 mL of 50/50 acetonitrile/water in an amber volumetric flask.

TABLE 28
Results from Forced Stress Screen

| Forced | % Recovery of | Sensitive for |
|---|---|---|
| Stress Sample | Compound A | Hydrolysis or Oxidation |
| AIBN stress | 97.7 | No sensitivity; <5% drug loss |
| Peroxide stress at RT | 44.6 | High sensitivity; >10% drug loss |
| Peroxide stress at 5° C. | 82.4 | High sensitivity; >10% drug loss |
| 0.1 N NaOH base stress | 100.0 | No sensitivity; <5% drug loss |
| | | in 0.1 N NaOH after 24h/RT |

No major degradation was observed under AIBN or 0.1N NaOH base stress. Compound A was shown to be highly sensitive to two-electron oxidative processes. The percent of Compound A was calculated based on the area count of active peak in the control samples compared to stress samples.

### Example 12: Formulation Stability in Presence of Iron after Terminal Sterilization

Methionine was evaluated as a sacrificial antioxidant, and EDTA was evaluated as a metal cheloator to mitigate degradation induced by the presence of metals, such as iron (III), which can be exacerbated by the heat treatment process of terminal sterilization. Iron (III) and other metals can be introduced into the formulation as an impurity in Compound A, as an impurity in excipients, and from the manufacturing process.

For this study, Compound A diluent solutions were prepared by dissolving target amounts of histidine buffer, sucrose, L-methionine and EDTA in HyClone™ Water for Injection (WFI) Quality Water (GE Healthcare Hyclone SH30221.10). Diluent solutions were adjusted to pH 6.5 with 1N NaOH or 1N HCl. Each diluent formulation was filtered using 0.22 μm PVDF membrane filters. Compound A 0.6 mg/mL drug product formulations were made by adding the appropriate volume of diluent solution to Compound A (Table 29). Compound A formulations were filtered using 0.22 μm PVDF membrane filters.

TABLE 29
Summary of Formulations

| Formu- | Compound A | L- | | L-Methi- | | |
|---|---|---|---|---|---|---|
| lation | (mg/mL) | Histidine | Sucrose | onine | EDTA | pH |
| E12-F1 | 0.6 | 50 mM | 184 mM | 10 mM | 0.5 mM | 6.5 |
| E12-F2 | 0.6 | 50 mM | 184 mM | — | — | 6.5 |
| E12-F3 | 0.6 | 50 mM | 184 mM | 10 mM | — | 6.5 |
| E12-F4 | 0.6 | 50 mM | 184 mM | — | 0.5 mM | 6.5 |

### Iron Spiking:

Iron (III) chloride hexahydrate was used to prepare an iron solution for all iron spiking studies. 15 mg of iron (III) chloride hexahydrate was transferred to a 20 mL scintillation vial. 15 mL HyClone™ Water for Injection (WFI) Quality Water (GE Healthcare Hyclone SH30221.10) was then added to generate a 1 mg/mL solution. To 100 mL plastic bottles, 30 mL of each formulation was added, followed by spiking in the appropriate amount of iron from the FeCl₃ (0.01 or 1 ppm). Control samples were also included in the formulation that were not spiked with iron (III).

TABLE 30
Summary of Experimental Conditions

| Formulation | Fe Spike |
|---|---|
| E12-F1 | 0 ppm |
| E12-F1 | 0.01 ppm |
| E12-F1 | 1 ppm |
| E12-F2 | 0 ppm |
| E12-F2 | 0.01 ppm |
| E12-F2 | 1 ppm |
| E12-F3 | 0 ppm |
| E12-F3 | 0.01 ppm |
| E12-F3 | 1 ppm |
| E12-F4 | 0 ppm |
| E12-F4 | 0.01 ppm |
| E12-F4 | 1 ppm |

Each formulation was aliquoted in 1 mL increments into 6R vials, stoppered and crimped. Samples were placed in an autoclave (Tuttnauer Brinkmann 2540EK) and steam sterilized for 15 minutes at 121° C. Autoclaved samples were staged, protected from light, and placed in a 2° C. to 8° C. environmental stability chamber for 4 weeks.

TABLE 31
Growth of Degradation Products Compared to Control at Initial and 5° C.

| | Total Degradation Growth (% of | | | |
|---|---|---|---|---|
| | Compound A) vs Control | | | |
| | Initial | | 4 Weeks, 5° C. | |
| Formulation | 0.01 ppm | 1 ppm | 0.01 ppm | 1 Ppm |
| E12-F1 | −0.04 | 0.32 | 0.02 | 0.53 |
| E12-F2 | 0.11 | 2.57 | 0.08 | NT |
| E12-F3 | 0.43 | 2.42 | 0.43 | NT |
| E12-F4 | 0.10 | 0.37 | 0.21 | 0.52 |

No significant growth in degradation products was observed in EDTA-containing formulations (E12-F1 and E12-F4) when compared to the control formulations (Table 31). However, an increase in total degradation was observed in formulations E12-F2 and E12-F3, containing no EDTA. Overall, formulations are relatively stable to low levels of iron (III) (0.01 ppm). However, formulations without EDTA (E12-F2 and E12-F3) show higher degradation growth level in the presence of 1 ppm iron (III). The highest level of degradation growth was observed in formulation E12-F2, which contained no functional excipients. In formulations containing EDTA (E12-F1 and E12-F4), minimal degradation was observed across all levels of iron. Overall, formulations containing EDTA provided more robust protection from higher levels of iron (III).

### Example 13: Stability of Compound a Under Peroxide Stress

Different levels of methionine were evaluated to determine the impact of peroxide stress and Compound A degradation. Compound A diluent solutions were prepared by dissolving target amounts of histidine buffer, mannitol, different levels of L-methionine and EDTA disodium dihydrate in HyClone™ Water for Injection (WFI) Quality Water (GE Healthcare Hyclone SH30221.10). Each diluent formulation was filtered using 0.22 nm PVDF membrane filters. Compound A 0.54 mg/ml drug product formulations were made by adding the appropriate volume of diluent solution to Compound A (Table 32). Compound A formulations were filtered using 0.22 nm PVDF membrane filters. Each formulation was spiked with 1 ppm peroxide solution prepared by taking 600 μl of 30% w/w hydrogen peroxide solution and diluting to 100 mL by adding WFI. Control samples (no spiking) and spiked samples were filled in a 6R vial at a volume of 1.2 ml, stoppered and climped. Samples were placed in an autoclave (Tuttnauer Brinkmann 2540EK) and steam sterilized for 15 minutes at 121° C. Autoclaved samples were staged, protected from light, on stability at 5° C., 30° C. and 40° C. for 1 month. Total degradation growth in the spiked formulations when compared to the control was in the range of 0.13-0.26% LC after 4 weeks at the different storage conditions. Given the low level of degradation, all formulations were considered stable in this study even with high level of peroxide in the formulation.

TABLE 32
Summary of Formulations

| | Com- | | | L- | | | | |
|---|---|---|---|---|---|---|---|---|
| Formu- | pound A | L-Histi- | Man- | Methi- | | | | Perox- |
| lation | (mg/mL) | dine | nitol | onine | | EDTA | pH | ide |
| E13-F1 | 0.54 | 50 mM | 220 | 1 | mM | 0.05 | 6.5 | 1 ppm |
| | | | mM | | | mM | | |
| E13-F2 | 0.54 | 50 mM | 220 | 2 | mM | 0.05 | 6.5 | 1 ppm |
| | | | mM | | | mM | | |
| E13-F3 | 0.54 | 50 mM | 220 | 2.5 | mM | 0.05 | 6.5 | 1 ppm |
| | | | mM | | | mM | | |
| E13-F4 | 0.54 | 50 mM | 220 | 1 | mM | 0.05 | 6.5 | — |
| | | | mM | | | mM | | |
| E13-F5 | 0.54 | 50 mM | 220 | 2 | mM | 0.05 | 6.5 | — |
| | | | mM | | | mM | | |
| E13-F6 | 0.54 | 50 mM | 220 | 2.5 | mM | 0.05 | 6.5 | — |
| | | | mM | | | mM | | |

It will be appreciated that various of the above-discussed and other features and functions, or alternatives thereof, may be desirably combined into many other different systems or applications. It will also be appreciated that various presently unforeseen or unanticipated alternatives, modifications, variations, or improvements therein may be subsequently made by those skilled in the art that are also intended to be encompassed by the following claims.

1. A pharmaceutical composition comprising:
(a) a compound of formula (I′): 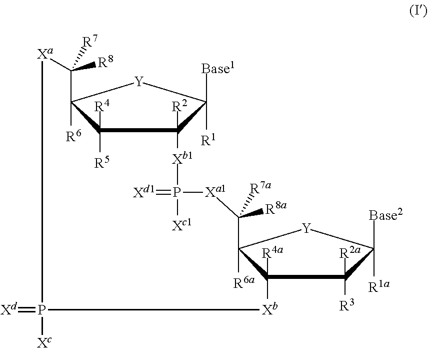
or a pharmaceutically acceptable salt thereof, wherein
Base¹ and Base² are each independently selected from the group consisting of 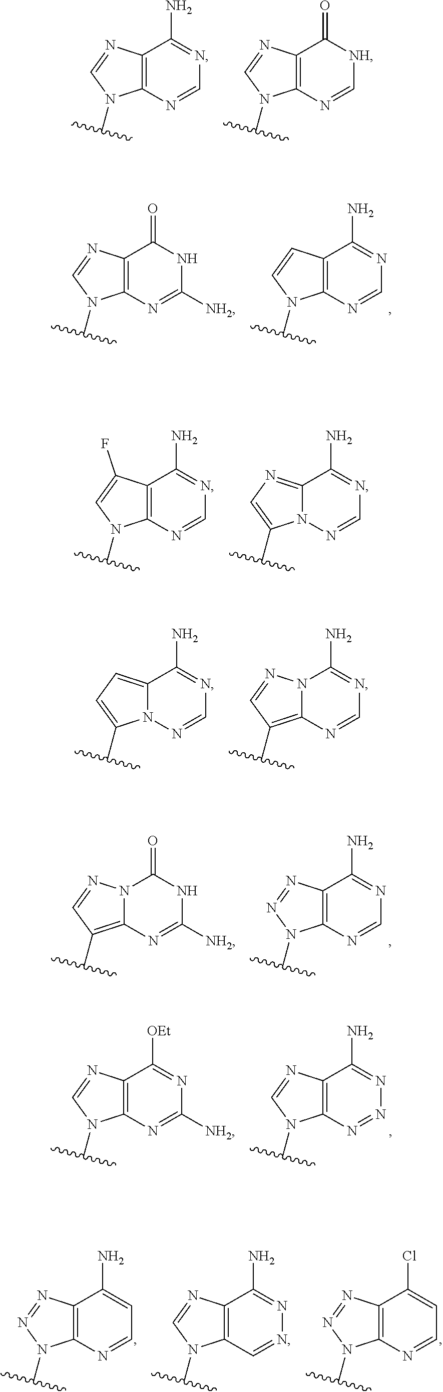 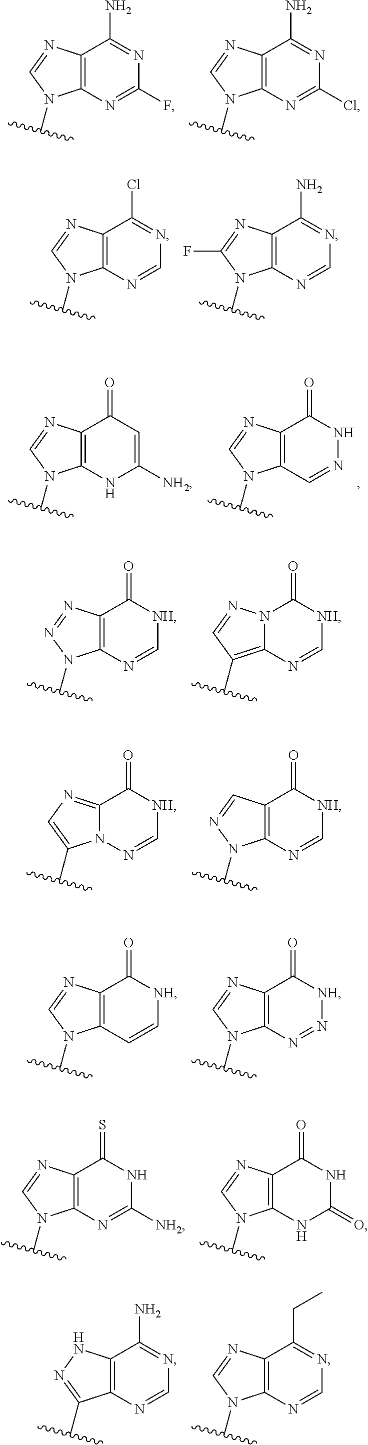 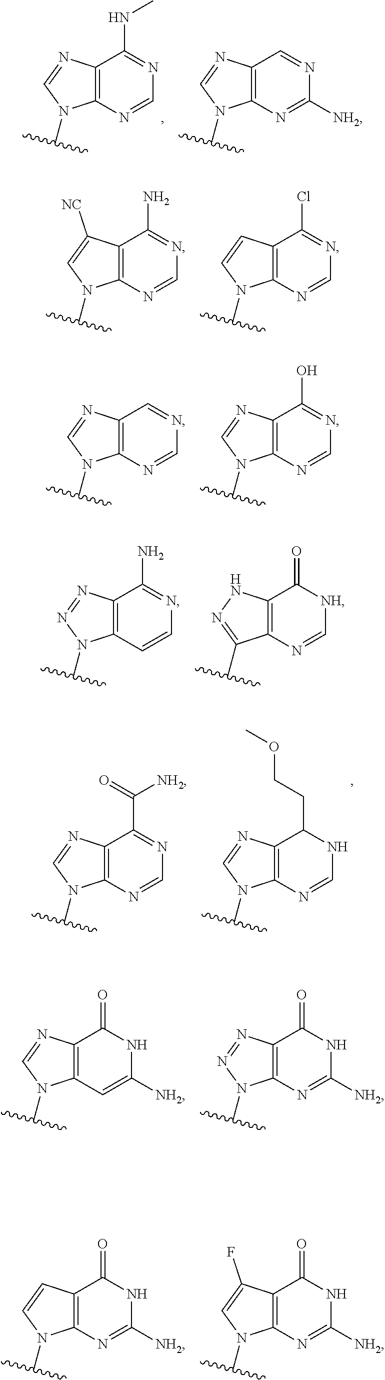 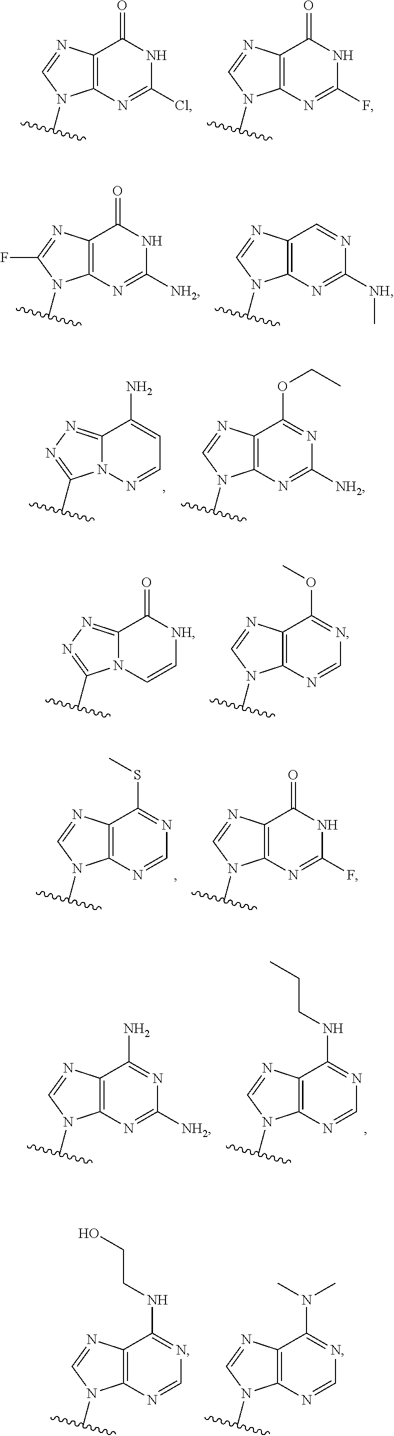 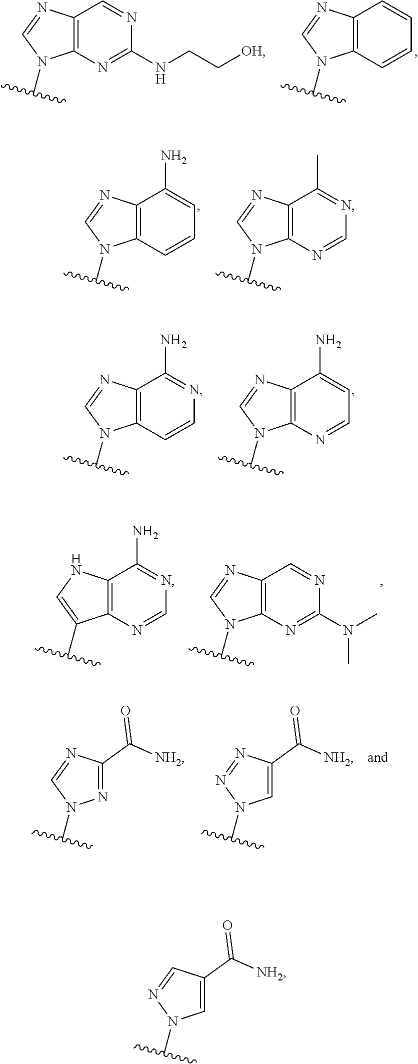
where Base¹ and Base¹ each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each independently selected from the group consisting of O, and S;
X^{b} and X^{b1} are each independently selected from the group consisting of O, and S;
X^{c} and X^{c1} are each independently selected from the group consisting of OR⁹, SR⁹, and NR⁹R⁹;
X^{d} and X^{d1} are each independently selected from the group consisting of O and S;
R¹ and R^{1a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R¹ and R^{1a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R² and R^{2a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R² and R^{2a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R³ is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N3;
R⁴ and R^{4a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁴ and R^{4a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N3;
R⁵ is selected from the group consisting of H, F, Cl, Br, I, OH, CN, NH₂, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁵ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, NR⁹R⁹, and N₃;
R⁶ and R^{6a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁶ and R^{6a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁷ and R^{7a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁷ and R^{7a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁸ and R^{8a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁸ and R^{8a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
each R⁹ is independently selected from the group consisting of H, C₁-C₂₀ alkyl, 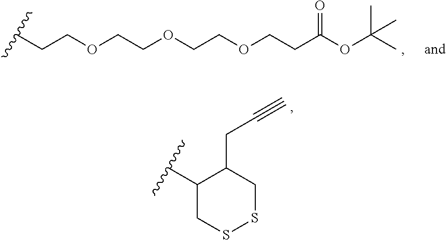
where each R⁹ C₁-C₂₀ alkyl is optionally substituted by 0 to 3 substituents independently selected from the group consisting of OH, —O—C₁-C₂₀ alkyl, —S—C(O)C₁-C₆ alkyl, and C(O)OC₁-C₆ alkyl;
optionally R^{1a} and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{1a} and R³ are connected to form —O—C₁-C₆ alkylene, —O—C₂-C₆ alkenylene, or —O—C₂-C₆ alkynylene, said O is bound at the R³ position;
optionally R^{2a} and R³ are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R^{2a} and R³ are connected to form —O—C₁-C₆ alkylene, —O—C₂-C₆ alkenylene, or —O—C₂-C₆ alkynylene, said O is bound at the R³ position;
optionally R³ and R^{6a} are connected to form —O—C₁-C₆ alkylene, —O—C₂-C₆ alkenylene, or —O—C₂-C₆ alkynylene, such that where R³ and R^{6a} are connected to form —O—C₁-C₆ alkylene, —O—C₂-C₆ alkenylene, or —O—C₂-C₆ alkynylene, said O is bound at the R³ position;
optionally R⁴ and R⁵ are connected to form are connected to form C₁-C₆ alkylene, C₂-C₆ alkenylene, C₂-C₆ alkynylene, —O—C₁-C₆ alkylene, —O—C₂-C₆ alkenylene, or —O—C₂-C₆ alkynylene, such that where R⁴ and R⁵ are connected to form —O—C₁-C₆ alkylene, —O—C₂-C₆ alkenylene, or —O—C₂-C₆ alkynylene, said O is bound at the R⁵ position;
optionally R⁵ and R⁶ are connected to form —O—C₁-C₆ alkylene, —O—C₂-C₆ alkenylene, or —O—C₂-C₆ alkynylene, such that where R⁵ and R⁶ are connected to form —O—C₁-C₆ alkylene, —O—C₂-C₆ alkenylene, or —O—C₂-C₆ alkynylene, said O is bound at the R⁵ position;
optionally R⁷ and R⁸ are connected to form C₁-C₆ alkylene, C₂-C₆ alkenylene, or C₂-C₆ alkynylene; and
optionally R^{7a} and R⁸ are connected to form C₁-C₆ alkylene, C₂-C₆ alkenylene, or C₂-C₆ alkynylene; and
providing that when Y and Y^{a} are each O, X^{a} and X^{a1} are each O, X^{b} and X^{b1} are each O, and X^{c} and X^{c1} are each OH or SH, X^{d} and X^{d1} are each O, R¹ and R^{1a} are each H, R² is H, R⁶ and R^{6a} are each H, R⁷ and R^{7a} are each H, R⁸ and R^{8a} are each H, and Base¹ and Base¹ are each selected from the group consisting of 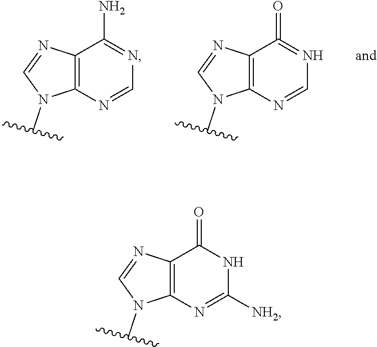
R⁵ and R³ are not both selected from the group consisting of H, F and OH;
(b) a pharmaceutically acceptable aqueous carrier;
(c) one or more pharmaceutically acceptable tonicity modifiers;
(d) one or more pharmaceutically acceptable buffering agents;
(e) one or more pharmaceutically acceptable antioxidants; and
(f) one or more pharmaceutically acceptable metal chelators.

2. The pharmaceutical composition according to claim 1, wherein the compound is selected from the group consisting of 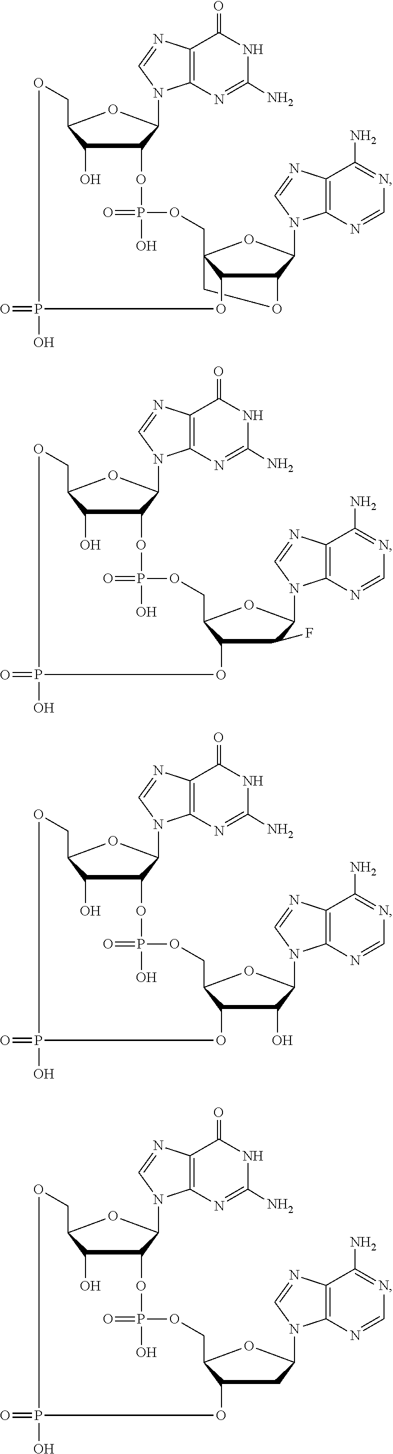 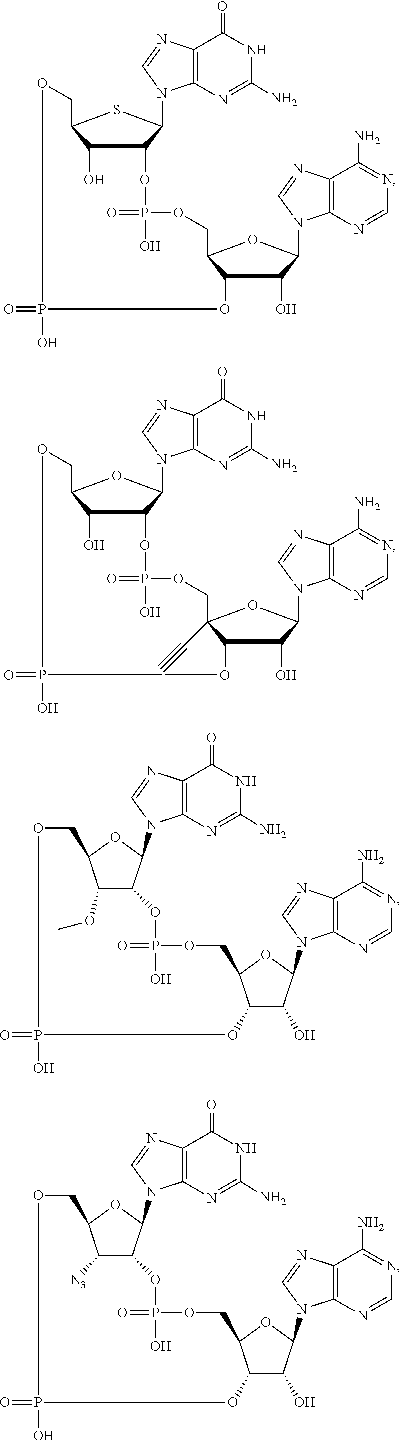 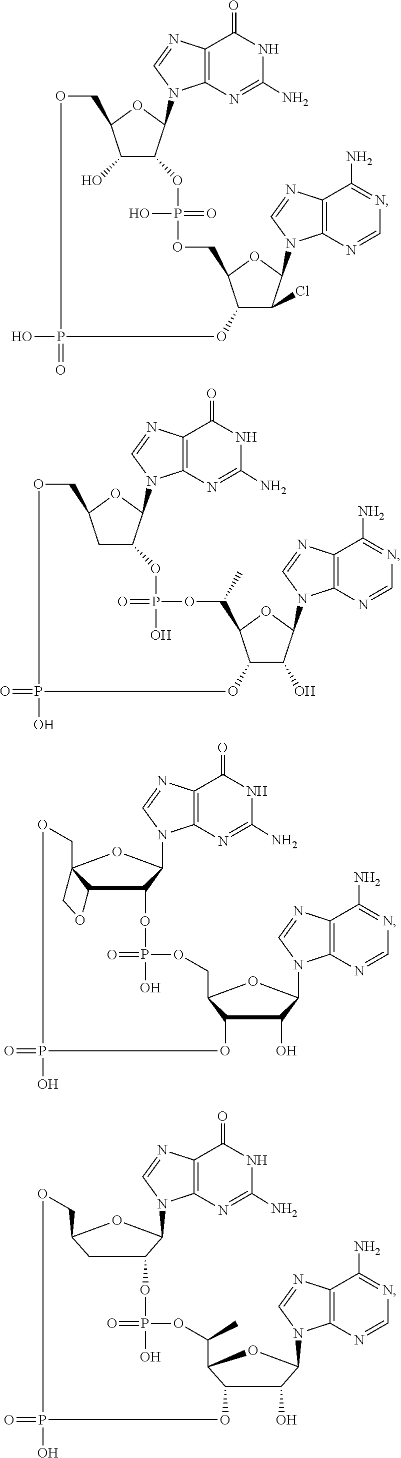 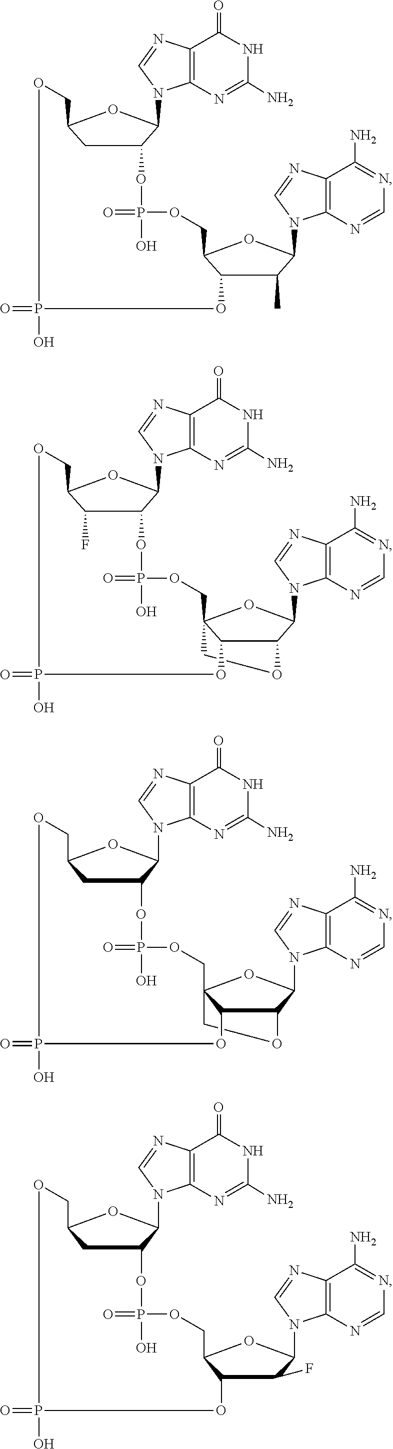 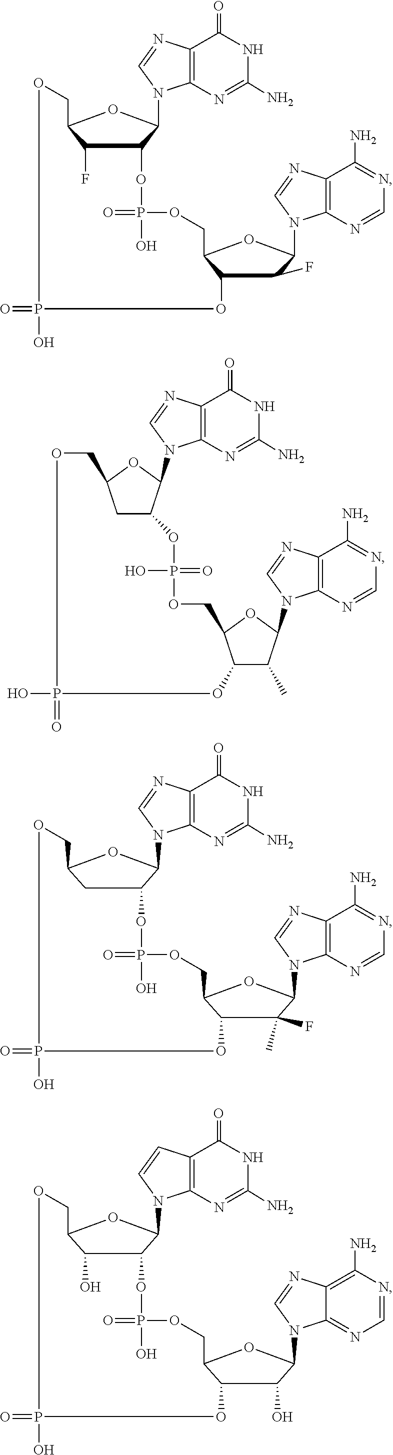 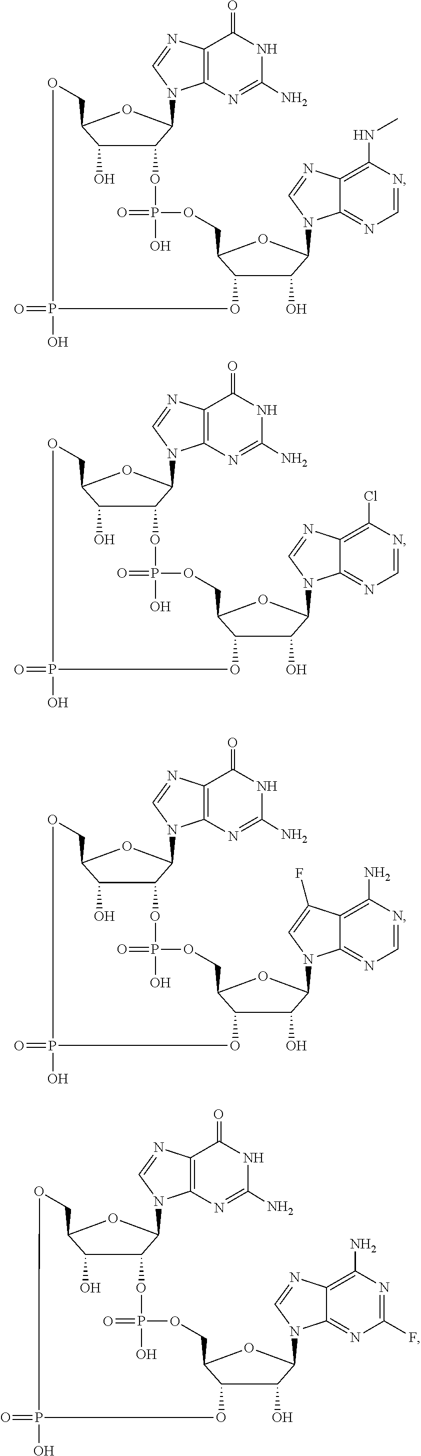 [SMILES: CNc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2O[C@@H]3COP(=O)(O)O[C@H]4[C@@H](O)[C@H](n5cnc6c(Cl)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cc(F)c6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)nc(F)nc65)O[C@@H]4COP(=O)(O)O[C@@H]2[C@@H]3O)c(=O)[nH]1] 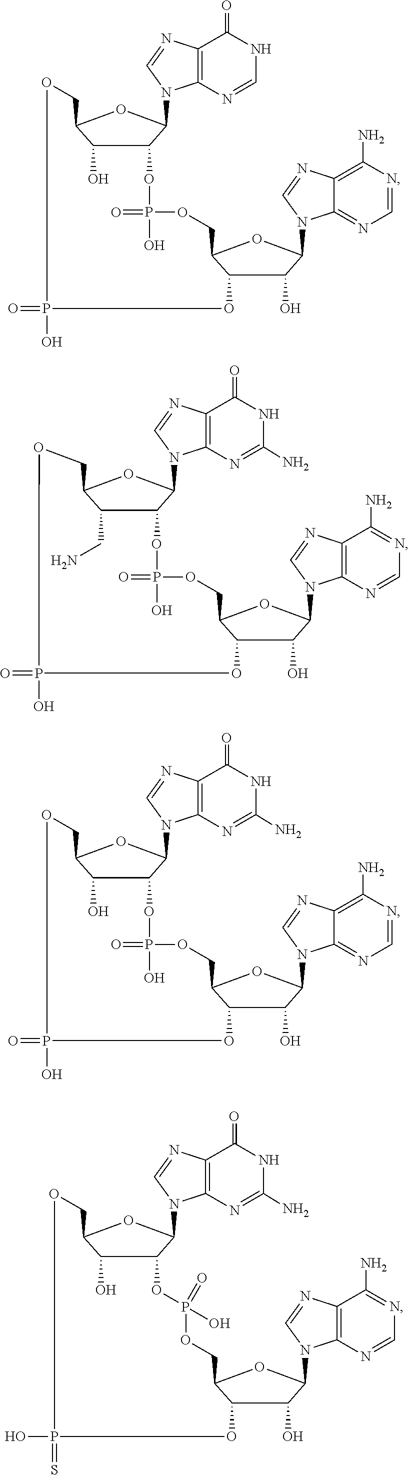 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21 ; NC[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(O)(=S)O[C@H]4[C@@H](O)[C@H](n5cnc6c(N)ncnc65)O[C@@H]4COP(=O)(O)O[C@@H]2[C@@H]3O)c(=O)[nH]1] 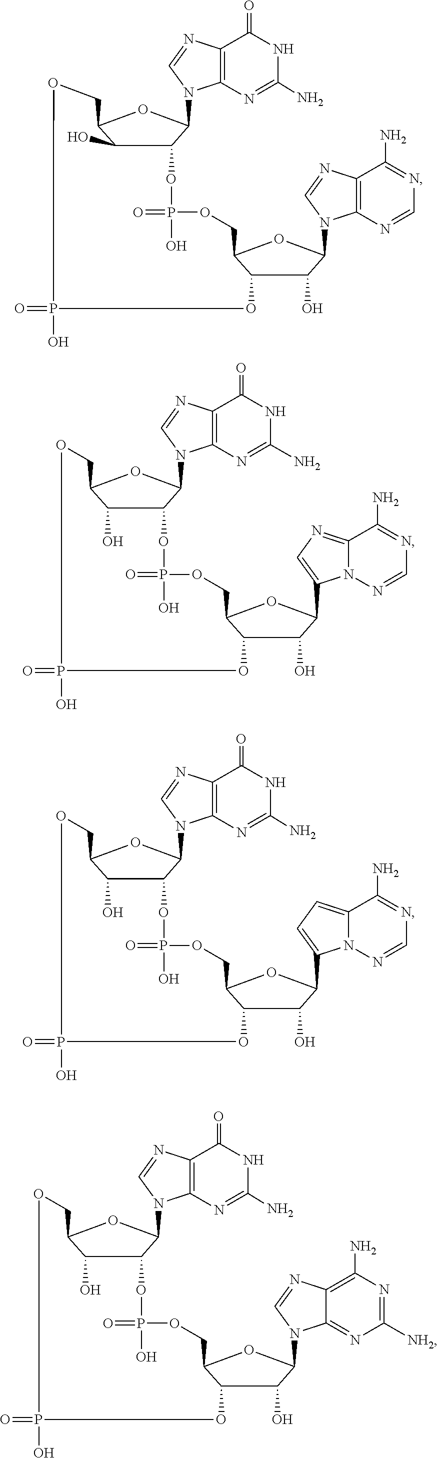 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3O)c(=O)[nH]1 ; Nc1nc2c(ncn2[C@@H]2O[C@@H]3COP(=O)(O)O[C@H]4[C@@H](O)[C@H](c5cnc6c(N)ncnn56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](c5ccc6c(N)ncnn56)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1] 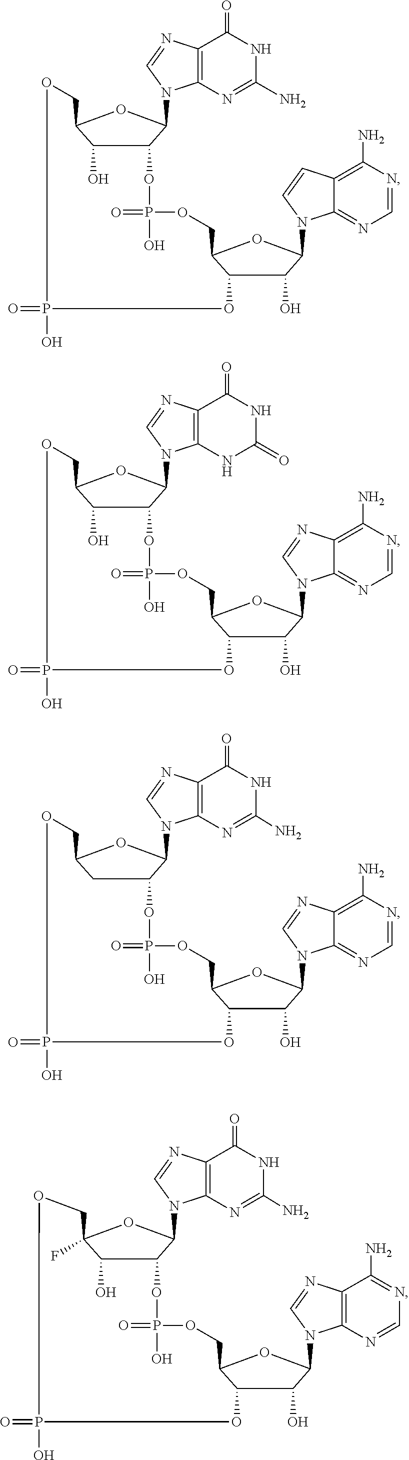 [SMILES: Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(=O)[nH]c21 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2C3)c(=O)[nH]1 ; Nc1nc2c(ncn2[C@@H]2O[C@]3(F)COP(=O)(O)O[C@H]4[C@@H](O)[C@H](n5cnc6c(N)ncnc65)O[C@@H]4COP(=O)(O)O[C@@H]2[C@@H]3O)c(=O)[nH]1] 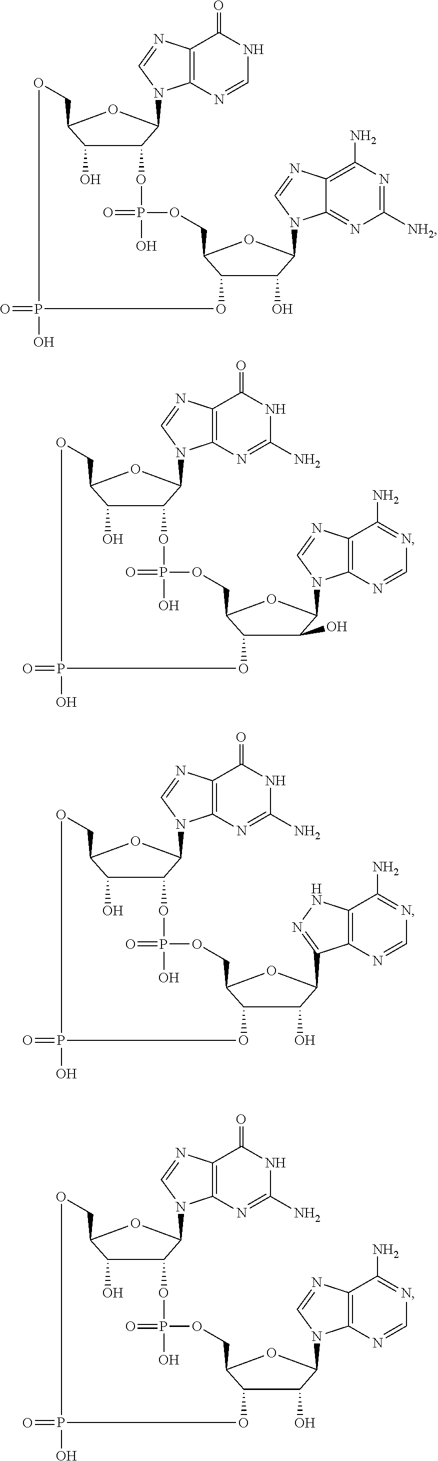 [SMILES: Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]cnc43)c2n1 ; Nc1nc2c(ncn2[C@@H]2O[C@@H]3COP(=O)(O)O[C@H]4[C@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](c5n[nH]c6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1] 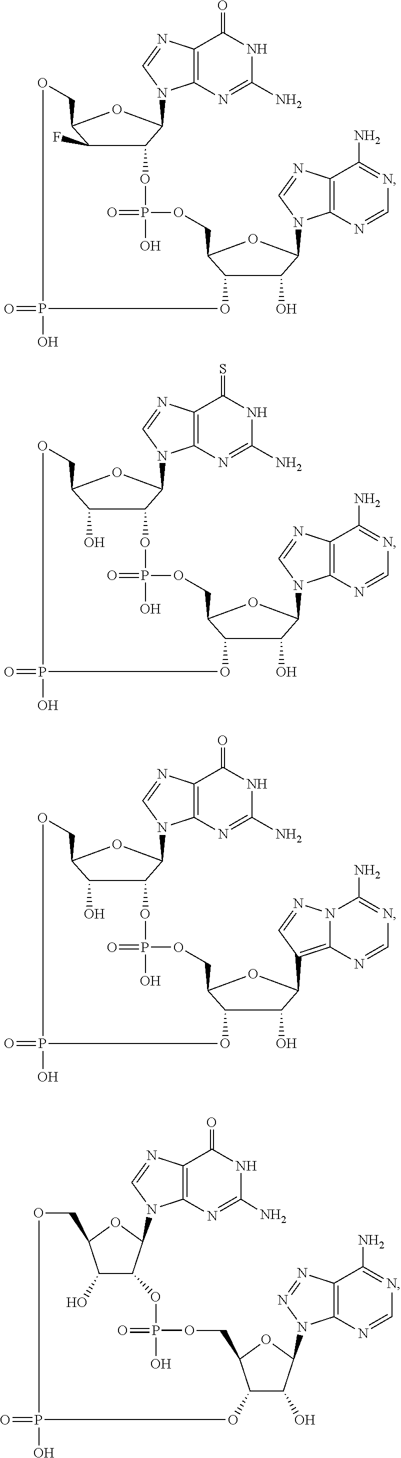 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3F)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=S)[nH]1 ; Nc1nc2c(ncn2[C@@H]2O[C@@H]3COP(=O)(O)O[C@H]4[C@@H](O)[C@H](c5cnn6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)ncnc65)O[C@@H]4COP(=O)(O)O[C@@H]2[C@@H]3O)c(=O)[nH]1] 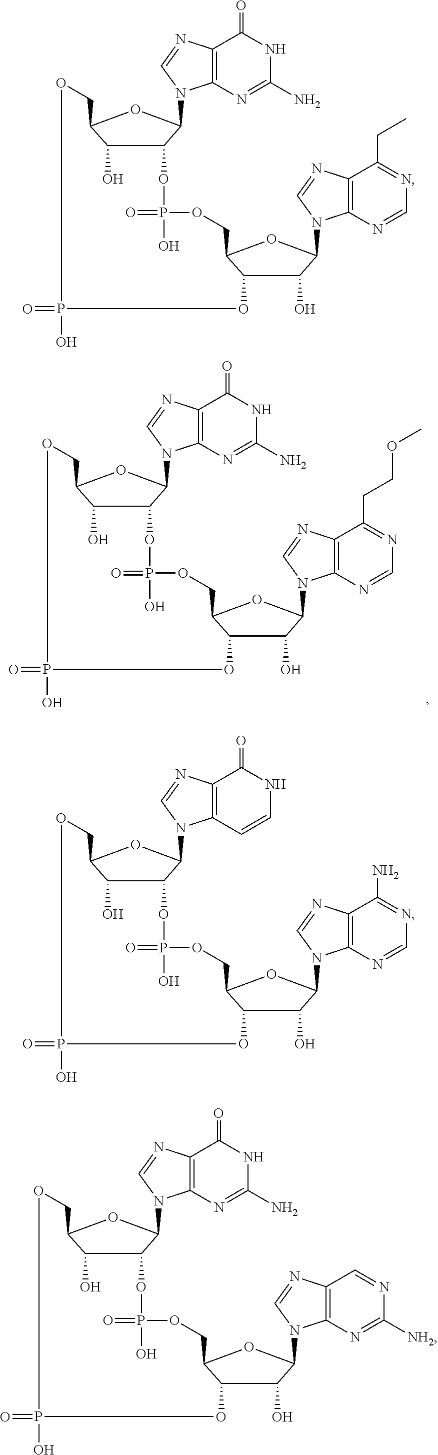 [SMILES: CCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; COCCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; Nc1ncc2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1] 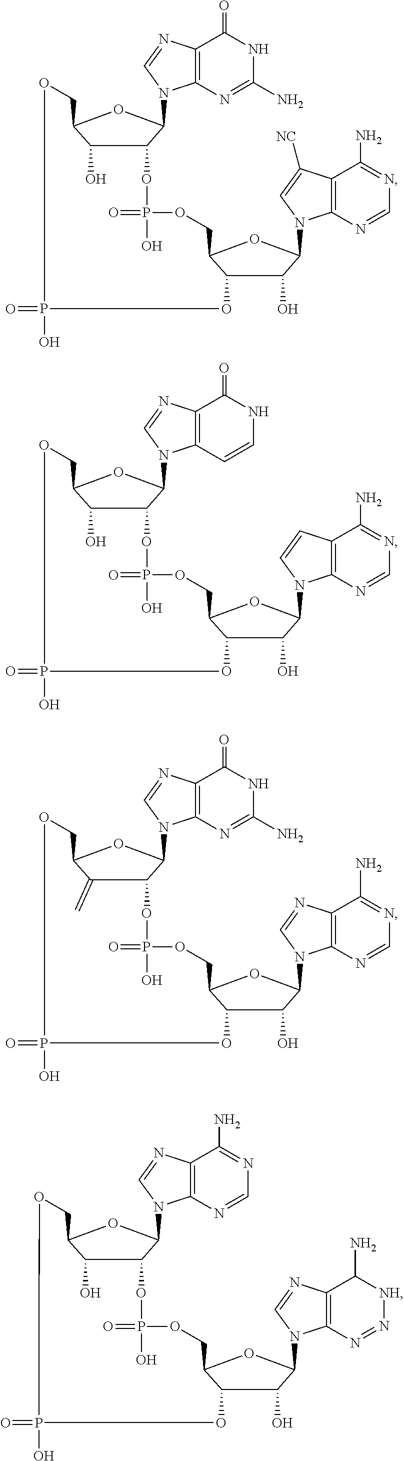 [SMILES: N#Cc1cn([C@@H]2O[C@@H]3COP(=O)(O)O[C@@H]4[C@H](O)[C@@H](COP(=O)(O)O[C@H]3[C@H]2O)O[C@H]4n2cnc3c(=O)[nH]c(N)nc32)c2ncnc(N)c12 ; Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; C=C1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1ncnc2c1ncn2[C@@H]1O[C@@H]2COP(=O)(O)O[C@H]3[C@@H](O)[C@H](n4cnc5c4N=NNC5N)O[C@@H]3COP(=O)(O)O[C@@H]1[C@@H]2O] 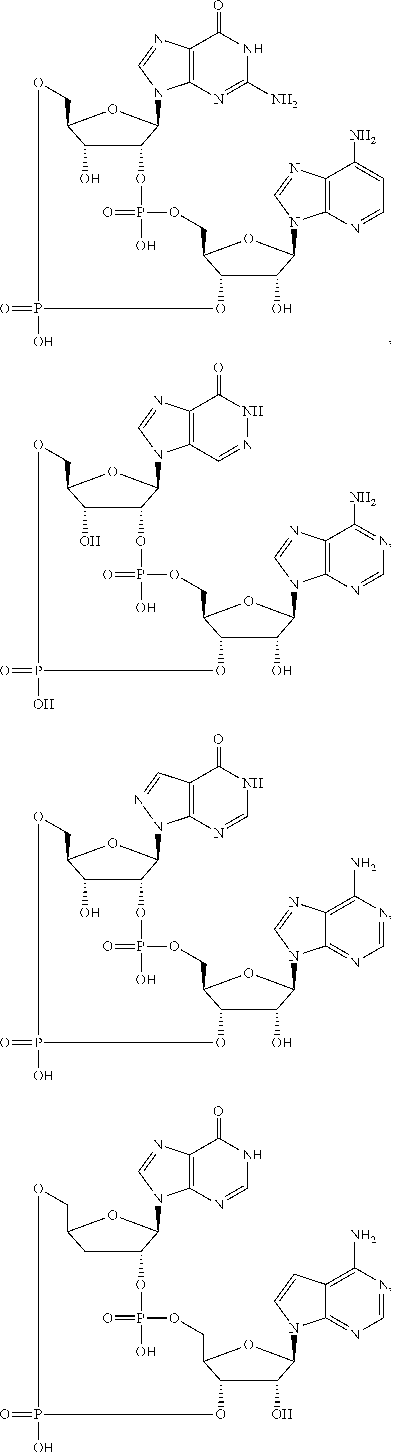 [SMILES: Nc1nc2c(ncn2[C@@H]2O[C@@H]3COP(=O)(O)O[C@H]4[C@@H](O)[C@H](n5cnc6c(N)ccnc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ncc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1ncc2c(=O)[nH]cnc21 ; Nc1ncnc2c1ccn2[C@@H]1OC2COP(=O)(O)O[C@@H]3CC(COP(=O)(O)O[C@H]2[C@H]1O)O[C@H]3n1cnc2c(=O)[nH]cnc21] 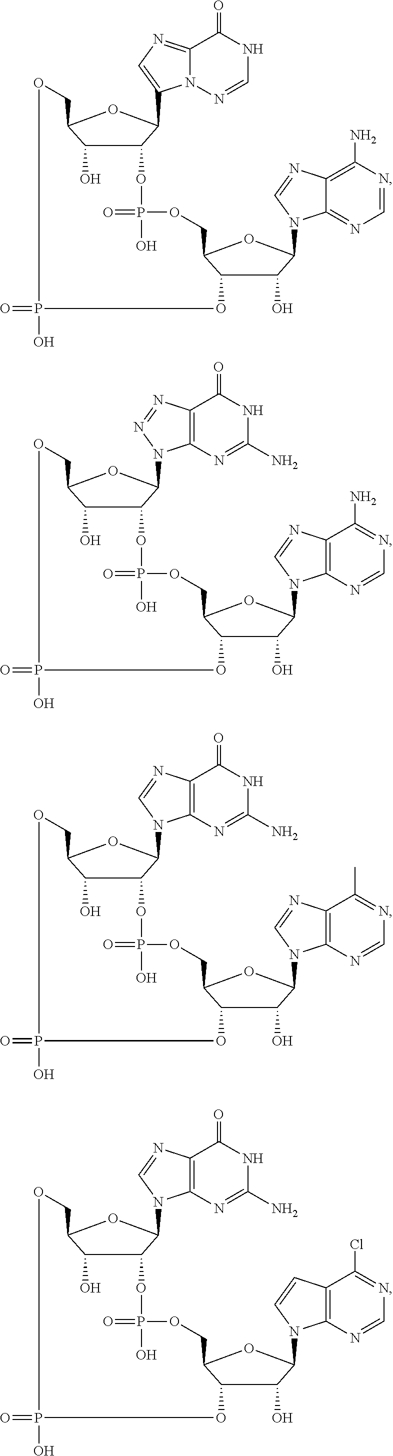 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1cnc2c(=O)[nH]cnn12 ; Nc1nc2c(nnn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Cc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2O[C@@H]3COP(=O)(O)O[C@H]4[C@@H](O)[C@H](n5ccc6c(Cl)ncnc65)O[C@@H]4COP(=O)(O)O[C@@H]2[C@@H]3O)c(=O)[nH]1] 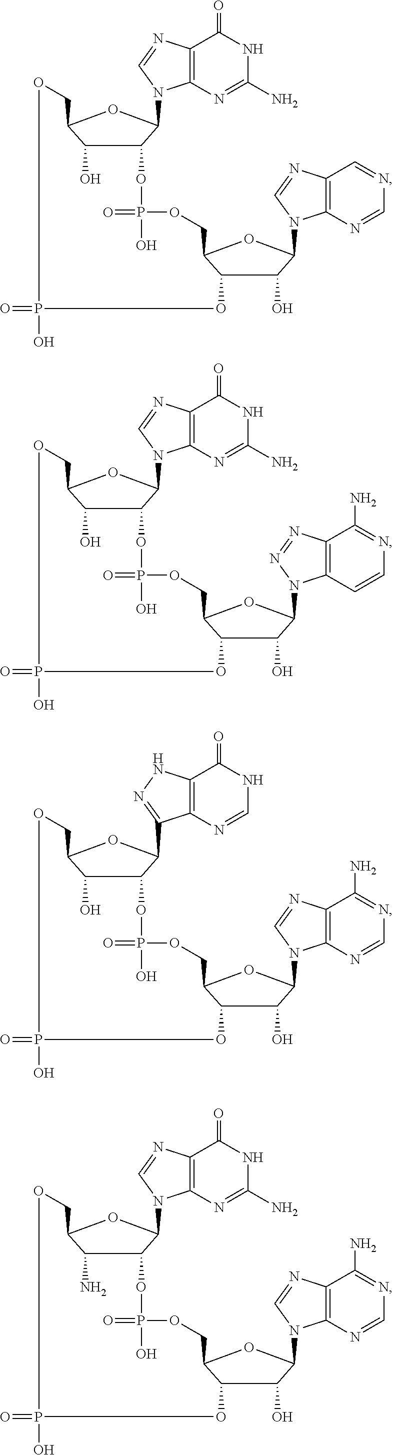 [SMILES: Nc1nc2c(ncn2[C@@H]2O[C@@H]3COP(=O)(O)O[C@H]4[C@@H](O)[C@H](n5cnc6cncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)nccc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1n[nH]c2c(=O)[nH]cnc12 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3N)c(=O)[nH]1] 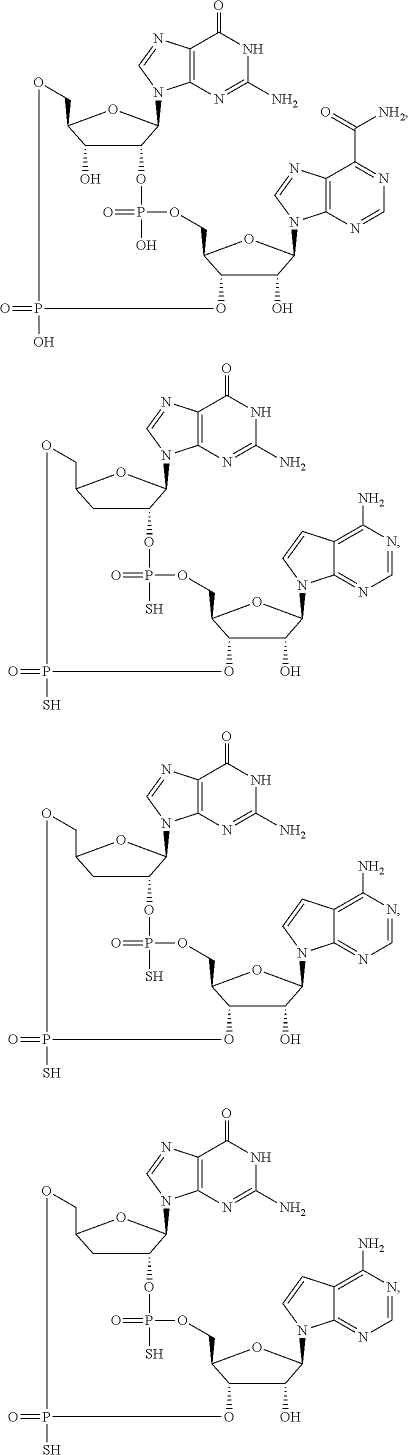 [SMILES: NC(=O)c1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1] 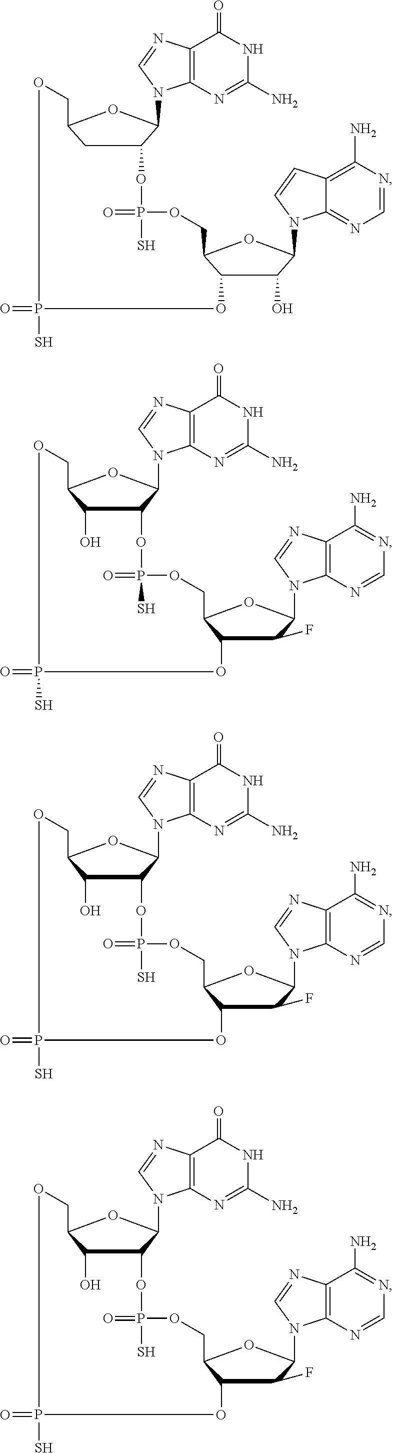 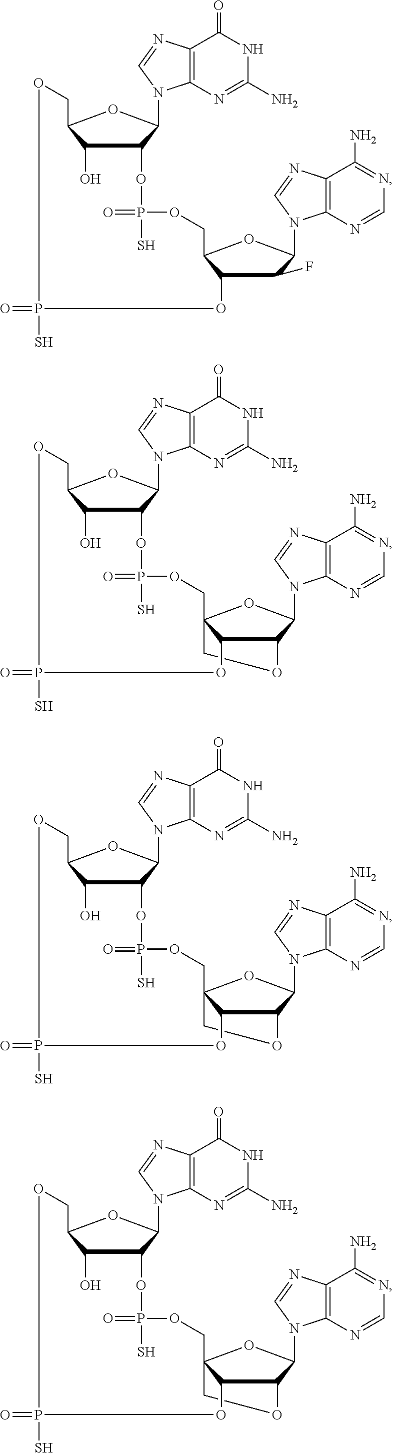 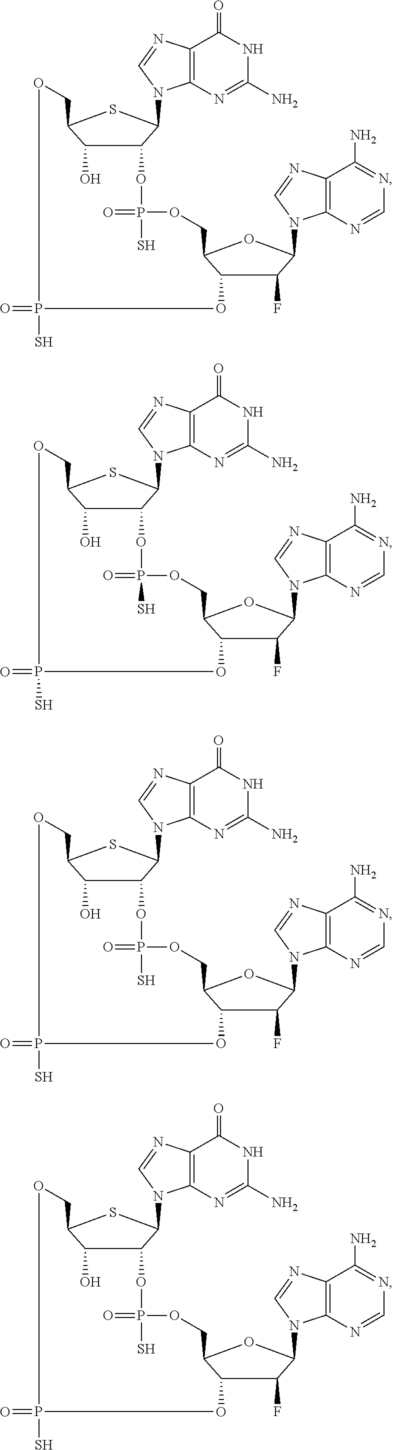 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1] 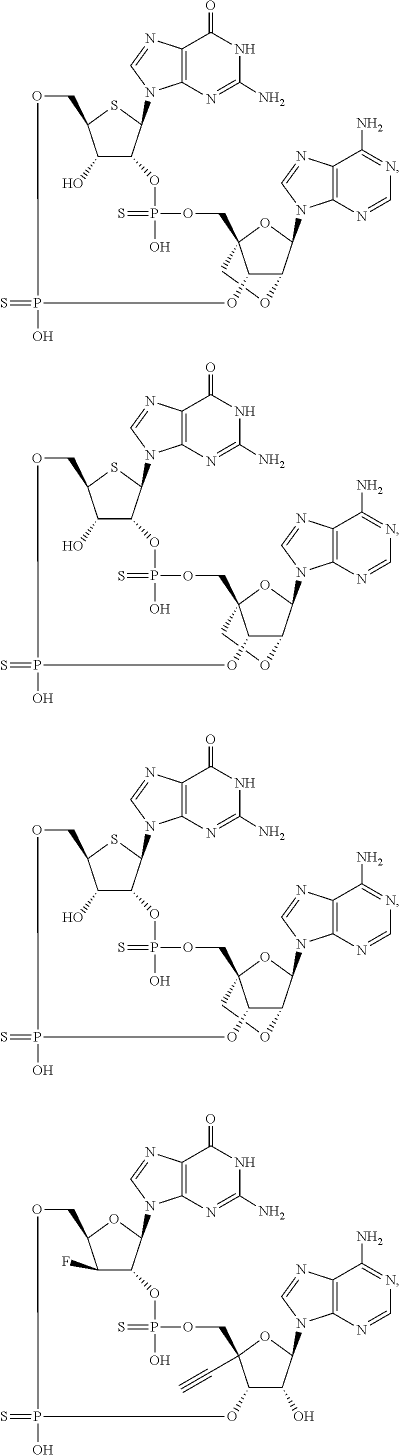 [SMILES: Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21] 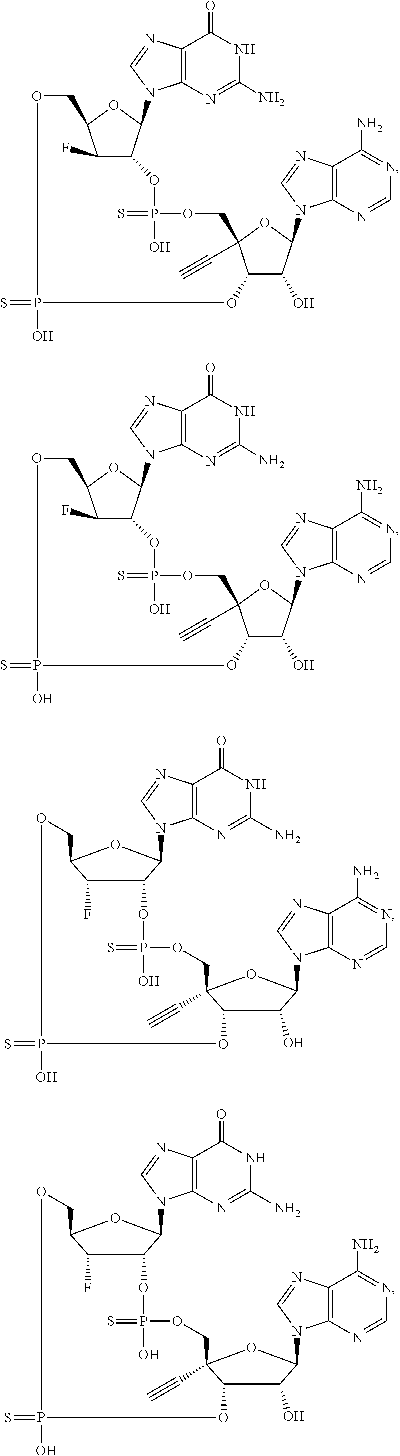 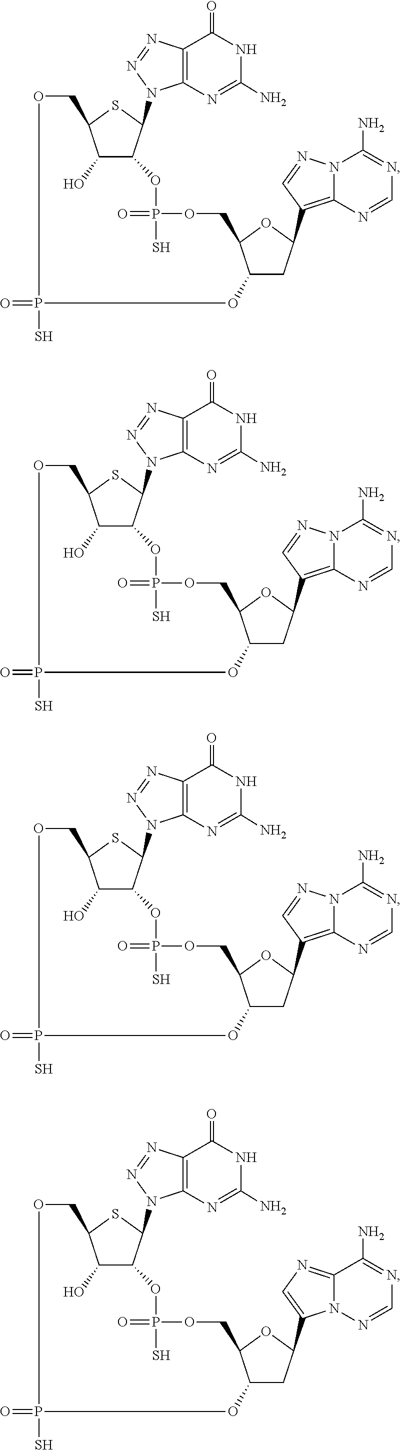 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] 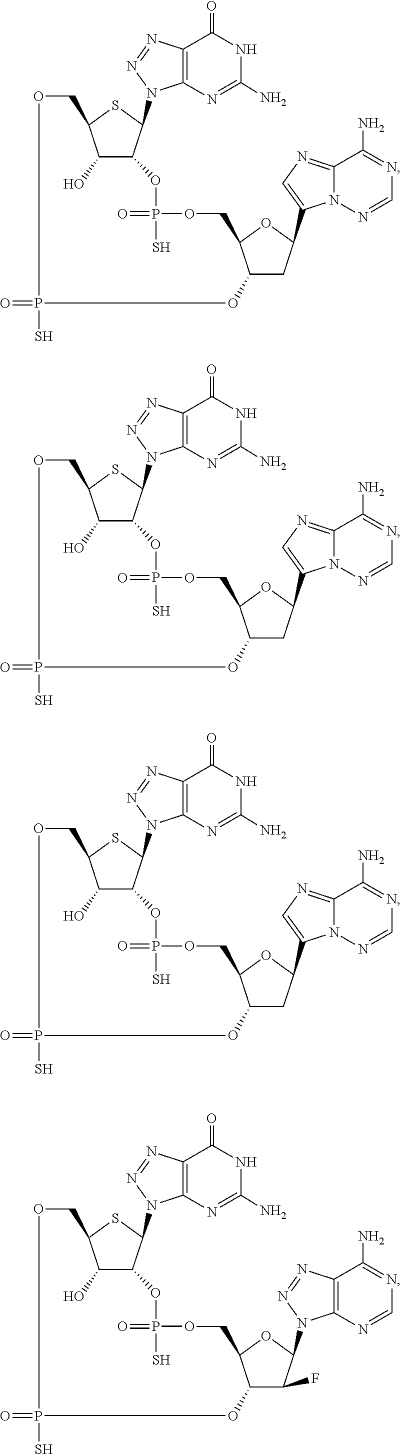 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1] 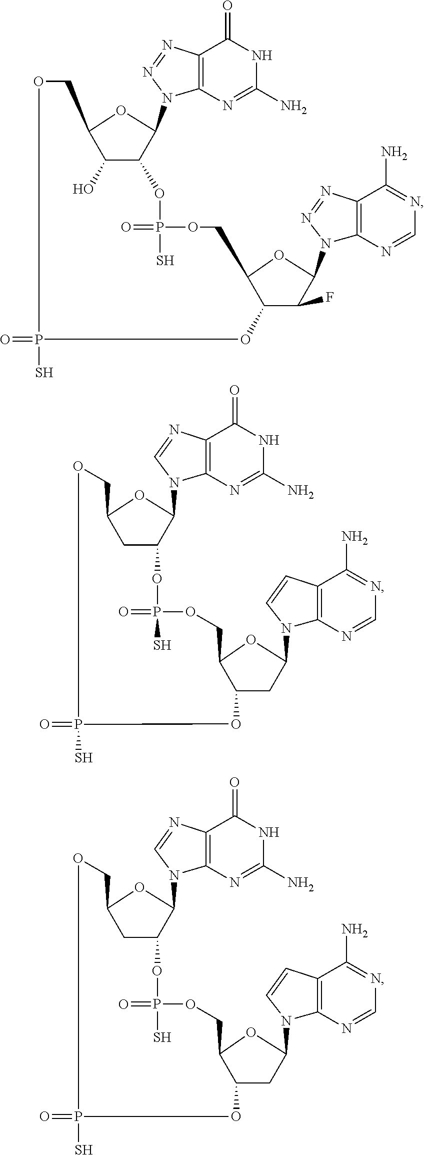 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(ncn2[C@@H]2O[C@@H]3CO[P@@](=O)(S)O[C@H]4C[C@H](n5ccc6c(N)ncnc65)O[C@@H]4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1] 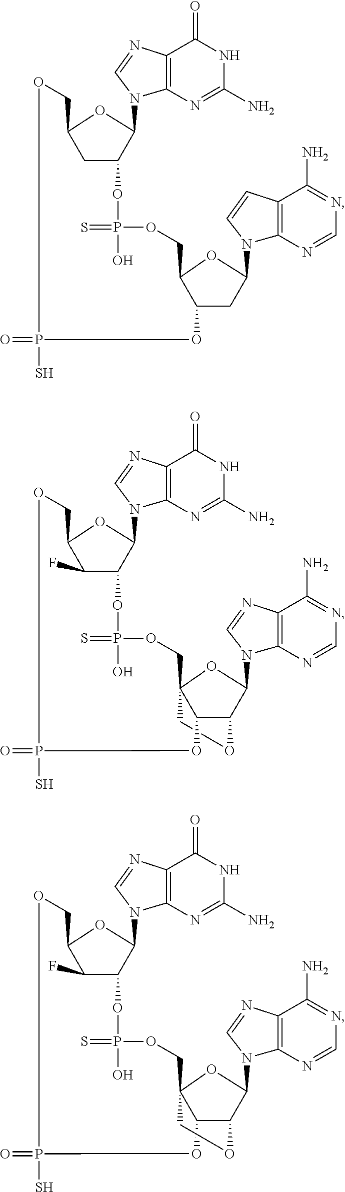 [SMILES: Nc1nc2c(ncn2[C@@H]2O[C@@H]3COP(=O)(S)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(O)(=S)O[C@@H]2[C@H]3F)O[C@H]5n2cnc3c(N)ncnc32)c(=O)[nH]1] 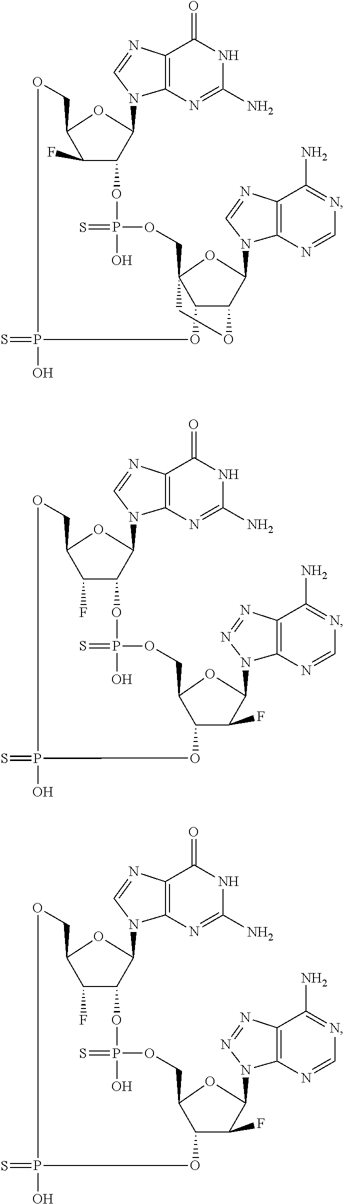 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] 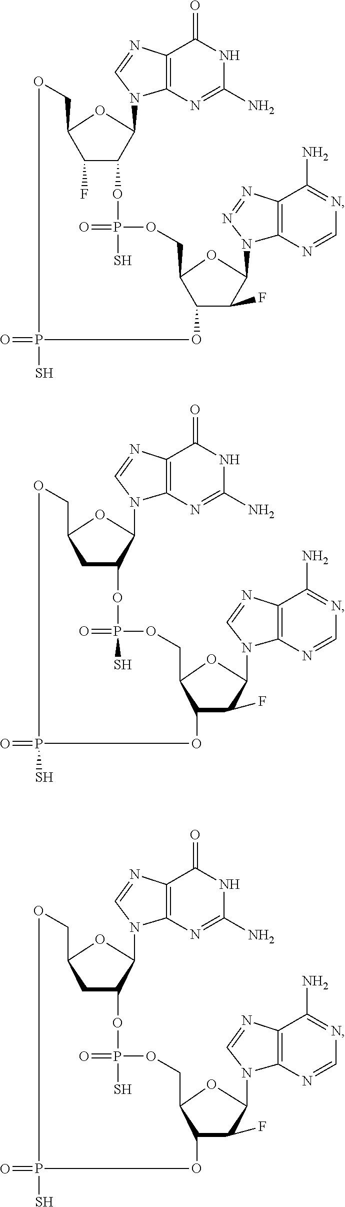 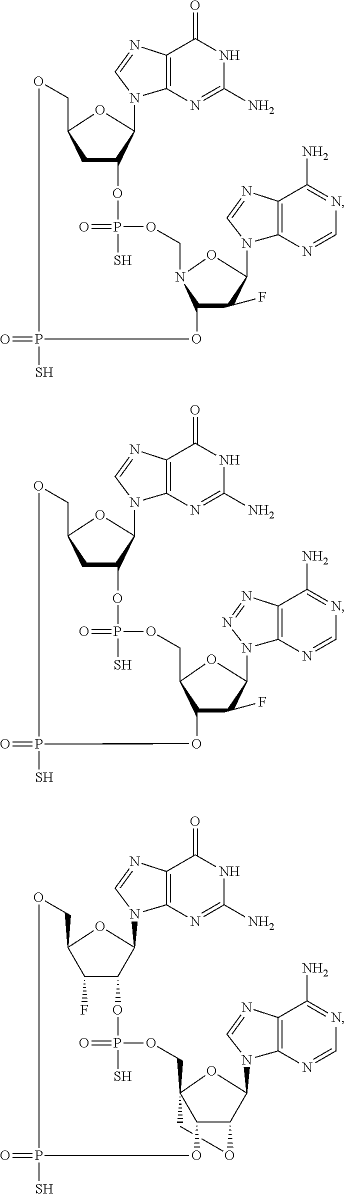 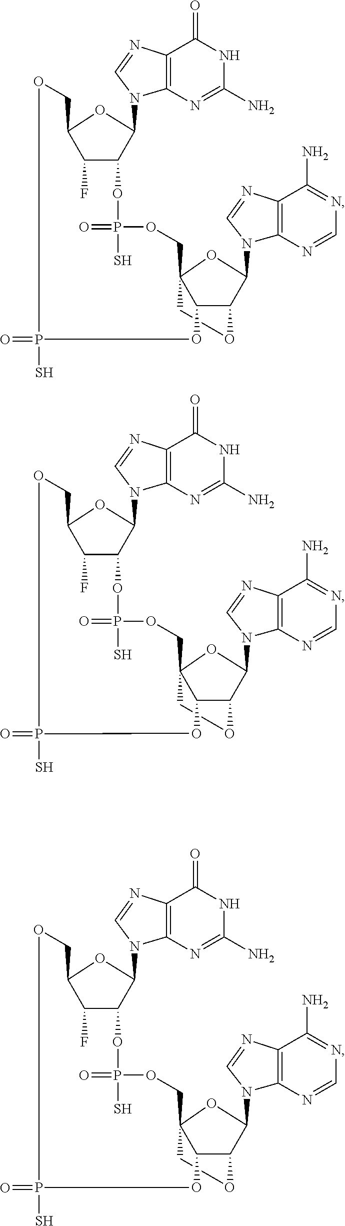 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] 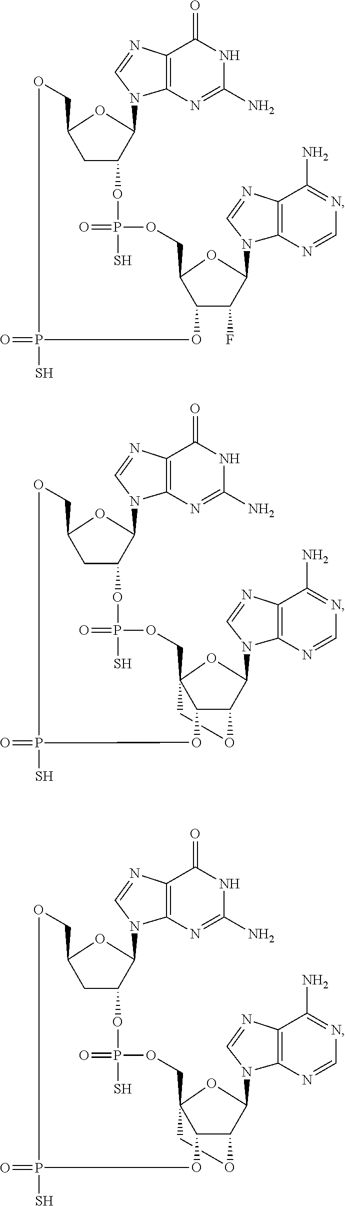 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@@H]2C3)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] 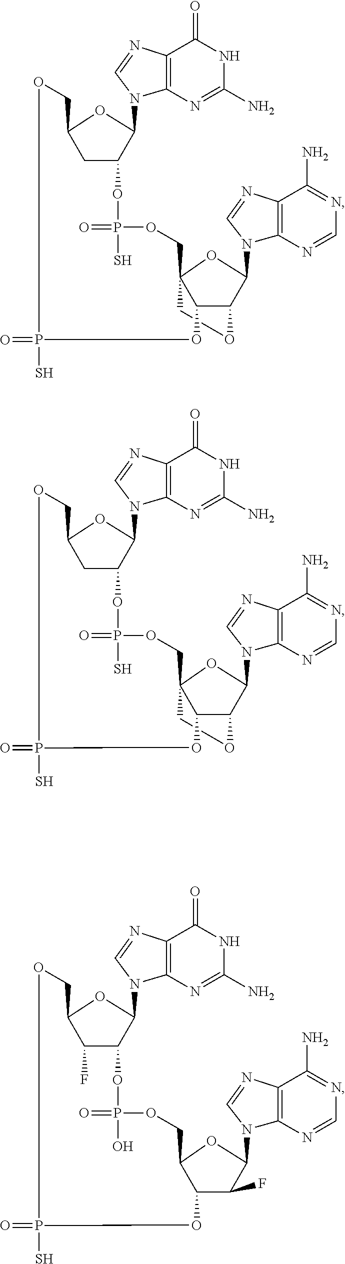 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(O)O[C@@H]2[C@@H]3F)c(=O)[nH]1] 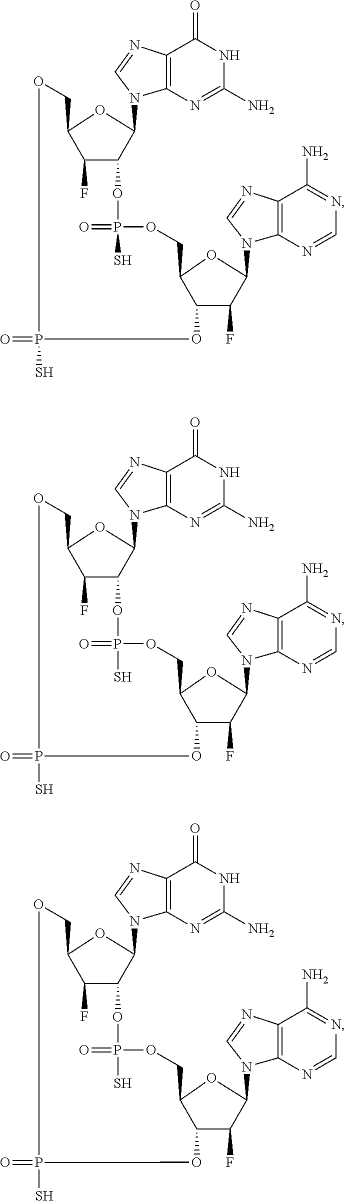 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] 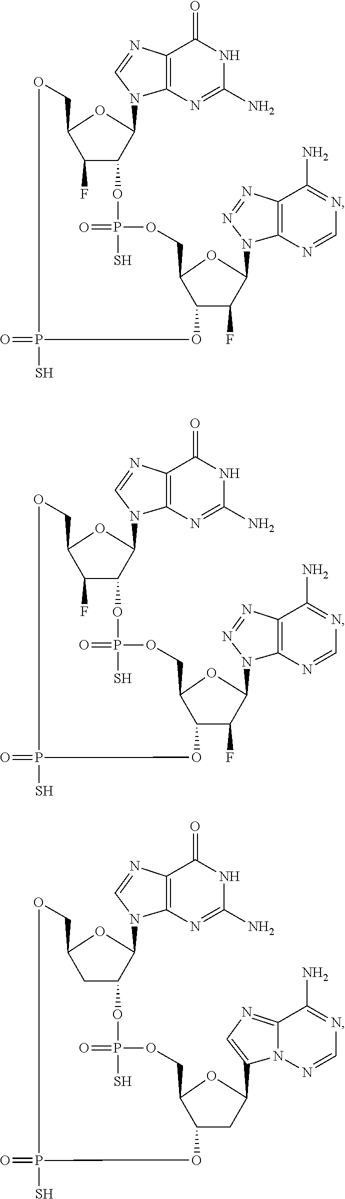 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1] 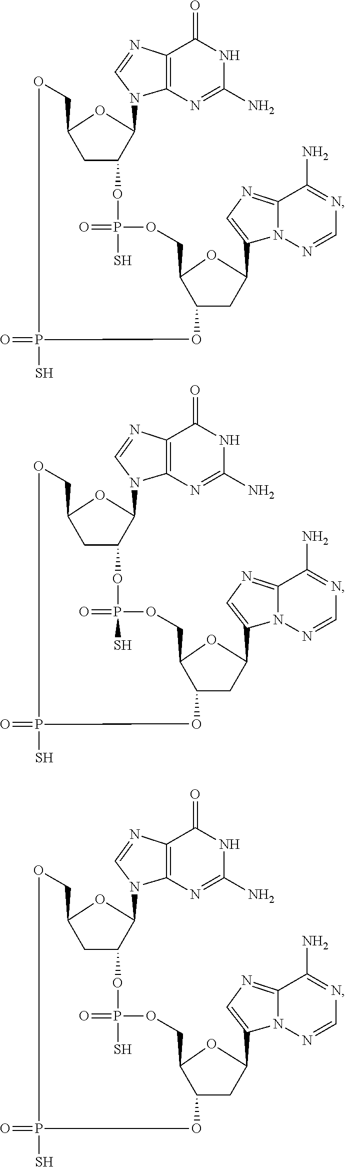 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2O[C@@H]3COP(=O)(S)O[C@H]4C[C@H](c5cnc6c(N)ncnn56)O[C@@H]4CO[P@@](=O)(S)O[C@@H]2C3)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1] 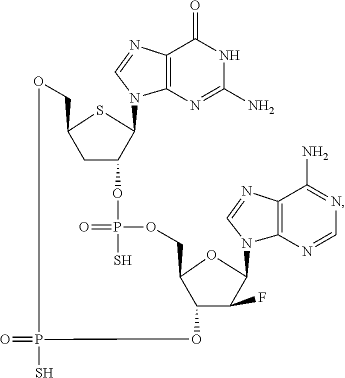 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2C3)c(=O)[nH]1] 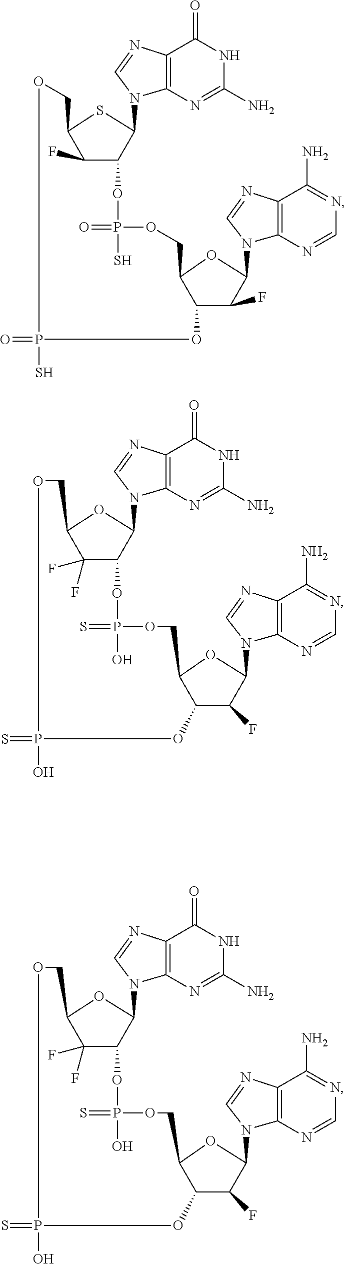 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1] 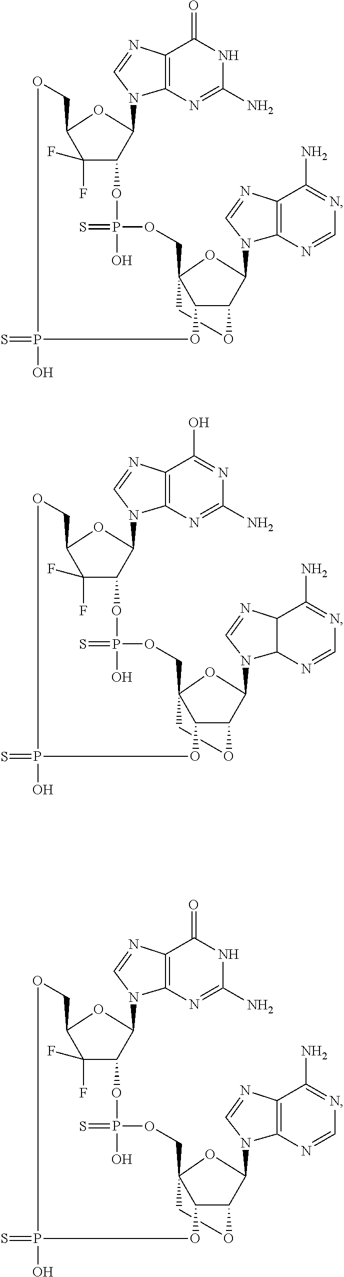 [SMILES: Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; NC1=NC=NC2C1N=CN2[C@@H]1O[C@@]23CO[C@@H]1[C@@H]2OP(O)(=S)OC[C@H]1O[C@@H](n2cnc4c(O)nc(N)nc42)[C@H](OP(O)(=S)OC3)C1(F)F ; Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1] 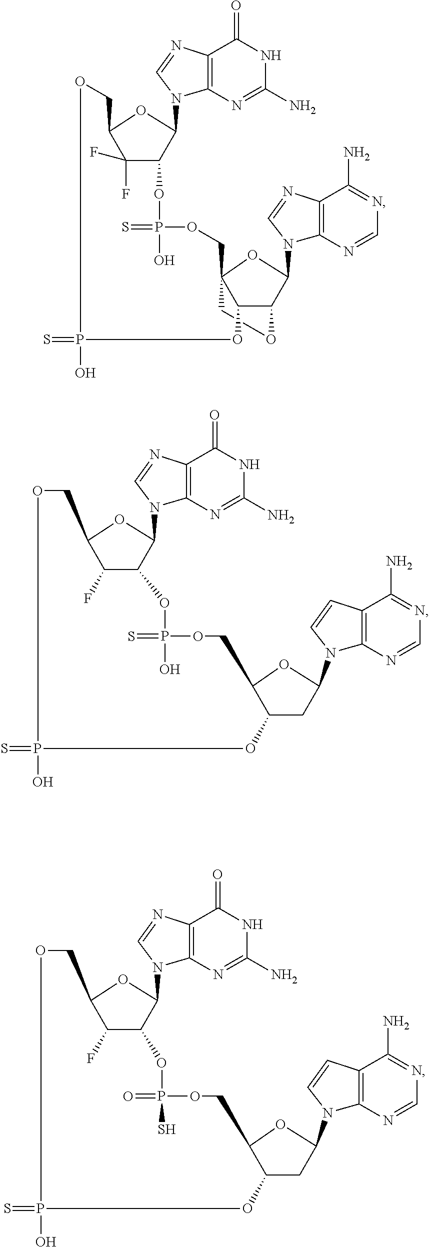 [SMILES: Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4CO[P@@](=O)(S)O[C@@H]2[C@@H]3F)c(=O)[nH]1] 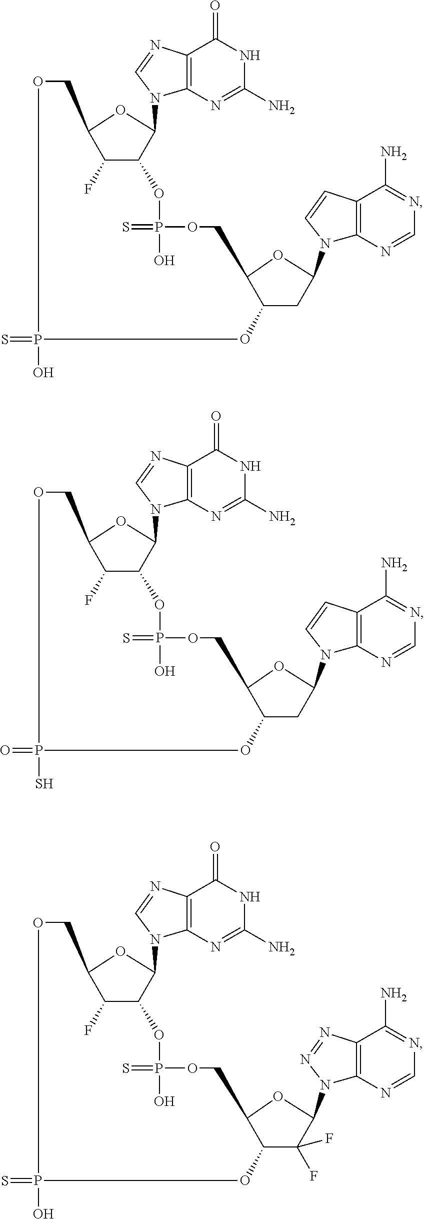 [SMILES: Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1] 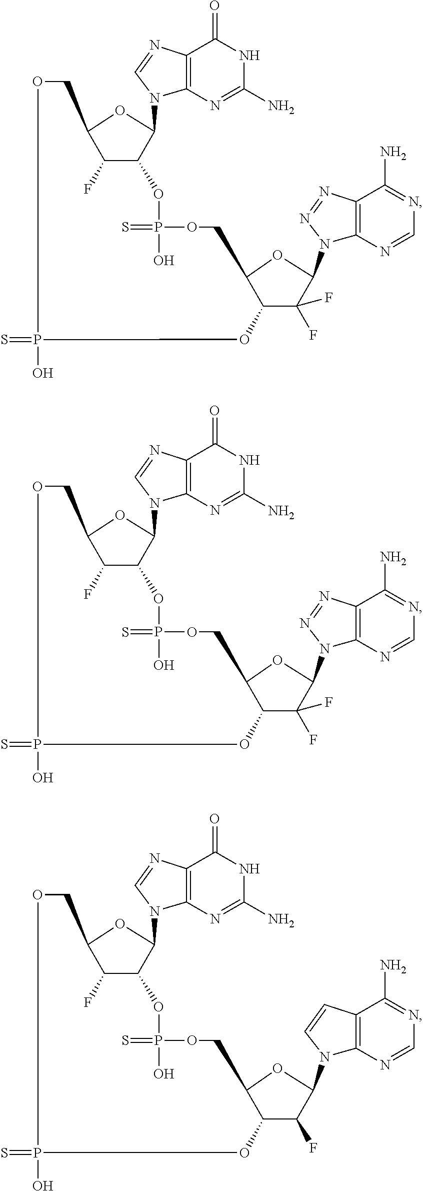 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1] 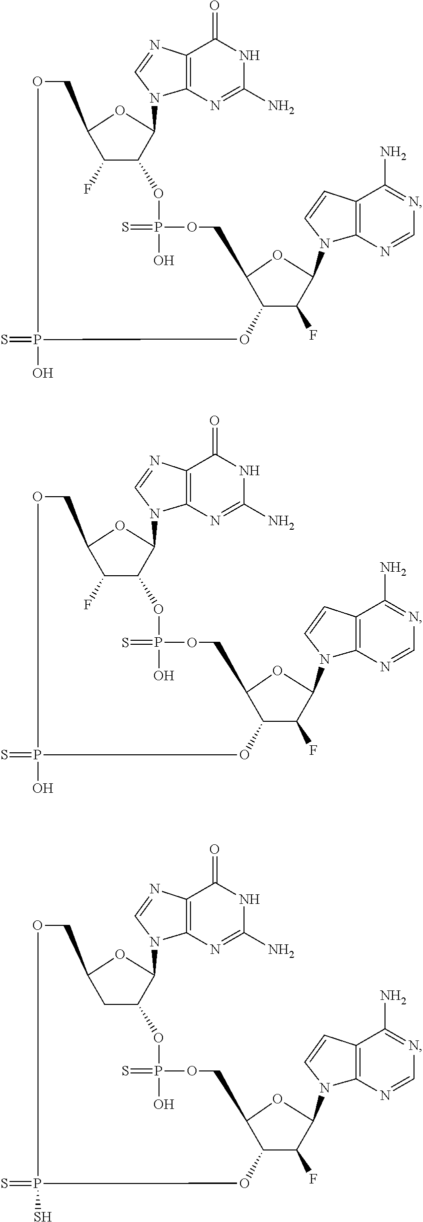 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=S)(S)O[C@H]4[C@H](F)[C@H](n5ccc6c(N)ncnc65)O[C@@H]4COP(O)(=S)O[C@@H]2C3)c(=O)[nH]1] 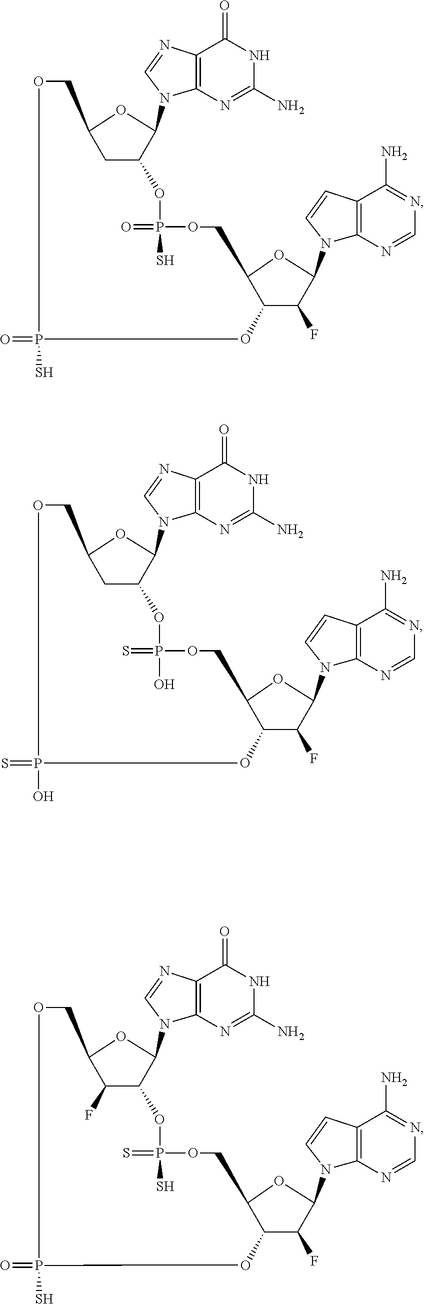 [SMILES: Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(S)O[C@@H]2C3)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=S)(S)O[C@@H]2[C@H]3F)c(=O)[nH]1] 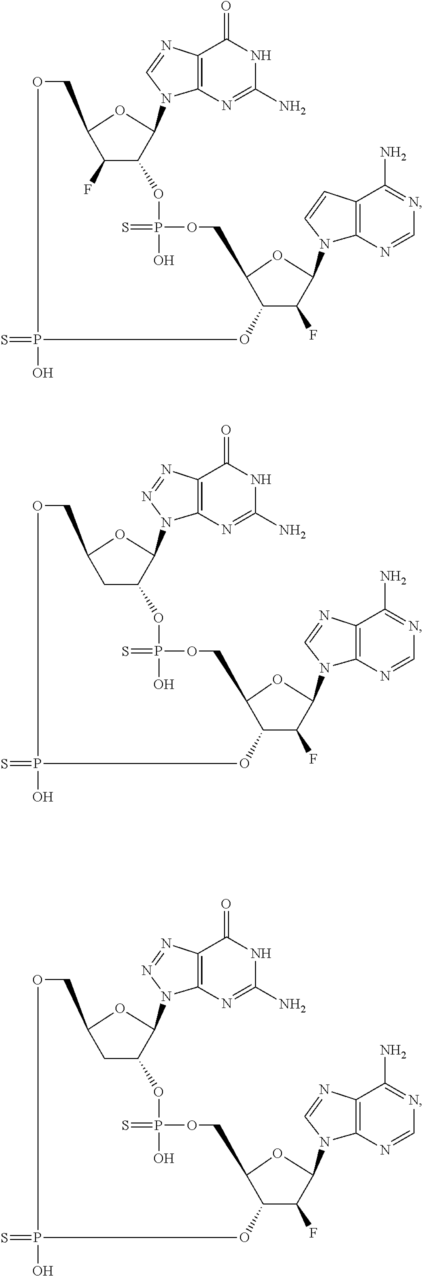 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1] 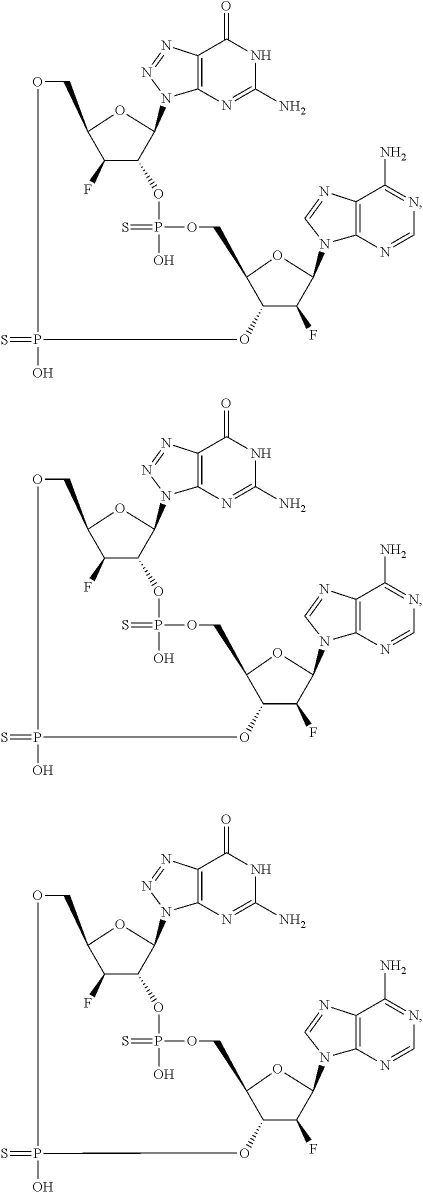 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1] 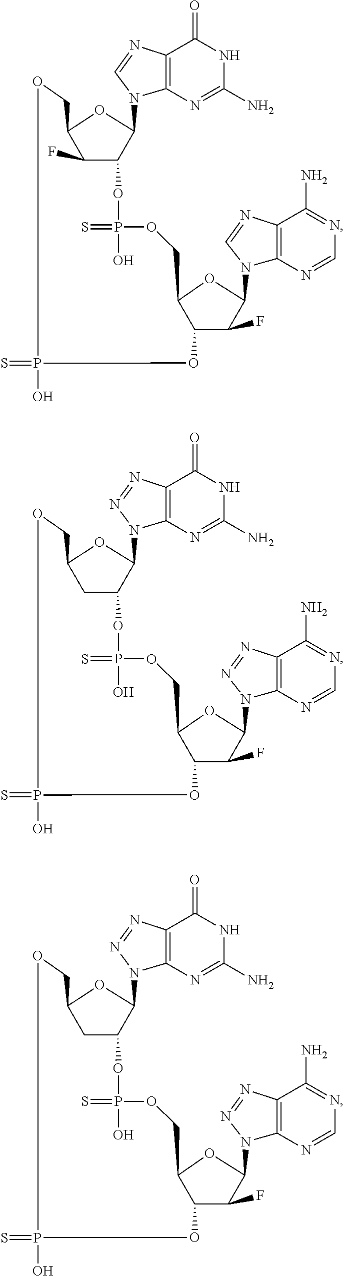 [SMILES: Nc1nc2c(ncn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] 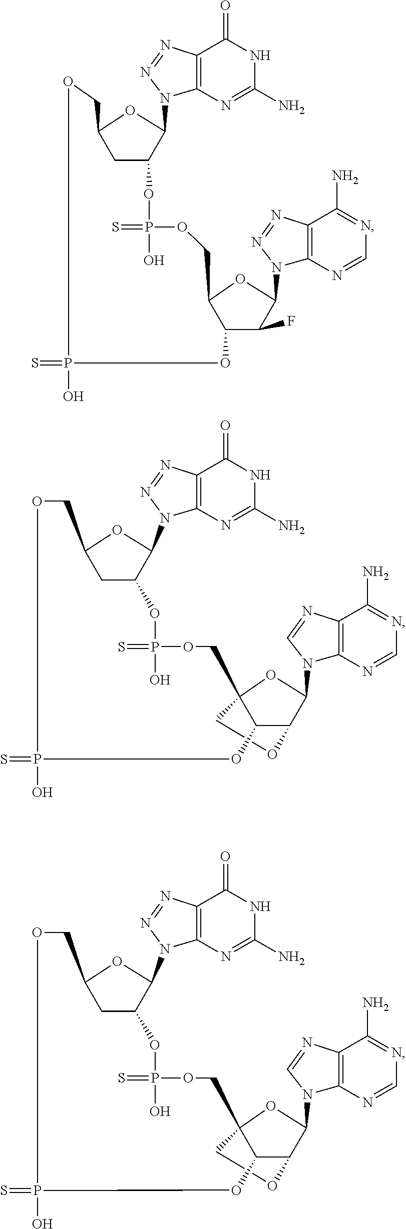 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1] 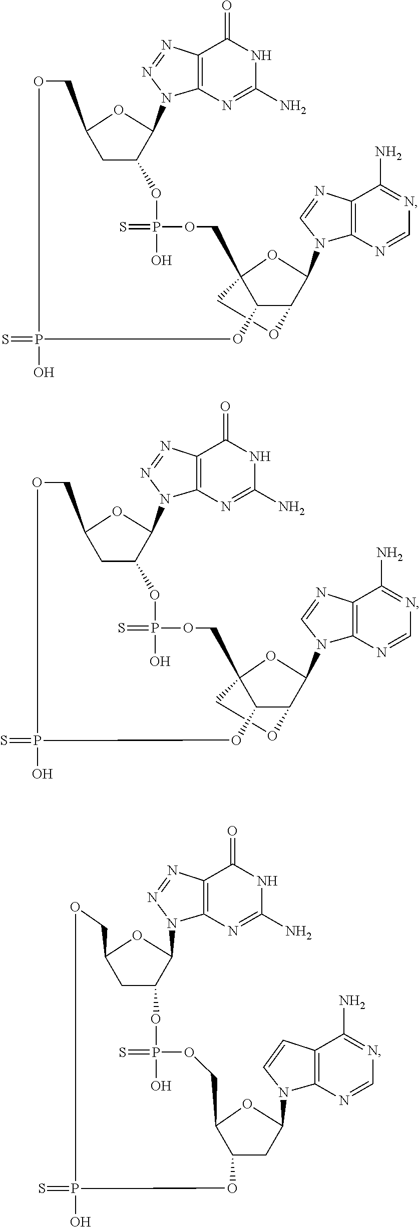 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1] 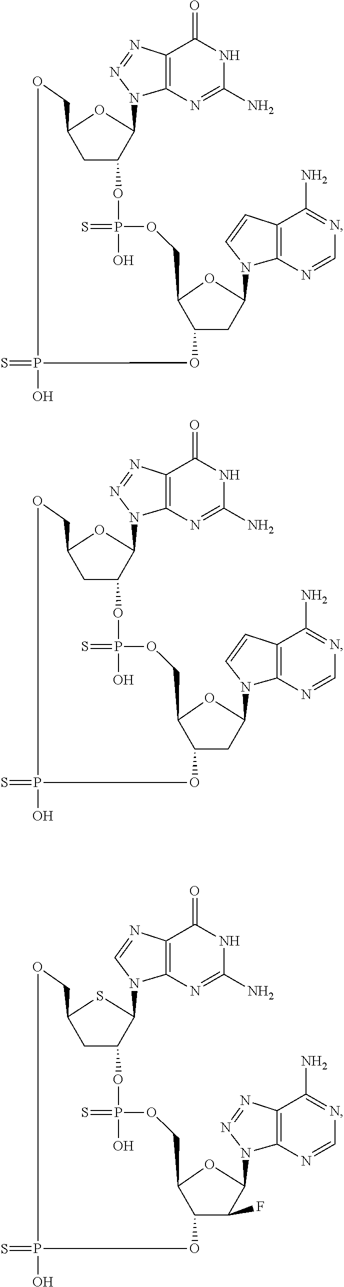 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] 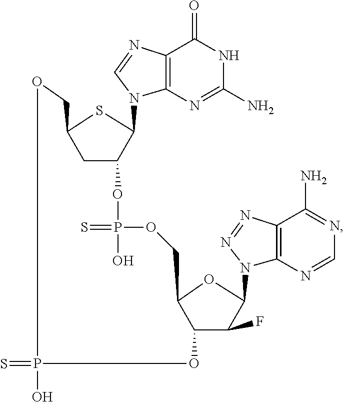 [SMILES: Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] 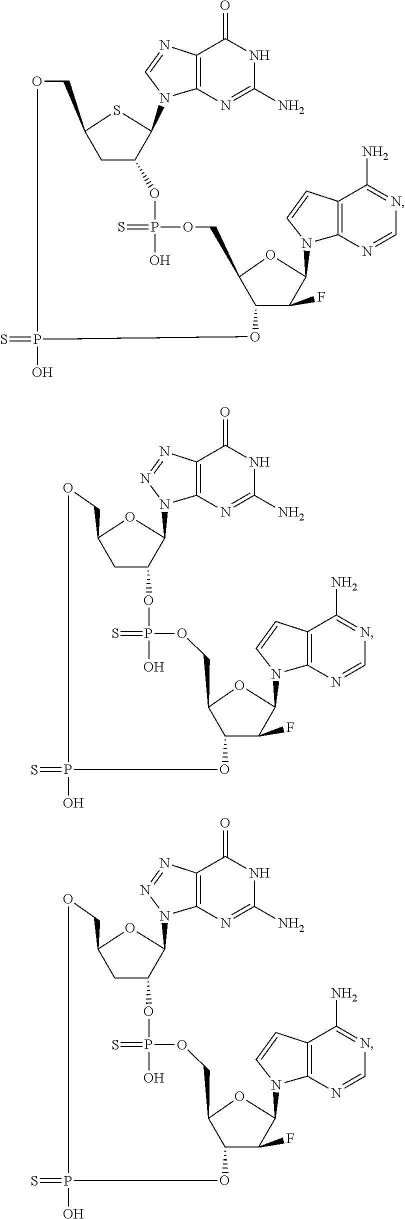 [SMILES: Nc1nc2c(ncn2[C@@H]2S[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1] 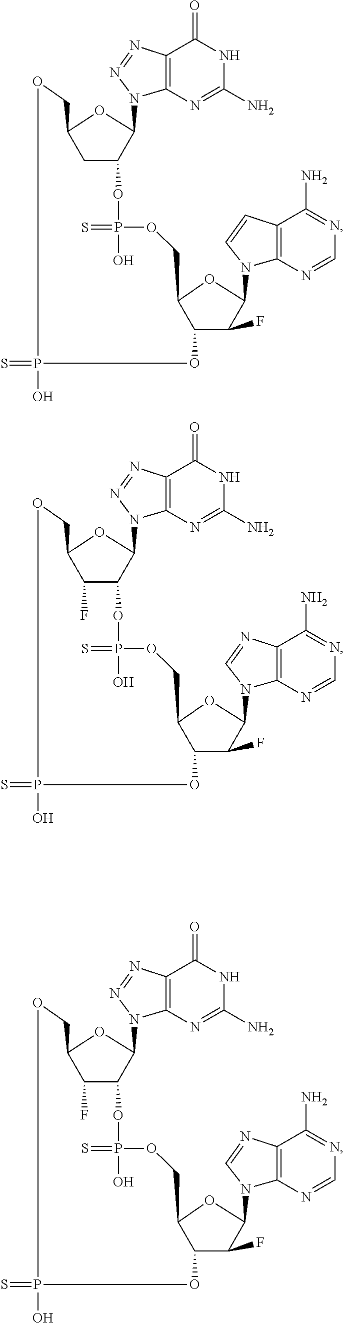 [SMILES: Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1] 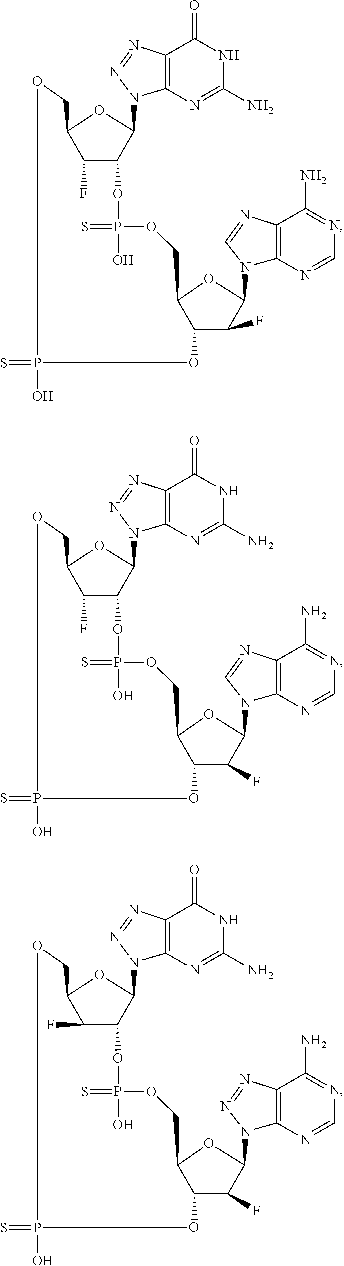 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@H]3F)c(=O)[nH]1] 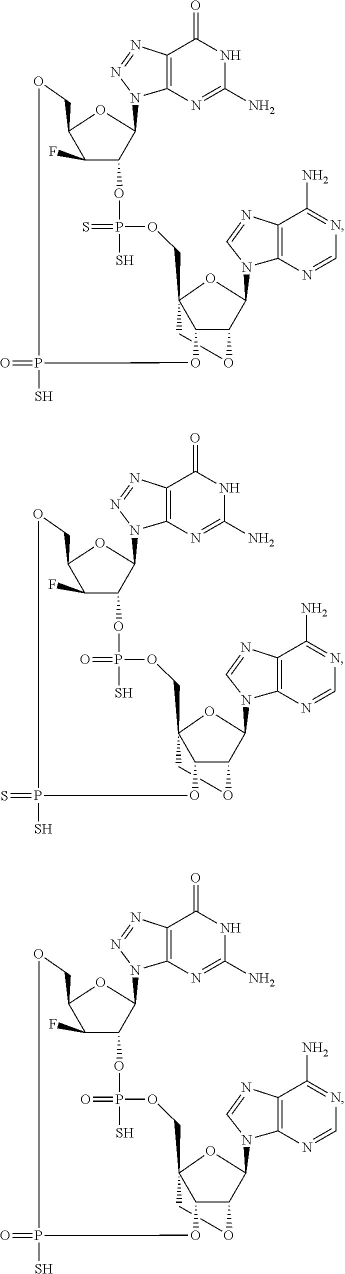 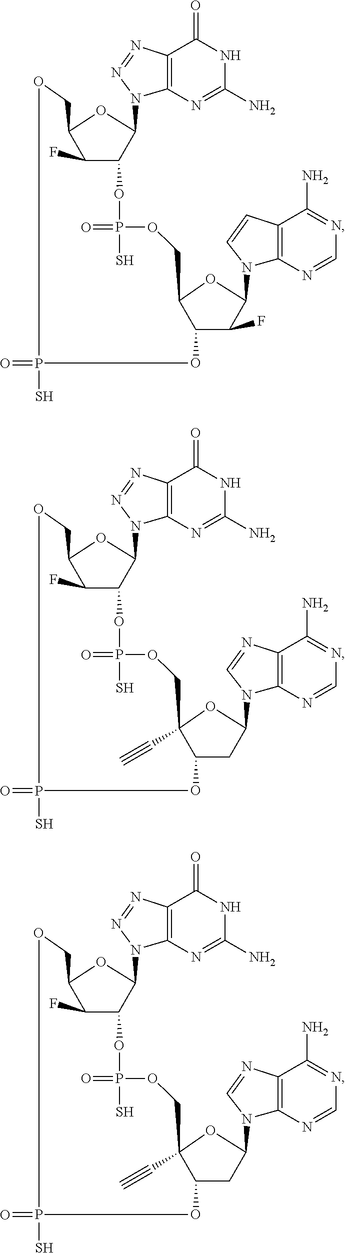 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21] 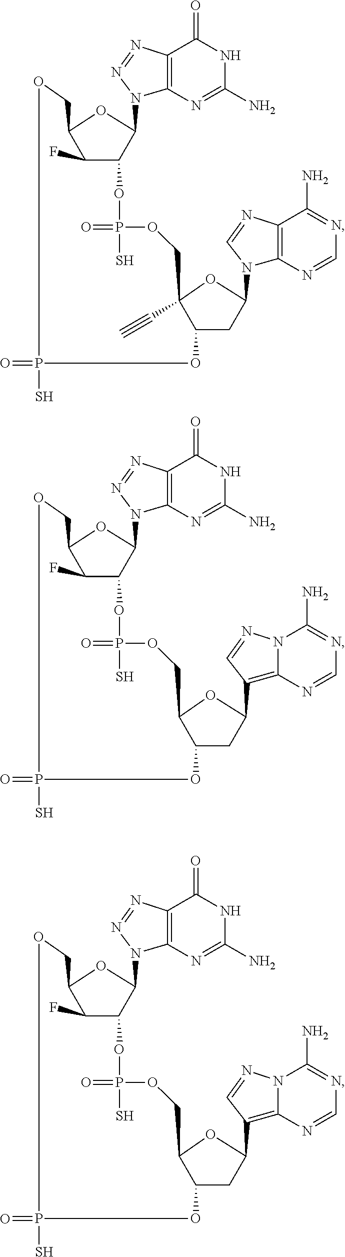 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1 ; Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1] 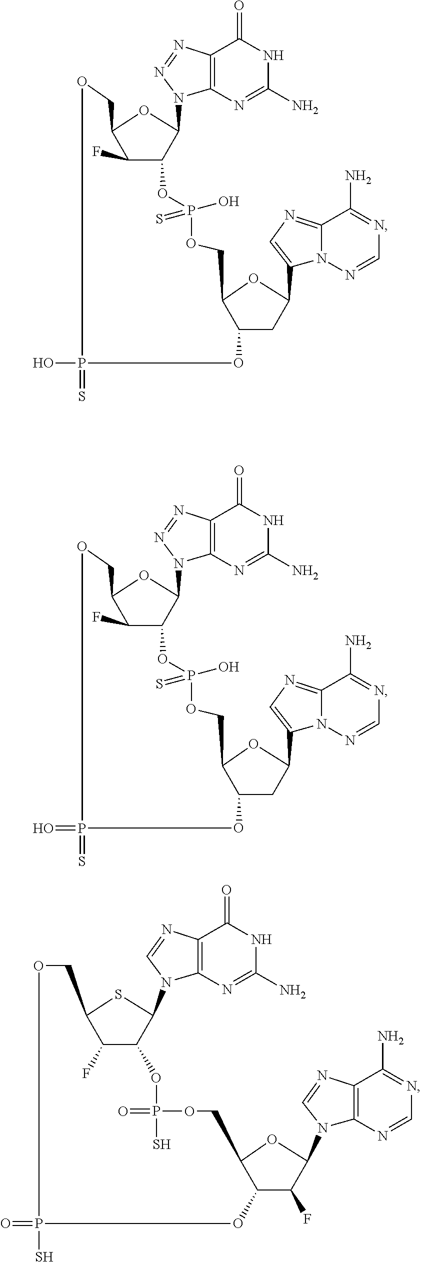 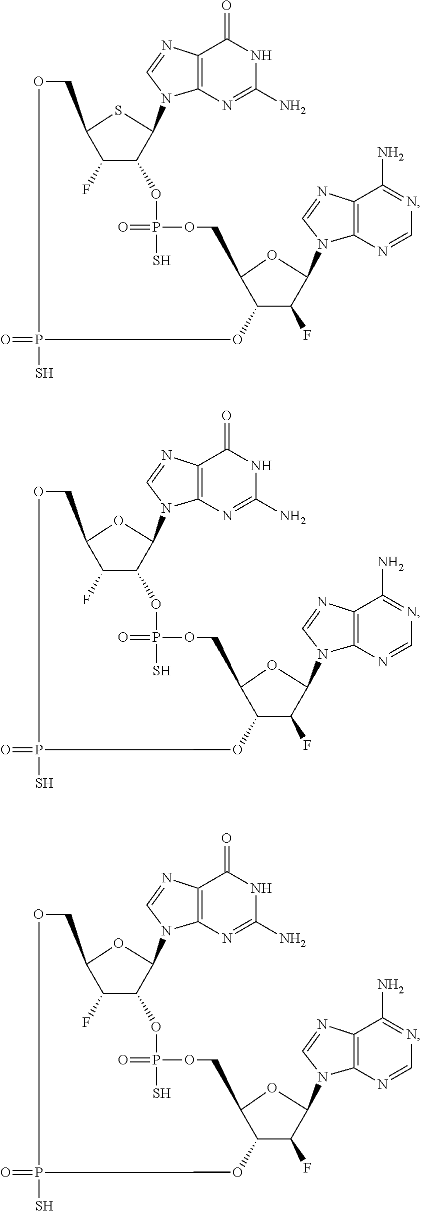 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] 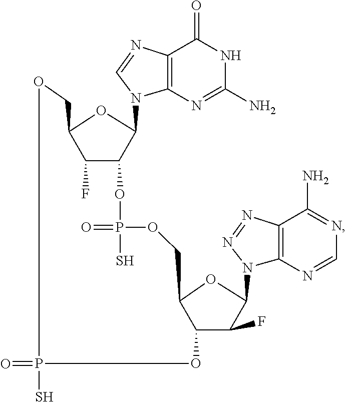 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] 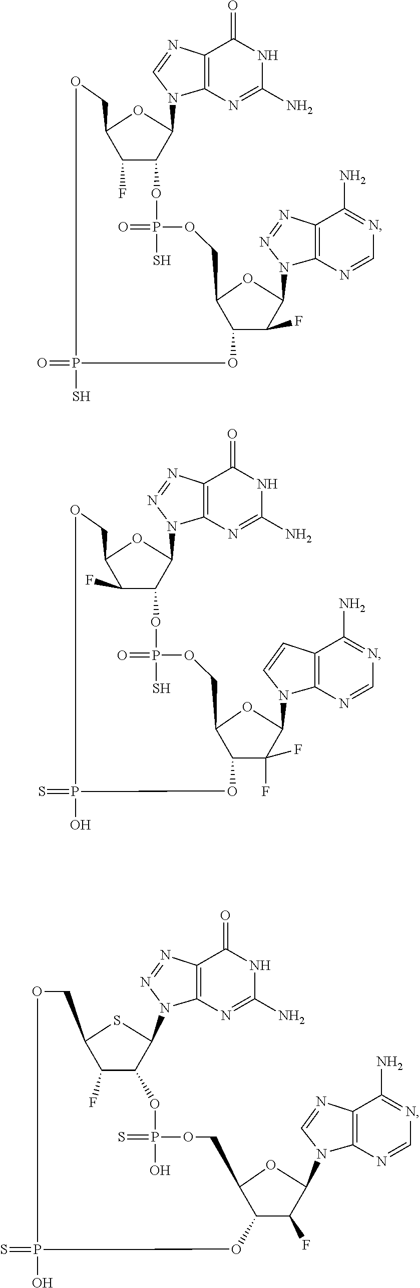 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2O[C@@H]3COP(O)(=S)O[C@@H]4[C@@H](COP(=O)(S)O[C@@H]2[C@H]3F)O[C@@H](n2ccc3c(N)ncnc32)C4(F)F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1] 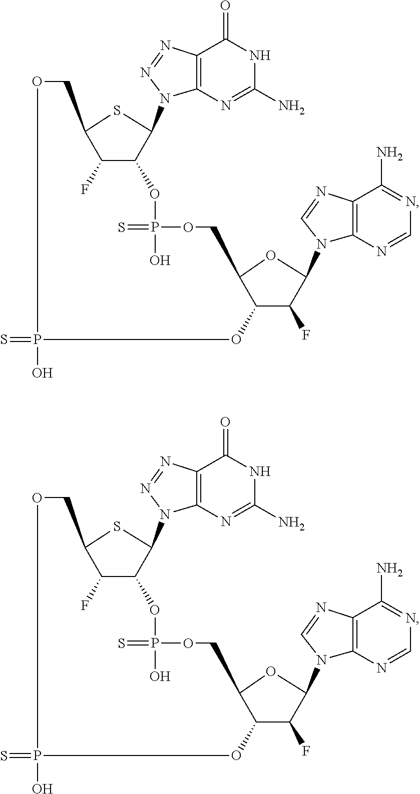 [SMILES: Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1] 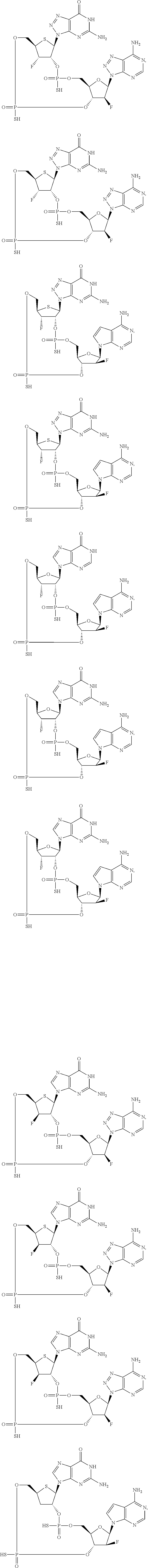 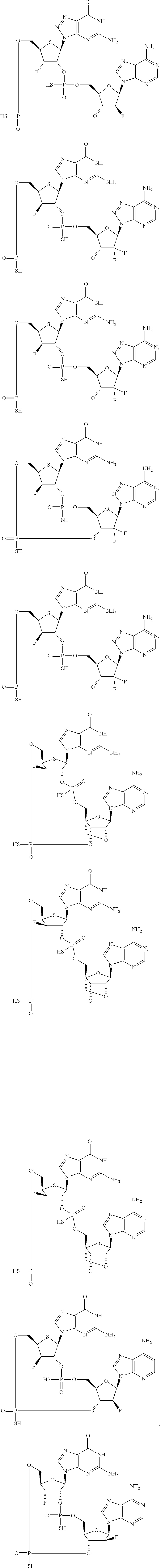 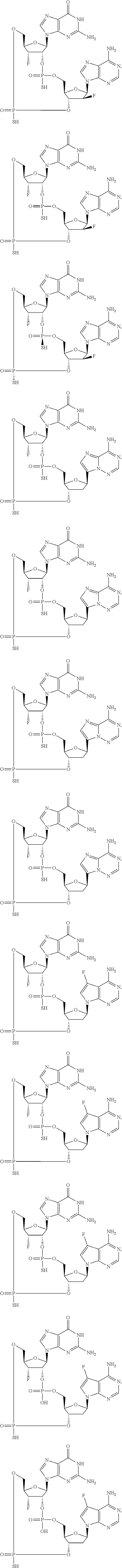 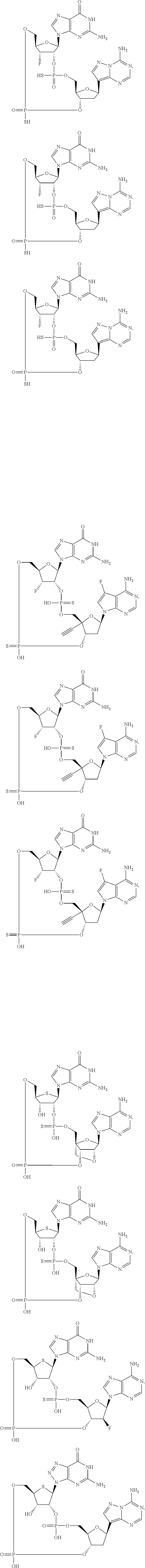 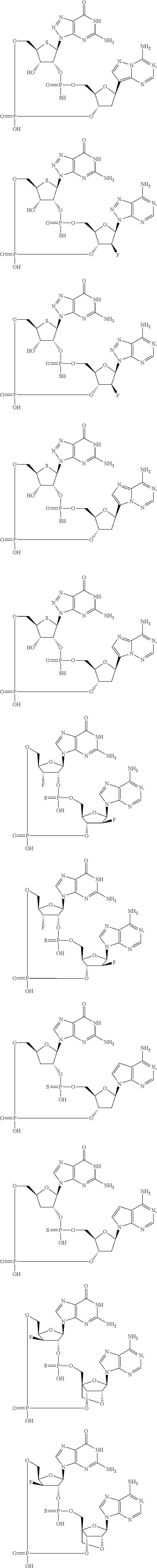 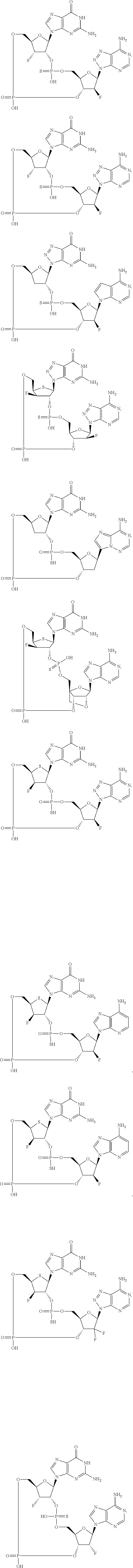 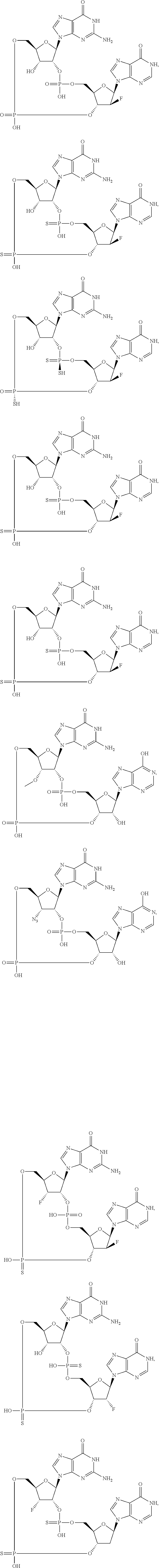 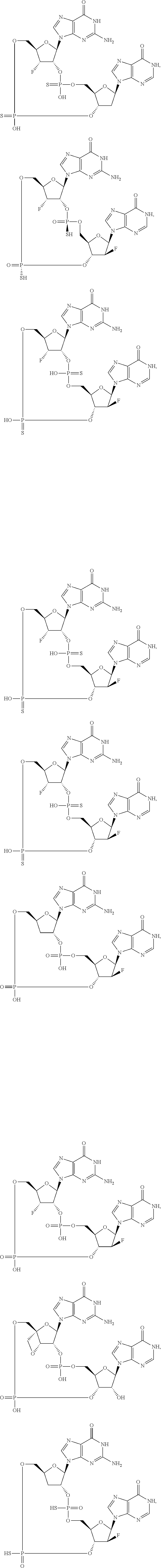 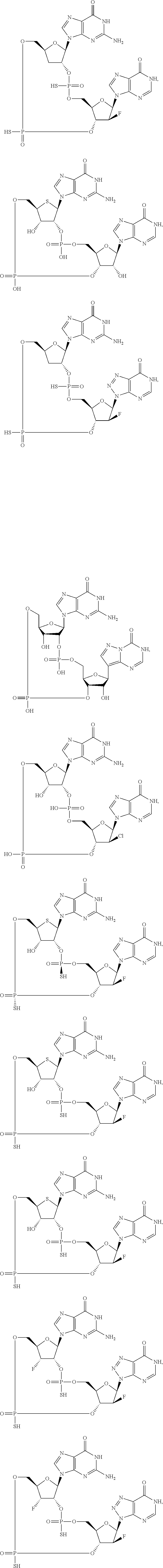 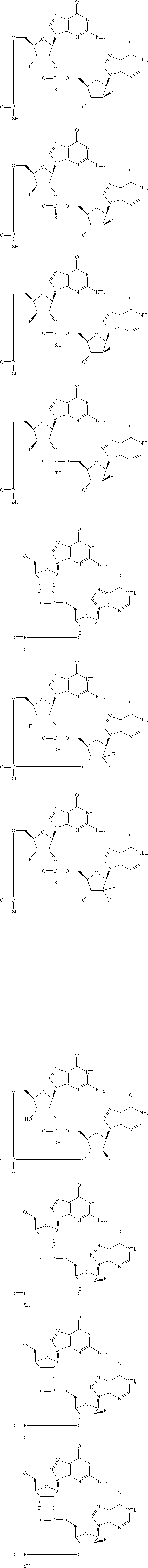 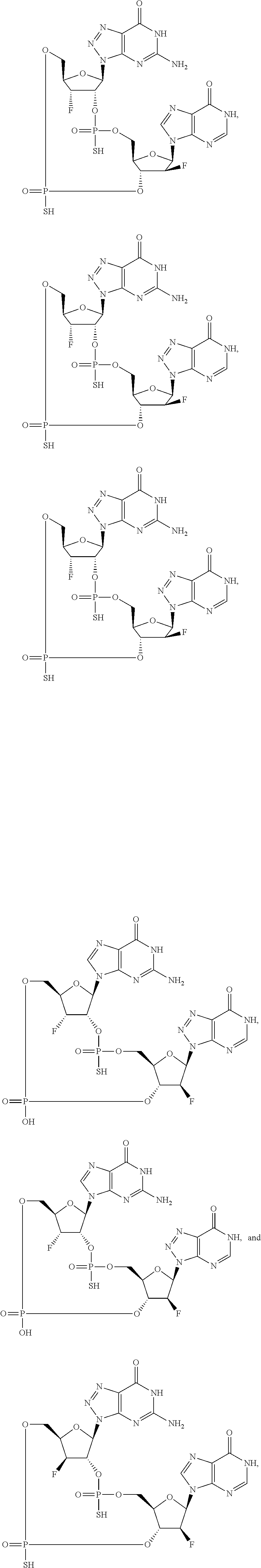
and
pharmaceutically acceptable salts thereof.

3. The pharmaceutical composition according to claim 1, wherein the pharmaceutically acceptable aqueous carrier is selected from the group consisting of water, about 30% captisol in water, about 30% hydroxypropyl beta-cyclodextrin in water, about 60% propylene glycol in water, about 10% polysorbate 80 in water, and about 10% dimethyl sulfoxide in water.

4. The pharmaceutical composition according to claim 1, wherein the pharmaceutically acceptable aqueous carrier is water.

5. The pharmaceutical composition according to claim 1, wherein the pharmaceutically acceptable tonicity modifier is selected from the group consisting of salts, sugar alcohols, polyols, and disaccharides.

6. The pharmaceutical composition according to claim 1, wherein the pharmaceutically acceptable tonicity modifier is selected from the group consisting of mannitol, sodium chloride, glycerol, sucrose, and trehalose.

7. The pharmaceutical composition according to claim 1, wherein the pharmaceutically acceptable tonicity modifier is mannitol.

8. The pharmaceutical composition according to claim 1, wherein the pharmaceutically acceptable buffer has a pKa of between about 5.5 and about 8.5.

9. The pharmaceutical composition according to claim 1, wherein the pharmaceutically acceptable buffer is selected from the group consisting of histidine, tris(hydroxymethyl)aminomethane (TRIS), sodium citrate, and sodium phosphate.

10. The pharmaceutical composition according to claim 1, wherein the pharmaceutically acceptable buffer is L-histidine.

11. The pharmaceutical composition according to claim 1, wherein the pharmaceutical composition has a pH of from about 6 to about 7.

12. The pharmaceutical composition according to claim 1, wherein the pharmaceutically acceptable antioxidant is selected from the group consisting of L-methionine, sodium metabisulfite, thiogylcerol, cysteine, and glutathione.

13. The pharmaceutical composition according to claim 1, wherein the pharmaceutically acceptable antioxidant is L-methionine.

14. The pharmaceutical composition according to claim 1, wherein the pharmaceutically acceptable metal chelator is selected from the group consisting of diethylenetriaminepentaacetic acid or edetate disodium dehydrate.

15. The pharmaceutical composition according to claim 1, wherein the pharmaceutically acceptable metal chelator is edetate disodium dehydrate.

16. A pharmaceutical composition comprising:
(a) a compound selected from the group consisting of 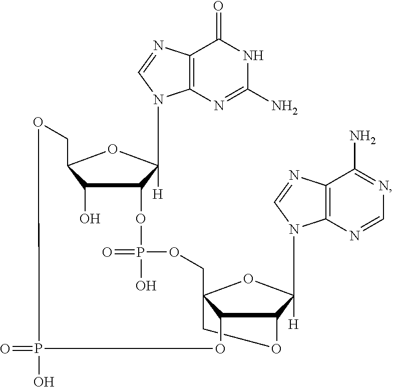 [SMILES: Nc1nc2c(ncn2[C@@H]2O[C@@H]3COP(=O)(O)O[C@@H]4C5OC[C@]4(COP(=O)(O)OC2[C@H]3O)O[C@H]5n2cnc3c(N)ncnc32)c(=O)[nH]1] 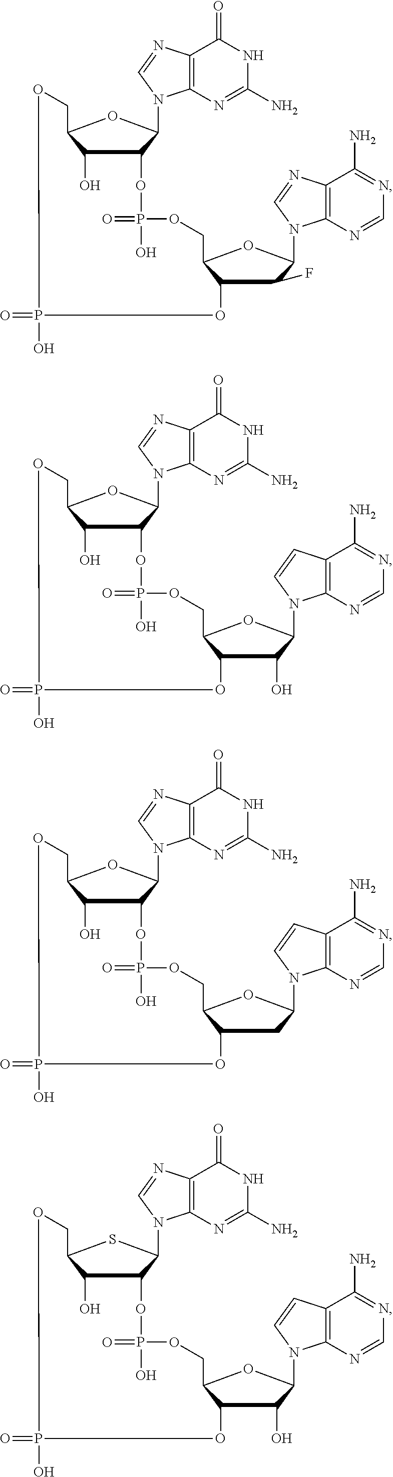 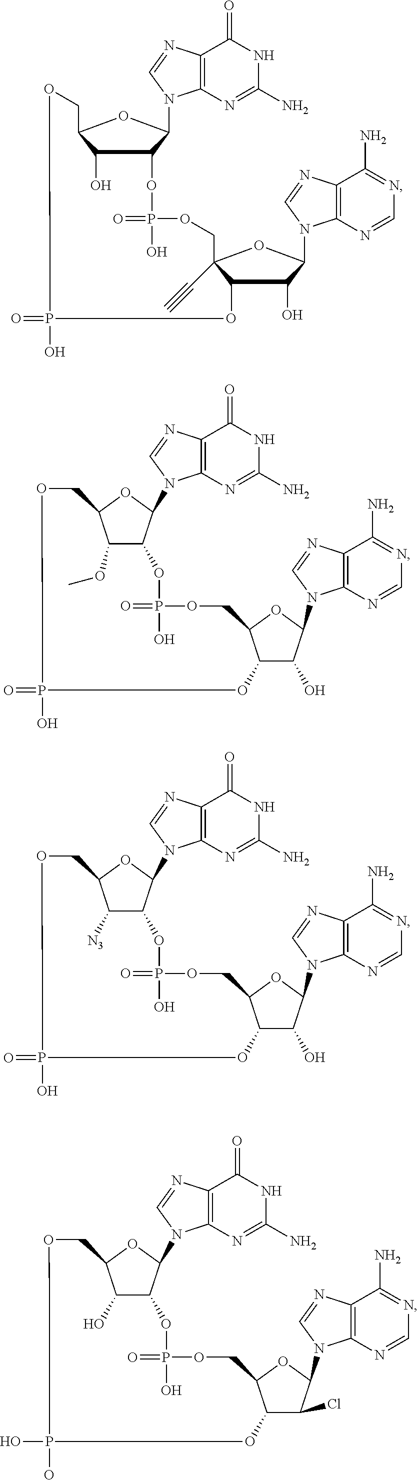 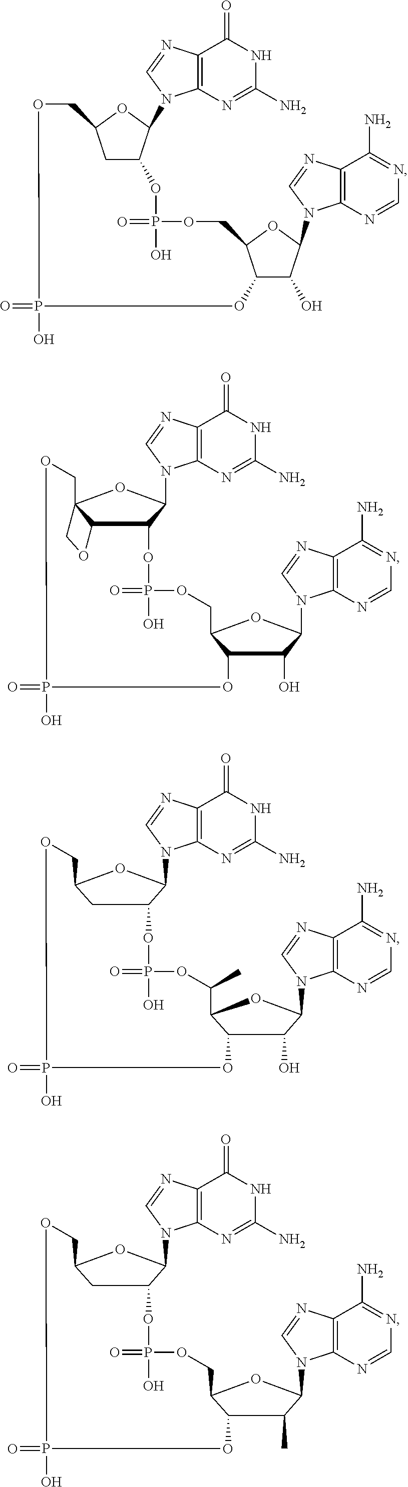 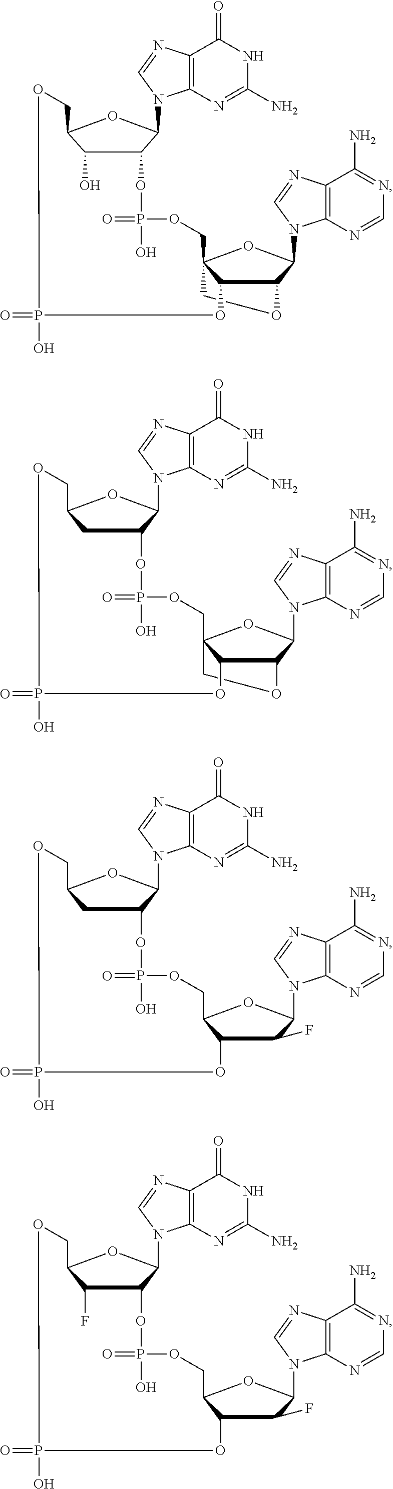 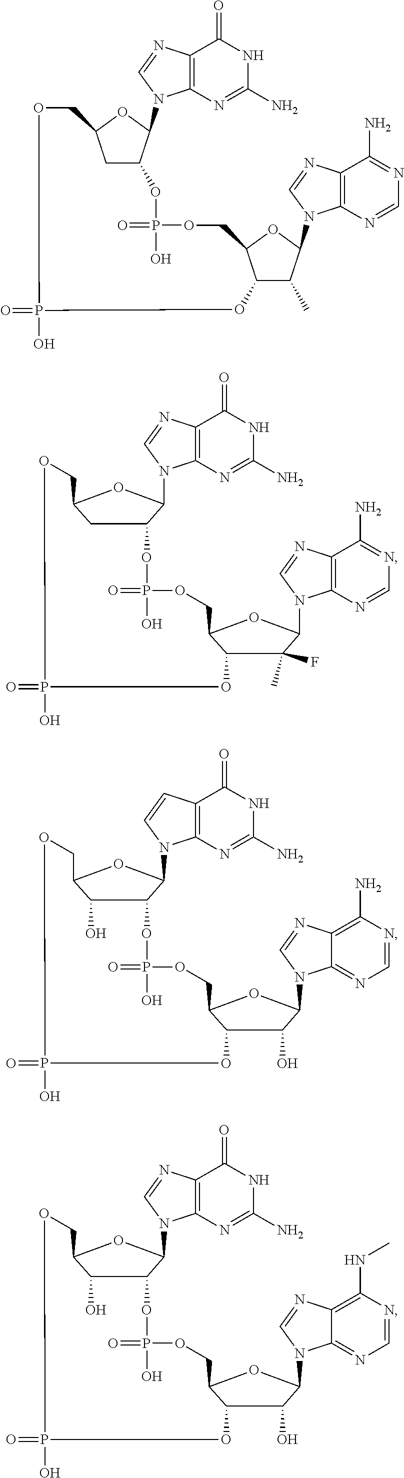 [SMILES: C[C@@H]1[C@@H]2OP(=O)(O)OC[C@@H]3C[C@@H](OP(=O)(O)OC[C@H]2O[C@H]1n1cnc2c(N)ncnc21)[C@H](n1cnc2c(=O)[nH]c(N)nc21)O3 ; C[C@@]1(F)C(n2cnc3c(N)ncnc32)O[C@@H]2COP(=O)(O)O[C@@H]3C[C@@H](COP(=O)(O)O[C@H]21)OC3n1cnc2c(=O)[nH]c(N)nc21 ; Nc1nc2c(ccn2[C@@H]2OC3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; CNc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] 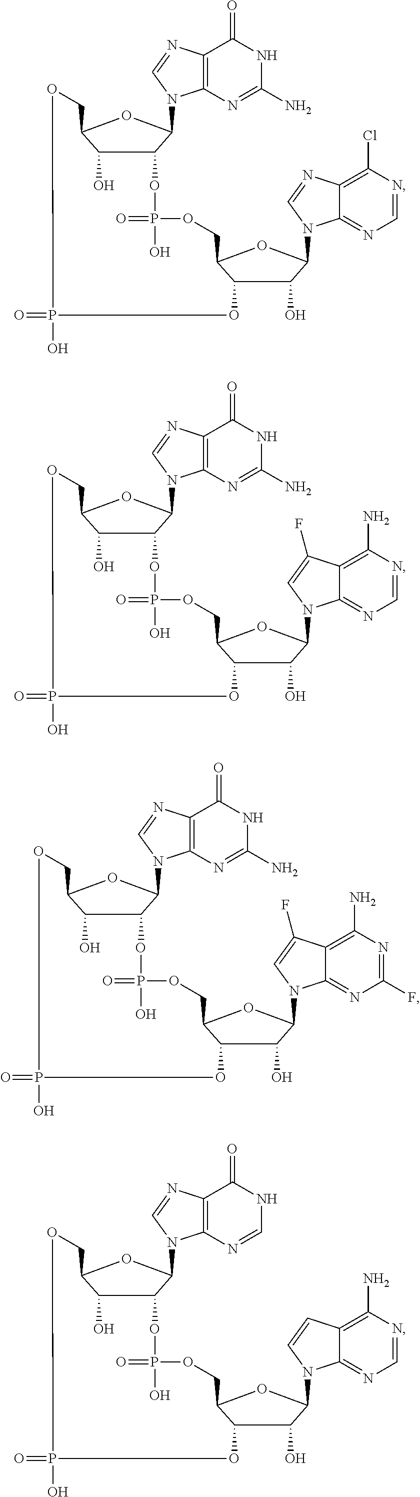 [SMILES: Nc1nc2c(ncn2[C@@H]2O[C@@H]3COP(=O)(O)O[C@H]4[C@@H](O)[C@H](n5cnc6c(Cl)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cc(F)c6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cc(F)c6c(N)nc(F)nc65)O[C@@H]4COP(=O)(O)O[C@@H]2[C@@H]3O)c(=O)[nH]1 ; Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21] 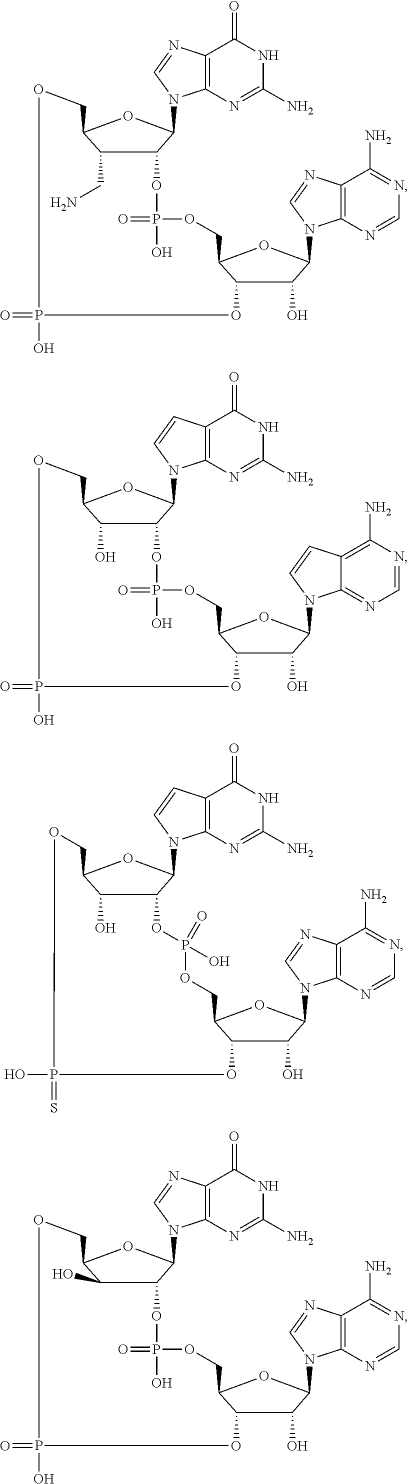 [SMILES: NC[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1nc2c(ccn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1 ; Nc1nc2c(ccn2[C@@H]2O[C@@H]3COP(O)(=S)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3O)c(=O)[nH]1] 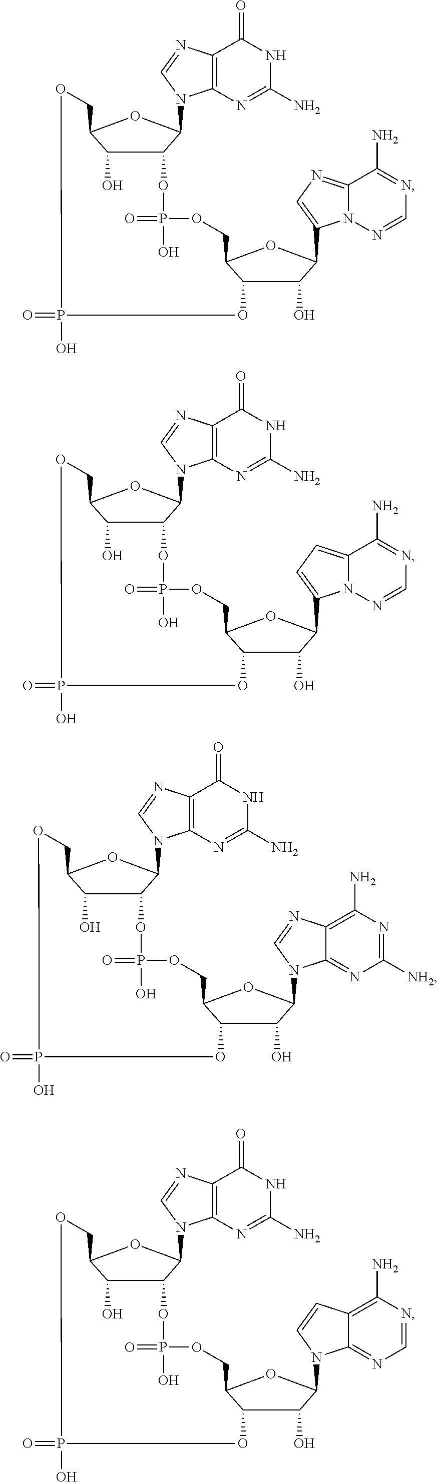 [SMILES: Nc1nc2c(ncn2[C@@H]2O[C@@H]3COP(=O)(O)O[C@H]4[C@@H](O)[C@H](c5cnc6c(N)ncnn56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](c5ccc6c(N)ncnn56)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1] 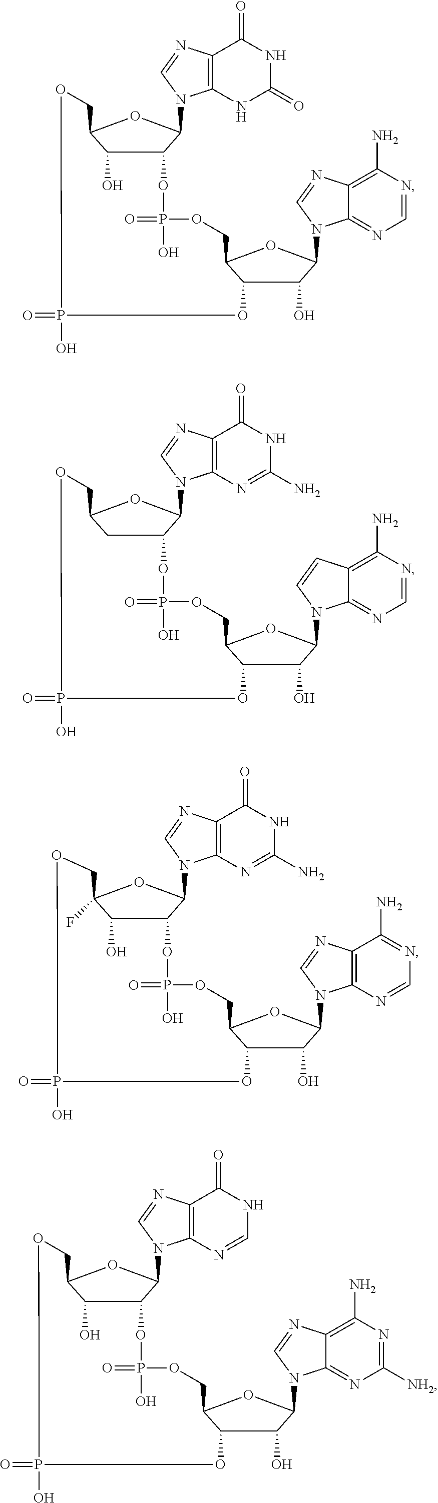 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(=O)[nH]c21 ; Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2C3)c(=O)[nH]1 ; Nc1nc2c(ncn2[C@@H]2O[C@]3(F)COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]cnc43)c2n1] 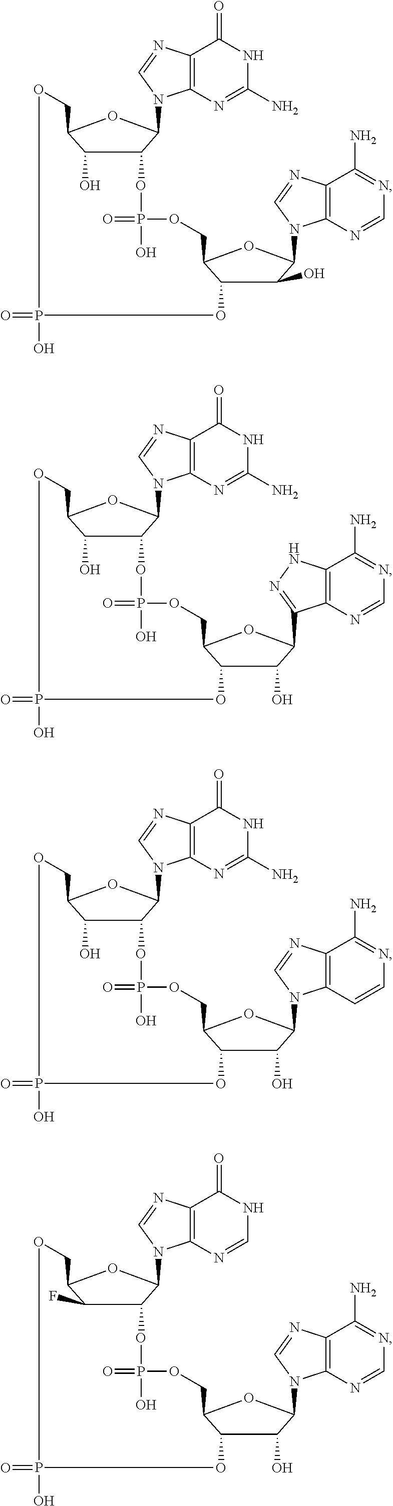 [SMILES: Nc1nc2c(ncn2[C@@H]2O[C@@H]3COP(=O)(O)O[C@H]4[C@H](O)[C@H](n5cnc6c(N)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](c5n[nH]c6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6c(N)nccc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@@H](F)[C@@H](COP(=O)(O)O[C@H]2[C@H]1O)O[C@H]3n1cnc2c(=O)[nH]cnc21] 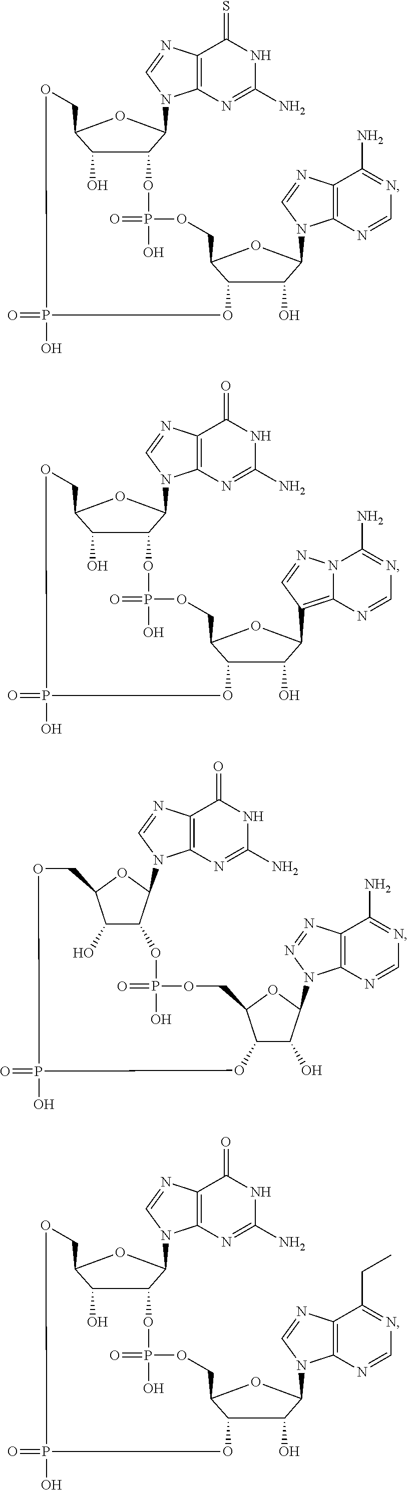 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=S)[nH]1 ; Nc1nc2c(ncn2[C@@H]2O[C@@H]3COP(=O)(O)O[C@H]4[C@@H](O)[C@H](c5cnn6c(N)ncnc56)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5nnc6c(N)ncnc65)O[C@@H]4COP(=O)(O)O[C@@H]2[C@@H]3O)c(=O)[nH]1 ; CCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] 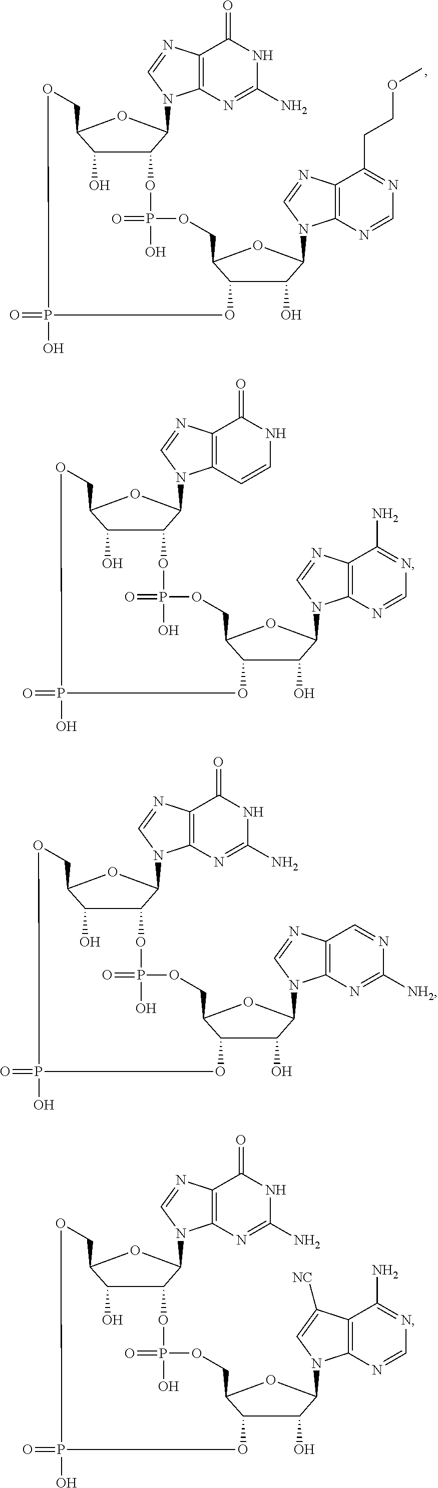 [SMILES: COCCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; Nc1ncc2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1 ; N#Cc1cn([C@@H]2O[C@@H]3COP(=O)(O)O[C@@H]4[C@H](O)[C@@H](COP(=O)(O)O[C@H]3[C@H]2O)O[C@H]4n2cnc3c(=O)[nH]c(N)nc32)c2ncnc(N)c12] 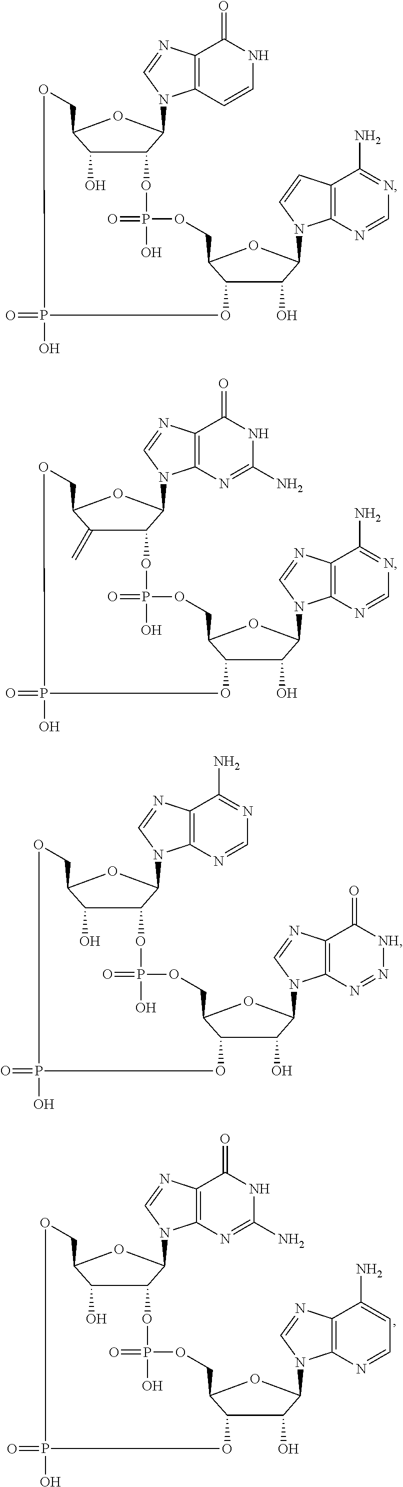 [SMILES: Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21 ; C=C1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21 ; Nc1ncnc2c1ncn2[C@@H]1O[C@@H]2COP(=O)(O)O[C@H]3[C@@H](O)[C@H](n4cnc5c(=O)[nH]nnc54)O[C@@H]3COP(=O)(O)O[C@@H]1[C@@H]2O ; Nc1nc2c(ncn2[C@@H]2O[C@@H]3COP(=O)(O)O[C@H]4[C@@H](O)[C@H](n5cnc6c(N)ccnc65)O[C@@H]4COP(=O)(O)O[C@@H]2[C@@H]3O)c(=O)[nH]1] 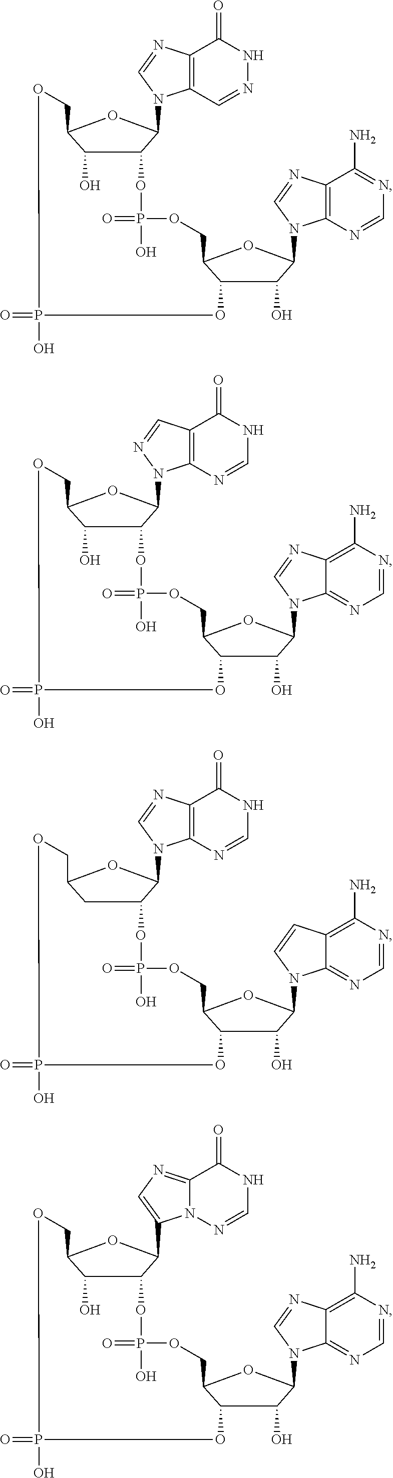 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ncc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1ncc2c(=O)[nH]cnc21 ; Nc1ncnc2c1ccn2[C@@H]1O[C@@H]2COP(=O)(O)O[C@@H]3CC(COP(=O)(O)O[C@H]2[C@H]1O)O[C@H]3n1cnc2c(=O)[nH]cnc21 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1cnc2c(=O)[nH]cnn12] 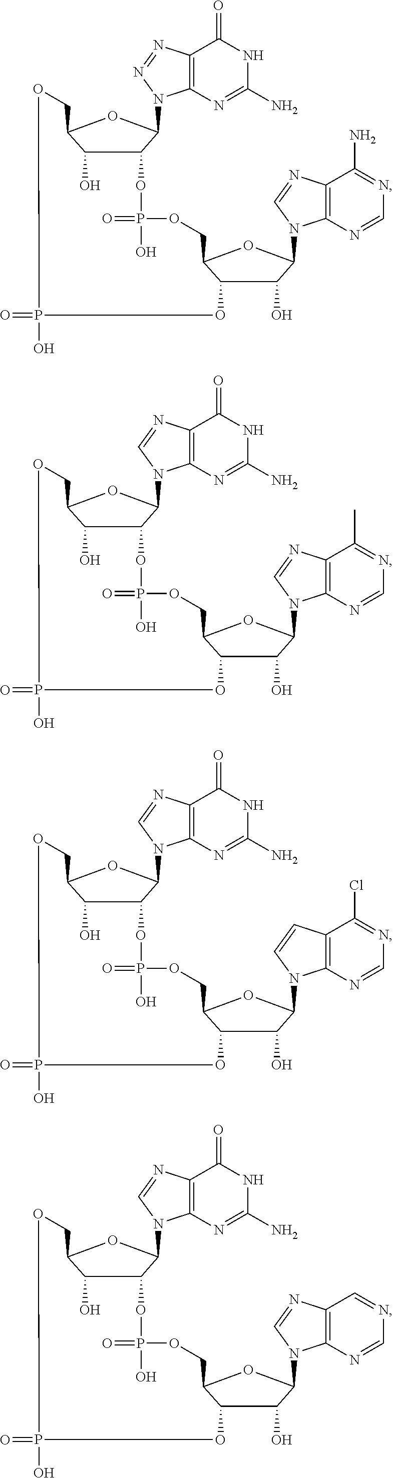 [SMILES: Nc1nc2c(nnn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1 ; Cc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21 ; Nc1nc2c(ncn2[C@@H]2O[C@@H]3COP(=O)(O)O[C@H]4[C@@H](O)[C@H](n5ccc6c(Cl)ncnc65)O[C@@H]4COP(=O)(O)O[C@@H]2[C@@H]3O)c(=O)[nH]1 ; Nc1nc2c(ncn2[C@@H]2O[C@@H]3COP(=O)(O)O[C@H]4[C@@H](O)[C@H](n5cnc6cncnc65)O[C@@H]4COP(=O)(O)O[C@@H]2[C@@H]3O)c(=O)[nH]1] 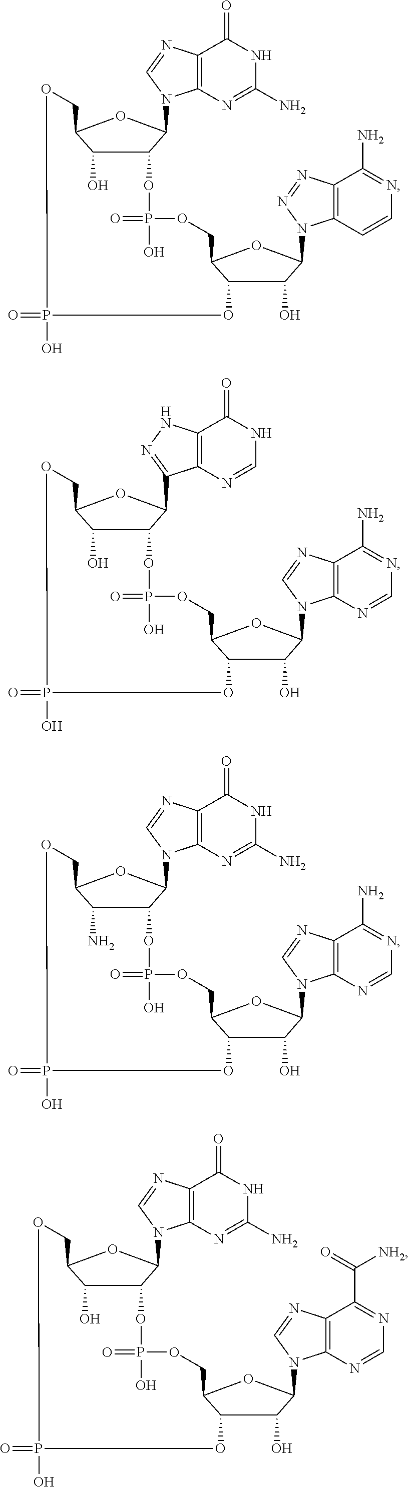 [SMILES: Nc1nc2c(ncn2[C@@H]2O[C@@H]3COP(=O)(O)O[C@H]4[C@@H](O)[C@H](n5nnc6c(N)nccc65)O[C@@H]4COP(=O)(O)O[C@@H]2[C@@H]3O)c(=O)[nH]1 ; Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1n[nH]c2c(=O)[nH]cnc12 ; Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3N)c(=O)[nH]1 ; NC(=O)c1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] 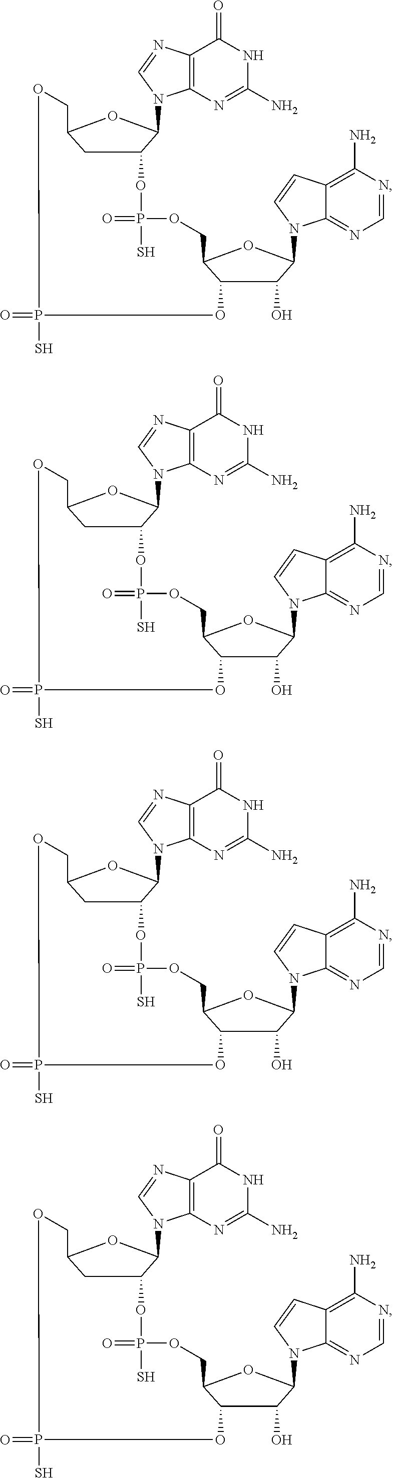 [SMILES: Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1 ; Nc1nc2c(ncn2[C@@H]2OC3COP(=O)(S)O[C@H]4[C@@H](O)[C@H](n5ccc6c(N)ncnc65)O[C@@H]4COP(=O)(S)O[C@@H]2C3)c(=O)[nH]1] 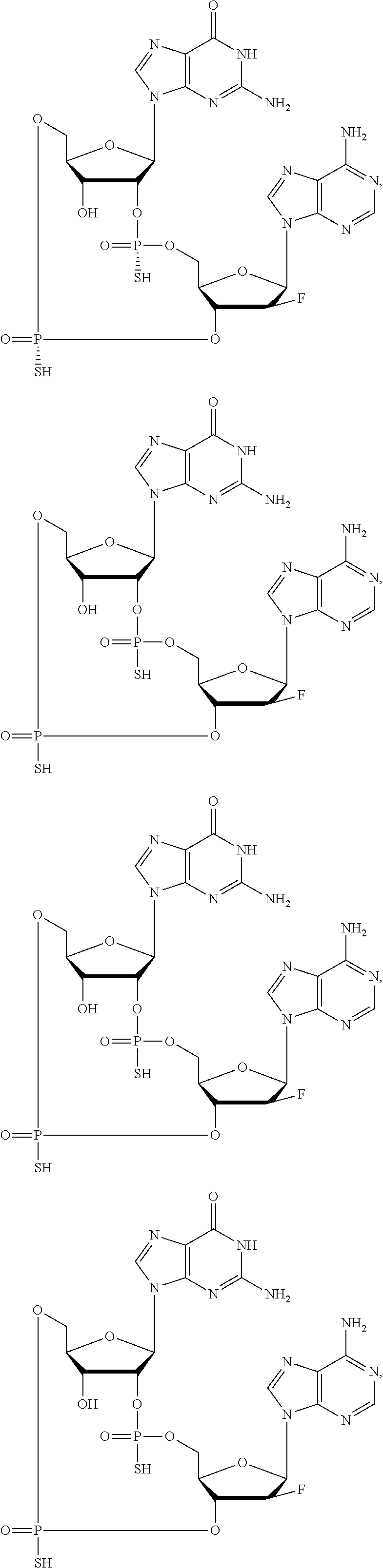 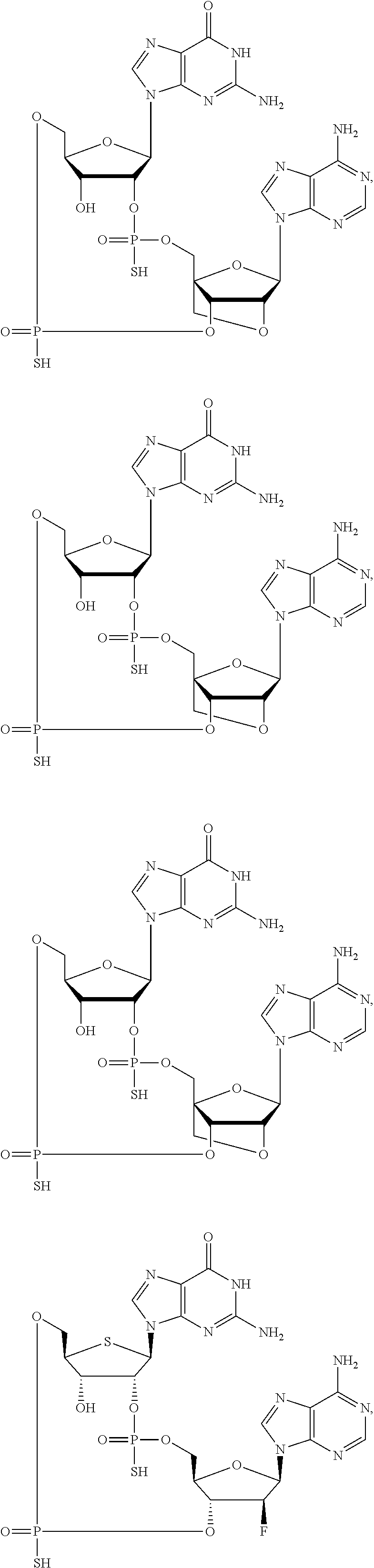 [SMILES: Nc1nc2c(ncn2[C@@H]2O[C@@H]3COP(=O)(S)O[C@@H]4C5OC[C@]4(COP(=O)(S)OC2[C@H]3O)O[C@H]5n2cnc3c(N)ncnc32)c(=O)[nH]1 ; Nc1nc2c(ncn2[C@@H]2O[C@@H]3COP(=O)(S)O[C@@H]4C5OC[C@]4(COP(=O)(S)OC2[C@H]3O)O[C@H]5n2cnc3c(N)ncnc32)c(=O)[nH]1 ; Nc1nc2c(ncn2[C@@H]2O[C@@H]3COP(=O)(S)O[C@@H]4C5OC[C@]4(COP(=O)(S)OC2[C@H]3O)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1] 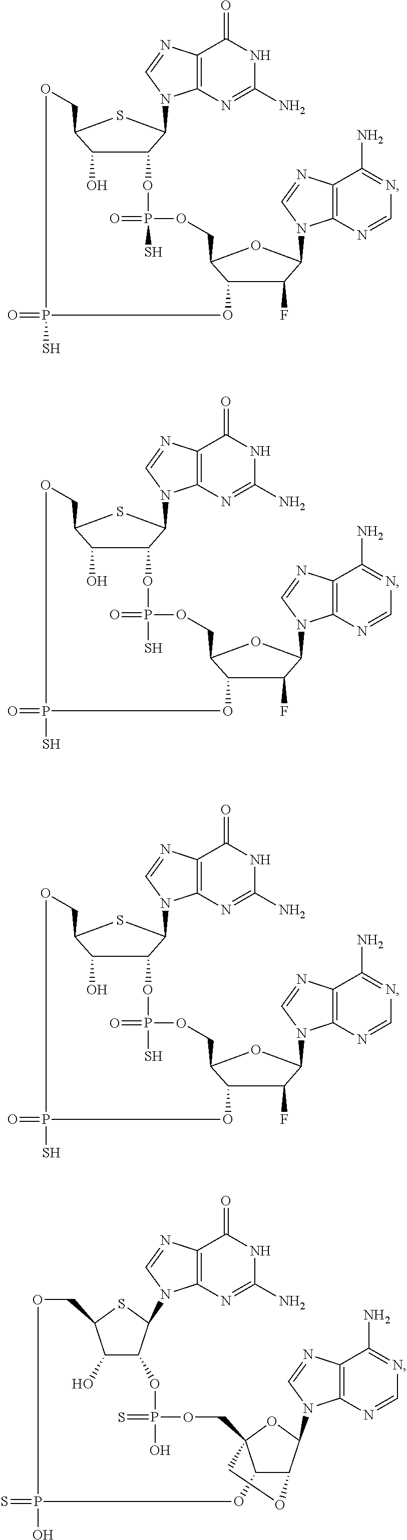 [SMILES: Nc1nc2c(ncn2[C@@H]2S[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3O)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1] 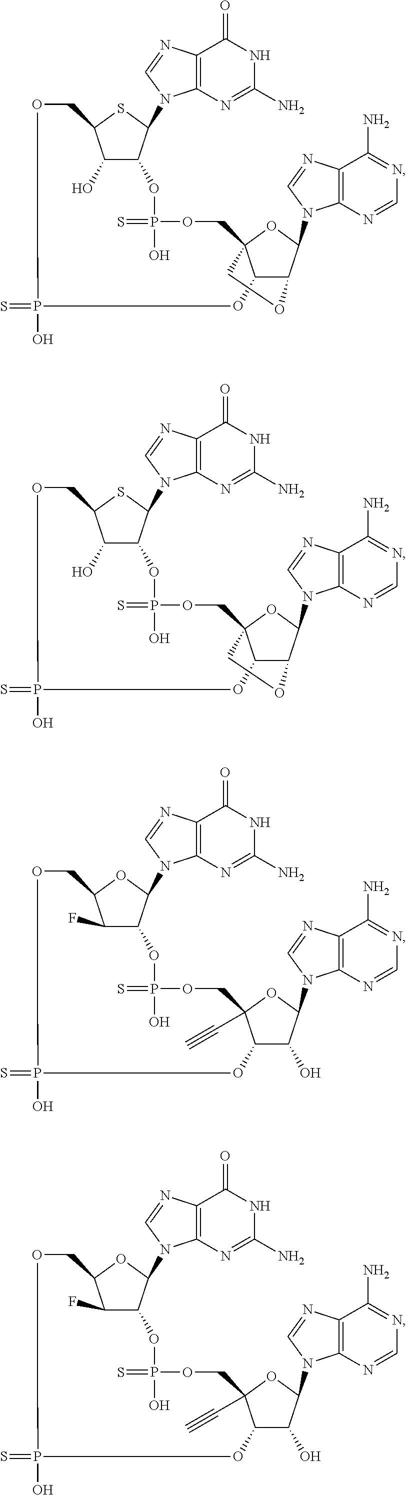 [SMILES: Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(O)(=S)O[C@H]4[C@H]5OC[C@]4(COP(O)(=S)O[C@@H]2[C@@H]3O)O[C@H]5n2cnc3c(N)ncnc32)c(=O)[nH]1 ; C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21] 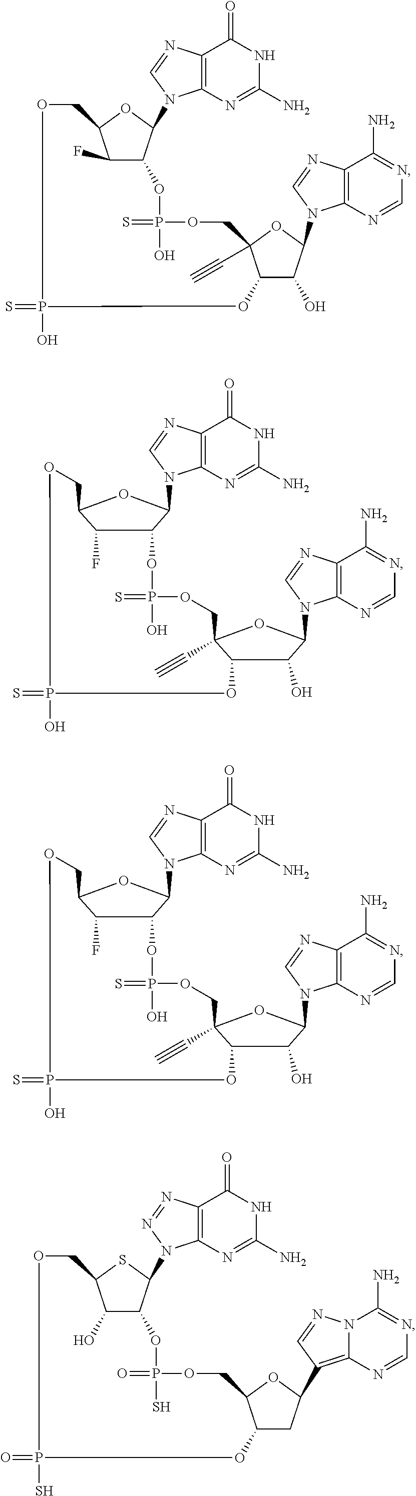 [SMILES: C#C[C@@]12COP(O)(=S)O[C@@H]3[C@@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1] 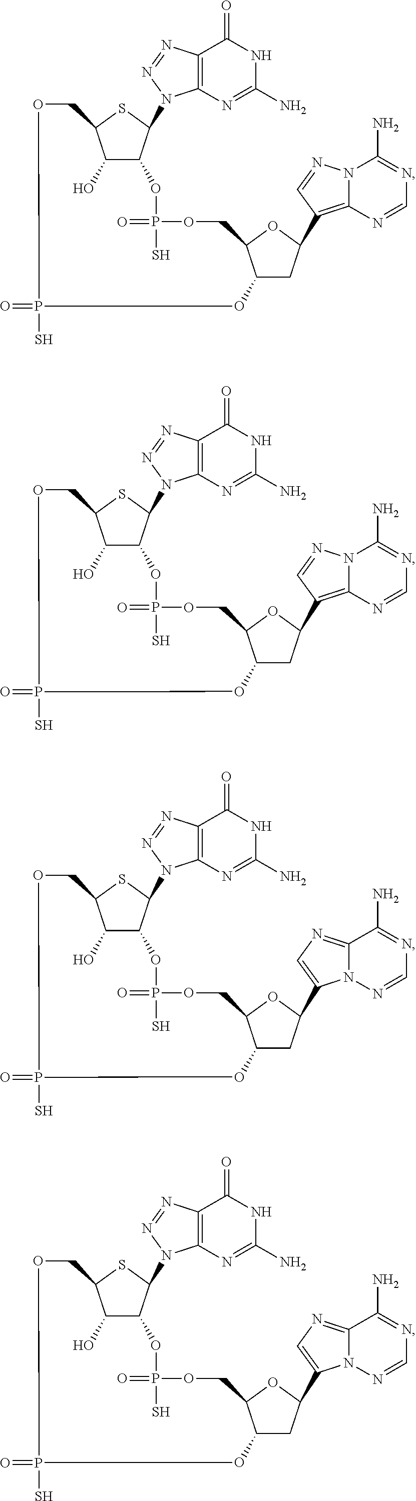 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] 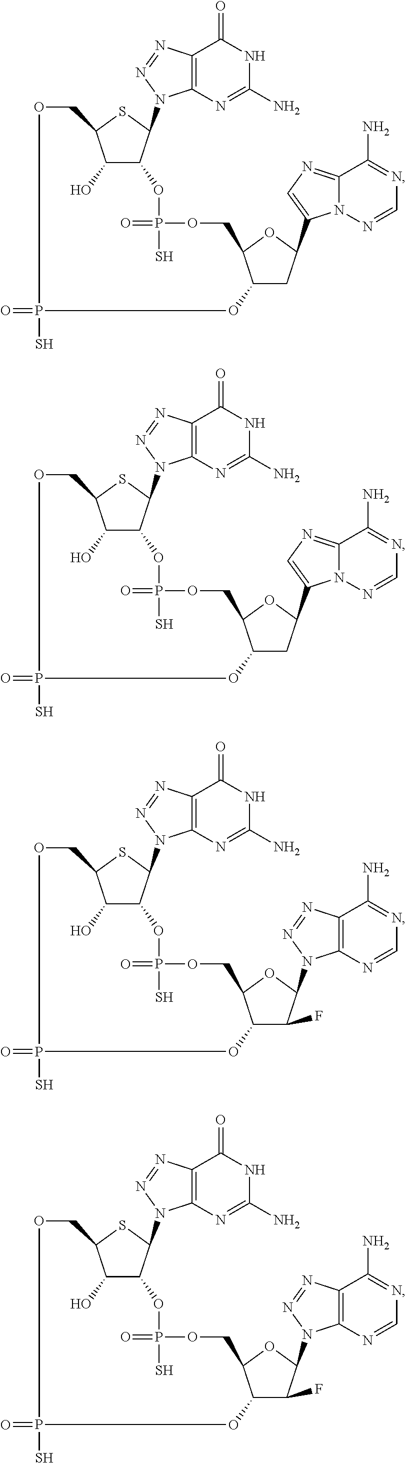 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1] 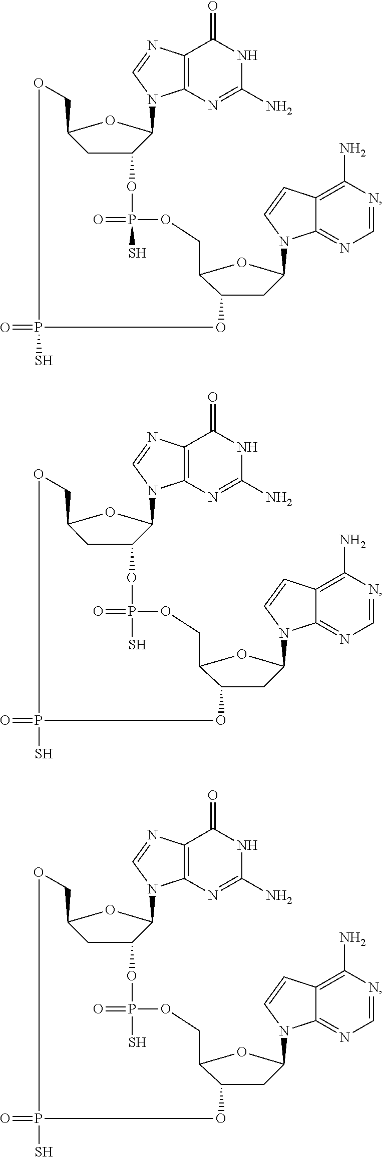 [SMILES: Nc1nc2c(ncn2[C@@H]2O[C@@H]3CO[P@@](=O)(S)O[C@H]4C[C@H](n5ccc6c(N)ncnc65)OC4CO[P@@](=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1 ; Nc1nc2c(ncn2[C@@H]2O[C@@H]3COP(=O)(S)O[C@H]4C[C@H](n5ccc6c(N)ncnc65)OC4COP(=O)(S)O[C@@H]2C3)c(=O)[nH]1] 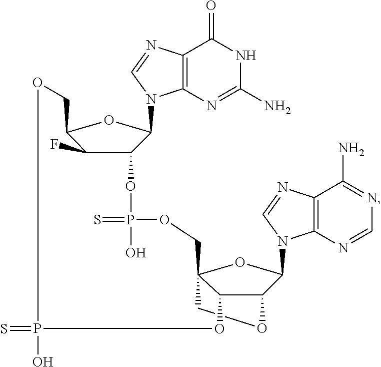 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1] 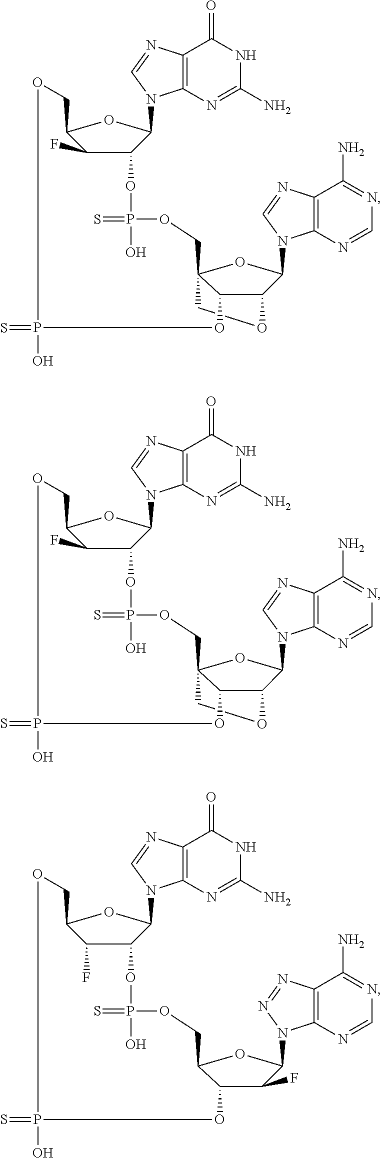 [SMILES: Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] 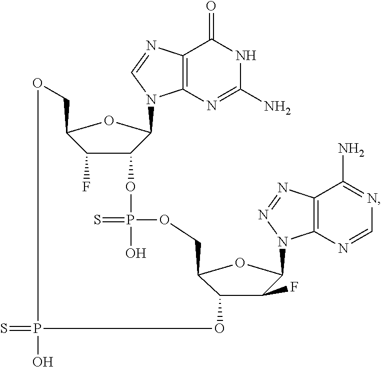 [SMILES: Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] 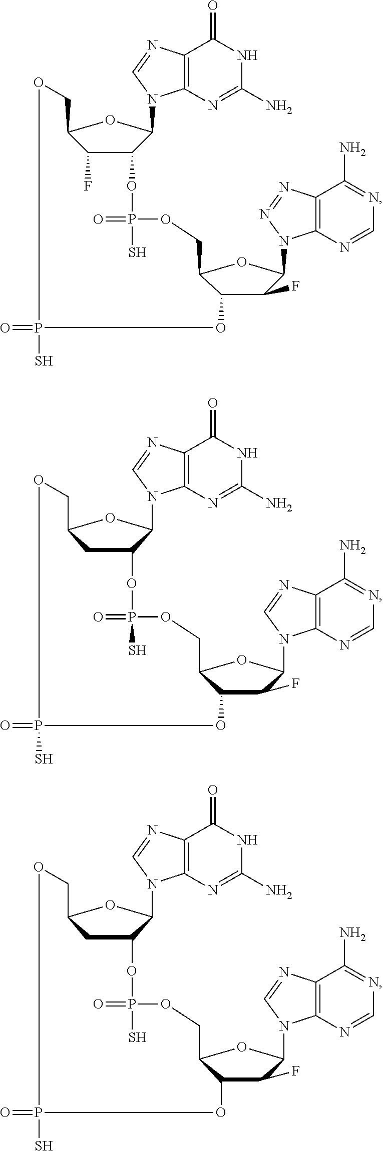 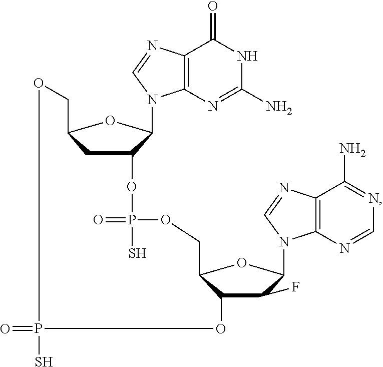 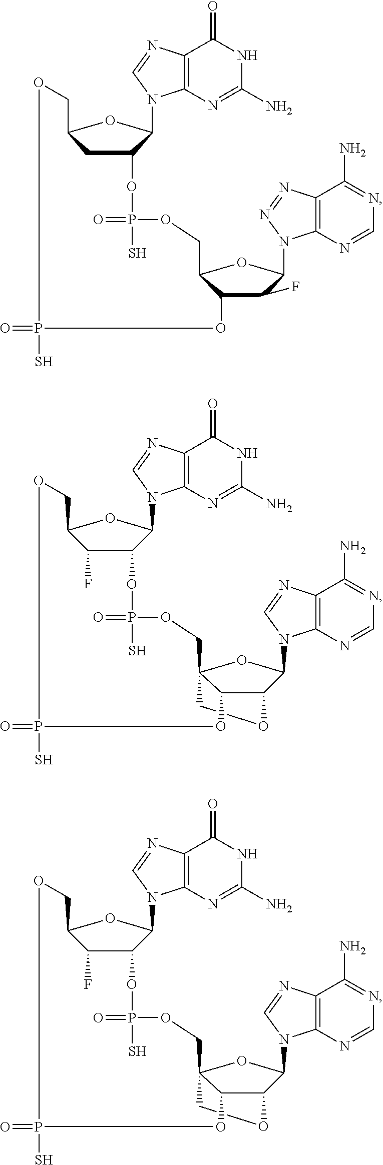 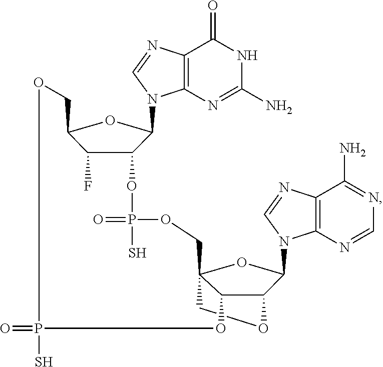 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] 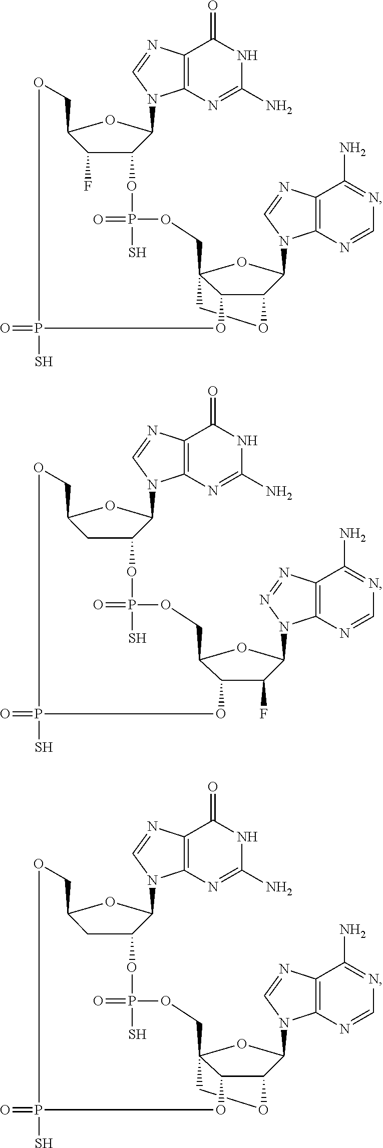 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] 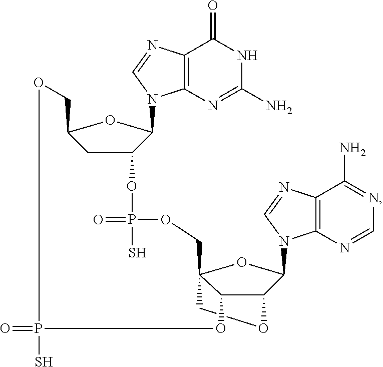 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] 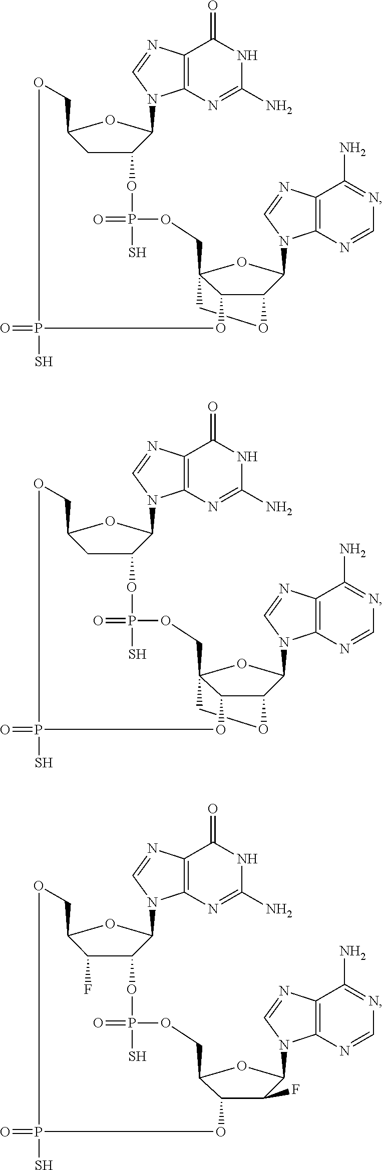 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] 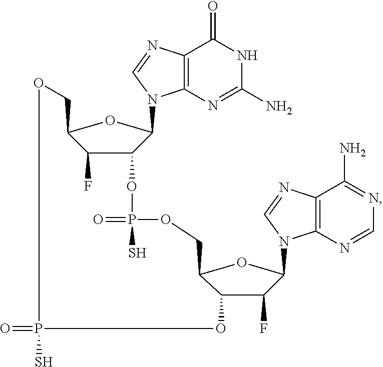 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@H]3F)c(=O)[nH]1] 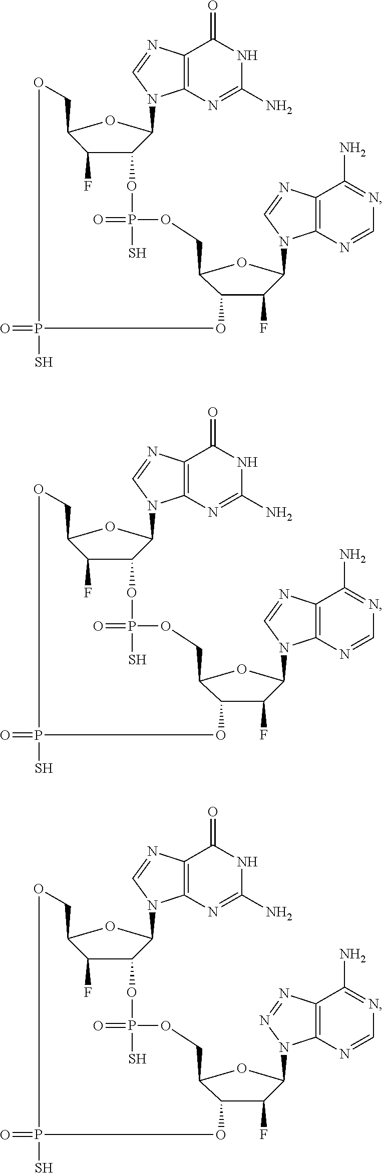 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] 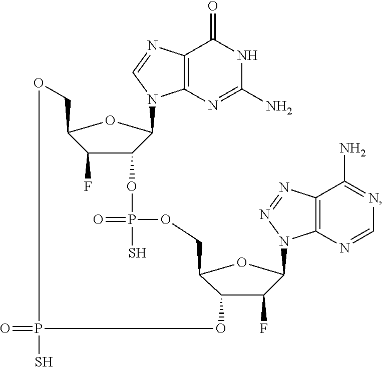 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] 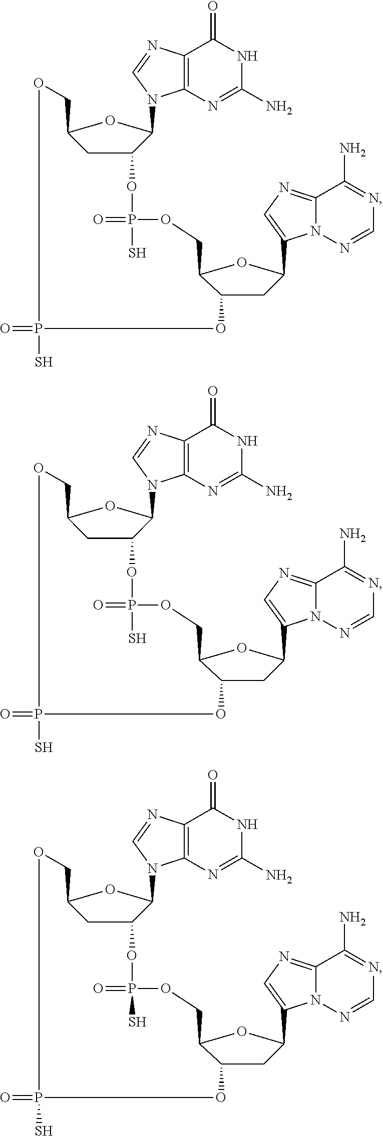 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2O[C@@H]3CO[P@@](=O)(S)O[C@H]4C[C@H](c5cnc6c(N)ncnn56)O[C@@H]4CO[P@@](=O)(S)O[C@@H]2C3)c(=O)[nH]1] 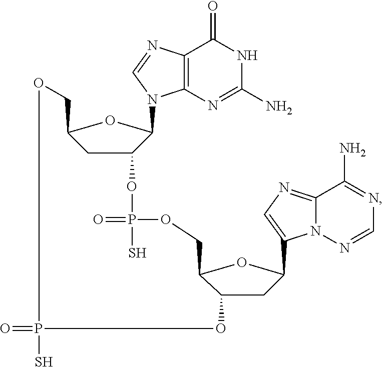 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1] 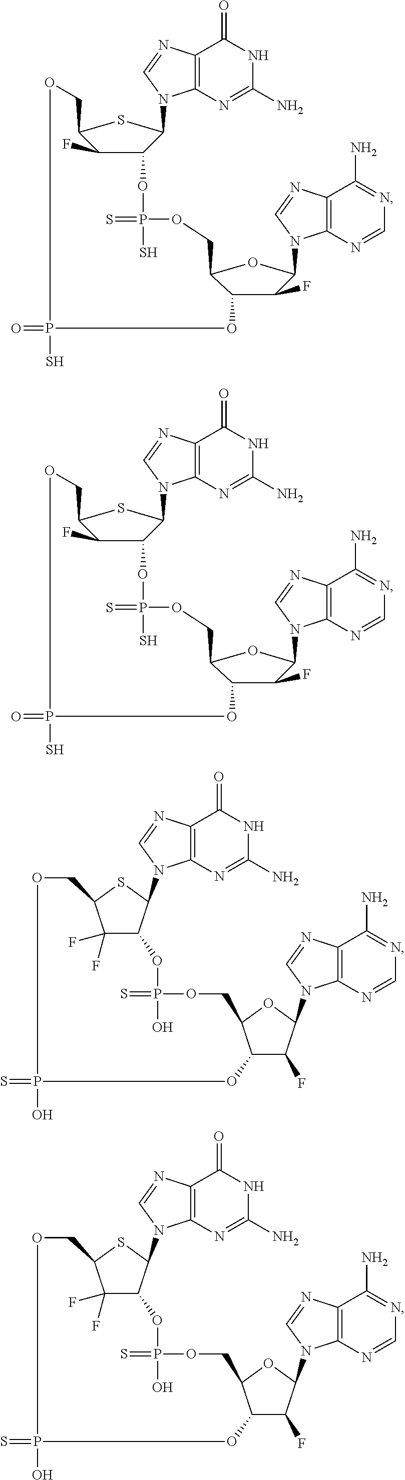 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=S)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=S)(S)O[C@@H]2[C@H]3F)c(=O)[nH]1 ; Nc1nc2c(ncn2[C@@H]2S[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1 ; Nc1nc2c(ncn2[C@@H]2S[C@@H]3COP(O)(=S)O[C@H]4[C@H](F)[C@H](n5cnc6c(N)ncnc65)O[C@@H]4COP(O)(=S)O[C@@H]2C3(F)F)c(=O)[nH]1] 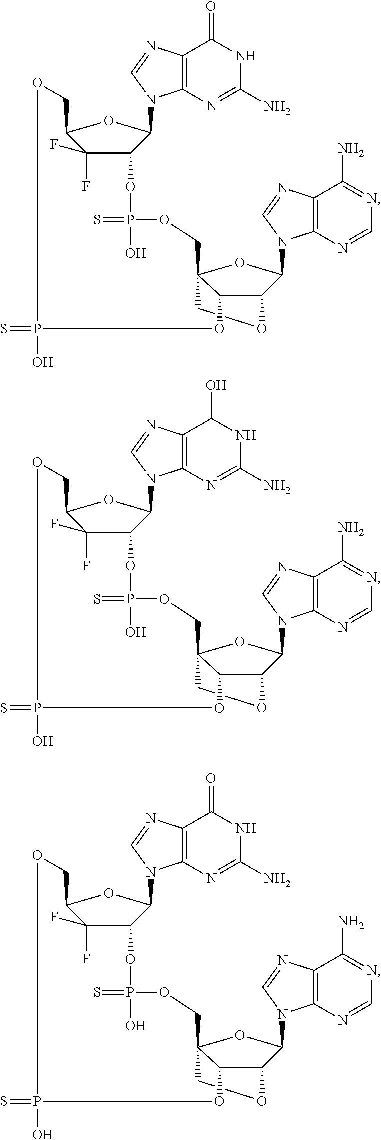 [SMILES: Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1 ; NC1=Nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)C(O)N1 ; Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1] 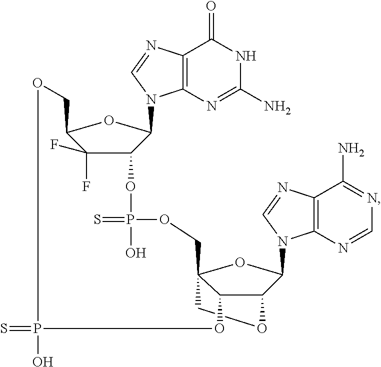 [SMILES: Nc1nc2c(ncn2[C@@H]2O[C@@H]3COP(O)(=S)O[C@H]4[C@H]5OC[C@]4(COP(O)(=S)O[C@@H]2C3(F)F)O[C@H]5n2cnc3c(N)ncnc32)c(=O)[nH]1] 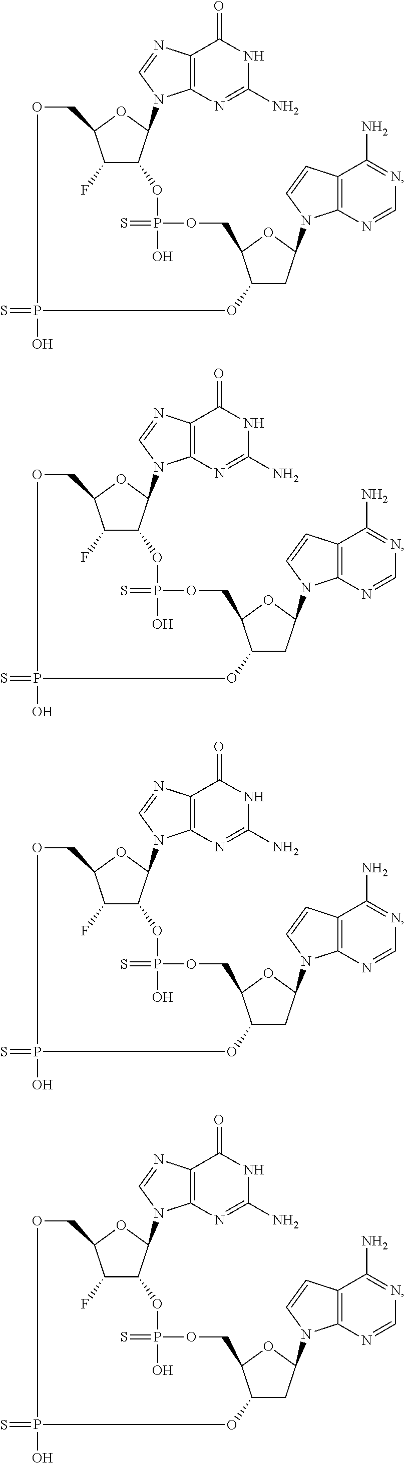 [SMILES: Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5ccc6c(N)ncnc65)O[C@@H]4COP(O)(=S)O[C@@H]2[C@@H]3F)c(=O)[nH]1] 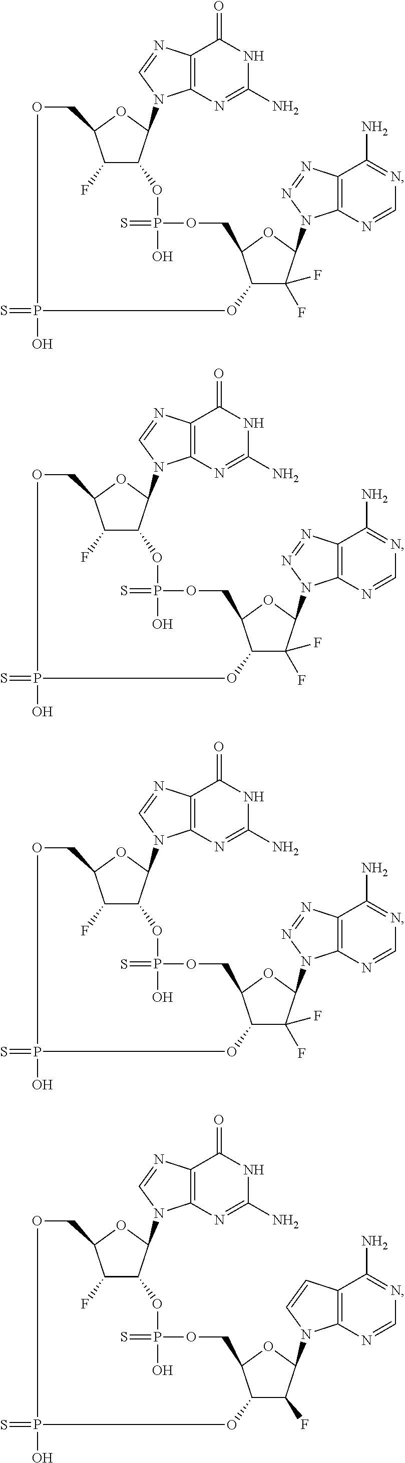 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1] 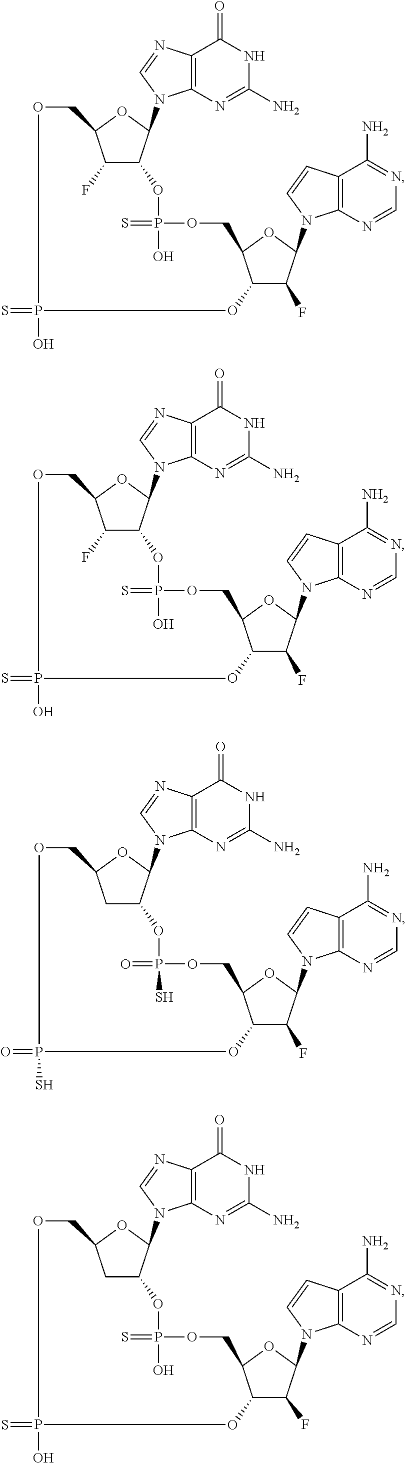 [SMILES: Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5ccc6c(N)ncnc65)O[C@@H]4CO[P@@](=O)(S)O[C@@H]2C3)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1] 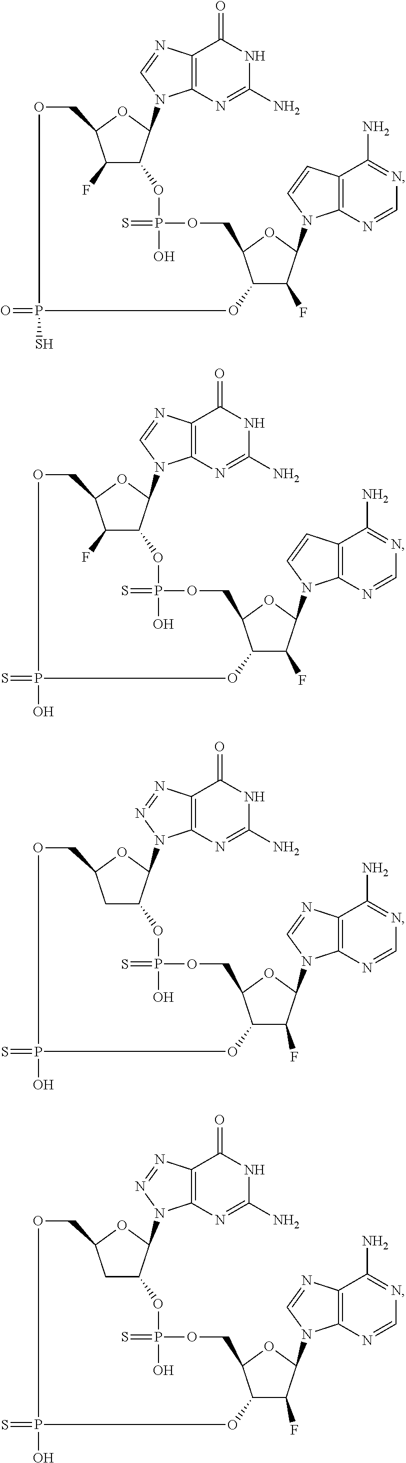 [SMILES: Nc1nc2c(ncn2[C@@H]2O[C@@H]3CO[P@@](=O)(S)O[C@H]4[C@H](F)[C@H](n5ccc6c(N)ncnc65)O[C@@H]4COP(O)(=S)O[C@@H]2[C@H]3F)c(=O)[nH]1 ; Nc1nc2c(ncn2[C@@H]2O[C@@H]3COP(O)(=S)O[C@H]4[C@H](F)[C@H](n5ccc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2C3)c(=O)[nH]1] 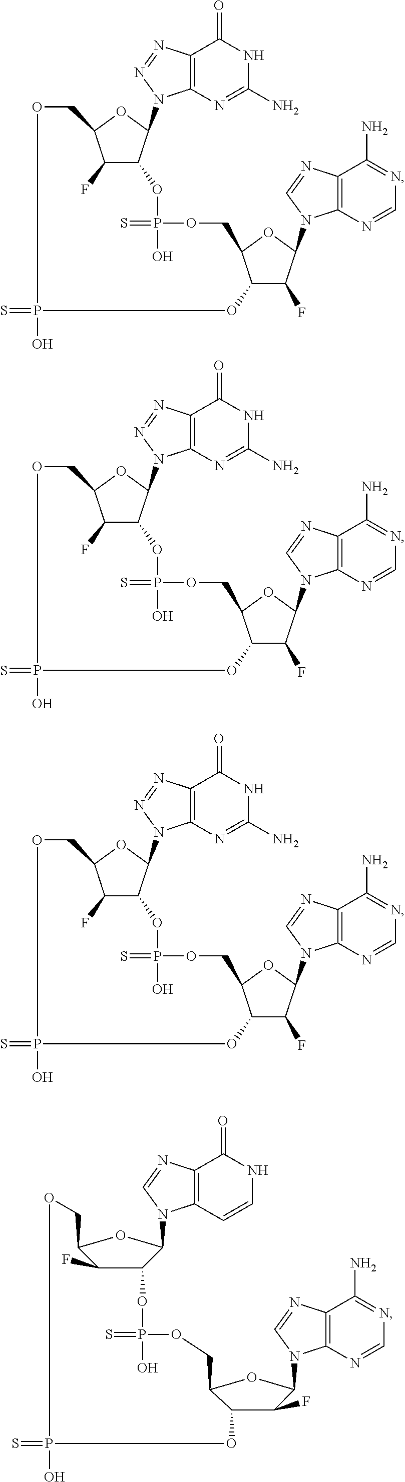 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@H]3F)c(=O)[nH]1 ; Nc1ncnc2c1ncn2[C@@H]1O[C@@H]2COP(O)(=S)O[C@@H]3[C@@H](F)[C@@H](COP(O)(=S)O[C@H]2[C@@H]1F)O[C@H]3n1cnc2c(=O)[nH]ccc21] 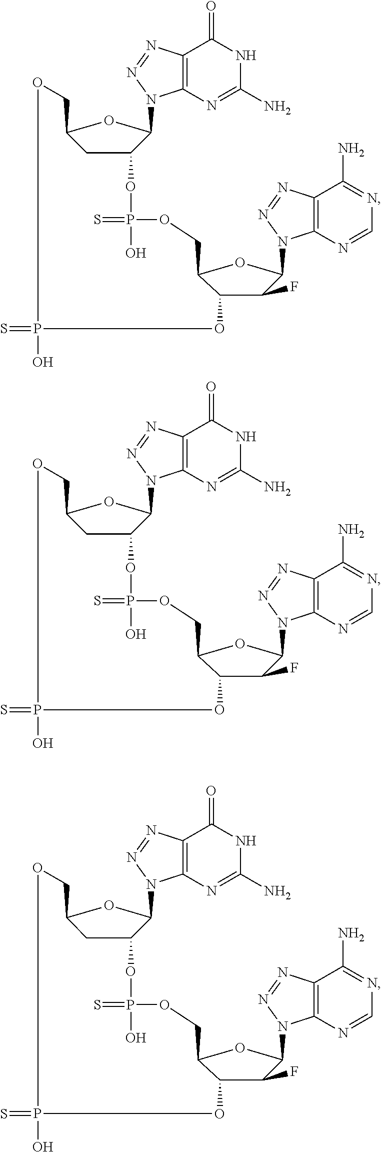 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] 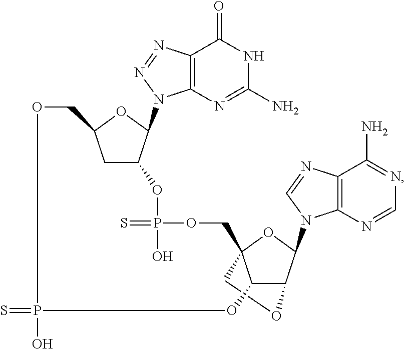 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1] 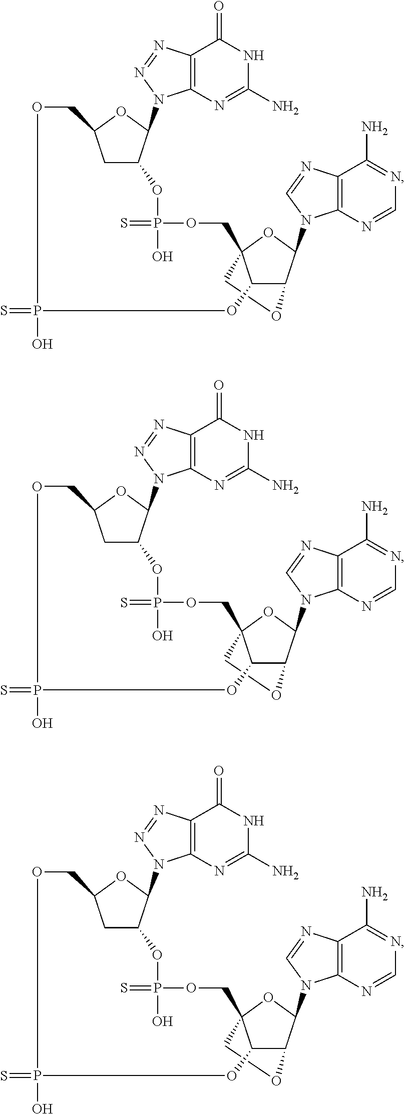 [SMILES: Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1 ; Nc1nc2c(nnn2[C@@H]2O[C@@H]3COP(O)(=S)O[C@H]4[C@H]5OC[C@]4(COP(O)(=S)O[C@@H]2C3)O[C@H]5n2cnc3c(N)ncnc32)c(=O)[nH]1] 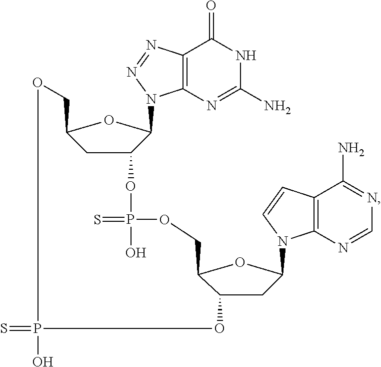 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1] 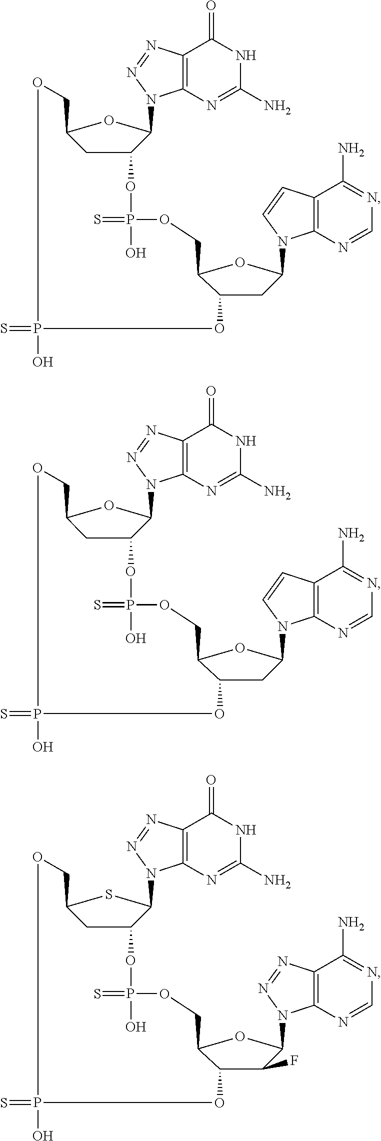 [SMILES: Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4C[C@H](n5ccc6c(N)ncnc65)O[C@@H]4COP(O)(=S)O[C@@H]2C3)c(=O)[nH]1 ; Nc1nc2c(nnn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] 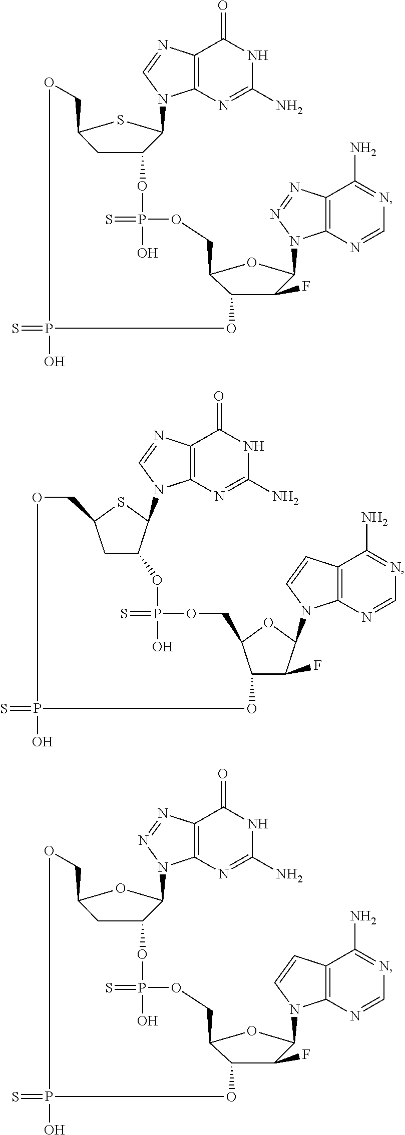 [SMILES: Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1 ; Nc1nc2c(ncn2[C@@H]2S[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1] 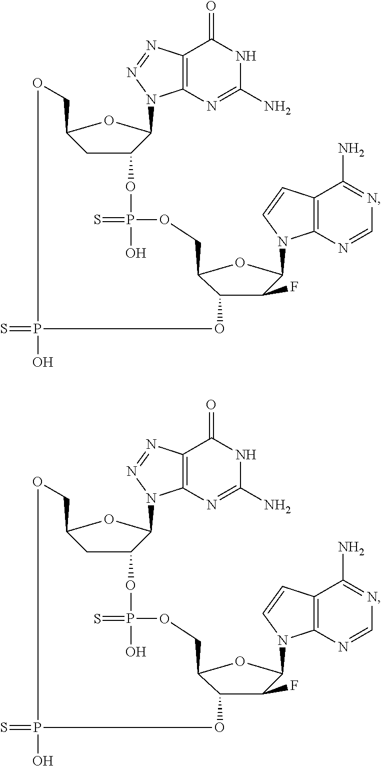 [SMILES: Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1 ; Nc1nc2c(nnn2[C@@H]2O[C@@H]3COP(O)(=S)O[C@H]4[C@H](F)[C@H](n5ccc6c(N)ncnc65)O[C@@H]4COP(O)(=S)O[C@@H]2C3)c(=O)[nH]1] 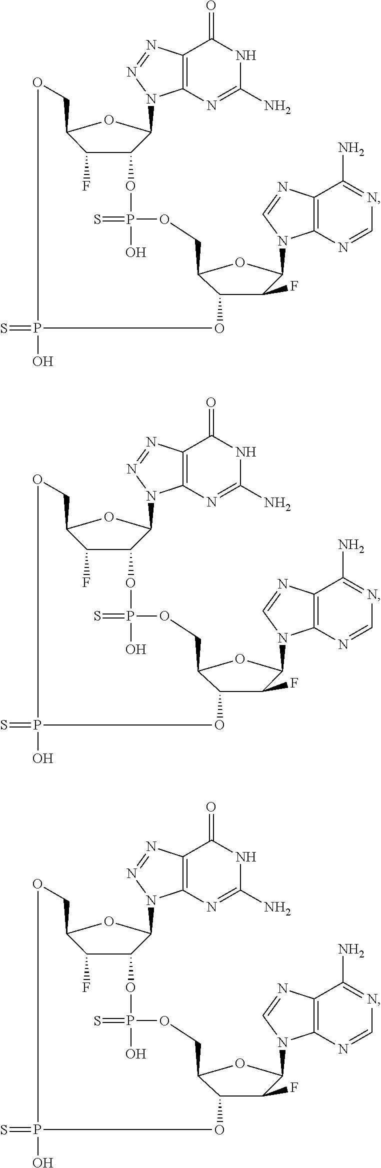 [SMILES: Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1] 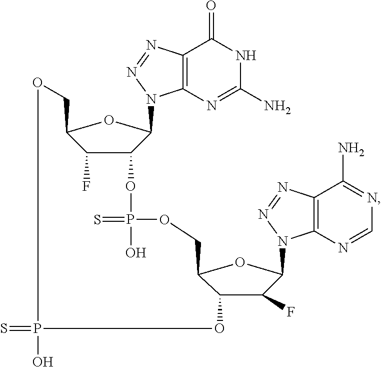 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] 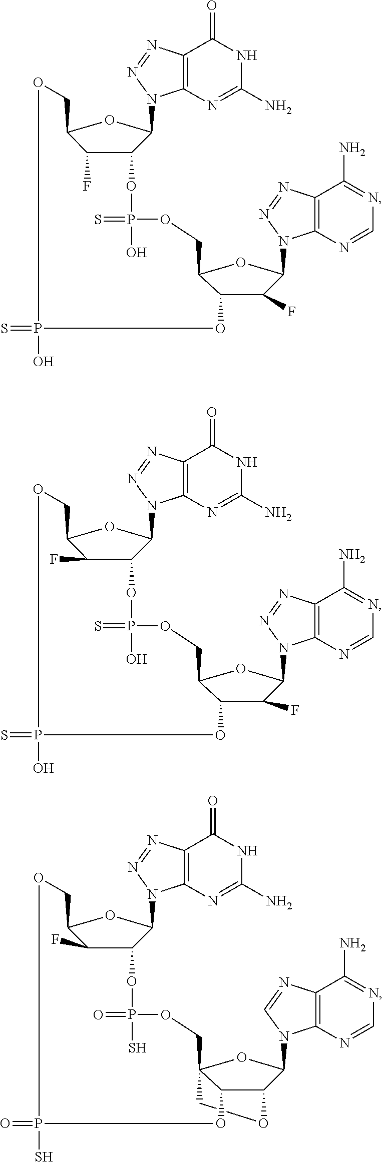 [SMILES: Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(O)(=S)O[C@H]4[C@H](F)[C@H](n5nnc6c(N)ncnc65)O[C@@H]4COP(O)(=S)O[C@@H]2[C@H]3F)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] 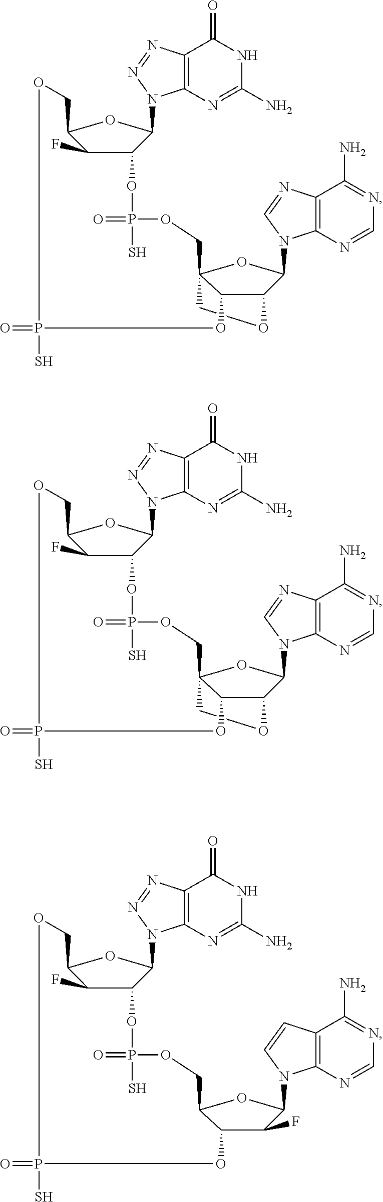 [SMILES: Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1] 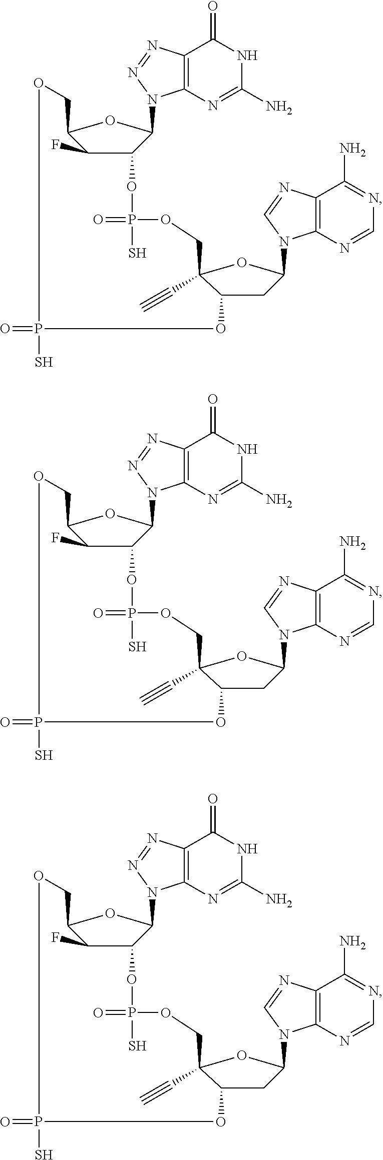 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21 ; C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21] 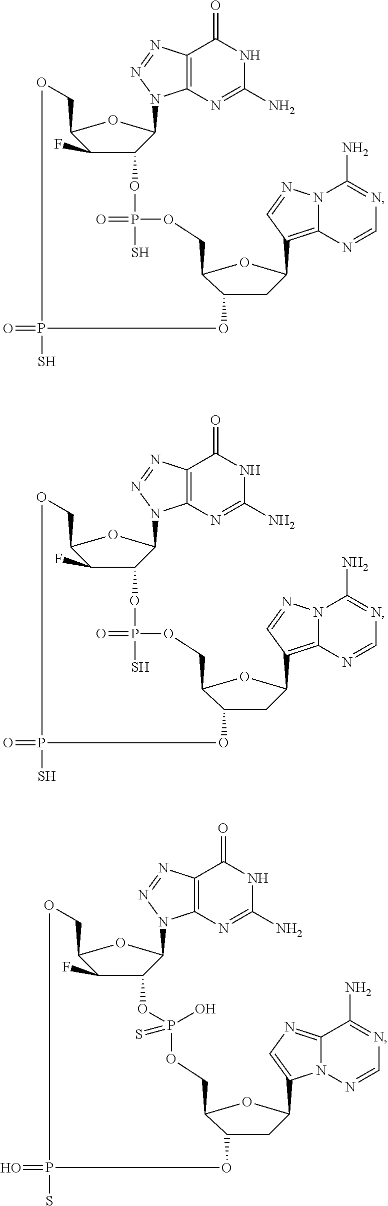 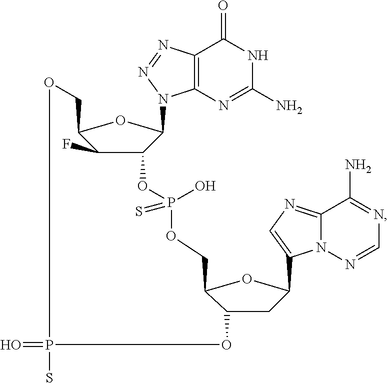 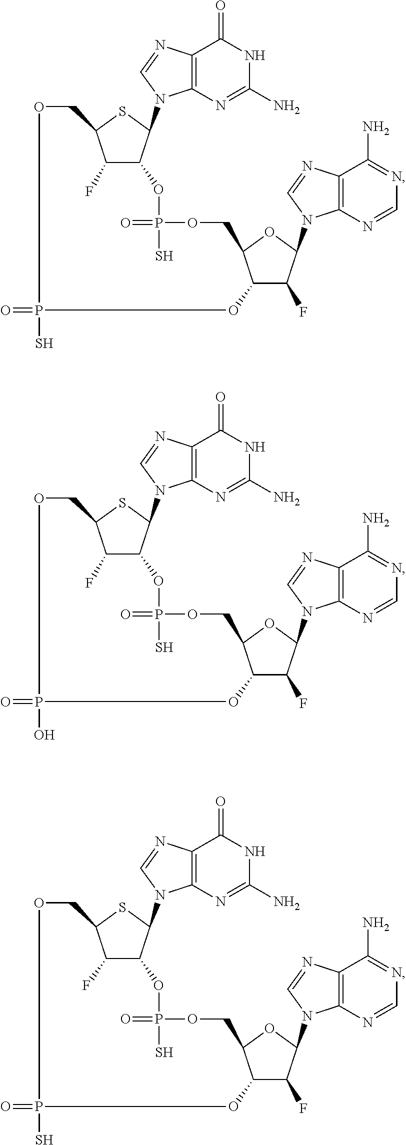 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(O)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] 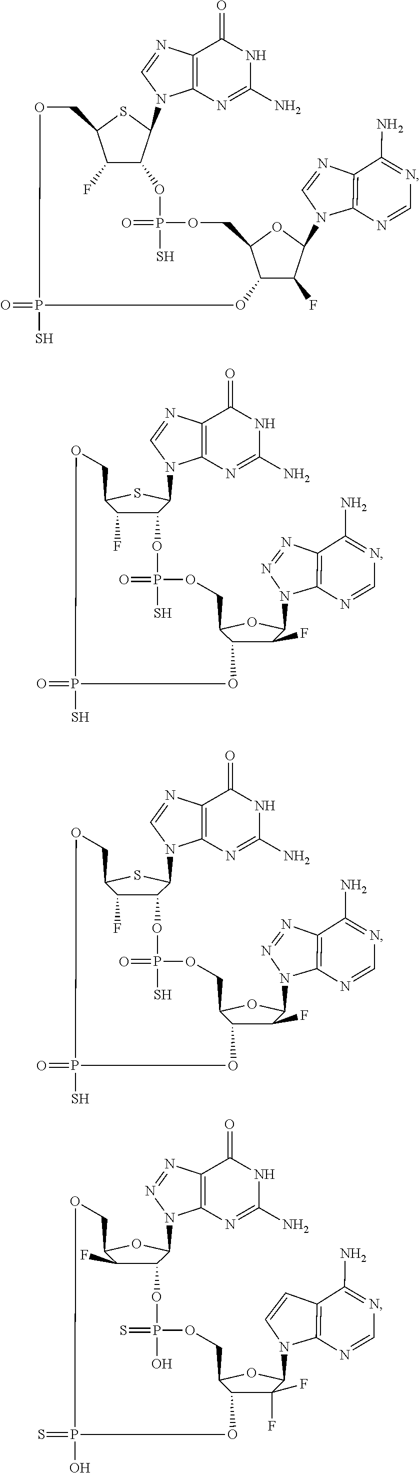 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2O[C@@H]3COP(O)(=S)O[C@@H]4[C@@H](COP(O)(=S)O[C@@H]2[C@H]3F)O[C@@H](n2ccc3c(N)ncnc32)C4(F)F)c(=O)[nH]1] 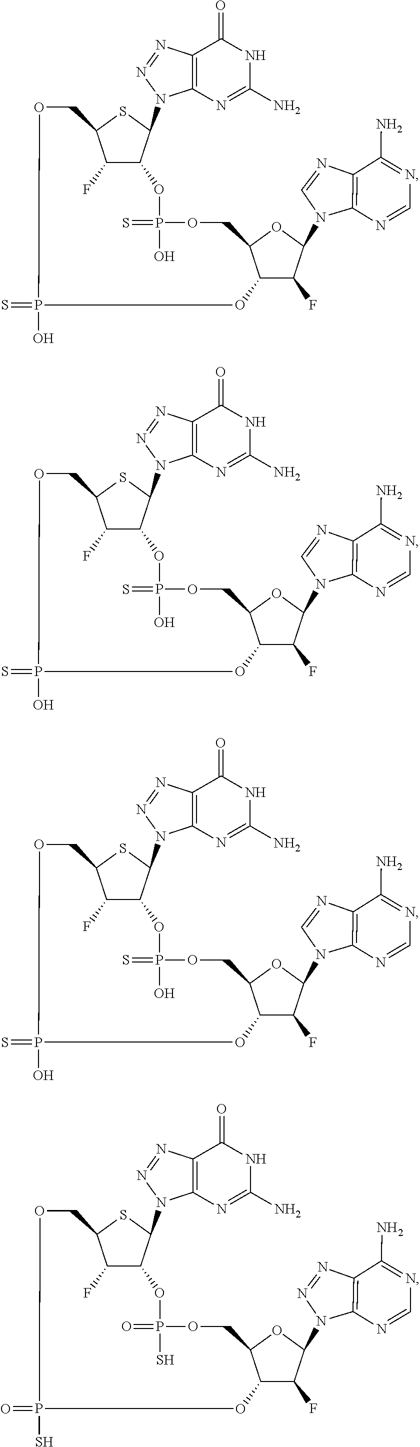 [SMILES: Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(O)(=S)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] 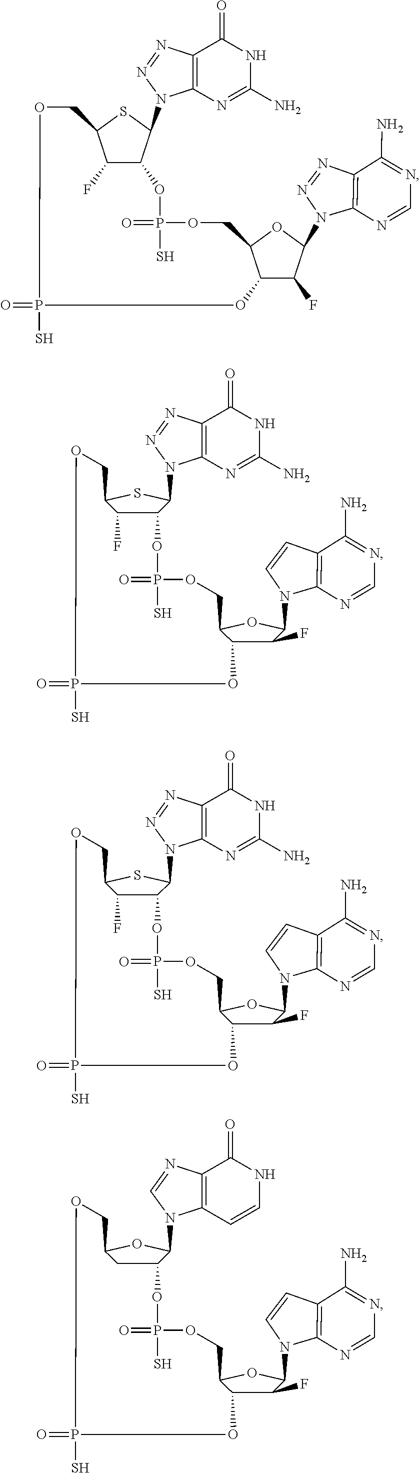 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(nnn2[C@@H]2S[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1ncnc2c1ccn2[C@@H]1O[C@@H]2COP(=O)(S)O[C@@H]3C[C@@H](COP(=O)(S)O[C@H]2[C@@H]1F)O[C@H]3n1cnc2c(=O)[nH]ccc21] 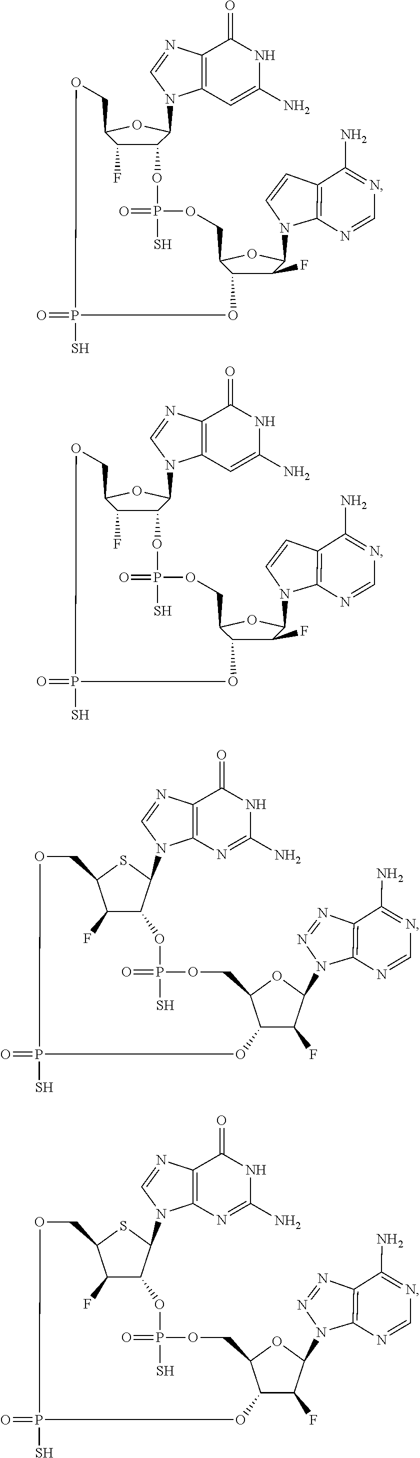 [SMILES: Nc1cc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1cc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] 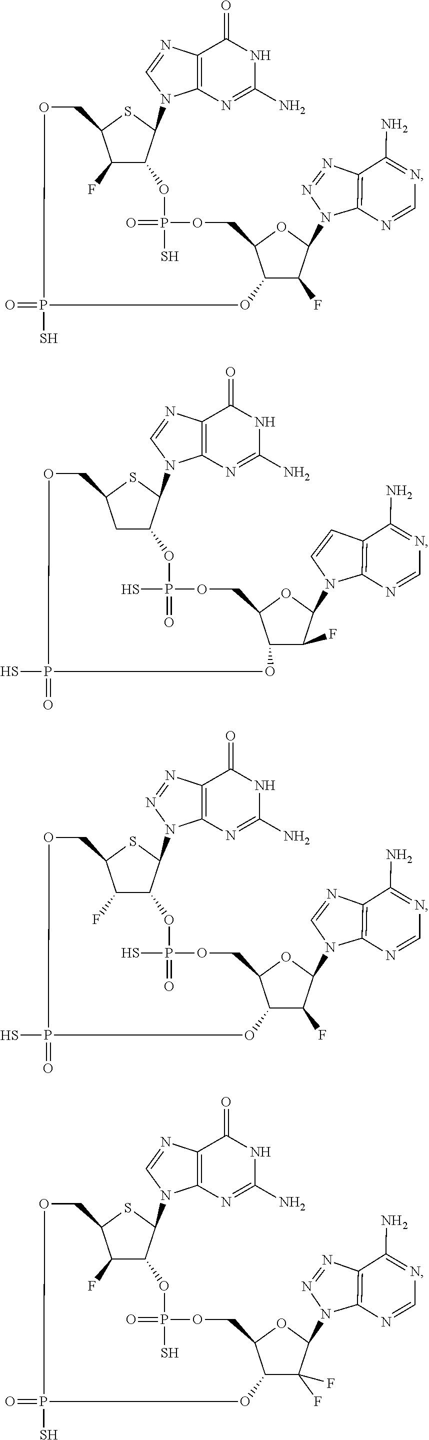 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1 ; Nc1nc2c(ncn2[C@@H]2S[C@@H]3COP(=O)(S)O[C@H]4[C@H](F)[C@H](n5ccc6c(N)ncnc65)O[C@@H]4COP(=O)(S)O[C@@H]2C3)c(=O)[nH]1 ; Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1] 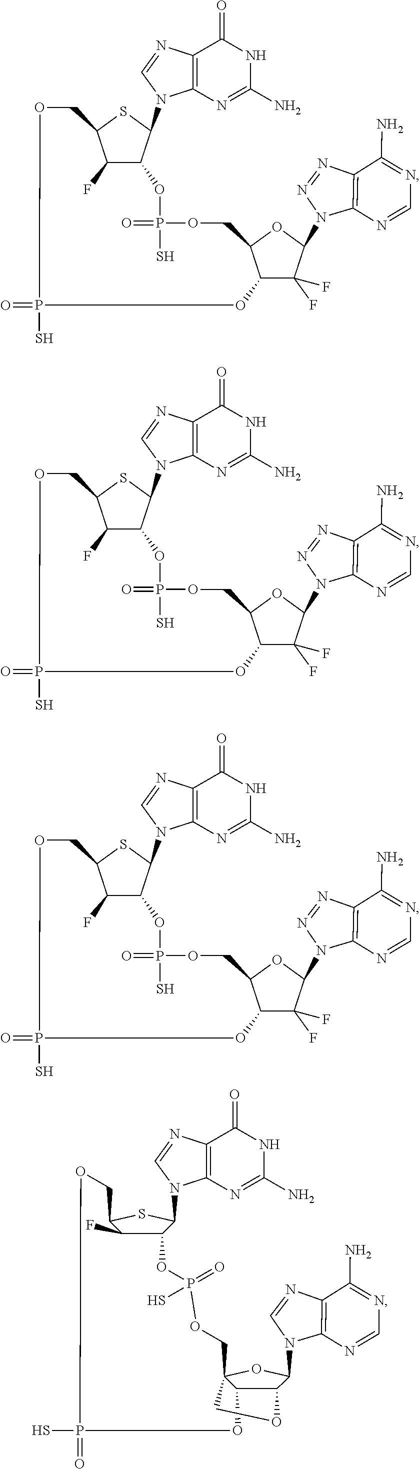 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] 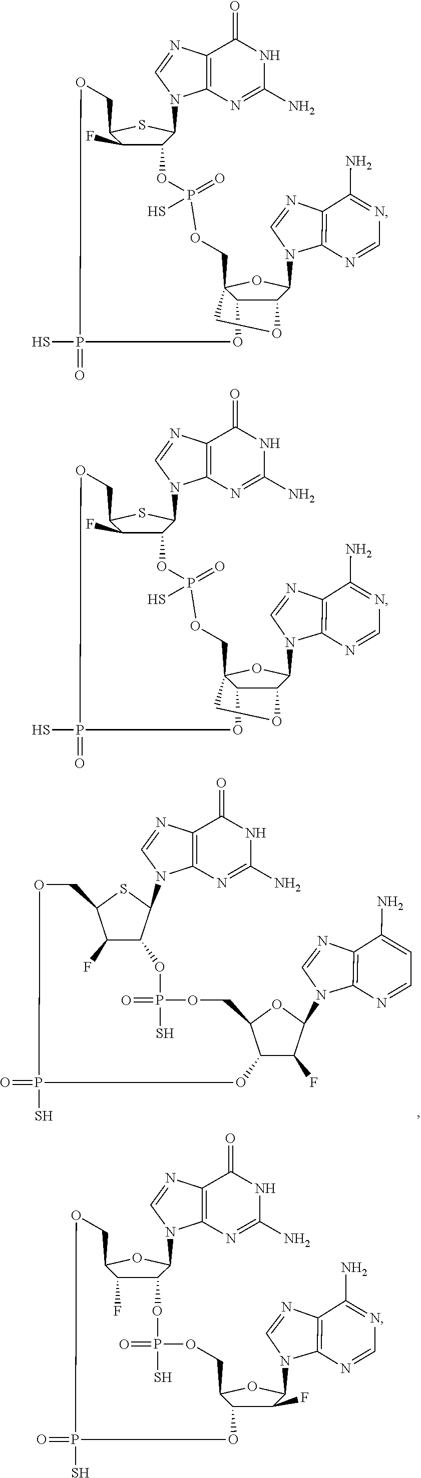 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1 ; Nc1nc2c(ncn2[C@@H]2S[C@@H]3COP(=O)(S)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] 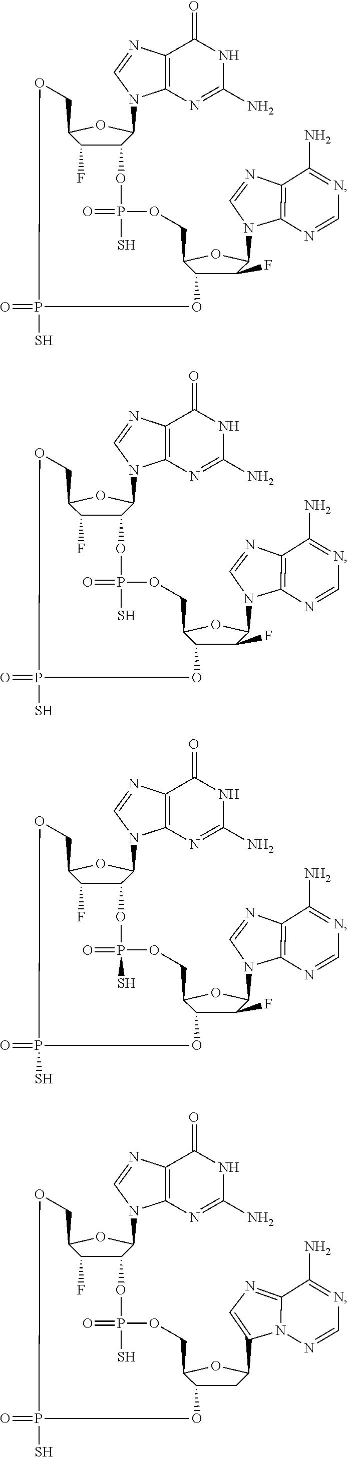 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1] 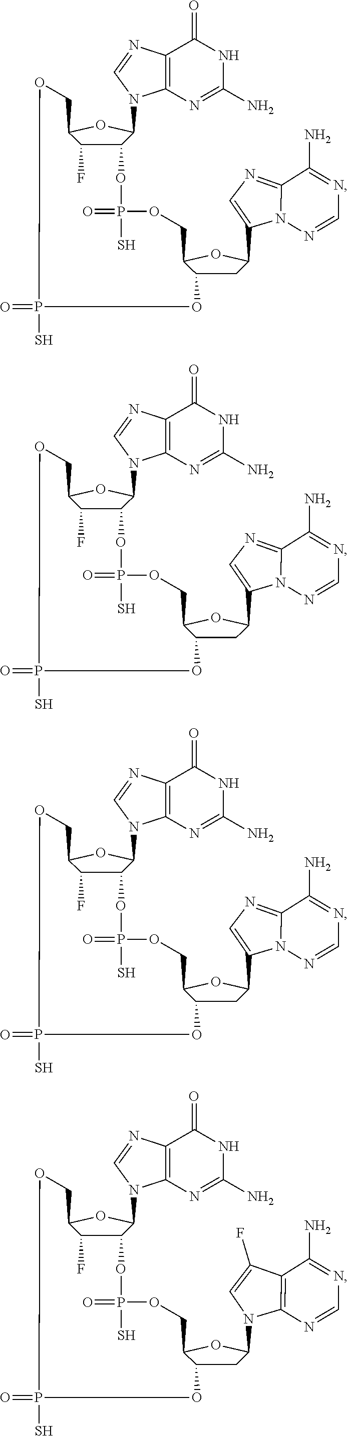 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1] 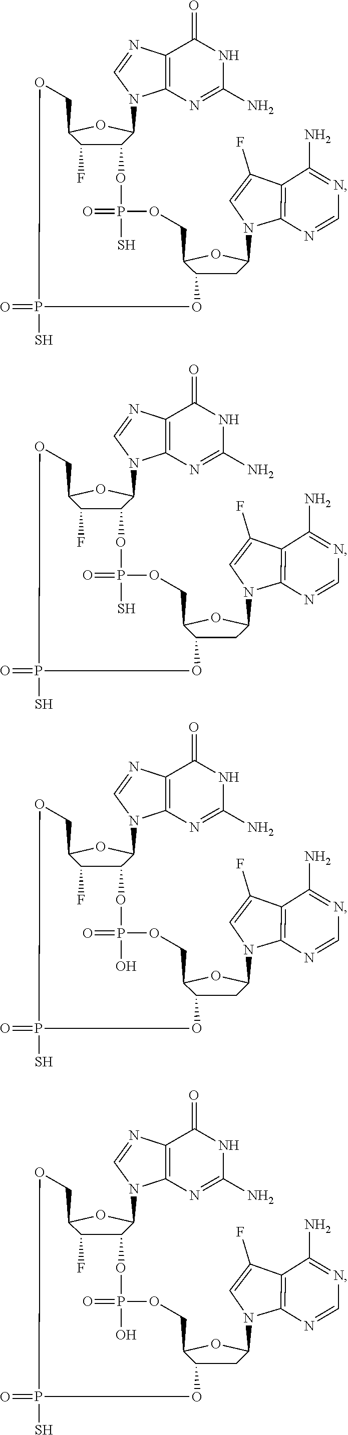 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(O)O[C@@H]2[C@@H]3F)c(=O)[nH]1 ; Nc1nc2c(ncn2[C@@H]2O[C@@H]3COP(=O)(S)O[C@H]4C[C@H](n5cc(F)c6c(N)ncnc65)O[C@@H]4COP(=O)(O)O[C@@H]2[C@@H]3F)c(=O)[nH]1] 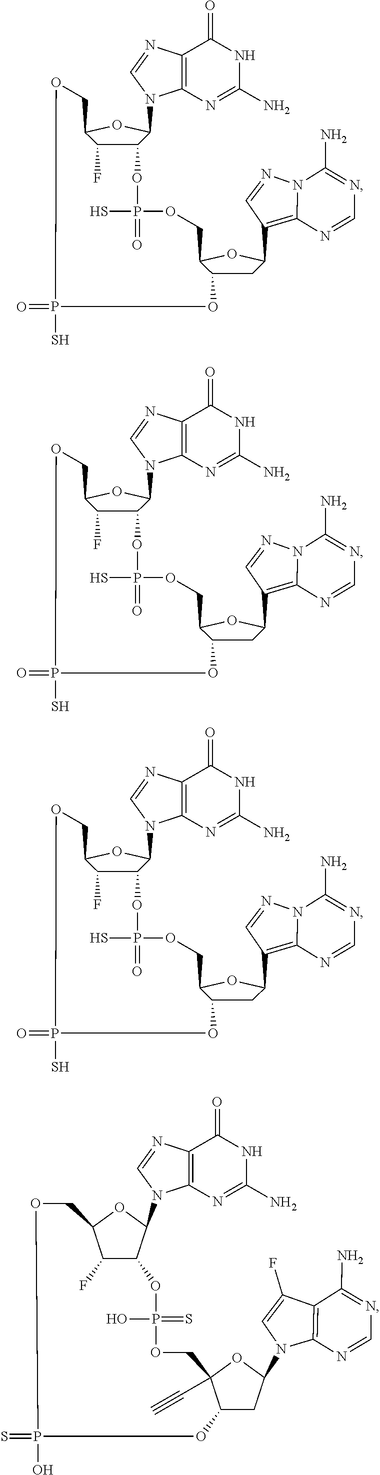 [SMILES: Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1 ; C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21] 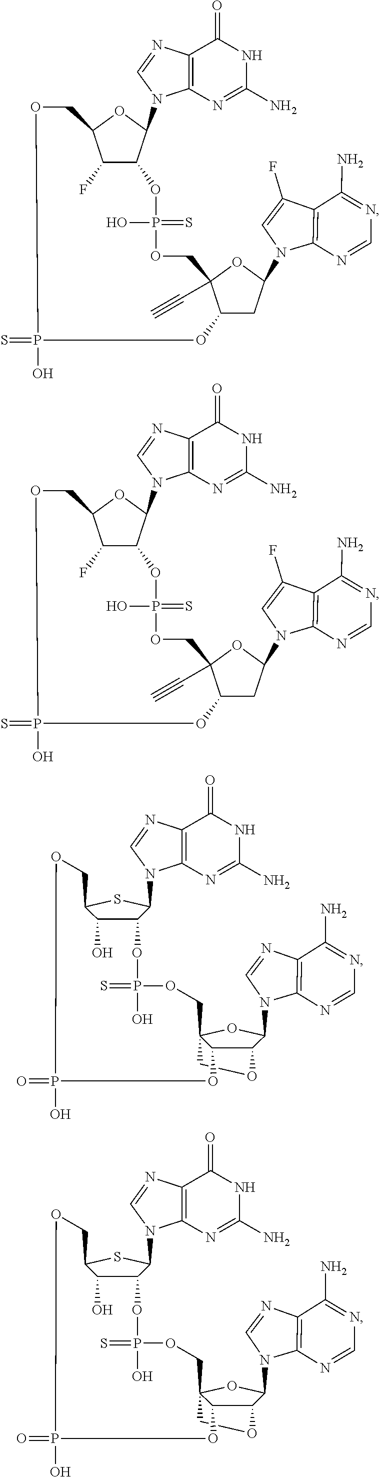 [SMILES: C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; C#C[C@@]12COP(O)(=S)O[C@@H]3[C@H](F)[C@@H](COP(O)(=S)O[C@H]1C[C@H](n1cc(F)c4c(N)ncnc41)O2)O[C@H]3n1cnc2c(=O)[nH]c(N)nc21 ; Nc1nc2c(ncn2[C@@H]2S[C@@H]3COP(=O)(O)O[C@H]4[C@H]5OC[C@]4(COP(O)(=S)O[C@@H]2[C@@H]3O)O[C@H]5n2cnc3c(N)ncnc32)c(=O)[nH]1 ; Nc1nc2c(ncn2[C@@H]2S[C@@H]3COP(=O)(O)O[C@H]4[C@H]5OC[C@]4(COP(O)(=S)O[C@@H]2[C@@H]3O)O[C@H]5n2cnc3c(N)ncnc32)c(=O)[nH]1] 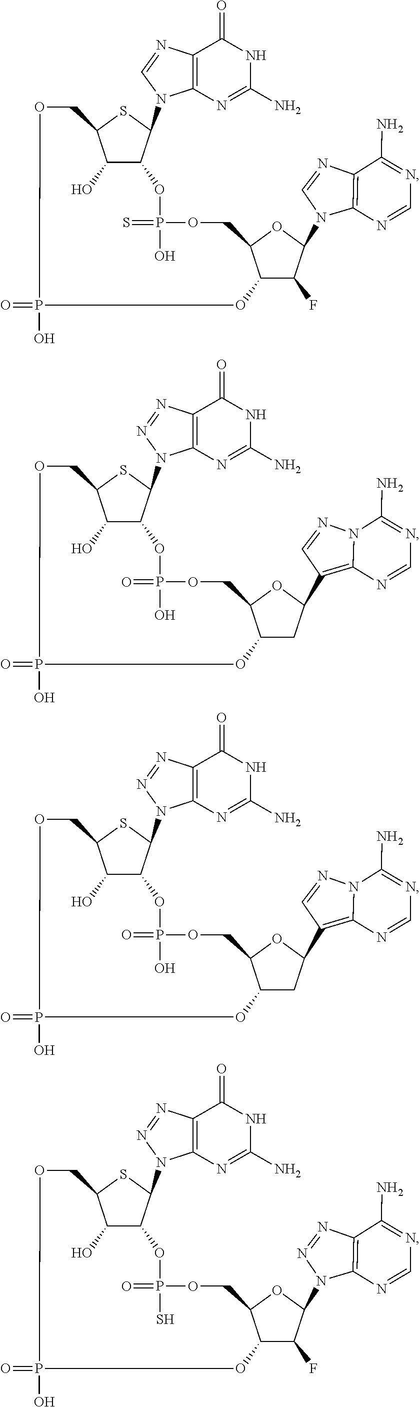 [SMILES: Nc1nc2c(ncn2[C@@H]2S[C@@H]3COP(=O)(O)O[C@H]4[C@H](F)[C@H](n5cnc6c(N)ncnc65)O[C@@H]4COP(O)(=S)O[C@@H]2[C@@H]3O)c(=O)[nH]1 ; Nc1nc2c(nnn2[C@@H]2S[C@@H]3COP(=O)(O)O[C@H]4C[C@H](c5cnn6c(N)ncnc56)O[C@@H]4COP(=O)(O)O[C@@H]2[C@@H]3O)c(=O)[nH]1 ; Nc1nc2c(nnn2[C@@H]2S[C@@H]3COP(=O)(O)O[C@H]4C[C@H](c5cnn6c(N)ncnc56)O[C@@H]4COP(=O)(O)O[C@@H]2[C@@H]3O)c(=O)[nH]1 ; Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1] 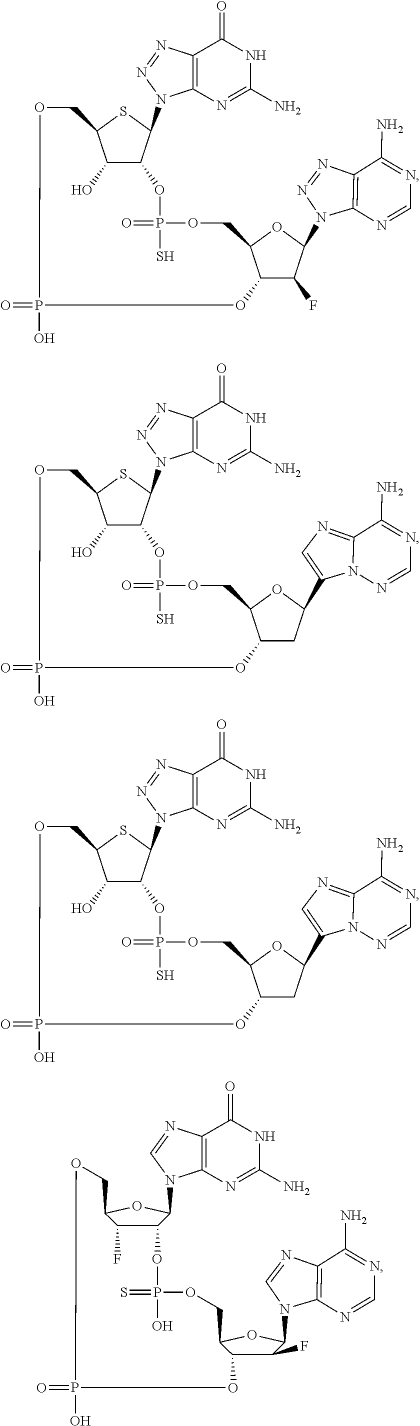 [SMILES: Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1 ; Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1] 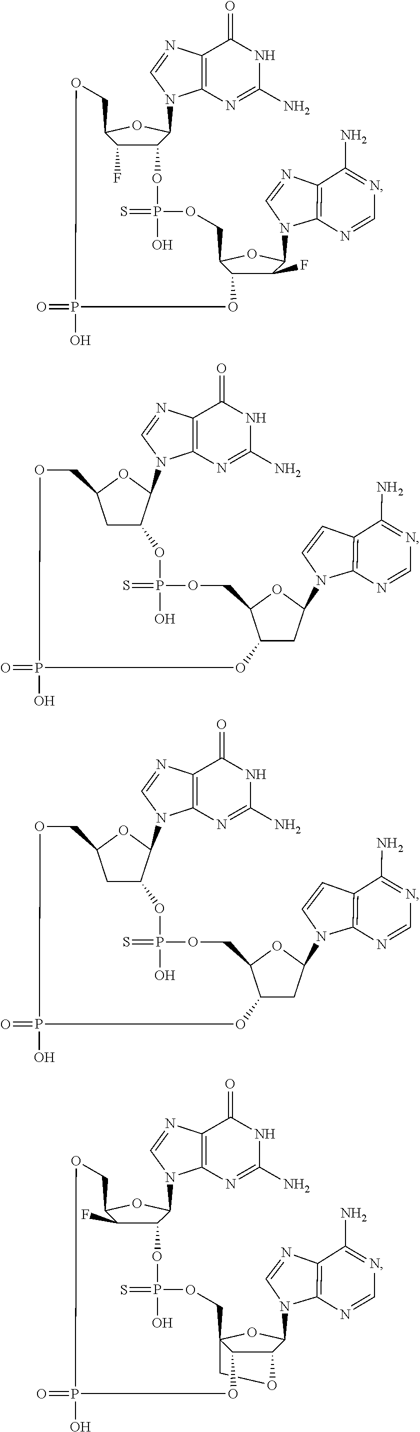 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(O)O[C@H]4C[C@H](n5ccc6c(N)ncnc65)O[C@@H]4COP(O)(=S)O[C@@H]2C3)c(=O)[nH]1 ; Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1] 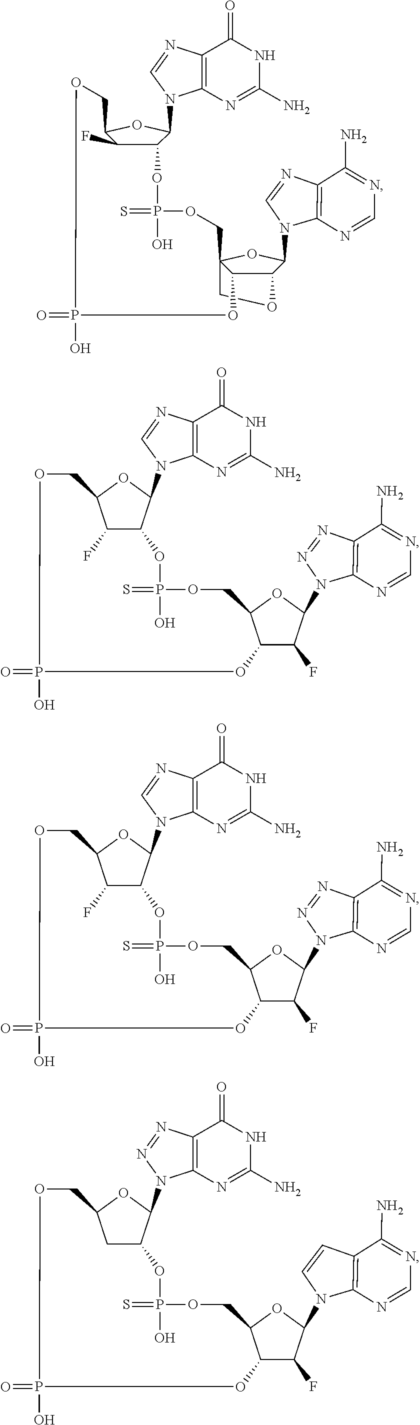 [SMILES: Nc1nc2c(ncn2[C@@H]2O[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5nnc6c(N)ncnc65)O[C@@H]4COP(O)(=S)O[C@@H]2[C@@H]3F)c(=O)[nH]1 ; Nc1nc2c(nnn2[C@@H]2O[C@@H]3COP(=O)(O)O[C@H]4[C@H](F)[C@H](n5ccc6c(N)ncnc65)O[C@@H]4COP(O)(=S)O[C@@H]2C3)c(=O)[nH]1] 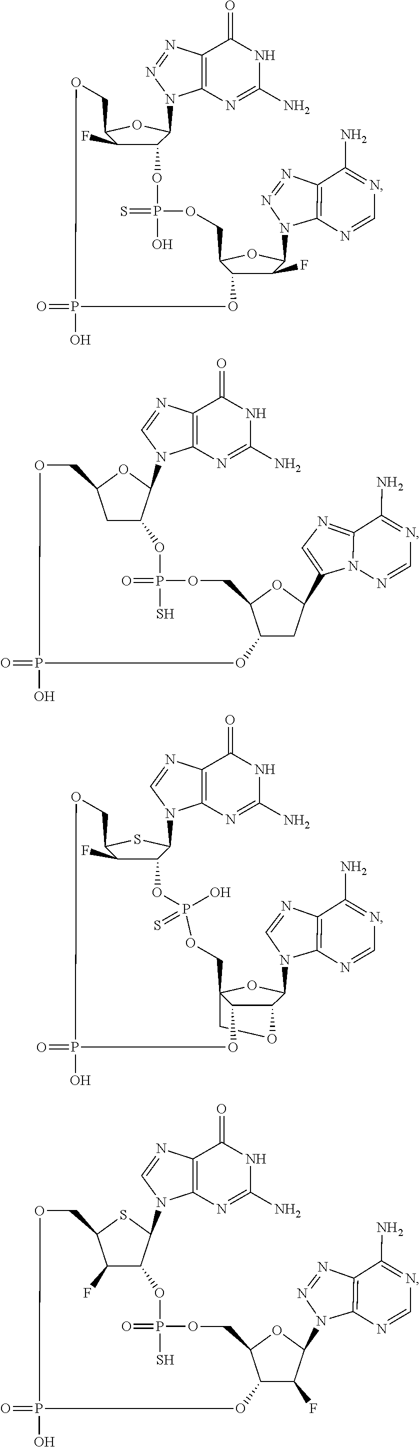 [SMILES: Nc1nc2c(nnn2[C@@H]2O[C@@H]3COP(=O)(O)O[C@H]4[C@H](F)[C@H](n5nnc6c(N)ncnc65)O[C@@H]4COP(O)(=S)O[C@@H]2[C@H]3F)c(=O)[nH]1 ; Nc1nc2c(ncn2[C@@H]2O[C@@H]3COP(=O)(O)O[C@H]4C[C@H](c5cnc6c(N)ncnn56)O[C@@H]4COP(=O)(S)O[C@@H]2C3)c(=O)[nH]1 ; Nc1nc2c(ncn2[C@@H]2S[C@@H]3COP(=O)(O)O[C@H]4[C@H]5OC[C@]4(COP(O)(=S)O[C@@H]2[C@H]3F)O[C@H]5n2cnc3c(N)ncnc32)c(=O)[nH]1 ; Nc1nc2c(ncn2[C@@H]2S[C@@H]3COP(=O)(O)O[C@H]4[C@H](F)[C@H](n5nnc6c(N)ncnc65)O[C@@H]4COP(=O)(S)O[C@@H]2[C@H]3F)c(=O)[nH]1] 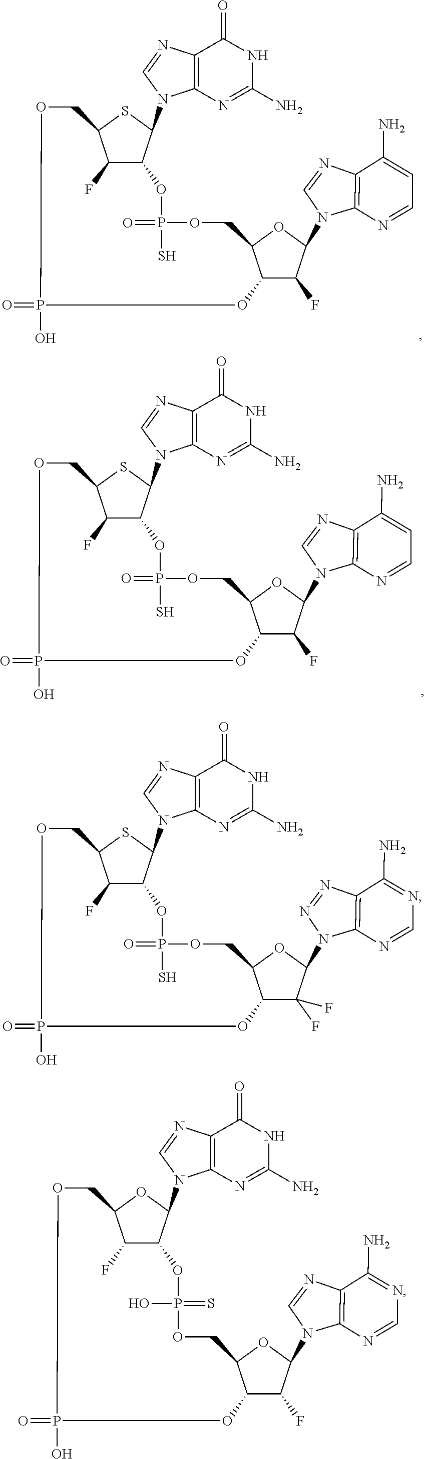 [SMILES: Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1 ; Nc1nc2c(ncn2[C@@H]2S[C@@H]3COP(=O)(O)O[C@@H]4[C@@H](COP(=O)(S)O[C@@H]2[C@H]3F)O[C@@H](n2nnc3c(N)ncnc32)C4(F)F)c(=O)[nH]1 ; Nc1nc2c(ncn2[C@@H]2O[C@@H]3COP(=O)(O)O[C@H]4[C@@H](F)[C@H](n5cnc6c(N)ncnc65)O[C@@H]4COP(O)(=S)O[C@@H]2[C@@H]3F)c(=O)[nH]1] 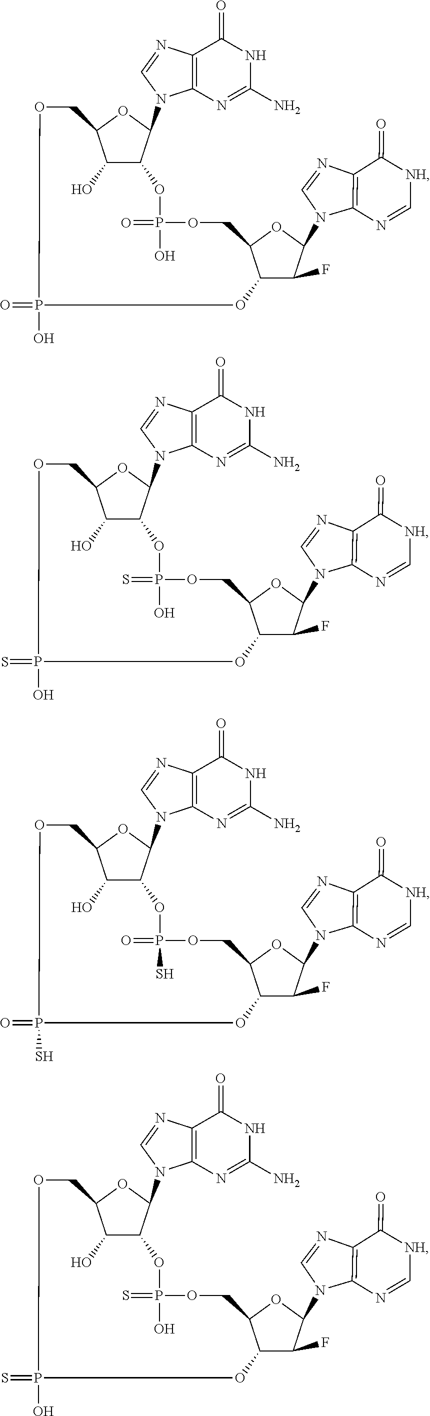 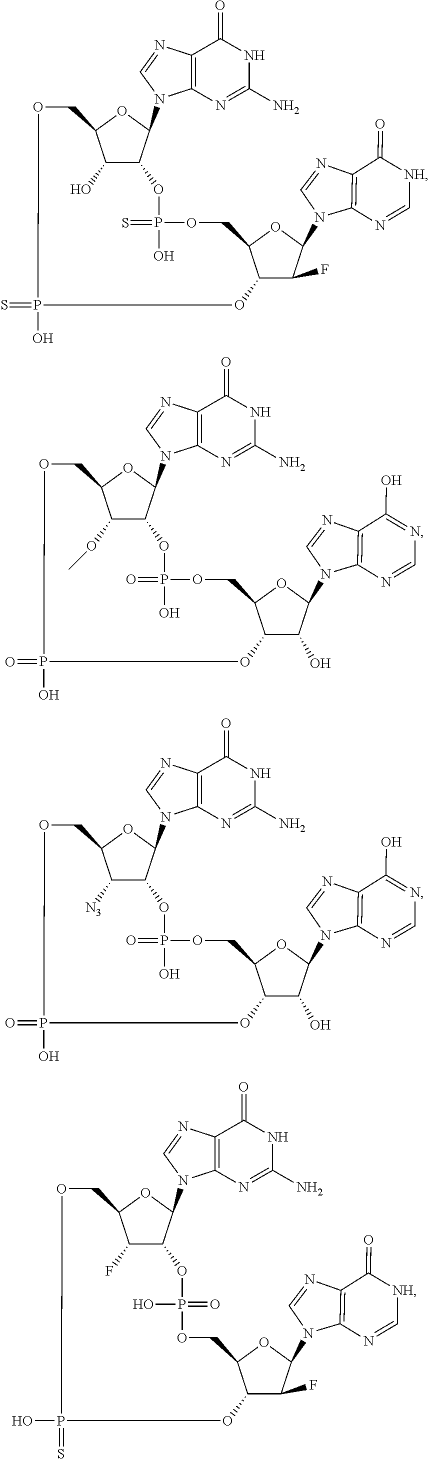 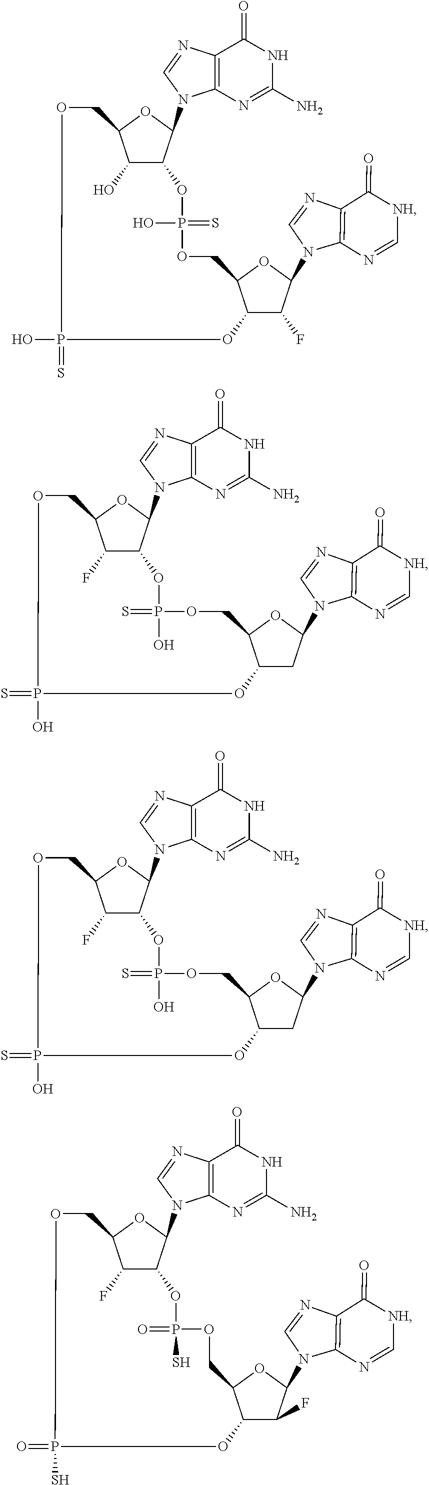 [SMILES: Nc1nc2c(ncn2[C@@H]2O[C@@H]3COP(O)(=S)O[C@H]4[C@@H](F)[C@H](n5cnc6c(=O)[nH]cnc65)O[C@@H]4COP(O)(=S)O[C@@H]2[C@@H]3O)c(=O)[nH]1 ; Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4C[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2[C@@H]3F)c(=O)[nH]1] 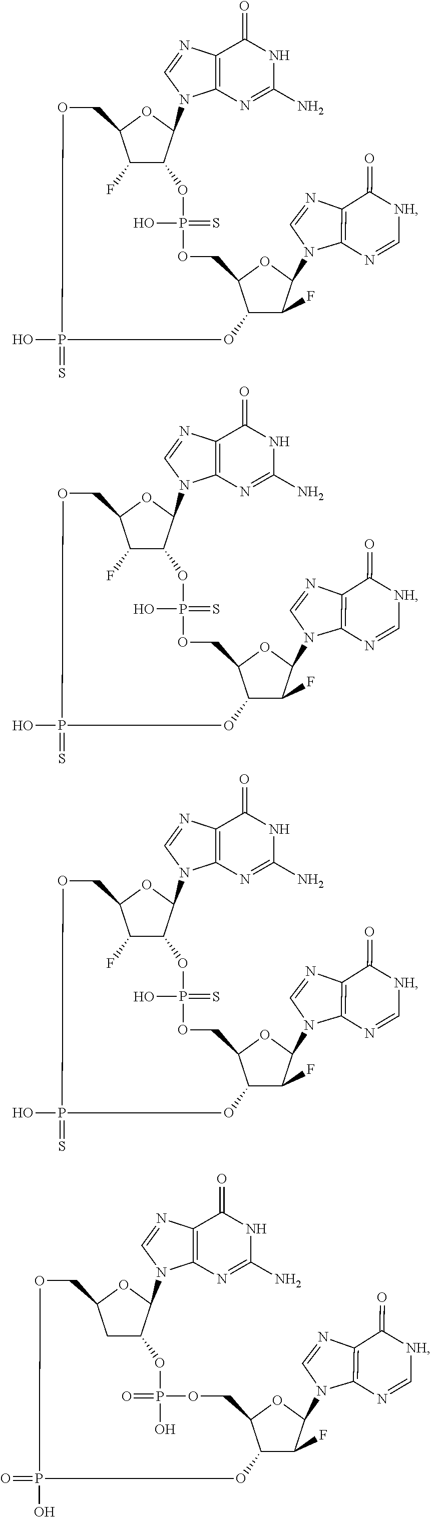 [SMILES: Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(O)(=S)O[C@H]4[C@H](F)[C@H](n5cnc6c(=O)[nH]cnc65)O[C@@H]4COP(O)(=S)O[C@@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2C3)c(=O)[nH]1] 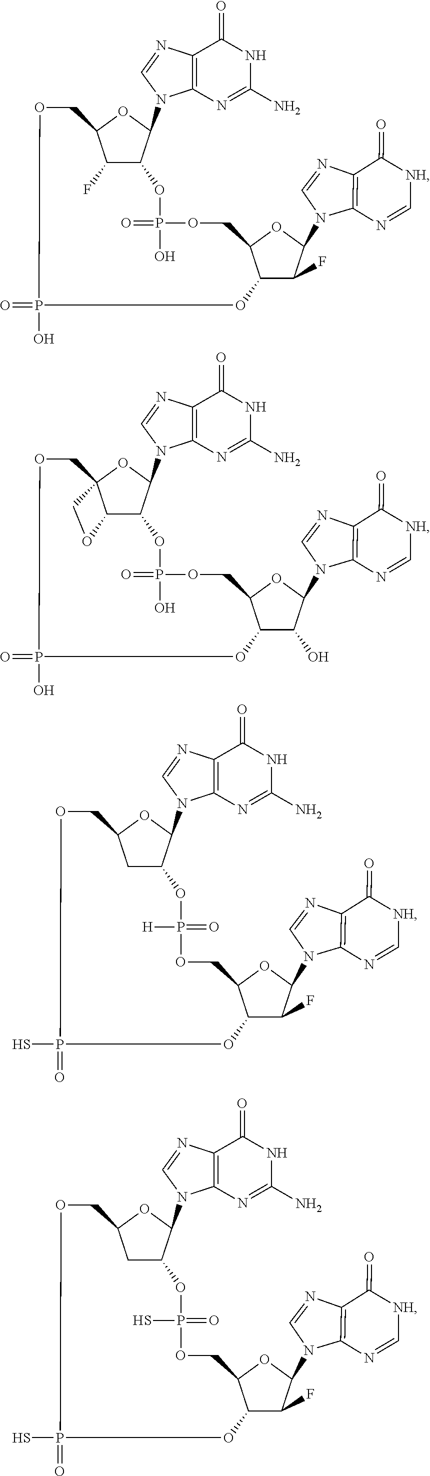 [SMILES: Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2[C@@H]3F)c(=O)[nH]1 ; Nc1nc2c(ncn2[C@@H]2O[C@@]34CO[C@H]3[C@H]2OP(=O)(O)OC[C@H]2O[C@@H](n3cnc5c(=O)[nH]cnc53)[C@H](O)[C@@H]2OP(=O)(O)OC4)c(=O)[nH]1 ; Nc1nc2c(ncn2[C@@H]2O[C@@H]3COP(=O)(S)O[C@H]4[C@H](F)[C@H](n5cnc6c(=O)[nH]cnc65)O[C@@H]4CO[PH](=O)O[C@@H]2C3)c(=O)[nH]1 ; Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2C3)c(=O)[nH]1] 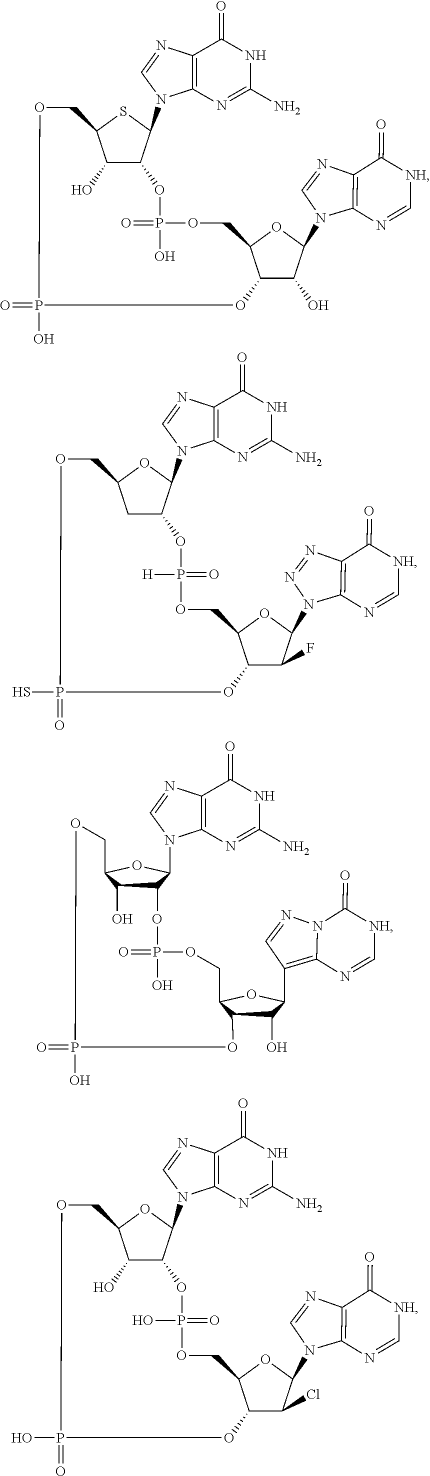 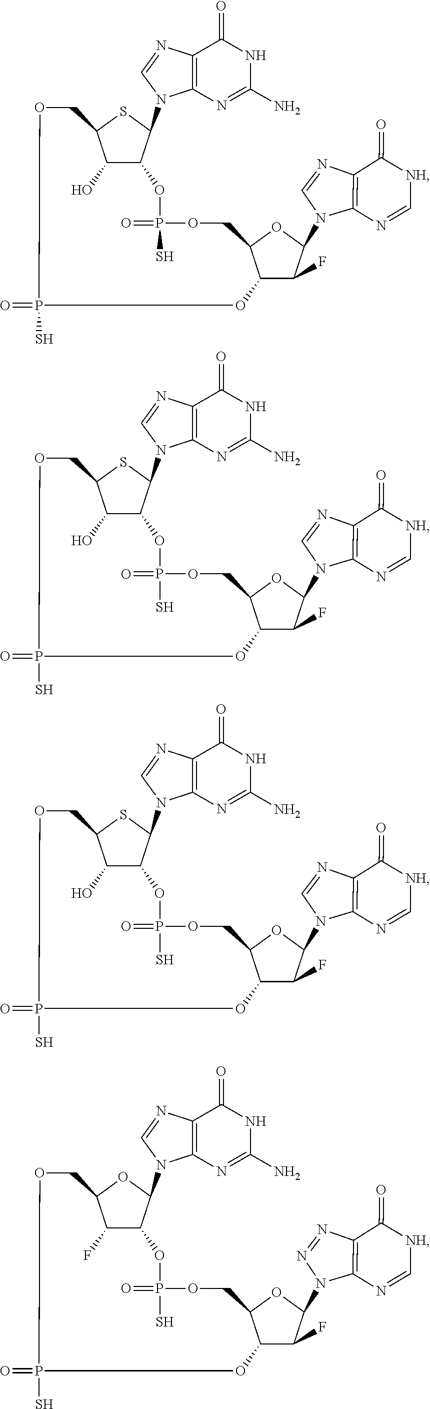 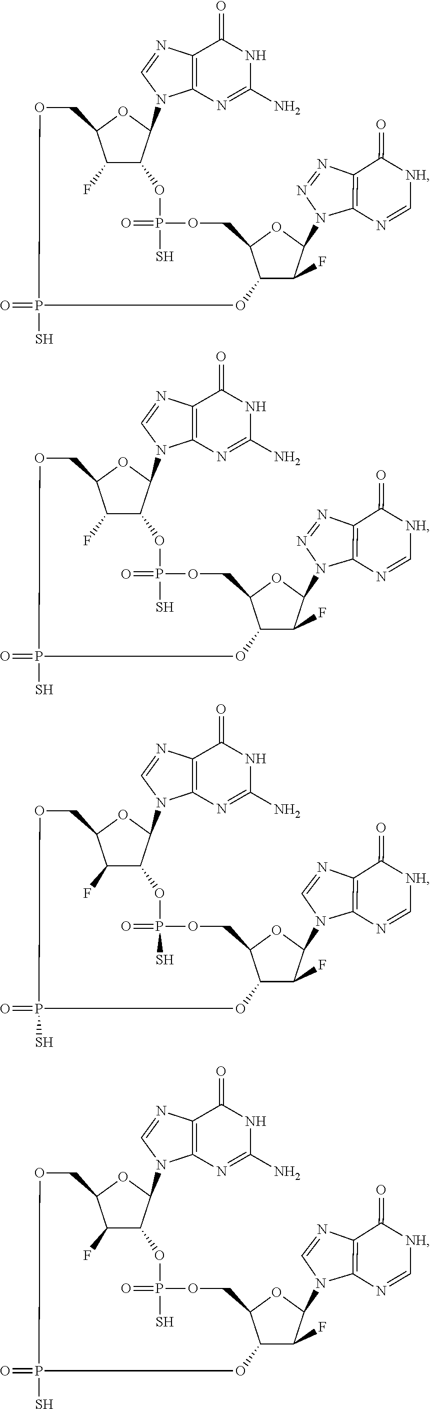 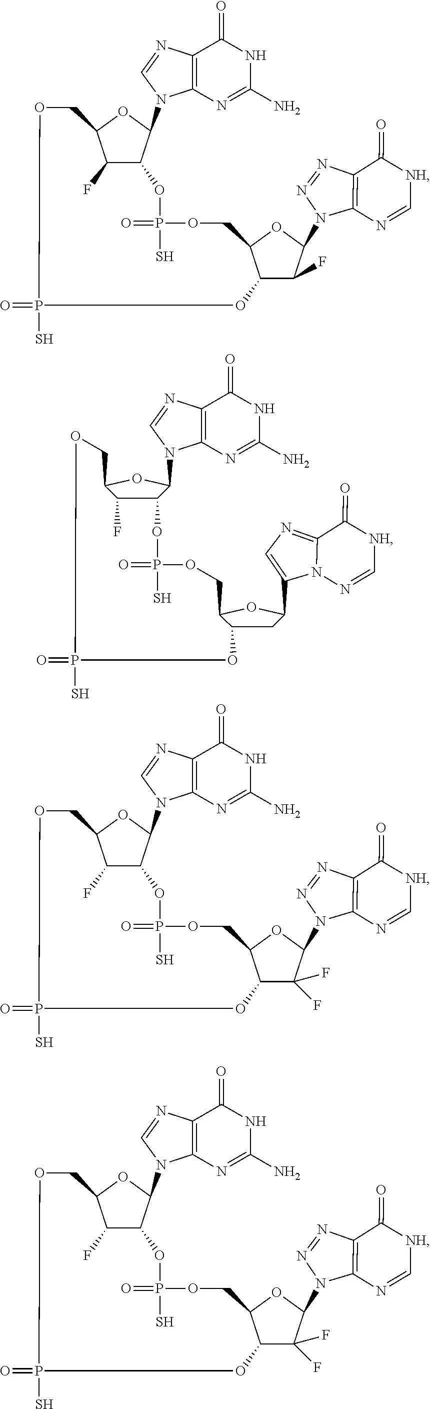 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@H]3F)c(=O)[nH]1 ; Nc1nc2c(ncn2[C@@H]2O[C@@H]3COP(=O)(S)O[C@H]4C[C@H](c5cnc6c(=O)[nH]cnn56)O[C@@H]4COP(=O)(S)O[C@@H]2[C@@H]3F)c(=O)[nH]1 ; Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1 ; Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1] 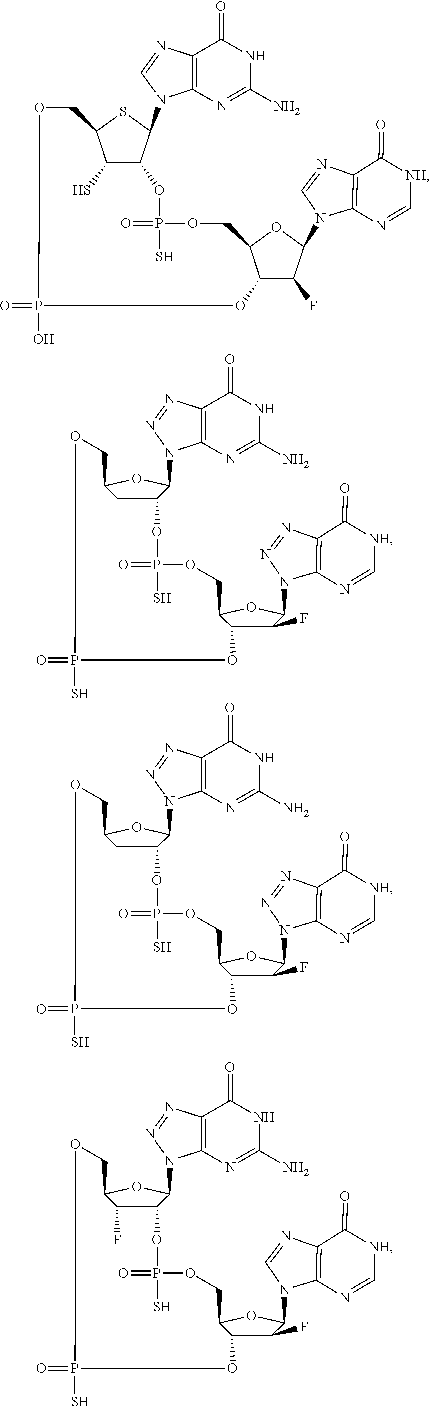 [SMILES: Nc1nc2c(ncn2[C@@H]2S[C@@H]3COP(=O)(O)O[C@H]4[C@H](F)[C@H](n5cnc6c(=O)[nH]cnc65)O[C@@H]4COP(=O)(S)O[C@@H]2[C@@H]3S)c(=O)[nH]1 ; Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1 ; Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1 ; Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] 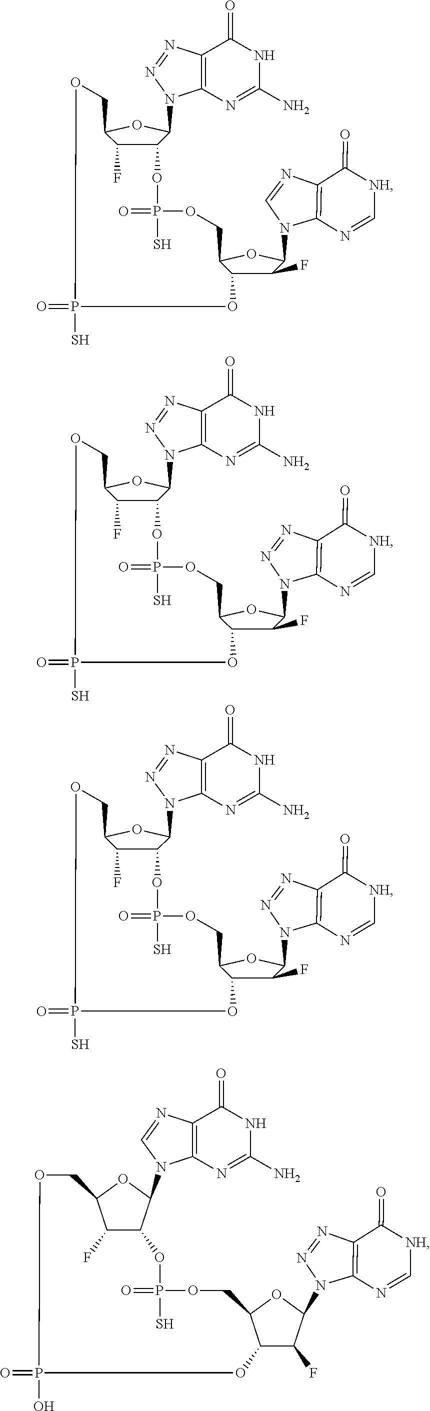 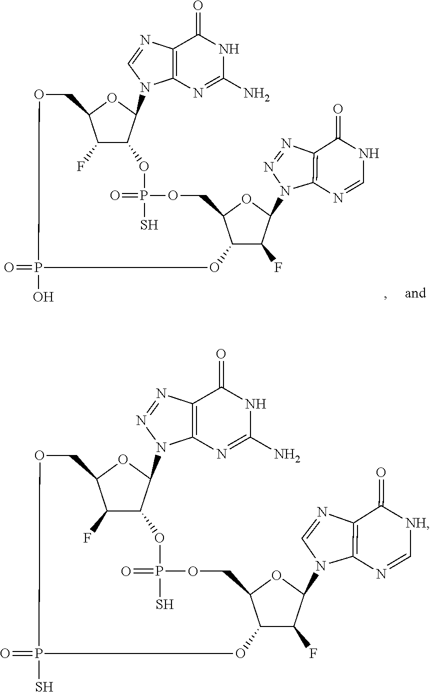 [SMILES: Nc1nc2c(ncn2[C@@H]2O[C@@H]3COP(=O)(O)O[C@H]4[C@H](F)[C@H](n5nnc6c(=O)[nH]cnc65)O[C@@H]4COP(=O)(S)O[C@@H]2[C@@H]3F)c(=O)[nH]1 ; Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1]
and pharmaceutically acceptable salts thereof;
(b) a pharmaceutically acceptable aqueous carrier;
(c) a pharmaceutically acceptable tonicity modifier;
(d) a pharmaceutically acceptable buffer;
(e) a pharmaceutically acceptable antioxidant; and
(f) a pharmaceutically acceptable metal chelator;
wherein said pharmaceutical composition has a pH from about 6 to about 7.5.

17. The pharmaceutical composition according to claim 16, wherein
(a) the compound is present in an amount of from about 0.1 to about 6.0 mg/mL;
(b) the pharmaceutically acceptable aqueous carrier is water;
(c) the pharmaceutically acceptable tonicity modifier is present in an amount of from about 30 mg/ml to about 70 mg/ml;
(d) the pharmaceutically acceptable buffer is present in an amount of from about 5 mg/ml to about 10 mg/ml;
(e) the pharmaceutically acceptable antioxidant is present in an amount of from about 0.15 mg/ml to about 1.0 mg/ml; and
(f) the pharmaceutically acceptable metal chelator is present in an amount of from about 0.01 mg/ml to about 0.04 mg/ml;
wherein said pharmaceutical composition has a pH from about 6 to about 7.

18. The pharmaceutical composition according to claim 16, wherein
(a) the compound is 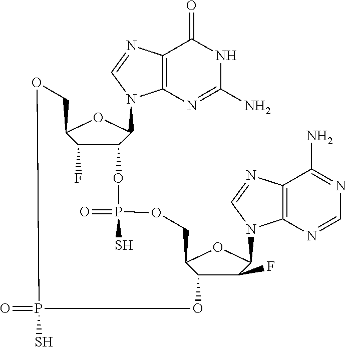
or a pharmaceutically acceptable salt thereof;
(b) the pharmaceutically acceptable aqueous carrier is water;
(c) the pharmaceutically acceptable tonicity modifier is mannitol, present in an amount of from about 30 mg/ml to about 70 mg/ml;
(d) the pharmaceutically acceptable buffer is histidine, present in an amount of from about 5 mg/ml to about 10 mg/ml;
(e) the pharmaceutically acceptable antioxidant is methionine, present in an amount of from about 0.15 mg/ml to about 1.0 mg/ml; and
(f) the pharmaceutically acceptable metal chelator is EDTA, present in an amount of from about 0.01 mg/ml to about 0.04 mg/ml;
wherein said pharmaceutical composition has a pH from about 6 to about 7.

19. The pharmaceutical composition according to claim 16, wherein
(a) the compound is present in a total amount of from about 0.25 mg/ml to about 6.0 mg/mL;
(b) the pharmaceutically acceptable aqueous carne is water;
(c) the pharmaceutically acceptable tonicity modifier is present in a total amount of from about 20 mg/ml to about 60 mg/ml;
(d) the pharmaceutically acceptable buffer is present in a total amount of from about 6 mg/ml to about 8 mg/ml;
(e) the pharmaceutically acceptable antioxidant is present in a total amount of from about 0.15 mg/ml to about 1.0 mg/ml; and
(f) the pharmaceutically acceptable metal chelator is present in a total amount of from about 0.01 mg/ml to about 0.04 mg/ml;
wherein said pharmaceutical composition has a pH from about 6 to about 7.

20. The pharmaceutical composition according to claim 16, wherein
(a) the compound is present in a total amount of from about 0.1 mg/ml to about 4.0 mg/mL;
(b) the pharmaceutically acceptable aqueous carrier is water;
(c) the pharmaceutically acceptable tonicity modifier is present in a total amount of from about 30 mg/ml to about 50 mg/ml;
(d) the pharmaceutically acceptable buffer is present in a total amount of from about 6 mg/ml to about 8 mg/ml;
(e) the pharmaceutically acceptable antioxidant is present in a total amount of from about 0.15 mg/ml to about 1.0 mg/ml; and
(f) the pharmaceutically acceptable metal chelator is present in a total amount of from about 0.01 mg/ml to about 0.03 mg/ml;
wherein said pharmaceutical composition has a pH from about 6 to about 7.

21. The pharmaceutical composition according to claim 16, comprising 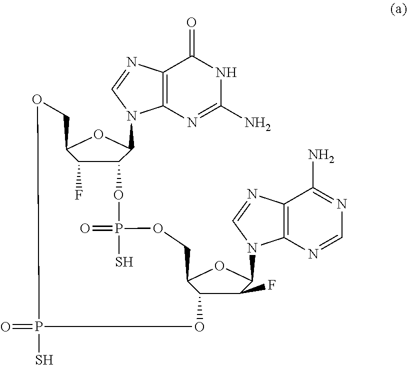
in an amount of from about 0.25 mg/ml to about 6.0 mg/mL;
(b) water;
(c) mannitol in an amount of from about 20 to about 60 mg/mL;
(d) histidine in an amount of about 5 mg/ml to about 10 mg/ml;
(e) methionine in an amount of from about 0.5 mg/ml to about 1.0 mg/ml; and
(f) EDTA in an amount of about 0.01 mg/ml to about 0.04 mg/ml;
wherein said pharmaceutical composition has a pH about 6.5.

22. The pharmaceutical composition according to claim 16, wherein 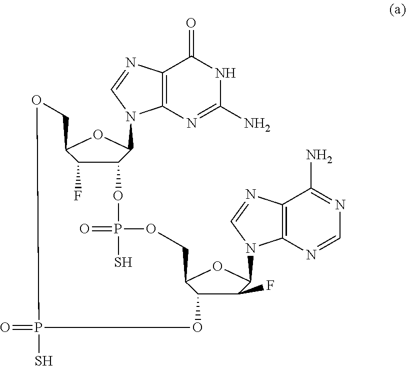
in an amount of from about 0.25 mg/ml to about 6.0 mg/mL;
(b) water;
(c) mannitol in an amount of from about 30 to about 40 mg/mL;
(d) histidine in an amount of about 6 mg/ml to about 8 mg/ml;
(e) methionine in an amount of from about 0.15 mg/ml to about 1.0 mg/ml; and
(f) EDTA in an amount of about 0.01 mg/ml to about 0.04 mg/ml;
wherein said pharmaceutical composition has a pH about 6.5.

23. The pharmaceutical composition according to claim 16, comprising 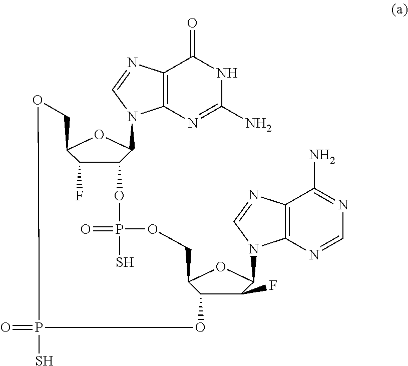
in an amount of from about 0.25 mg/ml to about 6.0 mg/mL;
(b) water;
(c) mannitol in an amount of about 34 mg/mL;
(d) histidine in an amount of about 7.75 mg/ml;
(e) methionine in an amount of from about 0.750 mg/ml; and
(f) EDTA in an amount of about 0.0175 mg/ml;
wherein said pharmaceutical composition has a pH about 6.5.

24. The pharmaceutical composition according to claim 16, comprising 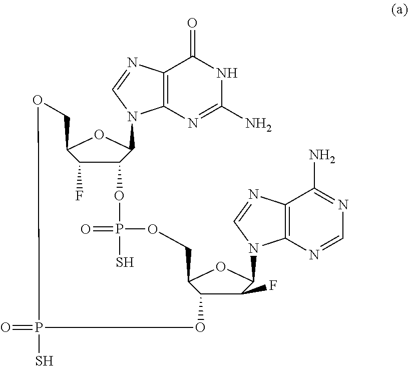
in an amount of from about 0.25 mg/ml to about 6.0 mg/mL;
(b) water;
(c) mannitol in an amount of about 40 mg/mL;
(d) histidine in an amount of about 7.75 mg/ml;
(e) methionine in an amount of from about 0.373 mg/ml; and
(f) EDTA in an amount of about 0.0175 mg/ml;
wherein said pharmaceutical composition has a pH about 6.5.

25. The pharmaceutical composition according to claim 16, comprising 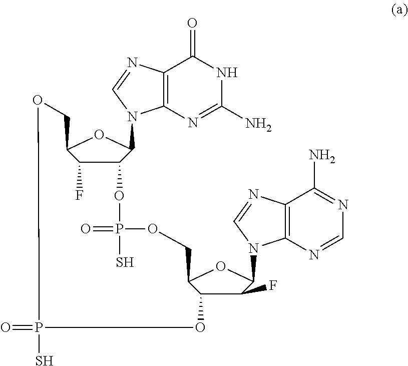
in an amount of from 0.25 mg/ml to about 6.0 mg/mL;
(b) water;
(c) mannitol in an amount of about 34 mg/mL;
(d) L-histidine in an amount of about 7.75 mg/ml;
(e) L-methionine in an amount of from about 0.750 mg/ml; and
(f) EDTA in an amount of about 0.0175 mg/ml; and
wherein said pharmaceutical composition has a pH about 6.5.

26. The pharmaceutical composition according to claim 16, comprising 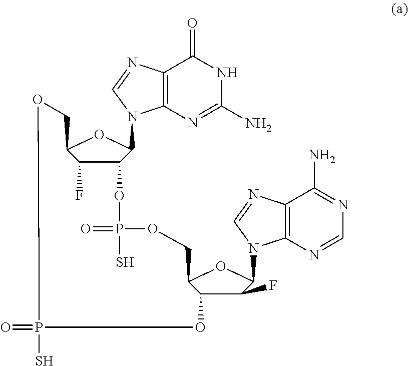
in an amount of about 0.54 mg/mL;
(b) water;
(c) mannitol in an amount of about 40 mg/mL;
(d) L-histidine in an amount of about 7.5 mg/ml;
(e) L-methionine in an amount of from about 0.373 mg/ml; and
(f) EDTA in an amount of about 0.0175 mg/ml;
wherein said pharmaceutical composition has a pH about 6.5.

27. A method of inducing an immune response in a subject, said method comprising administering a therapeutically effective amount of a pharmaceutical composition according to claim 1 to the subject.

28. A method of inducing a STING-dependent type I interferon production in a subject, said method comprising administering a therapeutically effective amount of a pharmaceutical composition according to claim 1 to the subject.

29. A method of treating a cell proliferation disorder in a subject, said method comprising administering a therapeutically effective amount of a pharmaceutical composition according to claim 1 to the subject.

30. The method of claim 29, wherein the cell proliferation disorder is cancer.

31. The method of claim 30, wherein the cancer is melanoma, non-small cell lung cancer, head and neck cancer, urothelial cancer, breast cancer, gastric cancer, gastroesophageal junction adenocarcinoma, multiple myeloma, hepatocellular cancer, renal cancer, mesothelioma, ovarian cancer, small cell lung cancer, esophageal cancer, anal cancer, biliary tract cancer, colorectal cancer, cervical cancer, thyroid cancer, salivary cancer, prostate cancer, or glioblastoma.

32. The method of claim 31, wherein the cancer is triple negative breast cancer or ER+/HER2− breast cancer.

33. The method of claim 31, wherein the cancer is a microsatellite instability-high (MSI-H) or mismatch repair deficient solid tumor.

34. The method of claim 31, wherein the cancer is non-small cell lung cancer, melanoma, urothelial cancer, head and neck cancer, gastric cancer, or MSI-H cancer.

35. The method of claim 27 wherein the pharmaceutical composition is administered by intratumoral administration.

36. The method of claim 27, wherein the pharmaceutical composition is administered by subcutaneous administration.
